SEQUENCE LISTING (1451 sequences)

SEQ ID NO: 1 - DNA, 50 bp, Artificial Sequence
  Synthetic primer
ttgtagattggagaacaccatgtaagagctactaagctcaccaccttccc

SEQ ID NO: 2 - DNA, 50 bp, Artificial Sequence
  Synthetic primer
agaatgagagcgacacagacgttaggcatttcctgctgaacgtttccccg

SEQ ID NO: 3 - DNA, 50 bp, Artificial Sequence
  Synthetic primer
gctggcggggaaccctgggagtagctagtttgctttttgcgtacacagag

SEQ ID NO: 4 - DNA, 50 bp, Artificial Sequence
  Synthetic primer
ccaccaacctgtatatcaagcctccttgccccacaaagcttccaaagccc

SEQ ID NO: 5 - DNA, 50 bp, Artificial Sequence
  Synthetic primer
acttcatcttccccaagtgcggggagtacaaggcatggcgtagagggtgc

SEQ ID NO: 6 - DNA, 50 bp, Artificial Sequence
  Synthetic primer
cactggagtgatgttgctgaccagccgtttcctgtacctctctaagttgg

SEQ ID NO: 7 - DNA, 50 bp, Artificial Sequence
  Synthetic primer
gtcctcaagttggcctccccaggcccagcaacggcctctcggcggccttt

SEQ ID NO: 8 - DNA, 50 bp, Artificial Sequence
  Synthetic primer
ggagttcaagtggaggaatgctggctttgagccctctacactgctggttg

SEQ ID NO: 9 - DNA, 50 bp, Artificial Sequence
  Synthetic primer
tgtcccagccagacttacagaattggggtctgtatcttaacaagaacccc

SEQ ID NO: 10 - DNA, 50 bp, Artificial Sequence
  Synthetic primer
cggaggcctgtgttctgtccactttgccgtttaatacccgatgagaatcc

SEQ ID NO: 11 - DNA, 50 bp, Artificial Sequence
  Synthetic primer
ttgagccacgcatagtgtcacgcacctgtgatcccagctacttaggaggt

SEQ ID NO: 12 - DNA, 50 bp, Artificial Sequence
  Synthetic primer
tccaacgcatccctgctgatccagaatgtcacccggaaggatgcaggaac

SEQ ID NO: 13 - DNA, 50 bp, Artificial Sequence
  Synthetic primer
aggcagtttccggaaacagtgaccctgagaaggcttcctcctgagtatgc

SEQ ID NO: 14 - DNA, 50 bp, Artificial Sequence
  Synthetic primer
cagttctgcatctgataccgtctcctttccctgaagtctggcacaccatg

SEQ ID NO: 15 - DNA, 50 bp, Artificial Sequence
  Synthetic primer
gcatctgcgtatcgtgggataattgacatgagggcttgagagaactccag

SEQ ID NO: 16 - DNA, 50 bp, Artificial Sequence
  Synthetic primer
tgcatccatacgatggcaaagtcatccccaggaacacagaggggtgcctg

SEQ ID NO: 17 - DNA, 50 bp, Artificial Sequence
  Synthetic primer
agcgctattccctggagactagggttgtcctgaatggccgagaggaaacc

SEQ ID NO: 18 - DNA, 50 bp, Artificial Sequence
  Synthetic primer
gcctctgcagacccaagtcgtcctgaagtcggcctctccaggcccagctc

SEQ ID NO: 19 - DNA, 50 bp, Artificial Sequence
  Synthetic primer
ctcaatcctgaacatctaggctggaacctgcacacctccccctcagctcc

SEQ ID NO: 20 - DNA, 50 bp, Artificial Sequence
  Synthetic primer
cagtggcctctttaggccaagctcatgcttcacaagggcctttccaggct

SEQ ID NO: 21 - DNA, 50 bp, Artificial Sequence
  Synthetic primer
gggagtcacttgatgctttcaggttaatcagagctatgggtgctacaggc

SEQ ID NO: 22 - DNA, 50 bp, Artificial Sequence
  Synthetic primer
tcttactctgcagcaacatggaggagagttttgtgtagtgagtgtgggcg

SEQ ID NO: 23 - DNA, 50 bp, Artificial Sequence
  Synthetic primer
gaagttctaagacattgatgtggccaaggtcaacaccgtgatttggcctg

SEQ ID NO: 24 - DNA, 50 bp, Artificial Sequence
  Synthetic primer
gtctttttgccagaagttaaaggctgtctccaagtccctgaactcagcag

SEQ ID NO: 25 - DNA, 50 bp, Artificial Sequence
  Synthetic primer
cttctccctccaccacccccagtcgtcagctccttccctcatttattttt

SEQ ID NO: 26 - DNA, 50 bp, Artificial Sequence
  Synthetic primer
tgctctccatcctggatcgtaaggaggcatcatcaggctgtgttcctgga

SEQ ID NO: 27 - DNA, 50 bp, Artificial Sequence
  Synthetic primer
gaatctgccgcccctccatcttctacctctgaatggccacccttagaccc

SEQ ID NO: 28 - DNA, 50 bp, Artificial Sequence
  Synthetic primer
aggccatttaataaaaagggcacaaagcctgtcagagttttcaacggtgc

SEQ ID NO: 29 - DNA, 50 bp, Artificial Sequence
  Synthetic primer
tcacactcctgggctctgaacacacacgccagctcctctctgaagcgact

SEQ ID NO: 30 - DNA, 50 bp, Artificial Sequence
  Synthetic primer
ttgagatgttcctgacggccaaagaggtggaggagtccctggagaggcgt

SEQ ID NO: 31 - DNA, 50 bp, Artificial Sequence
  Synthetic primer
cttccctcttggctgcccccaggtatttactgtggagaacattgcatagg

SEQ ID NO: 32 - DNA, 50 bp, Artificial Sequence
  Synthetic primer
gcatcaaccctgtgctctatgcagccatgaaccgccaattccgccaagca

SEQ ID NO: 33 - DNA, 50 bp, Artificial Sequence
  Synthetic primer
gctgcttttaggttaaccacaaaggaacaactcaggatcagtcgtgattg

SEQ ID NO: 34 - DNA, 50 bp, Artificial Sequence
  Synthetic primer
ctcgttagcaactgcgagcagaagctttccagtcacacatcaaggcgctt

SEQ ID NO: 35 - DNA, 50 bp, Artificial Sequence
  Synthetic primer
cctctgctcttggaagcccgtgcagaccccacaataaagaataagcatgg

SEQ ID NO: 36 - DNA, 50 bp, Artificial Sequence
  Synthetic primer
cccgtggacaccccagacctgcgaaggatgatcgcccgataaagacggat

SEQ ID NO: 37 - DNA, 50 bp, Artificial Sequence
  Synthetic primer
cctcgggcagcagagagcagatgaaacccccatgtggtaggcagggttgg

SEQ ID NO: 38 - DNA, 50 bp, Artificial Sequence
  Synthetic primer
aagctctcctggaccttggactcacagctgacccccagtgggcagttcca

SEQ ID NO: 39 - DNA, 50 bp, Artificial Sequence
  Synthetic primer
cccagcccctgaatgaggaaggatcagagaagaggctactgggggagaat

SEQ ID NO: 40 - DNA, 50 bp, Artificial Sequence
  Synthetic primer
agagtcctagtgtgtgccctaccctggctcaggcttctgggctctgagaa

SEQ ID NO: 41 - DNA, 50 bp, Artificial Sequence
  Synthetic primer
actgtgtttgatggcctctgctgtgttttaacatcgtgcttcttatatgg

SEQ ID NO: 42 - DNA, 50 bp, Artificial Sequence
  Synthetic primer
ggagcacctccaggcttgcaaagaaagtgaggcctcttggtatcctttcc

SEQ ID NO: 43 - DNA, 50 bp, Artificial Sequence
  Synthetic primer
aactgttggaggagaagctgaaggaggctgagacccgagcagagtttgcc

SEQ ID NO: 44 - DNA, 50 bp, Artificial Sequence
  Synthetic primer
tcaccagaccttggaccagaccctgctggaactcaacaacctgtgagggc

SEQ ID NO: 45 - DNA, 50 bp, Artificial Sequence
  Synthetic primer
ccatccacccattctcagcagactccagtattggcacagtcactcactgc

SEQ ID NO: 46 - DNA, 50 bp, Artificial Sequence
  Synthetic primer
tttgcaaagggccaaatttccccaaactgaacgggctcaggaaatgttcc

SEQ ID NO: 47 - DNA, 50 bp, Artificial Sequence
  Synthetic primer
ggatggagaatcctggctgccatgtggttgatgccagcccctccagagaa

SEQ ID NO: 48 - DNA, 50 bp, Artificial Sequence
  Synthetic primer
tccctggagtacgggaaggctgagctggagattcagaaagacgccctgga

SEQ ID NO: 49 - DNA, 50 bp, Artificial Sequence
  Synthetic primer
catcctgattgtgctgctggtcgtctttctccctcagagcagtgacagca

SEQ ID NO: 50 - DNA, 50 bp, Artificial Sequence
  Synthetic primer
ccactcctggggggaatctggtcaccctgagctgtgaaacaaagttgctc

SEQ ID NO: 51 - DNA, 50 bp, Artificial Sequence
  Synthetic primer
tccagcggtctcggcactcccctcacacctcccaagatgaagctcaatga

SEQ ID NO: 52 - DNA, 50 bp, Artificial Sequence
  Synthetic primer
gcctccagtgccttcccccgtggaataaacggtgtgtcctgagaaaccac

SEQ ID NO: 53 - DNA, 50 bp, Artificial Sequence
  Synthetic primer
tggggccttcacctgctccatccagaacatcagcttctcctccttcactc

SEQ ID NO: 54 - DNA, 50 bp, Artificial Sequence
  Synthetic primer
agctgctgcaagacagcaatgacagtccacctgccggcctgattcctgca

SEQ ID NO: 55 - DNA, 50 bp, Artificial Sequence
  Synthetic primer
gcaacagcgggttttgcagacgctcttctccagccggagctgggactgtt

SEQ ID NO: 56 - DNA, 50 bp, Artificial Sequence
  Synthetic primer
cctgggtccaccagcccctgacgcccctgtgcccactttgtaaataaact

SEQ ID NO: 57 - DNA, 50 bp, Artificial Sequence
  Synthetic primer
acattatgctcctacctcccggcagcatctccaggcccagaactttctcc

SEQ ID NO: 58 - DNA, 50 bp, Artificial Sequence
  Synthetic primer
taagatcatgtcactgcactccagactgagcaacagagtgacactttata

SEQ ID NO: 59 - DNA, 50 bp, Artificial Sequence
  Synthetic primer
aagctttcagaatatgtcagtgctgatgtagcatgcttgttgcaattgcc

SEQ ID NO: 60 - DNA, 50 bp, Artificial Sequence
  Synthetic primer
ggagcacggatctaccacaccattgcatatttgacaccccttccccagcc

SEQ ID NO: 61 - DNA, 50 bp, Artificial Sequence
  Synthetic primer
acaactggccgggcatagtcagtgtcccagcaccatgtcagtatgctcac

SEQ ID NO: 62 - DNA, 50 bp, Artificial Sequence
  Synthetic primer
acgcagaccccacactccatggagtctggaatgcatgggagctgcccccc

SEQ ID NO: 63 - DNA, 50 bp, Artificial Sequence
  Synthetic primer
ttcttgacaactctgctcctttggggctggctactactgcaggtctccag

SEQ ID NO: 64 - DNA, 50 bp, Artificial Sequence
  Synthetic primer
acagctattcccatattctaggagtggcctaagaaatgcgtgtttcagtg

SEQ ID NO: 65 - DNA, 50 bp, Artificial Sequence
  Synthetic primer
ctcttccgcgacaattcatatcttgtacgtcatcagagatttcatgccgg

SEQ ID NO: 66 - DNA, 50 bp, Artificial Sequence
  Synthetic primer
tgcatcatgtgctcctaccctgctctaccgcttttctgggtcacagaggc

SEQ ID NO: 67 - DNA, 50 bp, Artificial Sequence
  Synthetic primer
gagcgtgatgattgggtgttcatacgcttgtgtgagatgtgccacccttg

SEQ ID NO: 68 - DNA, 50 bp, Artificial Sequence
  Synthetic primer
ctgtccgtctcgaatgactggagtttcctgcttctgtcactacacctccc

SEQ ID NO: 69 - DNA, 50 bp, Artificial Sequence
  Synthetic primer
ggggtaacacagagtgcccttatgaaggagttggagatcctgcaaggaag

SEQ ID NO: 70 - DNA, 50 bp, Artificial Sequence
  Synthetic primer
gcaggaacaacagatgcaggaacaggctgcacagctcagcacaacattcc

SEQ ID NO: 71 - DNA, 50 bp, Artificial Sequence
  Synthetic primer
gtccaggattgcctcacttgagacttgctaggcctctgctgtgtgctggg

SEQ ID NO: 72 - DNA, 50 bp, Artificial Sequence
  Synthetic primer
gtaactggaaactgtgttgctctaaccctcctccagccctgcagcctccc

SEQ ID NO: 73 - DNA, 50 bp, Artificial Sequence
  Synthetic primer
ggaacacctcctcaaacctaccactcaggaatgtttgctggggccgaaag

SEQ ID NO: 74 - DNA, 50 bp, Artificial Sequence
  Synthetic primer
ctgagccaggatgtgcaatagaagcaggggttgtcccctctgccagatct

SEQ ID NO: 75 - DNA, 50 bp, Artificial Sequence
  Synthetic primer
aggaagccaggcaccgatagagcacccagccccacccctgtaaatggaat

SEQ ID NO: 76 - DNA, 50 bp, Artificial Sequence
  Synthetic primer
tactgaaagacttttgcctaaagtggcattattgactgctggtgtgatga

SEQ ID NO: 77 - DNA, 50 bp, Artificial Sequence
  Synthetic primer
gaggctctcttgtgagctcttggaccctcctcttcacggacccaactgtg

SEQ ID NO: 78 - DNA, 50 bp, Artificial Sequence
  Synthetic primer
agcggcgcccgggttccggcacagacggcctgcggctccagggggcggag

SEQ ID NO: 79 - DNA, 50 bp, Artificial Sequence
  Synthetic primer
cccataaaacagggtgtgaaaggcatctcagcggctgccccaccatggct

SEQ ID NO: 80 - DNA, 50 bp, Artificial Sequence
  Synthetic primer
cgcacacagatgggatttgggaataggtttggttatccaaggagcagtgc

SEQ ID NO: 81 - DNA, 50 bp, Artificial Sequence
  Synthetic primer
agggcctgttagccacctcatgcctttccctggacccctactgggctcac

SEQ ID NO: 82 - DNA, 50 bp, Artificial Sequence
  Synthetic primer
atgccagacataacatgtagcagccatacttgcatggaaactgactacac

SEQ ID NO: 83 - DNA, 50 bp, Artificial Sequence
  Synthetic primer
gaatacttctcttgctgagagccgatgcccgtccccgggccagcagggat

SEQ ID NO: 84 - DNA, 50 bp, Artificial Sequence
  Synthetic primer
gtcgacctcaccaggcccagctcatgcttctttgcagcctctccaggccc

SEQ ID NO: 85 - DNA, 50 bp, Artificial Sequence
  Synthetic primer
caaggccctcttcacactccatcttgtagccccagcaggagctattttct

SEQ ID NO: 86 - DNA, 50 bp, Artificial Sequence
  Synthetic primer
gggaagatgccctatctctcgggtgctgcctacaacgtggctgtcatctc

SEQ ID NO: 87 - DNA, 50 bp, Artificial Sequence
  Synthetic primer
aaacccgtcacccagatcgtcagcgccgaggcctggggtagagcaggtga

SEQ ID NO: 88 - DNA, 50 bp, Artificial Sequence
  Synthetic primer
ccacatgatcccactgcagatgtttttgtaacaccagctgaggagaaacc

SEQ ID NO: 89 - DNA, 50 bp, Artificial Sequence
  Synthetic primer
gccatgctgagagctgggctttcctctgtgaccatcccggcctgtaacat

SEQ ID NO: 90 - DNA, 50 bp, Artificial Sequence
  Synthetic primer
gctgagagctgggctttcctctgtgaccatcccggcctgtaacatatctg

SEQ ID NO: 91 - DNA, 50 bp, Artificial Sequence
  Synthetic primer
gccgcgcggacccaacgagcccgcgctcagacgccccagctccgccgaga

SEQ ID NO: 92 - DNA, 50 bp, Artificial Sequence
  Synthetic primer
ttgctcacgtctgtgccatgttgtcaatgggtcctttccaacccaagagg

SEQ ID NO: 93 - DNA, 50 bp, Artificial Sequence
  Synthetic primer
tgttcttccccatgtcctggatgccactggaagtgcacactgcttgtatg

SEQ ID NO: 94 - DNA, 50 bp, Artificial Sequence
  Synthetic primer
acgctcgtctatgcctactggaccatgtgagcctggcacttccccacaac

SEQ ID NO: 95 - DNA, 50 bp, Artificial Sequence
  Synthetic primer
ccctggaaagctccccgacaacctccactgccattacccactaggcaagt

SEQ ID NO: 96 - DNA, 50 bp, Artificial Sequence
  Synthetic primer
gaggacgtcccggctgggatgaagtctggtggtgggtcgtaagtttagga

SEQ ID NO: 97 - DNA, 50 bp, Artificial Sequence
  Synthetic primer
ccagtgggctctggcgcctatggctctgtgtgtgctgcttttgacacaaa

SEQ ID NO: 98 - DNA, 50 bp, Artificial Sequence
  Synthetic primer
tccaaggatcagccctgagagcaggttggtgactttgaggagggcagtcc

SEQ ID NO: 99 - DNA, 50 bp, Artificial Sequence
  Synthetic primer
ctccggtgagccatggagcccctcttatctgggcccaacctcaaataacc

SEQ ID NO: 100 - DNA, 50 bp, Artificial Sequence
  Synthetic primer
aactgtgggaccccttcagattccctgaggtatggcttggtcactctcag

SEQ ID NO: 101 - DNA, 50 bp, Artificial Sequence
  Synthetic primer
ggtcaaatggggtcacacagaatactaagagctgttcacccagacctcac

SEQ ID NO: 102 - DNA, 50 bp, Artificial Sequence
  Synthetic primer
cttcagctctgctgttgggctggtgtgtggacagaaggaatggaaagcca

SEQ ID NO: 103 - DNA, 50 bp, Artificial Sequence
  Synthetic primer
ctccttgccagccccacccccaggtttttgcccatcctcccaatctcaat

SEQ ID NO: 104 - DNA, 50 bp, Artificial Sequence
  Synthetic primer
tgacctggatctgacctcacaccatcagcagggggcacccaccatgcaca

SEQ ID NO: 105 - DNA, 50 bp, Artificial Sequence
  Synthetic primer
tcaggagagaagaagaggaaacacaagaggaaacgcctggtgcagagccc

SEQ ID NO: 106 - DNA, 50 bp, Artificial Sequence
  Synthetic primer
gcatggaggcaggtgtttccaagggtgtctcattaactgtagctgcaaag

SEQ ID NO: 107 - DNA, 50 bp, Artificial Sequence
  Synthetic primer
tgggtgcaacctctgcggcagccgactgcatgaaccaaagttctcaccta

SEQ ID NO: 108 - DNA, 50 bp, Artificial Sequence
  Synthetic primer
ctccaacaccgtccagttgtggcagctctccagaagtaatagcagctgac

SEQ ID NO: 109 - DNA, 50 bp, Artificial Sequence
  Synthetic primer
ttgccagagttttgcctgctgctttcctcgtggcctcttcttgggtagtg

SEQ ID NO: 110 - DNA, 50 bp, Artificial Sequence
  Synthetic primer
aagtttgtgggggccaaacctgggaccccgagcttcctcggtagcagagg

SEQ ID NO: 111 - DNA, 50 bp, Artificial Sequence
  Synthetic primer
ccatggtgatggatggtttggaaagggaatgttggtgccttttgtgccac

SEQ ID NO: 112 - DNA, 50 bp, Artificial Sequence
  Synthetic primer
acatggtgagcaggtatcctatgtcccgaggaagcccattccacctgagc

SEQ ID NO: 113 - DNA, 50 bp, Artificial Sequence
  Synthetic primer
gaaagcaggactcacagaacctgaagtcacccagactcccagccatcagg

SEQ ID NO: 114 - DNA, 50 bp, Artificial Sequence
  Synthetic primer
ccagccaagaaggcagccacactagcagagggtgacaaggacaatgacat

SEQ ID NO: 115 - DNA, 50 bp, Artificial Sequence
  Synthetic primer
gcacctgcagaggcaggcttcagagctccaccagccatcaatgccaggct

SEQ ID NO: 116 - DNA, 50 bp, Artificial Sequence
  Synthetic primer
gagggcgctgcatgtgctgggtacatttcttggcacttgggaatcagtag

SEQ ID NO: 117 - DNA, 50 bp, Artificial Sequence
  Synthetic primer
gccctgtgttcctggagcatttgttgaccgccaactgacaacatgctagg

SEQ ID NO: 118 - DNA, 50 bp, Artificial Sequence
  Synthetic primer
ccgcctcacaagtgcgaagtctggtaaggacgaagcggagagagccaaga

SEQ ID NO: 119 - DNA, 50 bp, Artificial Sequence
  Synthetic primer
tacatgactcagcatacctgctggtgcagagctgaagattttggagggtc

SEQ ID NO: 120 - DNA, 50 bp, Artificial Sequence
  Synthetic primer
cttgagagatgagcaccagttacacaaggacttctttatccgagcgctgg

SEQ ID NO: 121 - DNA, 50 bp, Artificial Sequence
  Synthetic primer
ccccaacttcgccctgcccacttgacttcaccaaatcccttcctggagac

SEQ ID NO: 122 - DNA, 50 bp, Artificial Sequence
  Synthetic primer
tcagactccctgccaccttttcccctgggttctgccgtcttgcctcactt

SEQ ID NO: 123 - DNA, 50 bp, Artificial Sequence
  Synthetic primer
gtatactctggaaggcagtccctggagccagtgccaggcggatgacagat

SEQ ID NO: 124 - DNA, 50 bp, Artificial Sequence
  Synthetic primer
gccaggcggatgacagatgggaccctcctctggccaaatgtacctctcgt

SEQ ID NO: 125 - DNA, 50 bp, Artificial Sequence
  Synthetic primer
ctgtgtacaaaccccccaaagtgtccctgatgccagcaacccttgcacgg

SEQ ID NO: 126 - DNA, 50 bp, Artificial Sequence
  Synthetic primer
aggctcgggggtccccgcgtcccaggcccagggggatgggggtcgcgaga

SEQ ID NO: 127 - DNA, 50 bp, Artificial Sequence
  Synthetic primer
tggccacaggagctgaaagcagaagagtgggatttgatgccaggcagtgg

SEQ ID NO: 128 - DNA, 50 bp, Artificial Sequence
  Synthetic primer
gcccacctcaggcagacacagagcacaatgctggggttctcttcacacta

SEQ ID NO: 129 - DNA, 50 bp, Artificial Sequence
  Synthetic primer
acatgagctcagagctaccccacaccttcggactgcctcggcccccacag

SEQ ID NO: 130 - DNA, 50 bp, Artificial Sequence
  Synthetic primer
gacgtggttcatgtgaaagatgccagtggcaacagatttgccactcgacc

SEQ ID NO: 131 - DNA, 50 bp, Artificial Sequence
  Synthetic primer
catgggctttggtctttttgactaaacctcttttataacatgttcaataa

SEQ ID NO: 132 - DNA, 50 bp, Artificial Sequence
  Synthetic primer
tgggaccagcagcacaagttccctgtcttcatggggcgagtatatgaccc

SEQ ID NO: 133 - DNA, 50 bp, Artificial Sequence
  Synthetic primer
cacaagatggcgctgaaagcaaagaaggaagctcctgcctctcctgaagc

SEQ ID NO: 134 - DNA, 50 bp, Artificial Sequence
  Synthetic primer
tctagggctagtacttagtttcacacccgggagctgggagaaaaaacctg

SEQ ID NO: 135 - DNA, 50 bp, Artificial Sequence
  Synthetic primer
gaattgggcagctcatgcttgagacccaatctccatgatgacctacaagc

SEQ ID NO: 136 - DNA, 50 bp, Artificial Sequence
  Synthetic primer
ggaaatatgagattacggagcagcgcaagattgatcagaaagctgtggac

SEQ ID NO: 137 - DNA, 50 bp, Artificial Sequence
  Synthetic primer
ccagtggaaataccgggaacccaaggaccggagcgaatgaggcctgtatc

SEQ ID NO: 138 - DNA, 50 bp, Artificial Sequence
  Synthetic primer
ctttctgcttgcattgccgtatctgtgcgttccttcatcctggtcctggc

SEQ ID NO: 139 - DNA, 50 bp, Artificial Sequence
  Synthetic primer
tgcagagaagaaagtgaggccgggagcctgagcctgggctggagccttct

SEQ ID NO: 140 - DNA, 50 bp, Artificial Sequence
  Synthetic primer
gccatgtcaccgagccccattgattcccagagggtcttagtcctggaaag

SEQ ID NO: 141 - DNA, 50 bp, Artificial Sequence
  Synthetic primer
agccgcaattgttctgaaaatgtcaaacgaggcttctgttttgcacctgc

SEQ ID NO: 142 - DNA, 50 bp, Artificial Sequence
  Synthetic primer
catgcctctgtgccttcgctcatgctgtttcttccgactggaatgccttc

SEQ ID NO: 143 - DNA, 50 bp, Artificial Sequence
  Synthetic primer
ggatcctgttgacaccccaaacccaacaaggaggaagcctgggaagtgcc

SEQ ID NO: 144 - DNA, 50 bp, Artificial Sequence
  Synthetic primer
tcatgagatctggttttttgaaagtgtgtggcaagtcccccttcgctctc

SEQ ID NO: 145 - DNA, 50 bp, Artificial Sequence
  Synthetic primer
ggcccctctgtgaaaacatgcccagcggcaaggccaacaagctgggggat

SEQ ID NO: 146 - DNA, 50 bp, Artificial Sequence
  Synthetic primer
gctgcaggctaacagggagcccgacttcagcagcctggtgtcacctctca

SEQ ID NO: 147 - DNA, 50 bp, Artificial Sequence
  Synthetic primer
gcaagaacacttctggaagggagagtggatttggctgggcctctggatgg

SEQ ID NO: 148 - DNA, 50 bp, Artificial Sequence
  Synthetic primer
ctacaggtcccctctgagccctctcaccttgtcctgtggaagaagcacag

SEQ ID NO: 149 - DNA, 50 bp, Artificial Sequence
  Synthetic primer
gccccgtctcgtgggattattgattcctgatccaagaagggtcctctggg

SEQ ID NO: 150 - DNA, 50 bp, Artificial Sequence
  Synthetic primer
ccaggcccaagtcgtcctcaagtcggcctggaagtgggcctggaagagct

SEQ ID NO: 151 - DNA, 50 bp, Artificial Sequence
  Synthetic primer
ggtgagagagaaggaaaccttgggaggaggacattactggttgttctggc

SEQ ID NO: 152 - DNA, 50 bp, Artificial Sequence
  Synthetic primer
tggatggcaccagaggctgcagaaggccaagaatcaagctagaaggccac

SEQ ID NO: 153 - DNA, 50 bp, Artificial Sequence
  Synthetic primer
gctgacgtatttcatggcagtcaagtccaatggcagcgtcttcgtccggg

SEQ ID NO: 154 - DNA, 50 bp, Artificial Sequence
  Synthetic primer
ccccgtttatccatgtgtccattgacggccatctatgttgcttcttcggc

SEQ ID NO: 155 - DNA, 50 bp, Artificial Sequence
  Synthetic primer
cgggaggcacggccgagatgtacacgaagacaggagtcaatggagattct

SEQ ID NO: 156 - DNA, 50 bp, Artificial Sequence
  Synthetic primer
gcggaagaggagccgctggaaccaagacacaatggaacagaagacagtga

SEQ ID NO: 157 - DNA, 50 bp, Artificial Sequence
  Synthetic primer
cctgagtaactggctgcttcaggatcagcttgcagagtcttgcttttagg

SEQ ID NO: 158 - DNA, 50 bp, Artificial Sequence
  Synthetic primer
gtgggtctgaagagcttggttgcagaaacttcggggtctacaaaggcagg

SEQ ID NO: 159 - DNA, 50 bp, Artificial Sequence
  Synthetic primer
gtttggacctgggcgtctgagggccccactttgggaaccgttgaaatagg

SEQ ID NO: 160 - DNA, 50 bp, Artificial Sequence
  Synthetic primer
gatctgtcactttctcccatcacgctcaggtaggaccatccagttgcaga

SEQ ID NO: 161 - DNA, 50 bp, Artificial Sequence
  Synthetic primer
cccccaacaaagactgtgcattcaataccttaatggaactcgaggtggag

SEQ ID NO: 162 - DNA, 50 bp, Artificial Sequence
  Synthetic primer
agtgtcctcgacatcccaggggaaagcaagagcagtgagcctgagcagtg

SEQ ID NO: 163 - DNA, 50 bp, Artificial Sequence
  Synthetic primer
agcaatcacaaagccagagaagctgtaagctgcctgccgggcctgaggag

SEQ ID NO: 164 - DNA, 50 bp, Artificial Sequence
  Synthetic primer
gagctggcttcaggggaagtgctattcacaggaccatatccaccaccctc

SEQ ID NO: 165 - DNA, 50 bp, Artificial Sequence
  Synthetic primer
ggagggacaagaatcaatggataagcgtgggtggaggaagatccaaacag

SEQ ID NO: 166 - DNA, 50 bp, Artificial Sequence
  Synthetic primer
ctaggctatcggtgctgcttctgcccactttcaggagaaccctgctctgc

SEQ ID NO: 167 - DNA, 50 bp, Artificial Sequence
  Synthetic primer
cagttttgcctccagtggaagcagaaagggttttttcagctgttaaatcc

SEQ ID NO: 168 - DNA, 50 bp, Artificial Sequence
  Synthetic primer
aggccaaggaccgcagtccttcagtaacaccagtgtaaaagcttgaggag

SEQ ID NO: 169 - DNA, 50 bp, Artificial Sequence
  Synthetic primer
gcctccaagaaatggggctgactttgccatcatggagatgaagaagctgc

SEQ ID NO: 170 - DNA, 50 bp, Artificial Sequence
  Synthetic primer
tcattcgacgtggttcgtgtgaaagatgccaatggcaacagctttgccgc

SEQ ID NO: 171 - DNA, 50 bp, Artificial Sequence
  Synthetic primer
ttcctgggagaccatcccccacctttctccggcctctgagactttgagtc

SEQ ID NO: 172 - DNA, 50 bp, Artificial Sequence
  Synthetic primer
actggcaaaggctaggcccggagcaccctaggcgctggattttgggacaa

SEQ ID NO: 173 - DNA, 50 bp, Artificial Sequence
  Synthetic primer
ctgggccagaatttcaaacggcctcactaggcttctggttgatgcctgtg

SEQ ID NO: 174 - DNA, 50 bp, Artificial Sequence
  Synthetic primer
cctccagtggtttaggcaggaccctgggaaaggtctcacatctctgttgc

SEQ ID NO: 175 - DNA, 50 bp, Artificial Sequence
  Synthetic primer
ggccattgtagccctctgtgtgttcacagccatcctgtactgcattccgc

SEQ ID NO: 176 - DNA, 50 bp, Artificial Sequence
  Synthetic primer
gttaaatgcggtgtagcaaagttatggggtctgcttgagggcactaacct

SEQ ID NO: 177 - DNA, 50 bp, Artificial Sequence
  Synthetic primer
atgagtgttataccctaagagtctgaggagtgtgggaagtgcttcgtgtg

SEQ ID NO: 178 - DNA, 50 bp, Artificial Sequence
  Synthetic primer
gagccgccaggaaccctcctcctgtcaatgggggtgtagtatttttgcca

SEQ ID NO: 179 - DNA, 50 bp, Artificial Sequence
  Synthetic primer
ccccttgagaatggtgatccacccagttacaggggcatttagggagcaga

SEQ ID NO: 180 - DNA, 50 bp, Artificial Sequence
  Synthetic primer
ttgccagggcgaggcggccccaatgacagatttccctttagacccagcag

SEQ ID NO: 181 - DNA, 50 bp, Artificial Sequence
  Synthetic primer
aggcgacgggaagcgcgggtggtcggctggggtccggctcctggagaaca

SEQ ID NO: 182 - DNA, 50 bp, Artificial Sequence
  Synthetic primer
gcatcctcctgtgtatggaagagacaggtgaccgctccaggttgggtgct

SEQ ID NO: 183 - DNA, 50 bp, Artificial Sequence
  Synthetic primer
gagtgtatagccccatcttgtggtaacttgctgcttctgcacttcatatc

SEQ ID NO: 184 - DNA, 50 bp, Artificial Sequence
  Synthetic primer
agcagctcacccaggagctagacaccctccgcaacctcttccgccagatt

SEQ ID NO: 185 - DNA, 50 bp, Artificial Sequence
  Synthetic primer
cgtgttctctttaagaacactcgtgaaactgcccaggccatcaagggtat

SEQ ID NO: 186 - DNA, 50 bp, Artificial Sequence
  Synthetic primer
ggaacacaggcaaacccgtgattttggtgctccttgtaactcagccctgc

SEQ ID NO: 187 - DNA, 50 bp, Artificial Sequence
  Synthetic primer
gggaatgagtacatggtccttgcctccccatcagtgcctcttctccaact

SEQ ID NO: 188 - DNA, 50 bp, Artificial Sequence
  Synthetic primer
ccaggccgacatgactaagtatctggaaggctcactgtaccccagcaccc

SEQ ID NO: 189 - DNA, 50 bp, Artificial Sequence
  Synthetic primer
ggtggacgactgtgttacagccttggctgcgctagtagctgcctttcatg

SEQ ID NO: 190 - DNA, 50 bp, Artificial Sequence
  Synthetic primer
gtcatccagcaatgagagaatcctgcctctgtagaccaacatccagtgtg

SEQ ID NO: 191 - DNA, 50 bp, Artificial Sequence
  Synthetic primer
gaggctggatgaaatgtcagctagggccattttggctgctgaggctctgg

SEQ ID NO: 192 - DNA, 50 bp, Artificial Sequence
  Synthetic primer
cttgccgatgctcccagctgaataaagccctgccttctacaactcagtgt

SEQ ID NO: 193 - DNA, 50 bp, Artificial Sequence
  Synthetic primer
gcaccatgcatggagtcagccatttctctaggaaccttgattcctgtctg

SEQ ID NO: 194 - DNA, 50 bp, Artificial Sequence
  Synthetic primer
gcaagaaagtctaacctattccggtgttctctctcccatgagacaagccg

SEQ ID NO: 195 - DNA, 50 bp, Artificial Sequence
  Synthetic primer
tgatccgaaggaggagtggcgctgggcgctggactcgctggtgtgaaaat

SEQ ID NO: 196 - DNA, 50 bp, Artificial Sequence
  Synthetic primer
ccccacgcctgtttgtattgggagctctggaccaatagtgtctctcctag

SEQ ID NO: 197 - DNA, 50 bp, Artificial Sequence
  Synthetic primer
tgacgagccctctcacagagcacggtctgaatctgcacagagcaagatgc

SEQ ID NO: 198 - DNA, 50 bp, Artificial Sequence
  Synthetic primer
atgatacgtaaacgctcctttgggaacccattcgagggcagccggcggga

SEQ ID NO: 199 - DNA, 50 bp, Artificial Sequence
  Synthetic primer
ccaaaaaattgtctctggcaatagttaccttcccagatacaggtcccccc

SEQ ID NO: 200 - DNA, 50 bp, Artificial Sequence
  Synthetic primer
tcaagcctagtgtaaatttctgcatctcacacgactttagtttggccagg

SEQ ID NO: 201 - DNA, 50 bp, Artificial Sequence
  Synthetic primer
ctatatctggaaggggcgaaaagcgaatgagaaggagcggcaggcagccc

SEQ ID NO: 202 - DNA, 50 bp, Artificial Sequence
  Synthetic primer
aatttggccgcggttctcgctcttgtcgcgtctgctcaaaccagcacggt

SEQ ID NO: 203 - DNA, 50 bp, Artificial Sequence
  Synthetic primer
ccactgcctcaagatgcccaagatctcctgccaaaacaagggagtcgtgg

SEQ ID NO: 204 - DNA, 50 bp, Artificial Sequence
  Synthetic primer
tggctcccaactcctccctatcctaaaggcccactggcattaaagtgctg

SEQ ID NO: 205 - DNA, 50 bp, Artificial Sequence
  Synthetic primer
tctgtgtggtgttttgtaccggcacgggatatggaacgaaaactgctttg

SEQ ID NO: 206 - DNA, 50 bp, Artificial Sequence
  Synthetic primer
cagagtggagaggcagaaaccatgtgcagaggctgggagatgctgctgtt

SEQ ID NO: 207 - DNA, 50 bp, Artificial Sequence
  Synthetic primer
gagggggagggcttggtggggagggggacttacaattcatccacgctgtt

SEQ ID NO: 208 - DNA, 50 bp, Artificial Sequence
  Synthetic primer
gggcattgtgctggccccactttcactggccttcttggttttggggggaa

SEQ ID NO: 209 - DNA, 50 bp, Artificial Sequence
  Synthetic primer
ctgcccttcccaggtgattctgtaagttgtccctcaactgtacttggaga

SEQ ID NO: 210 - DNA, 50 bp, Artificial Sequence
  Synthetic primer
ggctttattttcactggcccctgaggttgaagtcagagtctccaaaaaac

SEQ ID NO: 211 - DNA, 50 bp, Artificial Sequence
  Synthetic primer
gtaatagccaaaccccactctgttggtagcaattggcagccctatttcag

SEQ ID NO: 212 - DNA, 50 bp, Artificial Sequence
  Synthetic primer
gtggcttctctgtgaattgcctgtaacacatagtggcttctccgcccttg

SEQ ID NO: 213 - DNA, 50 bp, Artificial Sequence
  Synthetic primer
ggtgagccatggagcccctcttatctgggcccaacctctccttaaataac

SEQ ID NO: 214 - DNA, 50 bp, Artificial Sequence
  Synthetic primer
ccaaggttctcacactggcctgggcttgggtgcccatataggaggtctgt

SEQ ID NO: 215 - DNA, 50 bp, Artificial Sequence
  Synthetic primer
gcctcggaactgtgacacccccatcttcgacttcagagtcttcttccaag

SEQ ID NO: 216 - DNA, 50 bp, Artificial Sequence
  Synthetic primer
tgacgacatcgagtgcttccttatggagctggagcagcccgcctagaacc

SEQ ID NO: 217 - DNA, 50 bp, Artificial Sequence
  Synthetic primer
atgcatgtcactaagttgtcatcccacataaattgatgtgcagcataggg

SEQ ID NO: 218 - DNA, 50 bp, Artificial Sequence
  Synthetic primer
ggggctccacacctttgctgtgtgttctggggcaacctactaatcctctc

SEQ ID NO: 219 - DNA, 50 bp, Artificial Sequence
  Synthetic primer
gaaaagctgtgtcgtgttccctgtgaaactgagcaggtgtgtgttggcgc

SEQ ID NO: 220 - DNA, 50 bp, Artificial Sequence
  Synthetic primer
tctcaaaacccatggagggaggctgctggtgtggtaggcagaacctaggc

SEQ ID NO: 221 - DNA, 50 bp, Artificial Sequence
  Synthetic primer
acttgagctgaaaacccagatggtgttaactggccgccccactttccggc

SEQ ID NO: 222 - DNA, 50 bp, Artificial Sequence
  Synthetic primer
accaggtgcagacacgcagtgctgatgagcccatgactacctttgtctta

SEQ ID NO: 223 - DNA, 50 bp, Artificial Sequence
  Synthetic primer
agtgaggctgagatgacagaggtggtcatggctggcacagggctcaggta

SEQ ID NO: 224 - DNA, 50 bp, Artificial Sequence
  Synthetic primer
ccagggacttcgggggaaatctggagcattttttacaagccttccacttc

SEQ ID NO: 225 - DNA, 50 bp, Artificial Sequence
  Synthetic primer
ggcagccctggcaaatgaatcaaagacccattcctgttcctctccccacc

SEQ ID NO: 226 - DNA, 50 bp, Artificial Sequence
  Synthetic primer
gcattggtctggccaagtctacaatgtcccaatatcaaggacaaccaccc

SEQ ID NO: 227 - DNA, 50 bp, Artificial Sequence
  Synthetic primer
gttctccagaggaaggtggaagaaaccatgggcaggagtaggaattgagt

SEQ ID NO: 228 - DNA, 50 bp, Artificial Sequence
  Synthetic primer
gatagtcccttgaacacacatgctgccgagctctggaaaaaccccacagc

SEQ ID NO: 229 - DNA, 50 bp, Artificial Sequence
  Synthetic primer
ccccatcaaccacacagtgcgacgccttgttgccttcacctttcaccctt

SEQ ID NO: 230 - DNA, 50 bp, Artificial Sequence
  Synthetic primer
gcgcttgtgctcggatggcttcgcatttccccaataccccattaaaccgt

SEQ ID NO: 231 - DNA, 50 bp, Artificial Sequence
  Synthetic primer
tgctcttcaggtttcaggctcaacaaggggcatggtctgctcttgcagat

SEQ ID NO: 232 - DNA, 50 bp, Artificial Sequence
  Synthetic primer
cccatctctgccactttgatgctatttgggttatgatggggcaagatggc

SEQ ID NO: 233 - DNA, 50 bp, Artificial Sequence
  Synthetic primer
ctgacattgaacccagttgctcagggtcagcccattcttacttccctggg

SEQ ID NO: 234 - DNA, 50 bp, Artificial Sequence
  Synthetic primer
cagccctctagcagagcgtcagtgcagtcgtgttatcccggcttttacag

SEQ ID NO: 235 - DNA, 50 bp, Artificial Sequence
  Synthetic primer
gactcctgccccggttcaaccctaccagcttgtggtaacttactgtcaca

SEQ ID NO: 236 - DNA, 50 bp, Artificial Sequence
  Synthetic primer
caacctgccacattttgggagcttttctacatgtctgttttctcatctgt

SEQ ID NO: 237 - DNA, 50 bp, Artificial Sequence
  Synthetic primer
ttttgtctcatggcaaccttccctggccagattcctgcctgtctcccagc

SEQ ID NO: 238 - DNA, 50 bp, Artificial Sequence
  Synthetic primer
tgggtcacctctgggcttccgcagcaccctccaaggcggagtggagcctt

SEQ ID NO: 239 - DNA, 50 bp, Artificial Sequence
  Synthetic primer
gactgtgaaaccgtcagttcggaaggctggttagaacatgtgggagcaac

SEQ ID NO: 240 - DNA, 50 bp, Artificial Sequence
  Synthetic primer
ccagccactctactcaaggggcatatattttggcatgaggtgggatagag

SEQ ID NO: 241 - DNA, 50 bp, Artificial Sequence
  Synthetic primer
ggttggaaataccatcagccttccttgctcggcccaggtctgtttcaggc

SEQ ID NO: 242 - DNA, 50 bp, Artificial Sequence
  Synthetic primer
cgaaaagagaaagtgggaaaatgggaagtccctctgcctaaagtacgtgc

SEQ ID NO: 243 - DNA, 50 bp, Artificial Sequence
  Synthetic primer
gcactttcatacgcaggcatctcttgttacctacatctaagctgttcccg

SEQ ID NO: 244 - DNA, 50 bp, Artificial Sequence
  Synthetic primer
cctcgtgttgattgcaggaggagtcggaattaaccctctgctttccatcc

SEQ ID NO: 245 - DNA, 50 bp, Artificial Sequence
  Synthetic primer
ggggctacatttgttcatttccagcagtagcataaacttacggtgacatg

SEQ ID NO: 246 - DNA, 50 bp, Artificial Sequence
  Synthetic primer
tcaggtgcccttatgaaaaggcttgatagagggagtttgtcctgtggccc

SEQ ID NO: 247 - DNA, 50 bp, Artificial Sequence
  Synthetic primer
gtagattcccaagagactttagcagtcaccagccttaatgcatgtacagg

SEQ ID NO: 248 - DNA, 50 bp, Artificial Sequence
  Synthetic primer
ggatattgtcagtctttaggggttgggctggatgccgaggtaaaagttct

SEQ ID NO: 249 - DNA, 50 bp, Artificial Sequence
  Synthetic primer
catccgccgcggcctcacgtgcgttgtaacaagccctcatcacatgtgtg

SEQ ID NO: 250 - DNA, 50 bp, Artificial Sequence
  Synthetic primer
tgtgctccaggcgacaccatttgccatcctgcttctaacgcaaacccctg

SEQ ID NO: 251 - DNA, 50 bp, Artificial Sequence
  Synthetic primer
cagggagaccgtgtcagtagggatgtgtgcctggctgtgtacgtgggtgt

SEQ ID NO: 252 - DNA, 50 bp, Artificial Sequence
  Synthetic primer
ccagaggaggtcctacacattaaaggataaagccccccagtgatgctggc

SEQ ID NO: 253 - DNA, 50 bp, Artificial Sequence
  Synthetic primer
agcactgcagactcagctgagcaccgatacaaagaaagacaaacatcctg

SEQ ID NO: 254 - DNA, 50 bp, Artificial Sequence
  Synthetic primer
aggaaatttgggcaggaaagggaactcacagtgtcggaatgcctggagca

SEQ ID NO: 255 - DNA, 50 bp, Artificial Sequence
  Synthetic primer
ggggggattgcctctactcatcactacctgtttctgcctgttctgctgcc

SEQ ID NO: 256 - DNA, 50 bp, Artificial Sequence
  Synthetic primer
ggttcctccgatcttacaggctcatccaggttccaaagtgcttctgtctc

SEQ ID NO: 257 - DNA, 50 bp, Artificial Sequence
  Synthetic primer
gcatgtgtatgatgtgtgtgcgtcggaccgcttctaggctactaagtgtc

SEQ ID NO: 258 - DNA, 50 bp, Artificial Sequence
  Synthetic primer
agcctatggagtggcccgtaaatcagttgactgtgtagctcttgcctggc

SEQ ID NO: 259 - DNA, 50 bp, Artificial Sequence
  Synthetic primer
tgttatgacaccaagtgactacaagggaggcaagacccctccaggcctcc

SEQ ID NO: 260 - DNA, 50 bp, Artificial Sequence
  Synthetic primer
caggacttcctcacaaagcatcacgtcatcttgacagctatgaattcctt

SEQ ID NO: 261 - DNA, 50 bp, Artificial Sequence
  Synthetic primer
gagtgaggtggtaatcagtgcctcttggtttcagccgtggtcctagtggc

SEQ ID NO: 262 - DNA, 50 bp, Artificial Sequence
  Synthetic primer
ctctgtgtttcctgtttcaccgccaccctttcaggagagaactacaccag

SEQ ID NO: 263 - DNA, 50 bp, Artificial Sequence
  Synthetic primer
ccggtgatggacccaccatcaactgcatcctccaagctggattcctagac

SEQ ID NO: 264 - DNA, 50 bp, Artificial Sequence
  Synthetic primer
ccaagcctaaggtgaaagggcgggcagaacctgtggtgttaggaggcaac

SEQ ID NO: 265 - DNA, 50 bp, Artificial Sequence
  Synthetic primer
gattcaccctgtccaaactgcctaagccctccgccattctcaagccctgc

SEQ ID NO: 266 - DNA, 50 bp, Artificial Sequence
  Synthetic primer
tgcatggcaaatcctgtcggtctccagttggttatctgaatagtgtcacc

SEQ ID NO: 267 - DNA, 50 bp, Artificial Sequence
  Synthetic primer
gagaatgctgtggtccgcaacacccagatcgacaactcctgggggtctga

SEQ ID NO: 268 - DNA, 50 bp, Artificial Sequence
  Synthetic primer
tccagctgctggaatcctaccatcccaggaggcaggcacagccagggaga

SEQ ID NO: 269 - DNA, 50 bp, Artificial Sequence
  Synthetic primer
caggccctcatcaccctcagacccctcctaagcagttccctcattgctct

SEQ ID NO: 270 - DNA, 50 bp, Artificial Sequence
  Synthetic primer
ggagatgtgagattcccagcccacctggtattgccctcgagttagtggta

SEQ ID NO: 271 - DNA, 50 bp, Artificial Sequence
  Synthetic primer
gtagccaaagctcacctttgaacagatcccggtgacattctatttccagg

SEQ ID NO: 272 - DNA, 50 bp, Artificial Sequence
  Synthetic primer
ctggcttaggctcccgcaatgggaaaacaaccggagggccagagcttagt

SEQ ID NO: 273 - DNA, 50 bp, Artificial Sequence
  Synthetic primer
gctgtctaggtccgtccggtgtgtcagattttcctcagattagatgtgcc

SEQ ID NO: 274 - DNA, 50 bp, Artificial Sequence
  Synthetic primer
ctgagaccaagatgggtcaccagaagctatgctggagccacccgcgaaaa

SEQ ID NO: 275 - DNA, 50 bp, Artificial Sequence
  Synthetic primer
actcagcactcagctgcatgcgggacaggaagaggaagatcaaaacgttg

SEQ ID NO: 276 - DNA, 50 bp, Artificial Sequence
  Synthetic primer
gtgtccagcagacctcaaggcagaagggtcacctaacccaggagtccaca

SEQ ID NO: 277 - DNA, 50 bp, Artificial Sequence
  Synthetic primer
gtcaccccttcaactggagcaggacggaaggttgtctgtacttgggaggg

SEQ ID NO: 278 - DNA, 50 bp, Artificial Sequence
  Synthetic primer
gaatcggaatctctgggggttgggacccagcaagggcacttaacaaaccc

SEQ ID NO: 279 - DNA, 50 bp, Artificial Sequence
  Synthetic primer
ctgtggacacatcctctccaccctctgggcctcagtgtcttgatgtatca

SEQ ID NO: 280 - DNA, 50 bp, Artificial Sequence
  Synthetic primer
accgcacgacaccagcacggatctcacggaggtcatggagcagattcaca

SEQ ID NO: 281 - DNA, 50 bp, Artificial Sequence
  Synthetic primer
cccaggactgcttctgaatgctgctacttgatttctttctcatcccacgc

SEQ ID NO: 282 - DNA, 50 bp, Artificial Sequence
  Synthetic primer
gacttagagatgagaatgccaaaggtcagtggcgttgggtggaccagacg

SEQ ID NO: 283 - DNA, 50 bp, Artificial Sequence
  Synthetic primer
tttgcagcctgctctagtgggacagcccagagcctggctgcccatcatgt

SEQ ID NO: 284 - DNA, 50 bp, Artificial Sequence
  Synthetic primer
gcctaggtgtccttgagctgaaccgcactgaagaactcttgtcctcactg

SEQ ID NO: 285 - DNA, 50 bp, Artificial Sequence
  Synthetic primer
cactgcagatgcggtcacctgactgaccacctgggttttctattaccagt

SEQ ID NO: 286 - DNA, 50 bp, Artificial Sequence
  Synthetic primer
gggcttcaagcactgtcttccatctgcatcttccagatttatcatgagtg

SEQ ID NO: 287 - DNA, 50 bp, Artificial Sequence
  Synthetic primer
ttgaagactaattgggagtccatctctctattggcactgggttcgattgc

SEQ ID NO: 288 - DNA, 50 bp, Artificial Sequence
  Synthetic primer
aggcccacaatgcctgtgtcctcaccattagtcccgtgcagcctgaagac

SEQ ID NO: 289 - DNA, 50 bp, Artificial Sequence
  Synthetic primer
tgcactccgcagcttgtgctgcccgtctcgaatgactggagtttcctgct

SEQ ID NO: 290 - DNA, 50 bp, Artificial Sequence
  Synthetic primer
cccgctcagtgaagacacagtgttcagctacggaagccataaggcaggcc

SEQ ID NO: 291 - DNA, 50 bp, Artificial Sequence
  Synthetic primer
caagctggccatgggggtcccaaacagtcattccaggcttagatgatgac

SEQ ID NO: 292 - DNA, 50 bp, Artificial Sequence
  Synthetic primer
gactgttgttgattagttattattgcaaagcactgtcaccggcctcaggg

SEQ ID NO: 293 - DNA, 50 bp, Artificial Sequence
  Synthetic primer
aggaggctaagaagcaccagggacaaatcgtgaagagacgcagactgtcc

SEQ ID NO: 294 - DNA, 50 bp, Artificial Sequence
  Synthetic primer
tgaacctgcttatacctaagggcaggagcaagcccctcacaatgcagctg

SEQ ID NO: 295 - DNA, 50 bp, Artificial Sequence
  Synthetic primer
gaggccgaggtggtggaggggatgcaccccagtgtccacttttgggcaca

SEQ ID NO: 296 - DNA, 50 bp, Artificial Sequence
  Synthetic primer
tgacccccacagggaggcaggaaaacagccagaagccaccttgacacttt

SEQ ID NO: 297 - DNA, 50 bp, Artificial Sequence
  Synthetic primer
acagtacctgtacctgcacggtcacccgctccgtgtgtcgccctatattg

SEQ ID NO: 298 - DNA, 50 bp, Artificial Sequence
  Synthetic primer
gatggccatctgcagaccaagaagcaagccctcaacagaactgaatctac

SEQ ID NO: 299 - DNA, 50 bp, Artificial Sequence
  Synthetic primer
aggggcagtatactcttatcagtgcgaggtagctggggcctgtgatagtt

SEQ ID NO: 300 - DNA, 50 bp, Artificial Sequence
  Synthetic primer
tgttagccctgaagatctggctaccccaataggaaggctgaaggtttccc

SEQ ID NO: 301 - DNA, 50 bp, Artificial Sequence
  Synthetic primer
tgccccctgactgatagcatttcagaatgtgtcttttgaagggctatacc

SEQ ID NO: 302 - DNA, 50 bp, Artificial Sequence
  Synthetic primer
ccttacctggggcaagttagccattgtctcccagtcatgtttaccaccca

SEQ ID NO: 303 - DNA, 50 bp, Artificial Sequence
  Synthetic primer
gggcggagctgggtcagtcccgtatttattttgctttgagaagaggctcc

SEQ ID NO: 304 - DNA, 50 bp, Artificial Sequence
  Synthetic primer
aggcctaagaggctttctccaggaggattagcttggagttctctatactc

SEQ ID NO: 305 - DNA, 50 bp, Artificial Sequence
  Synthetic primer
ggttttctaaccctgacacggactgtgcatactttccctcatccatgctg

SEQ ID NO: 306 - DNA, 50 bp, Artificial Sequence
  Synthetic primer
agcaatcttggcctttctagctgtgtgaccccaggccggtcaatccctcc

SEQ ID NO: 307 - DNA, 50 bp, Artificial Sequence
  Synthetic primer
gtgggaaagggtccagattttaaaggtgctttaaggttgccctctgccga

SEQ ID NO: 308 - DNA, 50 bp, Artificial Sequence
  Synthetic primer
gtggaggtggggatctgcccaacagccaggaggcactgcagaagctctct

SEQ ID NO: 309 - DNA, 50 bp, Artificial Sequence
  Synthetic primer
cccatttcagacagcaacatgtagaagatgttcagcgtgcagggcttgct

SEQ ID NO: 310 - DNA, 50 bp, Artificial Sequence
  Synthetic primer
gatgtgggccaagtccactgtcctccttggcggcaaaagcccattgaaga

SEQ ID NO: 311 - DNA, 50 bp, Artificial Sequence
  Synthetic primer
ccacatcgtcttccctgtcccaatcgaccagtgtatcgacggctgagtgc

SEQ ID NO: 312 - DNA, 50 bp, Artificial Sequence
  Synthetic primer
ccaggatgtctagcacccccatccccttggcccttcctcatgcttctgaa

SEQ ID NO: 313 - DNA, 50 bp, Artificial Sequence
  Synthetic primer
cttgatggggcctgggtttgttcctggggctggaatgctgggtatgctct

SEQ ID NO: 314 - DNA, 50 bp, Artificial Sequence
  Synthetic primer
caaactacactcgctcaggaagccttctgtttagaaaactggtgtgtgcg

SEQ ID NO: 315 - DNA, 50 bp, Artificial Sequence
  Synthetic primer
ccataactggagaaagaagctccattgaccgaagccacagggcagcatgg

SEQ ID NO: 316 - DNA, 50 bp, Artificial Sequence
  Synthetic primer
tgcgccgacgatgcccagaatccagaactttgtctatcactctccccaac

SEQ ID NO: 317 - DNA, 50 bp, Artificial Sequence
  Synthetic primer
aaatgactcccctctgccctcgccccgcgctctgctaccatttccttacg

SEQ ID NO: 318 - DNA, 50 bp, Artificial Sequence
  Synthetic primer
ctcgatagttacccaaagttcagtctagatggcacaaaccacctctcagg

SEQ ID NO: 319 - DNA, 50 bp, Artificial Sequence
  Synthetic primer
caagcctggcagtaaatccgaatatccagaaccctgaccctgccgtgtac

SEQ ID NO: 320 - DNA, 50 bp, Artificial Sequence
  Synthetic primer
ccctccccagaagaggagaagaggaaacacaagaagaaacgcctggtgca

SEQ ID NO: 321 - DNA, 50 bp, Artificial Sequence
  Synthetic primer
acctccacgatttcgccaccaggaacacaaacagcttctaaagaggggtc

SEQ ID NO: 322 - DNA, 50 bp, Artificial Sequence
  Synthetic primer
ctcctgcccagctcccagcggccctggtagacccacaacttcctgaagcc

SEQ ID NO: 323 - DNA, 50 bp, Artificial Sequence
  Synthetic primer
atccttccttggctctttttagcttgtggcggcagtgggcagtccgtggc

SEQ ID NO: 324 - DNA, 50 bp, Artificial Sequence
  Synthetic primer
acttgcctgggtcacatagctaaggaagaggtggacttgcccagctttgc

SEQ ID NO: 325 - DNA, 50 bp, Artificial Sequence
  Synthetic primer
caggattgagcactgaattccaagctccactggcatttcagtctgacgtg

SEQ ID NO: 326 - DNA, 50 bp, Artificial Sequence
  Synthetic primer
aggccagagctttgttgcttcatcggcaggttgggacttagatggccgtg

SEQ ID NO: 327 - DNA, 50 bp, Artificial Sequence
  Synthetic primer
tcagaccgagaagcagggtgagagattctaacgactggatgctgctagta

SEQ ID NO: 328 - DNA, 50 bp, Artificial Sequence
  Synthetic primer
gagacagggacgcactctttacggcttcggcggctaatgctaccgcttaa

SEQ ID NO: 329 - DNA, 50 bp, Artificial Sequence
  Synthetic primer
agcagagggcagctgagaggctgggtggctgggcctgggagacacacaga

SEQ ID NO: 330 - DNA, 50 bp, Artificial Sequence
  Synthetic primer
gtcacctcagctgcccacacccacactctccagcatctggcacaataaac

SEQ ID NO: 331 - DNA, 50 bp, Artificial Sequence
  Synthetic primer
ctcctgctctttctacatgactgggcctcccttgggctggaagaattggg

SEQ ID NO: 332 - DNA, 50 bp, Artificial Sequence
  Synthetic primer
cctcaggacttgcaaaaacgggtctcagcatgccttcccagagccatgag

SEQ ID NO: 333 - DNA, 50 bp, Artificial Sequence
  Synthetic primer
catggctctaggctaccctatggccatgggcctcttcaagggccacaagg

SEQ ID NO: 334 - DNA, 50 bp, Artificial Sequence
  Synthetic primer
tcatcaccatcctggataagtacctgaagtcaggcgacggggagggcacg

SEQ ID NO: 335 - DNA, 50 bp, Artificial Sequence
  Synthetic primer
gggcacgcgctgcactccgtaactcaacatggcatgcctttctctccgta

SEQ ID NO: 336 - DNA, 50 bp, Artificial Sequence
  Synthetic primer
cagcatgtagggcagtgcttgcacgtagcatctggtgcctaaccagtgtt

SEQ ID NO: 337 - DNA, 50 bp, Artificial Sequence
  Synthetic primer
cagaggcaggaaaagcaaggagccagaattaagaggttgggtcagtctgc

SEQ ID NO: 338 - DNA, 50 bp, Artificial Sequence
  Synthetic primer
gagcgggaacagcttccacgtgttcgaccagggccagtttgccaaggagg

SEQ ID NO: 339 - DNA, 50 bp, Artificial Sequence
  Synthetic primer
caaacctcgggccacaagacaccacttcccctttaccctggacagcagga

SEQ ID NO: 340 - DNA, 50 bp, Artificial Sequence
  Synthetic primer
tcccagcatcccctcacccaaacactccttcctggcctatctcggactga

SEQ ID NO: 341 - DNA, 50 bp, Artificial Sequence
  Synthetic primer
ttctcaccgtggcaagaagggaccaaccccaccctggtctctgtccccaa

SEQ ID NO: 342 - DNA, 50 bp, Artificial Sequence
  Synthetic primer
aggacgtgatcctgcttggggacttcaatgctgactgcgcttcactgacc

SEQ ID NO: 343 - DNA, 50 bp, Artificial Sequence
  Synthetic primer
gaacttttccaccagcagggaaccccaagagcatccaatagaagctgtgc

SEQ ID NO: 344 - DNA, 50 bp, Artificial Sequence
  Synthetic primer
tggaggccgtggctatggttggaggtcagcttcaggccttctggaagcat

SEQ ID NO: 345 - DNA, 50 bp, Artificial Sequence
  Synthetic primer
acaagctgtccccgctgcacgagcgcatctactgtgcactctctggttca

SEQ ID NO: 346 - DNA, 50 bp, Artificial Sequence
  Synthetic primer
ctcccagcgttaacacaaaatccatgggcagtatgatggcaggtcctctg

SEQ ID NO: 347 - DNA, 50 bp, Artificial Sequence
  Synthetic primer
actgaaagcagtcatgcgaatggagggtgctcttgtgtagctggtcaggg

SEQ ID NO: 348 - DNA, 50 bp, Artificial Sequence
  Synthetic primer
caccttccaacaggccctctggacaaatacaagattcaggcgatcgacgc

SEQ ID NO: 349 - DNA, 50 bp, Artificial Sequence
  Synthetic primer
gccaagcccaggtcacagatcttcccccgaccatgctgggaatccagaaa

SEQ ID NO: 350 - DNA, 50 bp, Artificial Sequence
  Synthetic primer
tgagcccaaattccactggctcctccactgaaaactactaaaccggtgga

SEQ ID NO: 351 - DNA, 50 bp, Artificial Sequence
  Synthetic primer
gagttaaacaagctgctgggcaaagtgaccatcgctcagggcggcgtcct

SEQ ID NO: 352 - DNA, 50 bp, Artificial Sequence
  Synthetic primer
ggcattatgaaggcagcacagtccccgaaaagaagacaccgaagagccct

SEQ ID NO: 353 - DNA, 50 bp, Artificial Sequence
  Synthetic primer
ggtccccatgtgcctgttgttcagccctctctcttgttccctttctgagc

SEQ ID NO: 354 - DNA, 50 bp, Artificial Sequence
  Synthetic primer
aacaaggagatagggtagacattgtactcagtgggccttggggcctagcc

SEQ ID NO: 355 - DNA, 50 bp, Artificial Sequence
  Synthetic primer
cagcgaagtccgctcccgcgcccaagaagggctcgaagaaagccgtgact

SEQ ID NO: 356 - DNA, 50 bp, Artificial Sequence
  Synthetic primer
gctgatctggcaggatgctctcttcaagcatatccaaaaccagatgtgcc

SEQ ID NO: 357 - DNA, 50 bp, Artificial Sequence
  Synthetic primer
tggtggtctgggctatggctgtggctgtggaggctacagatatggctctg

SEQ ID NO: 358 - DNA, 50 bp, Artificial Sequence
  Synthetic primer
ggaagccaggtgcctttaatccactgtaacctcacaactccaagtccaca

SEQ ID NO: 359 - DNA, 50 bp, Artificial Sequence
  Synthetic primer
gagcaggggagaaatagcagaggggcttggagggtcacataggtagatgg

SEQ ID NO: 360 - DNA, 50 bp, Artificial Sequence
  Synthetic primer
ggtgtctagcttgtaaagttctggctgtagttatgaccttggcttccctg

SEQ ID NO: 361 - DNA, 50 bp, Artificial Sequence
  Synthetic primer
ggcccaccttcagtgtgtgaaactcgaagtgcttggtcacctgaattgtt

SEQ ID NO: 362 - DNA, 50 bp, Artificial Sequence
  Synthetic primer
ggtgttcaggactggtaagcgactgtcatcaagtaaggcccccgtgctgg

SEQ ID NO: 363 - DNA, 50 bp, Artificial Sequence
  Synthetic primer
gcactgctggaggaagaggaagatttgtgcagaccaagagcaccacagac

SEQ ID NO: 364 - DNA, 50 bp, Artificial Sequence
  Synthetic primer
acatggcccgcaaggacaatgaatccactcacattgcagaacaattccga

SEQ ID NO: 365 - DNA, 50 bp, Artificial Sequence
  Synthetic primer
cagctacccggaaggctgaggcaggagaattgcgtgaaccaggcaggaaa

SEQ ID NO: 366 - DNA, 50 bp, Artificial Sequence
  Synthetic primer
ggtggccagcctggatctcctcaagtccccaagattcacacctgactctg

SEQ ID NO: 367 - DNA, 50 bp, Artificial Sequence
  Synthetic primer
caccactggaagcaccagcataactgtgaggacccttccaaactgtgggc

SEQ ID NO: 368 - DNA, 50 bp, Artificial Sequence
  Synthetic primer
cagcagagttatggagtatcagaatctttccatggaaacctccatatggc

SEQ ID NO: 369 - DNA, 50 bp, Artificial Sequence
  Synthetic primer
gcacccgcttcaaatattgtgcccatgatgcacggtgctacacctaccac

SEQ ID NO: 370 - DNA, 50 bp, Artificial Sequence
  Synthetic primer
ctgaggttatgtacaaccaactctcagaattcagacttcctgcagctgcc

SEQ ID NO: 371 - DNA, 50 bp, Artificial Sequence
  Synthetic primer
aacacgcccactgattttacattatgctcctaccacccggcagcctctcc

SEQ ID NO: 372 - DNA, 50 bp, Artificial Sequence
  Synthetic primer
tcttgaggctgttgagaaacttaggacagaagcctacctcatcccaggcc

SEQ ID NO: 373 - DNA, 50 bp, Artificial Sequence
  Synthetic primer
gaaccaggaaaggcttcagacttctgaacccacatcctaagccaaacccc

SEQ ID NO: 374 - DNA, 50 bp, Artificial Sequence
  Synthetic primer
ttttttctgcaagggccgccacgaggacgagagccgtacagtgcttcacg

SEQ ID NO: 375 - DNA, 50 bp, Artificial Sequence
  Synthetic primer
gaccctgtggagaaattcgttgttcccactgaaatggactgactgtaacg

SEQ ID NO: 376 - DNA, 50 bp, Artificial Sequence
  Synthetic primer
gtctctggatgaatgaccaggaggctattcaagatctctggcagtggagg

SEQ ID NO: 377 - DNA, 50 bp, Artificial Sequence
  Synthetic primer
gtgagcccaaagttctgaaaggtgttgcggctccttcgccttcgtcaaat

SEQ ID NO: 378 - DNA, 50 bp, Artificial Sequence
  Synthetic primer
gcatgcatgggaggaattcatcttcagtctaccagcccccgctgtgtcgg

SEQ ID NO: 379 - DNA, 50 bp, Artificial Sequence
  Synthetic primer
ggcagttgtctgcattaacctgttcatacacccattttgtccctttattg

SEQ ID NO: 380 - DNA, 50 bp, Artificial Sequence
  Synthetic primer
cactttcctgctcctctctgtctctagcacacaactgtgaatgtcctgtg

SEQ ID NO: 381 - DNA, 50 bp, Artificial Sequence
  Synthetic primer
gctcttggttcagagcaggcagaacctcgtggctgaggagagactcagct

SEQ ID NO: 382 - DNA, 50 bp, Artificial Sequence
  Synthetic primer
ccctcatgaacccaacattgatagtcccttgaacacacatgctgccgagc

SEQ ID NO: 383 - DNA, 50 bp, Artificial Sequence
  Synthetic primer
gcctttgtgatctcacctgttggaatgcactaatgttttctgagcccctt

SEQ ID NO: 384 - DNA, 50 bp, Artificial Sequence
  Synthetic primer
cctcggtgcttacaacagaatgctgccctacatcgtcatgggtagtctga

SEQ ID NO: 385 - DNA, 50 bp, Artificial Sequence
  Synthetic primer
ggccagttttatgaagctttggaaggcactatggacagaagctggtggac

SEQ ID NO: 386 - DNA, 50 bp, Artificial Sequence
  Synthetic primer
gccacaaaaggcattctctgtcctacctagctgtcacttctctgtgcagc

SEQ ID NO: 387 - DNA, 50 bp, Artificial Sequence
  Synthetic primer
gagcaaaaagccaactgctgtcagggactggcgggtatagcacctgggtc

SEQ ID NO: 388 - DNA, 50 bp, Artificial Sequence
  Synthetic primer
cgttctctgcctcacatagcttacaagccagctggagaaatatggtactc

SEQ ID NO: 389 - DNA, 50 bp, Artificial Sequence
  Synthetic primer
tattggagataatgtcagcagtgagagatgaggatccactggaattagcc

SEQ ID NO: 390 - DNA, 50 bp, Artificial Sequence
  Synthetic primer
gtctgaggcttgggcgctgcctgggccgagcggagatcggggtttgcctc

SEQ ID NO: 391 - DNA, 50 bp, Artificial Sequence
  Synthetic primer
cggctccatgttggaactgtcgtggaagggaacgaagcccatagacctgg

SEQ ID NO: 392 - DNA, 50 bp, Artificial Sequence
  Synthetic primer
gtaggcccccaaagtgccgtctttccctagcattttactcaatgtttgcc

SEQ ID NO: 393 - DNA, 50 bp, Artificial Sequence
  Synthetic primer
cttgcctgaggacagcctgcaggacacagcactgtgatttgaacccagag

SEQ ID NO: 394 - DNA, 50 bp, Artificial Sequence
  Synthetic primer
aggtgctaaagttgaacgagctgagggatatgaaccaccagtccaagaat

SEQ ID NO: 395 - DNA, 50 bp, Artificial Sequence
  Synthetic primer
gcccccatacatagagtgggactcttggaatcaggagacaaagctaccac

SEQ ID NO: 396 - DNA, 50 bp, Artificial Sequence
  Synthetic primer
tgagactgtccaggcagccaggttaggccaggaggaccatgtgaatgggg

SEQ ID NO: 397 - DNA, 50 bp, Artificial Sequence
  Synthetic primer
ggccttactgggccacactgataccgtcacctgcgccacagcatcattag

SEQ ID NO: 398 - DNA, 50 bp, Artificial Sequence
  Synthetic primer
ctgtagcaacgtctgtcaggcccccttgtgtttcatctcctgcgcgcgta

SEQ ID NO: 399 - DNA, 50 bp, Artificial Sequence
  Synthetic primer
tgtcatggagtactgtgaactcgggaccctgagggagctgttggataggg

SEQ ID NO: 400 - DNA, 50 bp, Artificial Sequence
  Synthetic primer
gcatcgtctcagcccgtgcccaagaagctgctcatgatggctggtattga

SEQ ID NO: 401 - DNA, 50 bp, Artificial Sequence
  Synthetic primer
tcggggaaactgtgtgtgctgaagagtacgtgggagctctctgtgctatc

SEQ ID NO: 402 - DNA, 50 bp, Artificial Sequence
  Synthetic primer
ggaagtgcatgggactttccctctctgcactccagctcttactgtaccat

SEQ ID NO: 403 - DNA, 50 bp, Artificial Sequence
  Synthetic primer
tcctacctgaggtggccaagaagtttcccaacatgaagatacagatccat

SEQ ID NO: 404 - DNA, 50 bp, Artificial Sequence
  Synthetic primer
gagccaccatgcctggccatgattcttaagagaattgactgggcctcatg

SEQ ID NO: 405 - DNA, 50 bp, Artificial Sequence
  Synthetic primer
taacgcctgcaacgacatgggtaagatgctgcaattcgtgctgcccgtgg

SEQ ID NO: 406 - DNA, 50 bp, Artificial Sequence
  Synthetic primer
ggagccccaccctgggctgcctctctaggcttcgctgttcatcatacaat

SEQ ID NO: 407 - DNA, 50 bp, Artificial Sequence
  Synthetic primer
ctgcgtggtcacgctgagccagcgcctgggcctggaaccgggctgcagcc

SEQ ID NO: 408 - DNA, 50 bp, Artificial Sequence
  Synthetic primer
gggacaaatccctgacatcgtccttccactgtgatttggagatgacctcc

SEQ ID NO: 409 - DNA, 50 bp, Artificial Sequence
  Synthetic primer
ctggctaaacaacctgacaaactgtggccaggggaaccgacacaaccctc

SEQ ID NO: 410 - DNA, 50 bp, Artificial Sequence
  Synthetic primer
actttgtttccaggcttttgctactcttcactcagctacaataaacatcc

SEQ ID NO: 411 - DNA, 50 bp, Artificial Sequence
  Synthetic primer
gggcttaactttacctccttgaaaatgacatggacttgggtggatggttc

SEQ ID NO: 412 - DNA, 50 bp, Artificial Sequence
  Synthetic primer
gtgctctttgttcatcattggccctcattccaagcactttacgctgtctg

SEQ ID NO: 413 - DNA, 50 bp, Artificial Sequence
  Synthetic primer
gtgtctaccttctgcagactccaatggctcaggaactgggaatgcagtgc

SEQ ID NO: 414 - DNA, 50 bp, Artificial Sequence
  Synthetic primer
cacaatggaaccaggaaaagcagcccttgccccttcagtgtgtcagcagc

SEQ ID NO: 415 - DNA, 50 bp, Artificial Sequence
  Synthetic primer
tgctgtgacctcactctcttctgctgtcctgggacctcgtgggcctcctc

SEQ ID NO: 416 - DNA, 50 bp, Artificial Sequence
  Synthetic primer
tgtcccacttcctctatgtccctttccttggtgtctgtgtctgtggggcc

SEQ ID NO: 417 - DNA, 50 bp, Artificial Sequence
  Synthetic primer
gagatgatgtgtggtcccactgaatggtatcagagttgtagtcctagctc

SEQ ID NO: 418 - DNA, 50 bp, Artificial Sequence
  Synthetic primer
gctctgccacttgctagctgtgagactgtggacaaaccactcagcctctg

SEQ ID NO: 419 - DNA, 50 bp, Artificial Sequence
  Synthetic primer
gagccaccacagctggcccccttctgttttatgtttggtttttgagaagg

SEQ ID NO: 420 - DNA, 50 bp, Artificial Sequence
  Synthetic primer
cctggtgataggcctcgtcctcctgggtagaatcctctcggaatcactcc

SEQ ID NO: 421 - DNA, 50 bp, Artificial Sequence
  Synthetic primer
gcagacgccctgtacaatcaagatcacccacaagaaccagatgcccatgc

SEQ ID NO: 422 - DNA, 50 bp, Artificial Sequence
  Synthetic primer
accaaggcaactcaacaccttctttctctgggcttgaaccgccactgctc

SEQ ID NO: 423 - DNA, 50 bp, Artificial Sequence
  Synthetic primer
ctgcagggaaagttccttctgtcggtttttagacacagatctctctgccc

SEQ ID NO: 424 - DNA, 50 bp, Artificial Sequence
  Synthetic primer
ttgtgcatcaagctcatcgccgcctggttcgtgcccgacatccctcagtc

SEQ ID NO: 425 - DNA, 50 bp, Artificial Sequence
  Synthetic primer
tctctcttcagttcctccaggtaattcttactcaaacttgtaccaacttg

SEQ ID NO: 426 - DNA, 50 bp, Artificial Sequence
  Synthetic primer
cagatgctgatgtttgtaaggtccggtggggccatgaggaagaagaggag

SEQ ID NO: 427 - DNA, 50 bp, Artificial Sequence
  Synthetic primer
gccaccaccttaacaggtgtcatagatgtagctttggggtcaggtgccac

SEQ ID NO: 428 - DNA, 50 bp, Artificial Sequence
  Synthetic primer
ccctggtaggcccacaacttcccaaagccaagctccgcaggcccagctca

SEQ ID NO: 429 - DNA, 50 bp, Artificial Sequence
  Synthetic primer
tgtactgtaacctcacaactccaagtccacagaatatttcaaactgcagg

SEQ ID NO: 430 - DNA, 50 bp, Artificial Sequence
  Synthetic primer
ggcctgaggaccaggaagtagagcagcaggtgcagaaagagctggaacag

SEQ ID NO: 431 - DNA, 50 bp, Artificial Sequence
  Synthetic primer
gattacctggagcctggagccactttaagttgtacttctgactaaactgg

SEQ ID NO: 432 - DNA, 50 bp, Artificial Sequence
  Synthetic primer
gagttcctgaattttgaacaggattacctggagcctggagccactttaag

SEQ ID NO: 433 - DNA, 50 bp, Artificial Sequence
  Synthetic primer
ccctccaccttggacacaggacgctgtggtttctgagccaggtacaatga

SEQ ID NO: 434 - DNA, 50 bp, Artificial Sequence
  Synthetic primer
ctttgtcctggcggtgggtgttgtgctgtcccccttggtgtttatcctgc

SEQ ID NO: 435 - DNA, 50 bp, Artificial Sequence
  Synthetic primer
tgtcctgtgactgccccacagagataaggggccaggagggattgaaaggc

SEQ ID NO: 436 - DNA, 50 bp, Artificial Sequence
  Synthetic primer
caaccaaggacatgtaccataaactaaagaccagagccttcctgtcaccc

SEQ ID NO: 437 - DNA, 50 bp, Artificial Sequence
  Synthetic primer
gaatcaaggaggtcaagtaaggtcacaggggcacttgggttgagccaggg

SEQ ID NO: 438 - DNA, 50 bp, Artificial Sequence
  Synthetic primer
tgaggtccctgagtaatccaccccagctaaaggcaaacccttcaatcagg

SEQ ID NO: 439 - DNA, 50 bp, Artificial Sequence
  Synthetic primer
tgggattcggcctctggaaagtggtggtagttccagatttatgtgaatgt

SEQ ID NO: 440 - DNA, 50 bp, Artificial Sequence
  Synthetic primer
tccctgctgtcgctcctggttggtgcgtggctcaggctaggtcattgaac

SEQ ID NO: 441 - DNA, 50 bp, Artificial Sequence
  Synthetic primer
ccctgccctcatgttgctttgggtctagtggaggagagagacagataagc

SEQ ID NO: 442 - DNA, 50 bp, Artificial Sequence
  Synthetic primer
ccgatgggacacttgggggaccctgaggatgtggcagatgtggtcgcatt

SEQ ID NO: 443 - DNA, 50 bp, Artificial Sequence
  Synthetic primer
tccagctttcctggttagcgcaacgcggctccacgaccacacgcacttca

SEQ ID NO: 444 - DNA, 50 bp, Artificial Sequence
  Synthetic primer
gccctggacttggtacagtttcaggagcgtgaaggacttaaccgactgcc

SEQ ID NO: 445 - DNA, 50 bp, Artificial Sequence
  Synthetic primer
gctcttgatcccgatttcatgcacagccctgcagtaaggagcccagaagg

SEQ ID NO: 446 - DNA, 50 bp, Artificial Sequence
  Synthetic primer
ccgctgcccatctaaccttcgtaaagacctgtctcctgtttccagccgcc

SEQ ID NO: 447 - DNA, 50 bp, Artificial Sequence
  Synthetic primer
ggttaggctgccccatcccaccctactctgtgtggctgctttctttggtg

SEQ ID NO: 448 - DNA, 50 bp, Artificial Sequence
  Synthetic primer
cggcctgatggagagaaggaacatgttcgactggctcctgattacaatgc

SEQ ID NO: 449 - DNA, 50 bp, Artificial Sequence
  Synthetic primer
cacagagggtcagagtgttggcacctgtcttccgggtctgttcacccgtc

SEQ ID NO: 450 - DNA, 50 bp, Artificial Sequence
  Synthetic primer
ccccagatggttccaaatattccttacctcgtttggttcccaagtcacag

SEQ ID NO: 451 - DNA, 50 bp, Artificial Sequence
  Synthetic primer
ggccacggcgcttggaatcctggttgttgctggatgctcttttgcgatta

SEQ ID NO: 452 - DNA, 50 bp, Artificial Sequence
  Synthetic primer
actgggaagcagccaaccctctgtagagaaatcccagattgggagtctgg

SEQ ID NO: 453 - DNA, 50 bp, Artificial Sequence
  Synthetic primer
ctacgccatgggacatctaattcagaggaagaaggtccatgtctttgggg

SEQ ID NO: 454 - DNA, 50 bp, Artificial Sequence
  Synthetic primer
gaatagaaaccagacagcaattctttagttccagccaccattcgccccac

SEQ ID NO: 455 - DNA, 50 bp, Artificial Sequence
  Synthetic primer
tggccagttgcaggttaagccccagaaaagttcaccttggagaagctccg

SEQ ID NO: 456 - DNA, 50 bp, Artificial Sequence
  Synthetic primer
agtgcatgaggctgaaagttccaagcctcttaccatgtggtttcatggta

SEQ ID NO: 457 - DNA, 50 bp, Artificial Sequence
  Synthetic primer
ctgactgcccaggaatcaagaagtgctgtgaaggctcttgcgggatggcc

SEQ ID NO: 458 - DNA, 50 bp, Artificial Sequence
  Synthetic primer
gatatggtgccctgtgtatcccagagatgctagaaaactgttctttgctc

SEQ ID NO: 459 - DNA, 50 bp, Artificial Sequence
  Synthetic primer
ctcctgccacccagtggcctctttaggccaagctcatgcctcacaagggc

SEQ ID NO: 460 - DNA, 50 bp, Artificial Sequence
  Synthetic primer
gcccaacatcctcatctgtcttgtggacaacatctttcctcctgtggtca

SEQ ID NO: 461 - DNA, 50 bp, Artificial Sequence
  Synthetic primer
cacactgtcaccagggttatttgcatccaagggagctggaattgagtacc

SEQ ID NO: 462 - DNA, 50 bp, Artificial Sequence
  Synthetic primer
gcagaagcaatggagtgaacctctgctccccacacagctggctacactgc

SEQ ID NO: 463 - DNA, 50 bp, Artificial Sequence
  Synthetic primer
gaaattcctcaacagggcattatccgctccctgaatgtccatgtgagtgg

SEQ ID NO: 464 - DNA, 50 bp, Artificial Sequence
  Synthetic primer
gtcctctccatcatcctcagtgtgagtcctcagagcctccatctgccaag

SEQ ID NO: 465 - DNA, 50 bp, Artificial Sequence
  Synthetic primer
agggcccagtggttttacgaatatgggaatgccctggaagggtccacctt

SEQ ID NO: 466 - DNA, 50 bp, Artificial Sequence
  Synthetic primer
atgaacgtgctagaacagctcaccgcactgatgctgccctggggctgtga

SEQ ID NO: 467 - DNA, 50 bp, Artificial Sequence
  Synthetic primer
agtggcactcgacgaaaaggagactatgccagttactaccagggcctatg

SEQ ID NO: 468 - DNA, 50 bp, Artificial Sequence
  Synthetic primer
gaccggaagcaaccccttcacagacacgagcacatcggcaaaccctatga

SEQ ID NO: 469 - DNA, 50 bp, Artificial Sequence
  Synthetic primer
gatcagggaaacctttgggacttctgacaggcatctggtgaagagacaat

SEQ ID NO: 470 - DNA, 50 bp, Artificial Sequence
  Synthetic primer
ctcttacagtgaggagcacaccgcctggttgtgggagaatggctctgcac

SEQ ID NO: 471 - DNA, 50 bp, Artificial Sequence
  Synthetic primer
tcaacaaagaggtgctgacctgagagtagggcacataacctcagccactg

SEQ ID NO: 472 - DNA, 50 bp, Artificial Sequence
  Synthetic primer
gggaccagaggaaacaaaagcaccaagaggttgactagagctgaccagtg

SEQ ID NO: 473 - DNA, 50 bp, Artificial Sequence
  Synthetic primer
ctgcagagcagcgtccgaatgcctacatcccagaagcagatgccactctt

SEQ ID NO: 474 - DNA, 50 bp, Artificial Sequence
  Synthetic primer
gttccacaggctaaagcccagatgcccaccatagaacgagcaaccttgac

SEQ ID NO: 475 - DNA, 50 bp, Artificial Sequence
  Synthetic primer
ggcagacaaagggctccctggctactgggaagatacagaaccatcctgtg

SEQ ID NO: 476 - DNA, 50 bp, Artificial Sequence
  Synthetic primer
ggagtgggcagtgtccccgttaccagtgtcagcatctctgtgagtggcat

SEQ ID NO: 477 - DNA, 50 bp, Artificial Sequence
  Synthetic primer
cttggtcggcccacagcttcctgaagccaagctccccgggcccagctcag

SEQ ID NO: 478 - DNA, 50 bp, Artificial Sequence
  Synthetic primer
gtgaaacagttgacagacatcaccagttgacttctgcattaacctgcatg

SEQ ID NO: 479 - DNA, 50 bp, Artificial Sequence
  Synthetic primer
gcagcgacaacccgtcctctcccgagtttttcataactgagcccactcgc

SEQ ID NO: 480 - DNA, 50 bp, Artificial Sequence
  Synthetic primer
ccaacaggagctgtgcagattgtaaagccccagatcctgactgggcatcc

SEQ ID NO: 481 - DNA, 50 bp, Artificial Sequence
  Sythentic primer
ctcaccttggctgtgggctgtgaacattccgggaccctgggcatcttatc

SEQ ID NO: 482 - DNA, 50 bp, Artificial Sequence
  Sythentic primer
atatgcagcacgaggggctgcaagagcccctcaccctgagctgggagcca

SEQ ID NO: 483 - DNA, 50 bp, Artificial Sequence
  Sythentic primer
cagtacacatcctatgccataagtggtcccacagtacaaggtggcctccc

SEQ ID NO: 484 - DNA, 50 bp, Artificial Sequence
  Sythentic primer
ggtagcctagttatttgagcctggtttcaatgtgagaaccacgtttactg

SEQ ID NO: 485 - DNA, 50 bp, Artificial Sequence
  Sythentic primer
tccatgggcactggtctcagtggtgggaaacgtcctagtcaggaagagga

SEQ ID NO: 486 - DNA, 50 bp, Artificial Sequence
  Sythentic primer
gacccaaacttgtggaagtatcatgtgttaagttctctgtctctgtttct

SEQ ID NO: 487 - DNA, 50 bp, Artificial Sequence
  Sythentic primer
gatctccaggcttggcctccagagcagcccacaccaaccccaaaataaaa

SEQ ID NO: 488 - DNA, 50 bp, Artificial Sequence
  Sythentic primer
ctcctcttcacgattgatagcaaaatcagaaacgttagggcaccagtgag

SEQ ID NO: 489 - DNA, 50 bp, Artificial Sequence
  Sythentic primer
gcctagaatatcctgcagtggtagagtttgctccattccagaagatagcc

SEQ ID NO: 490 - DNA, 50 bp, Artificial Sequence
  Sythentic primer
tctggctggataatgttcagtgtcggggcacggagagtaccctgtggagc

SEQ ID NO: 491 - DNA, 50 bp, Artificial Sequence
  Sythentic primer
gagcagctccctcgctgcgatctattgaaagtcagatctccacacaaggg

SEQ ID NO: 492 - DNA, 50 bp, Artificial Sequence
  Sythentic primer
tctgctgctctggatggctgaaggctcctgggccatcttcatgtgctgct

SEQ ID NO: 493 - DNA, 50 bp, Artificial Sequence
  Sythentic primer
tacacgctcaccggctaccagatcgacatcaccacggagaaccagtaccg

SEQ ID NO: 494 - DNA, 50 bp, Artificial Sequence
  Sythentic primer
atgctggccactctcagtccagcgttcctcagtagtgaatagcgaacctg

SEQ ID NO: 495 - DNA, 50 bp, Artificial Sequence
  Sythentic primer
ttgccccaccccgccagccgcgatacggcgcagttcctatattcatgtta

SEQ ID NO: 496 - DNA, 50 bp, Artificial Sequence
  Sythentic primer
tctcactctttctctgctgttgcaaatacatggataacaccgttaattcc

SEQ ID NO: 497 - DNA, 50 bp, Artificial Sequence
  Sythentic primer
gagggtggaatggatgtgtttggcgctgcatgggatctggtgcccctctt

SEQ ID NO: 498 - DNA, 50 bp, Artificial Sequence
  Sythentic primer
ccagcttcagcagattatgcgtctgacaggaacaccccccgcttatctca

SEQ ID NO: 499 - DNA, 50 bp, Artificial Sequence
  Sythentic primer
gccatgtaagggagccatcttggaagcagatcctccagcctccagtcaag

SEQ ID NO: 500 - DNA, 50 bp, Artificial Sequence
  Sythentic primer
cagcgtgcatgtgttcaagccttagattggcgatgtcgtattttcctcac

SEQ ID NO: 501 - DNA, 50 bp, Artificial Sequence
  Synthetic primer
cctctgtgcctgagttctccctgttgtctcaaagcggtacccatcctccc

SEQ ID NO: 502 - DNA, 50 bp, Artificial Sequence
  Synthetic primer
tgcagccgtccatagcagtacccctaaaatcccaccagaatacgggtccc

SEQ ID NO: 503 - DNA, 50 bp, Artificial Sequence
  Synthetic primer
ccccaagacctaagggttttatctcctccccttgaatatgggtggctctg

SEQ ID NO: 504 - DNA, 50 bp, Artificial Sequence
  Synthetic primer
ccttgtagcaggtcccctgtcttgtgtgctgtgtccccccgaggtatgct

SEQ ID NO: 505 - DNA, 50 bp, Artificial Sequence
  Synthetic primer
gcctcagtgtccagcactgtgccctgcacatggaaagcccctacgtttgt

SEQ ID NO: 506 - DNA, 50 bp, Artificial Sequence
  Synthetic primer
caaatgtgccttctcctcccaggaaccgtacttcagctactctggtgcct

SEQ ID NO: 507 - DNA, 50 bp, Artificial Sequence
  Synthetic primer
caagcctttcagagccagagtgggtatagatgtgccagccaggagatggc

SEQ ID NO: 508 - DNA, 50 bp, Artificial Sequence
  Synthetic primer
gagagctcttgaccagtgaatcaccagccgatacgaacgtcttgccaaca

SEQ ID NO: 509 - DNA, 50 bp, Artificial Sequence
  Synthetic primer
tccctgatatacaccatccccaattgctccttctcacctcctctcaggcc

SEQ ID NO: 510 - DNA, 50 bp, Artificial Sequence
  Synthetic primer
agctctgaagacacagcacccagccttctcgcaccagccaagccttaact

SEQ ID NO: 511 - DNA, 50 bp, Artificial Sequence
  Synthetic primer
tcttcgggtcctttttcctctcttcagcgtggggcgcccacaatttgcgc

SEQ ID NO: 512 - DNA, 50 bp, Artificial Sequence
  Synthetic primer
ccgggtagtggagatgctggtgtctgggtagtcatggatttctgctggac

SEQ ID NO: 513 - DNA, 50 bp, Artificial Sequence
  Synthetic primer
cacttaccacctcatttgcttttctggaggccatgcaatataggcgggac

SEQ ID NO: 514 - DNA, 50 bp, Artificial Sequence
  Synthetic primer
ctctggtctgagtcaactttcgctggggaaaacctatggaacctactgca

SEQ ID NO: 515 - DNA, 50 bp, Artificial Sequence
  Synthetic primer
gctggtgaaaccccgaagatcaacacgcttcaaactcagccccttggaac

SEQ ID NO: 516 - DNA, 50 bp, Artificial Sequence
  Synthetic primer
gttctgacctgtcatcgggggaccaccttgaagaagcaggaaaacttgag

SEQ ID NO: 517 - DNA, 50 bp, Artificial Sequence
  Synthetic primer
agccttgcatgtgcagaaagtaaaagccagggtaggcttgtaacctgccc

SEQ ID NO: 518 - DNA, 50 bp, Artificial Sequence
  Synthetic primer
ccatcattagcagttgtcccgttgtccgaatcgcactgtatttccaccct

SEQ ID NO: 519 - DNA, 50 bp, Artificial Sequence
  Synthetic primer
cccaggacttttatcctcagcaaagggcgtgactggcttgcggtgcttgt

SEQ ID NO: 520 - DNA, 50 bp, Artificial Sequence
  Synthetic primer
gaagttctaaccccaggtacccatgaatgtgagcttaccagggtctttgc

SEQ ID NO: 521 - DNA, 50 bp, Artificial Sequence
  Synthetic primer
ctgttataggaattaacagtctttattgggctcagaccaggagtccgtgg

SEQ ID NO: 522 - DNA, 50 bp, Artificial Sequence
  Synthetic primer
ctccctgttgtgttctgtaccagtgcatgaaacccctgtcacatatcccc

SEQ ID NO: 523 - DNA, 50 bp, Artificial Sequence
  Synthetic primer
ttccctctaccactgggcactgagacgaagctccctgacagccgagactg

SEQ ID NO: 524 - DNA, 50 bp, Artificial Sequence
  Synthetic primer
ctgcactctggatcagtttttgctggcgtcgttggagttaaaatgccccg

SEQ ID NO: 525 - DNA, 50 bp, Artificial Sequence
  Synthetic primer
acccctgccctctagttggttctgggctttgatctcttccaacctgccca

SEQ ID NO: 526 - DNA, 50 bp, Artificial Sequence
  Synthetic primer
tggtgccggcctcacgagactccccatagaagaccacaagaggtttcatc

SEQ ID NO: 527 - DNA, 50 bp, Artificial Sequence
  Synthetic primer
ggtgccggcctcacgagactccccatagaagaccacaagaggtttcatcc

SEQ ID NO: 528 - DNA, 50 bp, Artificial Sequence
  Synthetic primer
ccgtgacatcctgaaccctggatagaaagcctgagcccattggatctgtg

SEQ ID NO: 529 - DNA, 50 bp, Artificial Sequence
  Synthetic primer
ggcagctctgtgtctgagaagttctacattgaccaggcccccttgttgcc

SEQ ID NO: 530 - DNA, 50 bp, Artificial Sequence
  Synthetic primer
cagtggctgcaggcgcagaacagagcgttacttgataacagcgttccatc

SEQ ID NO: 531 - DNA, 50 bp, Artificial Sequence
  Synthetic primer
ggattttggactggctcggcacacagatgatgaaatgacaggctacgtgg

SEQ ID NO: 532 - DNA, 50 bp, Artificial Sequence
  Synthetic primer
gccttccacactcacctctaaaacagtccttcaaacttccagtgcaacac

SEQ ID NO: 533 - DNA, 50 bp, Artificial Sequence
  Synthetic primer
atcggtgtctgctgctgtgtccccaggtcttgtgtgggtggcacagatct

SEQ ID NO: 534 - DNA, 50 bp, Artificial Sequence
  Synthetic primer
ttggcgcagcggtgtgcatcagaggcgtgtgctgagaagggtggtgttaa

SEQ ID NO: 535 - DNA, 50 bp, Artificial Sequence
  Synthetic primer
atgtagatggggagtgaccaccgccaacagaagtgtggccatcttgcccg

SEQ ID NO: 536 - DNA, 50 bp, Artificial Sequence
  Synthetic primer
accatactgccttgaattactgtagctgcatagatgctccttaagttggg

SEQ ID NO: 537 - DNA, 50 bp, Artificial Sequence
  Synthetic primer
cactgtcctgcccagtgctcagaggtgtggatcttgtgtgaagctcactg

SEQ ID NO: 538 - DNA, 50 bp, Artificial Sequence
  Synthetic primer
gcgcctttctcatcagcttcttccgagggtgacaggtgaaagacccctac

SEQ ID NO: 539 - DNA, 50 bp, Artificial Sequence
  Synthetic primer
gcatccatatgtgtgaggagttaagtctgtttctgactccaacagggacc

SEQ ID NO: 540 - DNA, 50 bp, Artificial Sequence
  Synthetic primer
agatgtggccattaaggagcccctagtggatgtcgtggaccccaaacagc

SEQ ID NO: 541 - DNA, 50 bp, Artificial Sequence
  Synthetic primer
gatccagccattactaacctattccttttttggggaaatctgagcctagc

SEQ ID NO: 542 - DNA, 50 bp, Artificial Sequence
  Synthetic primer
gctgcctctctccagaaatgaactgtgatggtggacacagctatgtgagg

SEQ ID NO: 543 - DNA, 50 bp, Artificial Sequence
  Synthetic primer
gaacccgcgtgcaacctgtcccgactctagccgcctcttcagcacgccat

SEQ ID NO: 544 - DNA, 50 bp, Artificial Sequence
  Synthetic primer
ttcaggtgggaacatccagttgcaggaaaacaagcttaacacgccccctg

SEQ ID NO: 545 - DNA, 50 bp, Artificial Sequence
  Synthetic primer
cacaaggagatgcgtgggctggcatgtgcaggccaaaagagccatggcct

SEQ ID NO: 546 - DNA, 50 bp, Artificial Sequence
  Synthetic primer
aaagaagttctgcaagaaggactatgcggtgcaggtggcggtgggtgcgc

SEQ ID NO: 547 - DNA, 50 bp, Artificial Sequence
  Synthetic primer
ggaggctgagagcccaaactgctgtcccaaacatgcacttccttgcttaa

SEQ ID NO: 548 - DNA, 50 bp, Artificial Sequence
  Synthetic primer
ggcagacctgactctcttaaggaggctgagagcccaaactgctgtcccaa

SEQ ID NO: 549 - DNA, 50 bp, Artificial Sequence
  Synthetic primer
ggggcatctgcctggagttaccttccatcatggctacctgctgtggtttg

SEQ ID NO: 550 - DNA, 50 bp, Artificial Sequence
  Synthetic primer
caggctgtttcccaatgtcaagttattagggtagactccaggaggtggga

SEQ ID NO: 551 - DNA, 50 bp, Artificial Sequence
  Synthetic primer
ctgccatagttacctggattgtcagccttggtagcctttgtctaaagtcc

SEQ ID NO: 552 - DNA, 50 bp, Artificial Sequence
  Synthetic primer
tggctagaatatggcagtaactacaaggcatgttctgctctggcacgaag

SEQ ID NO: 553 - DNA, 50 bp, Artificial Sequence
  Synthetic primer
agcctacaggaggaaaagcaaggcttacaagaaggatgttccttcagatg

SEQ ID NO: 554 - DNA, 50 bp, Artificial Sequence
  Synthetic primer
tctcatcagtccctctcctggagctgggggtccacacatctcccagccaa

SEQ ID NO: 555 - DNA, 50 bp, Artificial Sequence
  Synthetic primer
cctaggcaatccgccccaagcatcctttctgctttgtctccaggagtctc

SEQ ID NO: 556 - DNA, 50 bp, Artificial Sequence
  Synthetic primer
tccctggttctgttccattttgagcgaaattggccttgggaaaaaccacg

SEQ ID NO: 557 - DNA, 50 bp, Artificial Sequence
  Synthetic primer
cacgaggttggggccaggcaagttctcccaggagaaggccatgttcagtt

SEQ ID NO: 558 - DNA, 50 bp, Artificial Sequence
  Synthetic primer
caggccaccgacaagaactcactactgggcatggagggtgccaacagcat

SEQ ID NO: 559 - DNA, 50 bp, Artificial Sequence
  Synthetic primer
ctggacggctccctcatctcacggcgggcagtttatgtgtgacctggaca

SEQ ID NO: 560 - DNA, 50 bp, Artificial Sequence
  Synthetic primer
ccggcggaaatgaatgctgataccctagtagtccccagctcccaaacact

SEQ ID NO: 561 - DNA, 50 bp, Artificial Sequence
  Synthetic primer
cggagtccccgccgaagaacccgaggagccatatattgaagaccatgtct

SEQ ID NO: 562 - DNA, 50 bp, Artificial Sequence
  Synthetic primer
aggggcgcgtttgtgtctcgggttaaaataaggttccgtccgcgtgctgg

SEQ ID NO: 563 - DNA, 50 bp, Artificial Sequence
  Synthetic primer
gatgcacagaggacacagaaggacttggcagcagggtgatgacctgatca

SEQ ID NO: 564 - DNA, 50 bp, Artificial Sequence
  Synthetic primer
ggaagtgcccaccctcaaaccactctctgaagaccaggcccgtttctact

SEQ ID NO: 565 - DNA, 50 bp, Artificial Sequence
  Synthetic primer
gtgagagtgtcttcacggcaggacaacgaggaagccctaagacgtccctt

SEQ ID NO: 566 - DNA, 50 bp, Artificial Sequence
  Synthetic primer
cagagacagcggcttgcagtttcctcaagaggagagaacttagtccacca

SEQ ID NO: 567 - DNA, 50 bp, Artificial Sequence
  Synthetic primer
cccccacccccgaaaatgttcaataatgtcccatgtaaaacctgctacaa

SEQ ID NO: 568 - DNA, 50 bp, Artificial Sequence
  Synthetic primer
cggctttcggcttggaggaggccaaggtgcaactttctttggtcattccg

SEQ ID NO: 569 - DNA, 50 bp, Artificial Sequence
  Synthetic primer
tcccggtggcctctgcaggcccaagtcatcctcaggtcagcctggaagtg

SEQ ID NO: 570 - DNA, 50 bp, Artificial Sequence
  Synthetic primer
caccatcatcccagtagctgccctattcaactgcaacagtctccaggacc

SEQ ID NO: 571 - DNA, 50 bp, Artificial Sequence
  Synthetic primer
gcagcctccatgggtcagcgtgttccaagaggaaaccgtaaccttgcact

SEQ ID NO: 572 - DNA, 50 bp, Artificial Sequence
  Synthetic primer
gaattctgacttacttaaccaggtttgggatggagatggtctgcatcagc

SEQ ID NO: 573 - DNA, 50 bp, Artificial Sequence
  Synthetic primer
tggacgaggactaaatgtgggtgcccgagcttgggacaagggagaggtct

SEQ ID NO: 574 - DNA, 50 bp, Artificial Sequence
  Synthetic primer
gcatcagcgagtgcgacagtgttggcgtggatgttctttcgatggtactg

SEQ ID NO: 575 - DNA, 50 bp, Artificial Sequence
  Synthetic primer
tgggtccagttgattgtacgtagccacaggagccctgctatgaaaggaat

SEQ ID NO: 576 - DNA, 50 bp, Artificial Sequence
  Synthetic primer
cattgggcactttcccatctccaactcggaagtaagcccaagagaacaac

SEQ ID NO: 577 - DNA, 50 bp, Artificial Sequence
  Synthetic primer
cggcaagtcagtgtatcgatttctagtgctgtgaagtcagcccctgaaga

SEQ ID NO: 578 - DNA, 50 bp, Artificial Sequence
  Synthetic primer
ctcagatgatgccagctgtctcccacgtgtgtattatggttcaccagggg

SEQ ID NO: 579 - DNA, 50 bp, Artificial Sequence
  Synthetic primer
acggcgttcctggctctcctgcccacaggatgaacattttcggcttcctt

SEQ ID NO: 580 - DNA, 50 bp, Artificial Sequence
  Synthetic primer
tgtagactctgggactatcagaagagaactgacccatacctgtctagccc

SEQ ID NO: 581 - DNA, 50 bp, Artificial Sequence
  Synthetic primer
agaagtgtggcaagcaccaaccccacaaaacggcacaataccaggactcc

SEQ ID NO: 582 - DNA, 50 bp, Artificial Sequence
  Synthetic primer
caagcctcacttttctgtgccttcctgagggggttgggccggggaggaaa

SEQ ID NO: 583 - DNA, 50 bp, Artificial Sequence
  Synthetic primer
ctgtagattggtttccatacagggaagttctccgtcctatgcaatgtttc

SEQ ID NO: 584 - DNA, 50 bp, Artificial Sequence
  Synthetic primer
tgcagggcttggtcagctgcctgttcagtgtcaacacttccctgcacatc

SEQ ID NO: 585 - DNA, 50 bp, Artificial Sequence
  Synthetic primer
gggtttgagttcataccctgttaccattttggggtacccactgctctggt

SEQ ID NO: 586 - DNA, 50 bp, Artificial Sequence
  Synthetic primer
cagatctcaaggttgatagtggtgttcactaggagacgtggaattgagac

SEQ ID NO: 587 - DNA, 50 bp, Artificial Sequence
  Synthetic primer
tgcccagggcaggtgcttatctgaccttaacagtgctctcatcatggtgg

SEQ ID NO: 588 - DNA, 50 bp, Artificial Sequence
  Synthetic primer
cttgcttcagttgagacctacgttttggccagtcccagcaggaagatatc

SEQ ID NO: 589 - DNA, 50 bp, Artificial Sequence
  Synthetic primer
cgtcttcaaggacccagttgctgatcccaacaaaaggtccaaaaagggcc

SEQ ID NO: 590 - DNA, 50 bp, Artificial Sequence
  Synthetic primer
gggccccttgatctgtaaaggtgtcttccacgctatagtctctggaggtc

SEQ ID NO: 591 - DNA, 50 bp, Artificial Sequence
  Synthetic primer
gagcagccagatgataaagcacagatgcccagcactgaaagagttacttc

SEQ ID NO: 592 - DNA, 50 bp, Artificial Sequence
  Synthetic primer
gctgggactctcagcaggtccaacatctggaaatcaccttactacacaag

SEQ ID NO: 593 - DNA, 50 bp, Artificial Sequence
  Synthetic primer
tcgcctgaggagcacaggaagaaagaagaagcgctgcaggttgcctggtg

SEQ ID NO: 594 - DNA, 50 bp, Artificial Sequence
  Synthetic primer
ccgggacttgtggggtccttctcctgtcactggttatcaccctttactgc

SEQ ID NO: 595 - DNA, 50 bp, Artificial Sequence
  Synthetic primer
tgtccggcaccagcagcccttttcacccggcctcaccaatgcagatgctg

SEQ ID NO: 596 - DNA, 50 bp, Artificial Sequence
  Synthetic primer
ggcagctgtagatcttgatcttccaggtaccccatgtacctttattgagc

SEQ ID NO: 597 - DNA, 50 bp, Artificial Sequence
  Synthetic primer
ggaagtcgttcctcaactctccccaaggcacctgtctccaatcagagccc

SEQ ID NO: 598 - DNA, 50 bp, Artificial Sequence
  Synthetic primer
cagcgttaatcctgtatggccaggaaactgagtagactcctgtgtaaccc

SEQ ID NO: 599 - DNA, 50 bp, Artificial Sequence
  Synthetic primer
cgtgacttcagcttcacgattgtcgacccggtgacttccccagtgctgaa

SEQ ID NO: 600 - DNA, 50 bp, Artificial Sequence
  Synthetic primer
agaatccgtttcctggccgtagaggtggcctgcactcctcagcttgtgct

SEQ ID NO: 601 - DNA, 50 bp, Artificial Sequence
  Synthetic primer
gccggtgactggacatatcacctctacttaaatccgtcctgtttagcgac

SEQ ID NO: 602 - DNA, 50 bp, Artificial Sequence
  Synthetic primer
tgctaatgctagaccagtatttaagggctaatctcacacctccttagctg

SEQ ID NO: 603 - DNA, 50 bp, Artificial Sequence
  Synthetic primer
aacccccacccttctgcctcgtgctaaacgcacacagtatctagtcaggg

SEQ ID NO: 604 - DNA, 50 bp, Artificial Sequence
  Synthetic primer
cacgagtgtgaacctctctcccctgcgtcgccacctctgtaattgatgta

SEQ ID NO: 605 - DNA, 50 bp, Artificial Sequence
  Synthetic primer
cgatatgatagagaatcattccggcattacctaacctcttctgcagttgg

SEQ ID NO: 606 - DNA, 50 bp, Artificial Sequence
  Synthetic primer
acattgttaggggaactcaggctagtgtcagcgtcttggtttggggagaa

SEQ ID NO: 607 - DNA, 50 bp, Artificial Sequence
  Synthetic primer
agactcctccagaccaggaaccccagaaggagacagagcctgccacatcc

SEQ ID NO: 608 - DNA, 50 bp, Artificial Sequence
  Synthetic primer
ccggggatgtctggatgctgacaagggagagccaagtactgttaatgcca

SEQ ID NO: 609 - DNA, 50 bp, Artificial Sequence
  Synthetic primer
ctttgggcaccatttggatatagttagtggtggtttagctatggcgttcc

SEQ ID NO: 610 - DNA, 50 bp, Artificial Sequence
  Synthetic primer
gtagacttggacgatgcgagccttccggtcagtgacgtgtgaggcagaag

SEQ ID NO: 611 - DNA, 50 bp, Artificial Sequence
  Synthetic primer
aggacacagaggaaaggctgaaacaacgggaagaggttttgaggaaaatc

SEQ ID NO: 612 - DNA, 50 bp, Artificial Sequence
  Synthetic primer
cacccttcatcagaaatgggaccgcaagtggtaggcagtgtcccttctgc

SEQ ID NO: 613 - DNA, 50 bp, Artificial Sequence
  Synthetic primer
ctcagatcgagggtggatgataccatttcctgaccccgttttccagcatg

SEQ ID NO: 614 - DNA, 50 bp, Artificial Sequence
  Synthetic primer
gaactcagaacttgcctccatggttgagtaacaagctgtacaagaacccc

SEQ ID NO: 615 - DNA, 50 bp, Artificial Sequence
  Synthetic primer
gactgagaacagtctgaggcctggctccacttggaagtatctggggtgcg

SEQ ID NO: 616 - DNA, 50 bp, Artificial Sequence
  Synthetic primer
gcatgggaagtatgagaaggtgcttgtggaagggggcccggctccctagg

SEQ ID NO: 617 - DNA, 50 bp, Artificial Sequence
  Synthetic primer
gggccccagagctagttgctgtctggtgtttgctggtgaaaacatgagca

SEQ ID NO: 618 - DNA, 50 bp, Artificial Sequence
  Synthetic primer
actgctctttaagaggggacaagaaattggggggacccgaggccttccag

SEQ ID NO: 619 - DNA, 50 bp, Artificial Sequence
  Synthetic primer
agtggggtgcctccgtgccgcttcgtgcagcagcaagttcagcctgagat

SEQ ID NO: 620 - DNA, 50 bp, Artificial Sequence
  Synthetic primer
cggcatcatgagctcattgcacttaatacctgcaatgtttgctactgtac

SEQ ID NO: 621 - DNA, 50 bp, Artificial Sequence
  Synthetic primer
aagaggctgcggtgagccaatttagagcccaaagagccccgagggaacct

SEQ ID NO: 622 - DNA, 50 bp, Artificial Sequence
  Synthetic primer
gggattggtacttttggaaatcaggttggatttgtgaagctggcagaagg

SEQ ID NO: 623 - DNA, 50 bp, Artificial Sequence
  Synthetic primer
ctcagattcctggacctggtgtcctggttgggtccaaggtgattttacag

SEQ ID NO: 624 - DNA, 50 bp, Artificial Sequence
  Synthetic primer
ggtggctgctagagctttggcccctgcttaggattaggtggtcctcacag

SEQ ID NO: 625 - DNA, 50 bp, Artificial Sequence
  Synthetic primer
ctgtgcctttcatcacattcctagcccactctcatcattactgcagaagg

SEQ ID NO: 626 - DNA, 50 bp, Artificial Sequence
  Synthetic primer
gggtttacagccagatcacccggcttgaattgcttacagtgtgtagtctc

SEQ ID NO: 627 - DNA, 50 bp, Artificial Sequence
  Synthetic primer
ttggagtggtcaccagggggacagggagccccccaccaatgtatcaatgg

SEQ ID NO: 628 - DNA, 50 bp, Artificial Sequence
  Synthetic primer
cccagacaggaaggagaagaggcaggtgagcagggcttgttagattgtgg

SEQ ID NO: 629 - DNA, 50 bp, Artificial Sequence
  Synthetic primer
gcccttctgtaaagcgaatgaggatggacgcgtgggtgacctaaggcttc

SEQ ID NO: 630 - DNA, 50 bp, Artificial Sequence
  Synthetic primer
gaggcgctcagggaaagctggactactaaagtgaactggatggcccacac

SEQ ID NO: 631 - DNA, 50 bp, Artificial Sequence
  Synthetic primer
agttgaggctgtgcgccttggtgggcttcacgtcttcccctggatttgct

SEQ ID NO: 632 - DNA, 50 bp, Artificial Sequence
  Synthetic primer
gaggtgtagggaattttgcctgccttcctgcagacctgagctggctttgg

SEQ ID NO: 633 - DNA, 50 bp, Artificial Sequence
  Synthetic primer
gctggcccaagacccttagaaccctgagtgctggcaaatctcactgctcc

SEQ ID NO: 634 - DNA, 50 bp, Artificial Sequence
  Synthetic primer
tcaacgcctggaggacgccttatggagccagcatatcccagtctaaagaa

SEQ ID NO: 635 - DNA, 50 bp, Artificial Sequence
  Synthetic primer
ggaccccaccactgtgggttgcaggcccaatgcagaagagtattaagaaa

SEQ ID NO: 636 - DNA, 50 bp, Artificial Sequence
  Synthetic primer
agtaagtctctgccctaggcacatcagatcacctggggaccactccagag

SEQ ID NO: 637 - DNA, 50 bp, Artificial Sequence
  Synthetic primer
ggcaaattccgggtatgcactcaacttcggcaaaggcacctcgctgttgg

SEQ ID NO: 638 - DNA, 50 bp, Artificial Sequence
  Synthetic primer
ggcacttagacacgtgctgttccaccttccctcatgctgttccacctccc

SEQ ID NO: 639 - DNA, 50 bp, Artificial Sequence
  Synthetic primer
ctccgtacatgtgggtgtcgccatgtgtgccctgtcactatctgtggctg

SEQ ID NO: 640 - DNA, 50 bp, Artificial Sequence
  Synthetic primer
ctacatccaccactgggacaagccatcttgtaaaatgtgcggagaaggag

SEQ ID NO: 641 - DNA, 50 bp, Artificial Sequence
  Synthetic primer
gcccagttttctctggtgggtccagtgttttgttcctaggtgtctgctgc

SEQ ID NO: 642 - DNA, 50 bp, Artificial Sequence
  Synthetic primer
ctgggaacaaagggtccacacccagcaccactgagcaagtttgtgagcag

SEQ ID NO: 643 - DNA, 50 bp, Artificial Sequence
  Synthetic primer
cctggcccagctggctttcctgtcccaaatcagccagtgtataatcagcc

SEQ ID NO: 644 - DNA, 50 bp, Artificial Sequence
  Synthetic primer
tgggcagcgcgggctcgatgtgtgtcagccaagtagctggtcccactgga

SEQ ID NO: 645 - DNA, 50 bp, Artificial Sequence
  Synthetic primer
ccccgttctagggccgtggcctttgccgagactgtagcagagaaaacgta

SEQ ID NO: 646 - DNA, 50 bp, Artificial Sequence
  Synthetic primer
aaggttaggtaacacacatagggctgttacggactgagaagtgtccctgc

SEQ ID NO: 647 - DNA, 50 bp, Artificial Sequence
  Synthetic primer
ttacgcctcctgatccttccgattggggcagactaggagaggaaagacgg

SEQ ID NO: 648 - DNA, 50 bp, Artificial Sequence
  Synthetic primer
tcccacgctctcaggggggacatgctctctcttaccctgtcacttggtct

SEQ ID NO: 649 - DNA, 50 bp, Artificial Sequence
  Synthetic primer
ttctgcagccctgtctacttgggcacccccaccacctgttagctgtggtt

SEQ ID NO: 650 - DNA, 50 bp, Artificial Sequence
  Synthetic primer
gaagtggaggaggctagggggccacagttgctgcttcattaacatagagg

SEQ ID NO: 651 - DNA, 50 bp, Artificial Sequence
  Synthetic primer
cctctcaacacacagcaagcatgtccctgcctcccatgaatcttaccatg

SEQ ID NO: 652 - DNA, 50 bp, Artificial Sequence
  Synthetic primer
agcaaaaggtggcaacccatggaggcagaaagaaggacgccagcctgacc

SEQ ID NO: 653 - DNA, 50 bp, Artificial Sequence
  Synthetic primer
gatttggatcattgcccaaagttgcacgcactgcctccttacctgtgagg

SEQ ID NO: 654 - DNA, 50 bp, Artificial Sequence
  Synthetic primer
ttgcttgccttcctcccttctgtccgctcttactccctccttcttttatt

SEQ ID NO: 655 - DNA, 50 bp, Artificial Sequence
  Synthetic primer
gctctgcaccatccctcacccagaccgtagacaccagggaaccacatcta

SEQ ID NO: 656 - DNA, 50 bp, Artificial Sequence
  Synthetic primer
cctttgcacagcatccttggaggggattcgcaagtggcagccctgttgat

SEQ ID NO: 657 - DNA, 50 bp, Artificial Sequence
  Synthetic primer
cttggggttggggtaggtagataccaaatgaaacactttcaggaccttcc

SEQ ID NO: 658 - DNA, 50 bp, Artificial Sequence
  Synthetic primer
agagtcctggggagcttctgtccacctgtcctgcagaggagtcgtttcca

SEQ ID NO: 659 - DNA, 50 bp, Artificial Sequence
  Synthetic primer
caaaggagacggtggctcagggagtaaaggaaggccagttgagcaaacag

SEQ ID NO: 660 - DNA, 50 bp, Artificial Sequence
  Synthetic primer
gagggcaaagagttggagttctatcttaggaaaatcaaggcccggaaggg

SEQ ID NO: 661 - DNA, 50 bp, Artificial Sequence
  Synthetic primer
agcaagtgaggatttgcccagagttcagtcctggctggaggagccacagc

SEQ ID NO: 662 - DNA, 50 bp, Artificial Sequence
  Synthetic primer
caaggtccttcaaatgttcctttggccaaaagctgaagggagttactgag

SEQ ID NO: 663 - DNA, 50 bp, Artificial Sequence
  Synthetic primer
gtggaggaaacgcagctgcaactgaagcggagactctaaacccagcttgc

SEQ ID NO: 664 - DNA, 50 bp, Artificial Sequence
  Synthetic primer
gcctgctgcatttgcgtgatctcaagacctccagccagaagtcccttcca

SEQ ID NO: 665 - DNA, 50 bp, Artificial Sequence
  Synthetic primer
tccttttatgcattggaggaaaaacatgttggcttttctctcgacgtggg

SEQ ID NO: 666 - DNA, 50 bp, Artificial Sequence
  Synthetic primer
agaaccagttcatgctggccgaaacgacaatggtttatggacttgcatcc

SEQ ID NO: 667 - DNA, 50 bp, Artificial Sequence
  Synthetic primer
ggggtctgtgagagtacatgtattatatacaagcacaacagggcttgcac

SEQ ID NO: 668 - DNA, 50 bp, Artificial Sequence
  Synthetic primer
tcaccgctgttgcctgccgccttgcgcagttgactcgtgtctgaggaaac

SEQ ID NO: 669 - DNA, 50 bp, Artificial Sequence
  Synthetic primer
agcagtactggcagaaccagaaatgccgctggaagcacgtcaaactgctc

SEQ ID NO: 670 - DNA, 50 bp, Artificial Sequence
  Synthetic primer
aaagccttcagagggtttggaccacatctctttggaaaatagtttgcaac

SEQ ID NO: 671 - DNA, 50 bp, Artificial Sequence
  Synthetic primer
cagactgggaacagggcccaggaatctgtgtggtacaaacctgcatggtg

SEQ ID NO: 672 - DNA, 50 bp, Artificial Sequence
  Synthetic primer
gtcacattgtcagaacaggtggctgctgtgtggtgccatcgagtccctgc

SEQ ID NO: 673 - DNA, 50 bp, Artificial Sequence
  Synthetic primer
gtgacaccatttcccatgaacacccctgggaggtcatgcctcagctctgc

SEQ ID NO: 674 - DNA, 50 bp, Artificial Sequence
  Synthetic primer
gggccctcatgctggcttttcaccccagaggacacaggcagcttccaaaa

SEQ ID NO: 675 - DNA, 50 bp, Artificial Sequence
  Synthetic primer
gtgactcatgggcgaggagcatagacagtgtggagacattgggcaagggg

SEQ ID NO: 676 - DNA, 50 bp, Artificial Sequence
  Synthetic primer
gtgaccatcactggacagctttctctcaaaactttccttcaacgccatgg

SEQ ID NO: 677 - DNA, 50 bp, Artificial Sequence
  Synthetic primer
ggggaatcggcgatgaacaaagcagatagaaatccccactcttgtggagc

SEQ ID NO: 678 - DNA, 50 bp, Artificial Sequence
  Synthetic primer
gctctaggagttctgaacggaaggcagacgagaggcactttatccagtcc

SEQ ID NO: 679 - DNA, 50 bp, Artificial Sequence
  Synthetic primer
ggcgctggtacgtaaatagaccaatgcagttaggtggctctttccaagac

SEQ ID NO: 680 - DNA, 50 bp, Artificial Sequence
  Synthetic primer
gggggtgtccatgtggcgtgtgtgtgagtgagacttttttactgcgtccc

SEQ ID NO: 681 - DNA, 50 bp, Artificial Sequence
  Synthetic primer
gccaacaccaactcgtagctttagtggcagatgggagtggtcacagactc

SEQ ID NO: 682 - DNA, 50 bp, Artificial Sequence
  Synthetic primer
gatgcatgacacaaggtaagactgccaaaaatcttgttcttgctctcctc

SEQ ID NO: 683 - DNA, 50 bp, Artificial Sequence
  Synthetic primer
acaccctactgtccttgtgcctcacgccccctcctcatcctgcacccctt

SEQ ID NO: 684 - DNA, 50 bp, Artificial Sequence
  Synthetic primer
gggtaagtacgcaacttacttttccaccaaagaactgtcagcagctgcct

SEQ ID NO: 685 - DNA, 50 bp, Artificial Sequence
  Synthetic primer
ctcgtagcaggtcccctgtcttgtgtgctgtgtccccccgaggtatgctg

SEQ ID NO: 686 - DNA, 50 bp, Artificial Sequence
  Synthetic primer
ggcaagcgaccagcgtgatctcacagagcacaaaccctcagtctccaacc

SEQ ID NO: 687 - DNA, 50 bp, Artificial Sequence
  Synthetic primer
cctgcgtcacagggaagcaacctacagagaagcagcagctccccaagaga

SEQ ID NO: 688 - DNA, 50 bp, Artificial Sequence
  Synthetic primer
gcagaactagacccccgccacagcagcctctgaagttggacagcaaaacc

SEQ ID NO: 689 - DNA, 50 bp, Artificial Sequence
  Synthetic primer
tctgaggtcctggaggtgccatttccctctctgttgaagcaaaggccggg

SEQ ID NO: 690 - DNA, 50 bp, Artificial Sequence
  Synthetic primer
ctcccaccaagccagacacgttgtaaatgctgcttcctattggagcccag

SEQ ID NO: 691 - DNA, 50 bp, Artificial Sequence
  Synthetic primer
tctgggccctacagcctggagaacctggagaatcctacacccacagaacc

SEQ ID NO: 692 - DNA, 50 bp, Artificial Sequence
  Synthetic primer
ctccgtctccgctaaaaatacgaaaattagccaggcgtggtggcgagcac

SEQ ID NO: 693 - DNA, 50 bp, Artificial Sequence
  Synthetic primer
tgccccagcccaacctctgggaacaggcagctcctatctgcaaactgtgt

SEQ ID NO: 694 - DNA, 50 bp, Artificial Sequence
  Synthetic primer
cgtcagaactgcacagagaaaattccccagtattccaagccggtgcgccc

SEQ ID NO: 695 - DNA, 50 bp, Artificial Sequence
  Synthetic primer
gcctctgccacttggagcactagaggatgacggaatgtcttgtatatgcg

SEQ ID NO: 696 - DNA, 50 bp, Artificial Sequence
  Synthetic primer
ccggaatgagaagctaaagaaggttgtgaggcttgcccgggttcgagatc

SEQ ID NO: 697 - DNA, 50 bp, Artificial Sequence
  Synthetic primer
ggcccaagggttatggctttcatgtctaggtgtggggacagaggagggag

SEQ ID NO: 698 - DNA, 50 bp, Artificial Sequence
  Synthetic primer
caccacctaggccactcctccaaaggaagtctaggagctgggaggaaaag

SEQ ID NO: 699 - DNA, 50 bp, Artificial Sequence
  Synthetic primer
ggggcctgagatttctgcactttaaacaagctcctcctaggtgaggatgc

SEQ ID NO: 700 - DNA, 50 bp, Artificial Sequence
  Synthetic primer
gcctccttgttccctgtggctgctgataacccaacattccatctctaccc

SEQ ID NO: 701 - DNA, 50 bp, Artificial Sequence
  Synthetic primer
cgagaaccgtcagtttgagccagatggaagctgagctgaacacattacga

SEQ ID NO: 702 - DNA, 50 bp, Artificial Sequence
  Synthetic primer
caggatcagcattgcctggtgcaagcggttcagggtgccagtggagaaga

SEQ ID NO: 703 - DNA, 50 bp, Artificial Sequence
  Synthetic primer
atcaagaagaaactgagcaaggcctgagcgctgccctgcacctccgcaga

SEQ ID NO: 704 - DNA, 50 bp, Artificial Sequence
  Synthetic primer
gggtctggtacccttgcaggacctttgtaggttgtgggaaaaagtcgcag

SEQ ID NO: 705 - DNA, 50 bp, Artificial Sequence
  Synthetic primer
atcagcaagcctccagtggtccaaaccagccgcctgttcgccgtggatac

SEQ ID NO: 706 - DNA, 50 bp, Artificial Sequence
  Synthetic primer
cagtctagctgattgttccaccaaagaatccagcctgctgcttattcgag

SEQ ID NO: 707 - DNA, 50 bp, Artificial Sequence
  Synthetic primer
atccccgactcggaaagcaatctggatcagaactgtttgtctcgctgcag

SEQ ID NO: 708 - DNA, 50 bp, Artificial Sequence
  Synthetic primer
gccaggatccttggtgtcctaggagaaacagacaagcaaaacaaagtgaa

SEQ ID NO: 709 - DNA, 50 bp, Artificial Sequence
  Synthetic primer
gaagagtaatacgtggtcctgggggattttgaagatgttaaagggaaaag

SEQ ID NO: 710 - DNA, 50 bp, Artificial Sequence
  Synthetic primer
aaggccagtgcagccaaaagcagcctcatccaccaaggcatccgctgaag

SEQ ID NO: 711 - DNA, 50 bp, Artificial Sequence
  Synthetic primer
cctaatctctagctgccgtcttgtagatatgaacgaatgttcaccaagca

SEQ ID NO: 712 - DNA, 50 bp, Artificial Sequence
  Synthetic primer
atgagtccctgtgcagaccagtgggcaaggcagctgggccagatctcagg

SEQ ID NO: 713 - DNA, 50 bp, Artificial Sequence
  Synthetic primer
aactagcgaaccccaggggaaggtgccgtgtggagagcactttcggattc

SEQ ID NO: 714 - DNA, 50 bp, Artificial Sequence
  Synthetic primer
atgaagtataagcctctactgggtctatattgtgaatcatcctgcctttc

SEQ ID NO: 715 - DNA, 50 bp, Artificial Sequence
  Synthetic primer
cagaagagccacaggaacatcattcctctcccgcaaccactcccacccca

SEQ ID NO: 716 - DNA, 50 bp, Artificial Sequence
  Synthetic primer
ggtgctttggccctaatagcactggacaacaccacgaccacatggaaaca

SEQ ID NO: 717 - DNA, 50 bp, Artificial Sequence
  Synthetic primer
gcccagtttttcccgtccggcggcctctccgggcccagaagctcctcaag

SEQ ID NO: 718 - DNA, 50 bp, Artificial Sequence
  Synthetic primer
caacaaggtcccaaacaagattgagagccaggggacagaagtgccaccgg

SEQ ID NO: 719 - DNA, 50 bp, Artificial Sequence
  Synthetic primer
ccatccctcatgtgttctcagctggcactggctctgccatcctgcctcat

SEQ ID NO: 720 - DNA, 50 bp, Artificial Sequence
  Synthetic primer
tcccggagtggtagcgctgtctcctggtgaaggagtatttgtcacactgg

SEQ ID NO: 721 - DNA, 50 bp, Artificial Sequence
  Synthetic primer
cagcctctccaggcccagaactttctccagtcagcctctacagaccaaga

SEQ ID NO: 722 - DNA, 50 bp, Artificial Sequence
  Synthetic primer
catatccaagttcactctgtcttcctgagcagtggaagatcatattgctg

SEQ ID NO: 723 - DNA, 50 bp, Artificial Sequence
  Synthetic primer
gtaacggagtttagagccagggctgatgctttggtgtggccagcactctg

SEQ ID NO: 724 - DNA, 50 bp, Artificial Sequence
  Synthetic primer
cttcagcgcttgatattggtctaggaggggtttctgtctcttcctggttc

SEQ ID NO: 725 - DNA, 50 bp, Artificial Sequence
  Synthetic primer
acccaagtcttctcccgtccattccagtcaaatctgggctcactcacccc

SEQ ID NO: 726 - DNA, 50 bp, Artificial Sequence
  Synthetic primer
tgggcatctcctgaggaaatgtagcatgacattggtactaactgcatgtg

SEQ ID NO: 727 - DNA, 50 bp, Artificial Sequence
  Synthetic primer
ttttttgaagatcagctccgtggggctggttttggtccacagcataacag

SEQ ID NO: 728 - DNA, 50 bp, Artificial Sequence
  Synthetic primer
gcacaaagtaagcctcacctttgccttccaactgccagaacatggattcc

SEQ ID NO: 729 - DNA, 50 bp, Artificial Sequence
  Synthetic primer
aggggtccataccgtgttgttctggattcccgttgtaacttaaagggaaa

SEQ ID NO: 730 - DNA, 50 bp, Artificial Sequence
  Synthetic primer
ccgggcccagaagctcctcaagtcggcctctccagacccacttgcagcct

SEQ ID NO: 731 - DNA, 50 bp, Artificial Sequence
  Synthetic primer
agtcttgggacttgggtctgtcctgctgtgcatccatagtgggtgcttta

SEQ ID NO: 732 - DNA, 50 bp, Artificial Sequence
  Synthetic primer
ccctgcccgaggtgctgacccaattcgctgccaaaagagtgtcaatcaga

SEQ ID NO: 733 - DNA, 50 bp, Artificial Sequence
  Synthetic primer
agggagcaaccccaggatagctaagtgttatgtaatatgcctagaaggtg

SEQ ID NO: 734 - DNA, 50 bp, Artificial Sequence
  Synthetic primer
gcatctagtgataggagtagtgtggagcgagaagtggcagaacacctagc

SEQ ID NO: 735 - DNA, 50 bp, Artificial Sequence
  Synthetic primer
ctgtggttgccacctgccatctccaagcatttggacaactagccctgatg

SEQ ID NO: 736 - DNA, 50 bp, Artificial Sequence
  Synthetic primer
acccgatggaaataacaggcgggcttcacggtgcggctctgtccgcccat

SEQ ID NO: 737 - DNA, 50 bp, Artificial Sequence
  Synthetic primer
tgtaactactgcagcgccattggacctacgctgcttgaggctctggactg

SEQ ID NO: 738 - DNA, 50 bp, Artificial Sequence
  Synthetic primer
agaatctgtttcctggccgtagaggtggcctgcactcctcagcctgtgct

SEQ ID NO: 739 - DNA, 50 bp, Artificial Sequence
  Synthetic primer
ctgtcaccttcctcctcccacttctgtatcctcagagatgctgcgtggat

SEQ ID NO: 740 - DNA, 50 bp, Artificial Sequence
  Synthetic primer
ttgtcatgctccccacagagagcccaggacatttgcctgatgtatggtgc

SEQ ID NO: 741 - DNA, 50 bp, Artificial Sequence
  Synthetic primer
gtgggccagcttctgcggcaaccatgaaaccgcatccaacagtccaaaag

SEQ ID NO: 742 - DNA, 50 bp, Artificial Sequence
  Synthetic primer
cagttatggaggacttgtatggagaaatttaagtcttcactgagggccac

SEQ ID NO: 743 - DNA, 50 bp, Artificial Sequence
  Synthetic primer
gagccagggaagcaaggaacggtgtatgcgcaagtgaaagagaacccagg

SEQ ID NO: 744 - DNA, 50 bp, Artificial Sequence
  Synthetic primer
atgtcccctgcttgaaccctgagaggcaggtggtaggccatggccacaat

SEQ ID NO: 745 - DNA, 50 bp, Artificial Sequence
  Synthetic primer
gtggcttcttcatgatggtggttggggtggttgtcatgtgacgggtcccc

SEQ ID NO: 746 - DNA, 50 bp, Artificial Sequence
  Synthetic primer
gccatacctggtgtccaaagaactcatgagccttgtgtctgggctgctgc

SEQ ID NO: 747 - DNA, 50 bp, Artificial Sequence
  Synthetic primer
ccatgaacccccttaaagccaagacagcctccctctttgagcagaagtcc

SEQ ID NO: 748 - DNA, 50 bp, Artificial Sequence
  Synthetic primer
cagaaatggaggagaagtatggggaagtagaggagatgaacgtctgtgac

SEQ ID NO: 749 - DNA, 50 bp, Artificial Sequence
  Synthetic primer
gatgtattgctgtcatcagtgtggcactctgtcctcatgaggagggacag

SEQ ID NO: 750 - DNA, 50 bp, Artificial Sequence
  Synthetic primer
ctggtctcctcagcttccctatccatataggcacagagaggcagcatttg

SEQ ID NO: 751 - DNA, 50 bp, Artificial Sequence
  Synthetic primer
ggtccttgtctagcaggagacaactgcacaggtatactaccagcgtttgg

SEQ ID NO: 752 - DNA, 50 bp, Artificial Sequence
  Synthetic primer
gaaaactgagcttccggtcacagagaacgtgcagacgattcccccgccat

SEQ ID NO: 753 - DNA, 50 bp, Artificial Sequence
  Synthetic primer
ctctctgtggccgtgggcagcaccgtctattaccagggcaaatgtctgac

SEQ ID NO: 754 - DNA, 50 bp, Artificial Sequence
  Synthetic primer
gaggctttcaggtaggaggacaatggtagcactgtaggtccccagtgtcg

SEQ ID NO: 755 - DNA, 50 bp, Artificial Sequence
  Synthetic primer
ggccgtctactggaaaaatcgagcctctcccaccctcagtcgcatagact

SEQ ID NO: 756 - DNA, 50 bp, Artificial Sequence
  Synthetic primer
ggggtggatctttccatggcttctccaaagctggtgacttccgacacagc

SEQ ID NO: 757 - DNA, 50 bp, Artificial Sequence
  Synthetic primer
gatgcagcaagaaggagccatctgcaatccaggaagaaattccttgccag

SEQ ID NO: 758 - DNA, 50 bp, Artificial Sequence
  Synthetic primer
ccgggtacagatctcagcagtgcatagcgaggaagacattgaccgctgcg

SEQ ID NO: 759 - DNA, 50 bp, Artificial Sequence
  Synthetic primer
gcagcatctcctgtcccccagccatgtcattctagctgtgaactggcacg

SEQ ID NO: 760 - DNA, 50 bp, Artificial Sequence
  Synthetic primer
gtcaacttttcttcccctacttccctgcatttctcctctgtgctcactgc

SEQ ID NO: 761 - DNA, 50 bp, Artificial Sequence
  Synthetic primer
gtctgcgctgaccccagtgagtcctgggtccaggagtacgtgtatgacct

SEQ ID NO: 762 - DNA, 50 bp, Artificial Sequence
  Synthetic primer
ctgcctaccgcacatccccacggctgaaggacaagaagatggatgcccat

SEQ ID NO: 763 - DNA, 50 bp, Artificial Sequence
  Synthetic primer
gcgggccgccggtgaaataccactactctgatcgttttttcactgacccg

SEQ ID NO: 764 - DNA, 50 bp, Artificial Sequence
  Synthetic primer
cacccactaatagggaacgtgagctgggtttagaccgtcgtgagacaggt

SEQ ID NO: 765 - DNA, 50 bp, Artificial Sequence
  Synthetic primer
gagagctattggagtgagagctgaggtgtccttaaccactttttctctcc

SEQ ID NO: 766 - DNA, 50 bp, Artificial Sequence
  Synthetic primer
gctacgcctagacctgctggaaaggttccacaccatgtccatcatgctgg

SEQ ID NO: 767 - DNA, 50 bp, Artificial Sequence
  Synthetic primer
aactcaagttgggagtgcagagggaatggggagaaaaccctcccactgcc

SEQ ID NO: 768 - DNA, 50 bp, Artificial Sequence
  Synthetic primer
agcagcacttgcccattccttacaccccttccccatcctgctccgcttca

SEQ ID NO: 769 - DNA, 50 bp, Artificial Sequence
  Synthetic primer
tgtcaggacgcggaaagcagggaggcaaggcccgcgctaaggccaagtcg

SEQ ID NO: 770 - DNA, 50 bp, Artificial Sequence
  Synthetic primer
gagttcatttacaagcaccagcagaagaagggcgagtacgtcaaccgctg

SEQ ID NO: 771 - DNA, 50 bp, Artificial Sequence
  Synthetic primer
tttgcctatgatgccttcaagatctaccggactgagatggcacccggggc

SEQ ID NO: 772 - DNA, 50 bp, Artificial Sequence
  Synthetic primer
ggcctgtgtctgcctccttgggactgtggagtgggtgccctcagtaaagc

SEQ ID NO: 773 - DNA, 50 bp, Artificial Sequence
  Synthetic primer
gagctctcatgttctgcacctcaggagggaattcagcctcagtgatgtcc

SEQ ID NO: 774 - DNA, 50 bp, Artificial Sequence
  Synthetic primer
ctctcagagaaagtagctttgtgacatgtcatgaacccatgtttgcaatc

SEQ ID NO: 775 - DNA, 50 bp, Artificial Sequence
  Synthetic primer
gatcactgtccttgccttcctggttctttctctcggttggttgccagggc

SEQ ID NO: 776 - DNA, 50 bp, Artificial Sequence
  Synthetic primer
cgctggaaccttgggcaaggaagaatgtgagcaagagtagagagagtgcc

SEQ ID NO: 777 - DNA, 50 bp, Artificial Sequence
  Synthetic primer
ctccagcccacgttctctctgcctgtgagccagtctagttcctgatgacc

SEQ ID NO: 778 - DNA, 50 bp, Artificial Sequence
  Synthetic primer
acgaagtccgggaggatgatgtcggctgggactgggaccatctgttcact

SEQ ID NO: 779 - DNA, 50 bp, Artificial Sequence
  Synthetic primer
ccatgagtcttgcttttggctcatacaggagagagggaaggctgccaggc

SEQ ID NO: 780 - DNA, 50 bp, Artificial Sequence
  Synthetic primer
cgtgtatccccacacaaatgcaagctcaccaaggtcccctctcagtcccc

SEQ ID NO: 781 - DNA, 50 bp, Artificial Sequence
  Synthetic primer
aagaaccccacagacgaatacctggagggcatgatgagcgaggccccggg

SEQ ID NO: 782 - DNA, 50 bp, Artificial Sequence
  Synthetic primer
actagagctaaggaggatagcttgagcttctcactgtgccagggccagcc

SEQ ID NO: 783 - DNA, 50 bp, Artificial Sequence
  Synthetic primer
atgaaggcccaggctacagctgcagcttcagttccagttcgggctcccag

SEQ ID NO: 784 - DNA, 50 bp, Artificial Sequence
  Synthetic primer
tctggatcagtttgtgctgcagtcgcacgcatggttcaacgtgtcctccc

SEQ ID NO: 785 - DNA, 50 bp, Artificial Sequence
  Synthetic primer
gggaagagagtttgcaggcctgtgcttcaaagaactcttctagtctgctt

SEQ ID NO: 786 - DNA, 50 bp, Artificial Sequence
  Synthetic primer
tcaagtgaacagcacgtccaggaggcgacctcgtccgcgggtttgcattc

SEQ ID NO: 787 - DNA, 50 bp, Artificial Sequence
  Synthetic primer
actgtttccggccctggaggagaatgagggaggagagggttttctatgac

SEQ ID NO: 788 - DNA, 50 bp, Artificial Sequence
  Synthetic primer
accggtcatgcccatccggaatgtctataagaaggagaaagctcgagtga

SEQ ID NO: 789 - DNA, 50 bp, Artificial Sequence
  Synthetic primer
aactcggtggctgcctcgctgatgagctggtctgcgatcctgaatggggg

SEQ ID NO: 790 - DNA, 50 bp, Artificial Sequence
  Synthetic primer
agagagacttcctgaagcagcgccccaccaagctcaagagcctcatccgc

SEQ ID NO: 791 - DNA, 50 bp, Artificial Sequence
  Synthetic primer
aaagggttggaggcagctggcacaagaggctgaggcctggctgaattacc

SEQ ID NO: 792 - DNA, 50 bp, Artificial Sequence
  Synthetic primer
ctgccagttctgctcaccaccatccgtgcttggcacagaagtagctgcat

SEQ ID NO: 793 - DNA, 50 bp, Artificial Sequence
  Synthetic primer
ccaccgggatctgaactcgcacaactgcctcatcaagttggacaagactg

SEQ ID NO: 794 - DNA, 50 bp, Artificial Sequence
  Synthetic primer
ccgggatctgaactcgcacaactgcctcatcaagttggacaagactgtgg

SEQ ID NO: 795 - DNA, 50 bp, Artificial Sequence
  Synthetic primer
aggctgtgcaagtgtcattatatcgaggatgactgtttgctgagacttag

SEQ ID NO: 796 - DNA, 50 bp, Artificial Sequence
  Synthetic primer
gcgcaacacctaggagcccaaaaataagcagcacgacggaactttcagcc

SEQ ID NO: 797 - DNA, 50 bp, Artificial Sequence
  Synthetic primer
gttgtcagcttctcccccttcagcctgtttctgcagcagccagggttctc

SEQ ID NO: 798 - DNA, 50 bp, Artificial Sequence
  Synthetic primer
aggacgcaccctcactcccctgctgccttcactcctttctgaccaagcaa

SEQ ID NO: 799 - DNA, 50 bp, Artificial Sequence
  Synthetic primer
cccatttcctgagaccaccagagagaggggagaagcctgggattgacaga

SEQ ID NO: 800 - DNA, 50 bp, Artificial Sequence
  Synthetic primer
agtaaacccatatatccagaaccctgaccctgccgtgtaccagctgagag

SEQ ID NO: 801 - DNA, 50 bp, Artificial Sequence
  Synthetic primer
gtcttccttgaccaccatcattcctgcctgattagagggcttcctcatgg

SEQ ID NO: 802 - DNA, 50 bp, Artificial Sequence
  Synthetic primer
gccagaaacaagccctgtggtttgaaggtgagctgtgaggatgggactaa

SEQ ID NO: 803 - DNA, 50 bp, Artificial Sequence
  Synthetic primer
ccggcctggtttcgcctggtcgtgtctttatgagcaccatgtaagcctcc

SEQ ID NO: 804 - DNA, 50 bp, Artificial Sequence
  Synthetic primer
ctaggtgagcgcattgagccagtggtgctaaatgctacatactccaactg

SEQ ID NO: 805 - DNA, 50 bp, Artificial Sequence
  Synthetic primer
ccggccctgcgggccgccggtgaaataccactactctgatcgttttttca

SEQ ID NO: 806 - DNA, 50 bp, Artificial Sequence
  Synthetic primer
ccccagccctagccctttagcctttcaccctgtgctctggaaaggctacc

SEQ ID NO: 807 - DNA, 50 bp, Artificial Sequence
  Synthetic primer
caaggtcaggaggagcaaaaatgagccagcaccagcgccttggctttgtg

SEQ ID NO: 808 - DNA, 50 bp, Artificial Sequence
  Synthetic primer
ggctcctgttgctccttcacacagacctggatgccccagagcaaggtctt

SEQ ID NO: 809 - DNA, 50 bp, Artificial Sequence
  Synthetic primer
tctatgagtatcgccgggcccaggaggccgtgtgaggatcccgcaataaa

SEQ ID NO: 810 - DNA, 50 bp, Artificial Sequence
  Synthetic primer
ggctcggcggaatacctcttagacaagcacaccctgggggacagtgataa

SEQ ID NO: 811 - DNA, 50 bp, Artificial Sequence
  Synthetic primer
ctgagaatgtgcaggaggggttagtgaaggggaattaagggctagagaag

SEQ ID NO: 812 - DNA, 50 bp, Artificial Sequence
  Synthetic primer
tcacttgccgggagagactttttacaactcatctgcagctccgggtgcgg

SEQ ID NO: 813 - DNA, 50 bp, Artificial Sequence
  Synthetic primer
agtagctccacctgaagagggatggaacctctgggtcaggaaacagctgg

SEQ ID NO: 814 - DNA, 50 bp, Artificial Sequence
  Synthetic primer
tgcgggccgcgccaggcaccaactcagtgtttgtcagtgtttgtttttcc

SEQ ID NO: 815 - DNA, 50 bp, Artificial Sequence
  Synthetic primer
gacccagaccccacaagccatggagactcaggaccccagaaggcatggaa

SEQ ID NO: 816 - DNA, 50 bp, Artificial Sequence
  Synthetic primer
ggccctgcacaggtggctgtttcttgtctgttatgtcagaggaacagtcc

SEQ ID NO: 817 - DNA, 50 bp, Artificial Sequence
  Synthetic primer
ggggtctgaccaggcaagctgtgatctgaaaggagcaaggaacagcaagg

SEQ ID NO: 818 - DNA, 50 bp, Artificial Sequence
  Synthetic primer
ctcctggtgctccctctgggtccccctggttgttgtagcagcttaactgt

SEQ ID NO: 819 - DNA, 50 bp, Artificial Sequence
  Synthetic primer
acaaagttgttgagccttgcttcttccgttttgccctttgtctcgctcct

SEQ ID NO: 820 - DNA, 50 bp, Artificial Sequence
  Synthetic primer
cccaggtctcatccttcaggaattttgcagttcaatgagaagagggagac

SEQ ID NO: 821 - DNA, 50 bp, Artificial Sequence
  Synthetic primer
gttttcatcgccttggtaaagctccagaaatggacaatgctagtgactgc

SEQ ID NO: 822 - DNA, 50 bp, Artificial Sequence
  Synthetic primer
ccaagaagtccttagaaatagtggcagggaacaggtgtggaagctcatgc

SEQ ID NO: 823 - DNA, 50 bp, Artificial Sequence
  Synthetic primer
gcccattcaaaatgatctttaccgtggcctgccccatgcttatggtcccc

SEQ ID NO: 824 - DNA, 50 bp, Artificial Sequence
  Synthetic primer
ggactctgtcctgtgtcacctctctccaggtgtccagctgtctcatgcct

SEQ ID NO: 825 - DNA, 50 bp, Artificial Sequence
  Synthetic primer
ctgaaaccagtggcagcccaagttagagcccagcattaagtccacctcat

SEQ ID NO: 826 - DNA, 50 bp, Artificial Sequence
  Synthetic primer
tgattttggggtagcaatccaggagaaggtgctggagagggtgaatgccg

SEQ ID NO: 827 - DNA, 50 bp, Artificial Sequence
  Synthetic primer
caagttcttaaccatcccgggttccagtggttacagagttctgccctggg

SEQ ID NO: 828 - DNA, 50 bp, Artificial Sequence
  Synthetic primer
cctggagcttcagtgccttgttcatggtggccaagtgaattgggatatgg

SEQ ID NO: 829 - DNA, 50 bp, Artificial Sequence
  Synthetic primer
gtatgacaccttattctcactcgcctcggacagattccagttacacgggg

SEQ ID NO: 830 - DNA, 50 bp, Artificial Sequence
  Synthetic primer
cctcatggctctggcacagtgtaatccaggggtgtagatgggggaactgt

SEQ ID NO: 831 - DNA, 50 bp, Artificial Sequence
  Synthetic primer
tgtggtttaatggtcacaaaaatgttagcactgctaccactcagcacgtg

SEQ ID NO: 832 - DNA, 50 bp, Artificial Sequence
  Synthetic primer
actgctgcgtcattacagggcacaggccatggatggaaaacgctctctgc

SEQ ID NO: 833 - DNA, 50 bp, Artificial Sequence
  Synthetic primer
ggaaatgggagtgctcagtctgtgcaagtcagaatccttgaaactgggcc

SEQ ID NO: 834 - DNA, 50 bp, Artificial Sequence
  Synthetic primer
gtagcactgttctggttctgtttgcacgccagtggggagagaataaagag

SEQ ID NO: 835 - DNA, 50 bp, Artificial Sequence
  Synthetic primer
gtccaagctgtgcctcgacacatcctcatccccagcatgggacacctcaa

SEQ ID NO: 836 - DNA, 50 bp, Artificial Sequence
  Synthetic primer
acctgaagcagcaagtgagcgggctggagggtgtgcaggacgacctgttc

SEQ ID NO: 837 - DNA, 50 bp, Artificial Sequence
  Synthetic primer
cctgtggcaagccagcaagatggccctggtgacagcaaaagaaactgcac

SEQ ID NO: 838 - DNA, 50 bp, Artificial Sequence
  Synthetic primer
ctacatggctgcggtcctcgagtatctgaccgccgagatcctggagctgg

SEQ ID NO: 839 - DNA, 50 bp, Artificial Sequence
  Synthetic primer
catatacgtgtgccgggtccaggagggcaacgagtcataccagcagtcct

SEQ ID NO: 840 - DNA, 50 bp, Artificial Sequence
  Synthetic primer
gtgcctggtccaatgtgctgagaggcatgggcggtgcttttgtattggtg

SEQ ID NO: 841 - DNA, 50 bp, Artificial Sequence
  Synthetic primer
gaaccttttccaagttgatctatccagtgacgtggcctggtgggcgtttc

SEQ ID NO: 842 - DNA, 50 bp, Artificial Sequence
  Synthetic primer
acttgtggacatcatggattgtctaacaccatcacagtccctggctcagg

SEQ ID NO: 843 - DNA, 50 bp, Artificial Sequence
  Synthetic primer
ctgctggtgcaggtgctcgtctgctttatgcccttccacatctgtttcgc

SEQ ID NO: 844 - DNA, 50 bp, Artificial Sequence
  Synthetic primer
ccaacaaagatgaagttccctatctacggaaaggcatgactgggaggccc

SEQ ID NO: 845 - DNA, 50 bp, Artificial Sequence
  Synthetic primer
ggcaactccagtcagaacagcagaaataagcgtgccgttcagggtccaga

SEQ ID NO: 846 - DNA, 50 bp, Artificial Sequence
  Synthetic primer
gcagtactcctagggaaatggcctctgaagtatatcagtttcatctctta

SEQ ID NO: 847 - DNA, 50 bp, Artificial Sequence
  Synthetic primer
ccacacactcttgcagagctttcaggtgttactcagctgcatagttacgc

SEQ ID NO: 848 - DNA, 50 bp, Artificial Sequence
  Synthetic primer
acgggaggagaccagtcccccacccagccgtaccagaaataaaggcttct

SEQ ID NO: 849 - DNA, 50 bp, Artificial Sequence
  Synthetic primer
ccaagacatggaactgtgttctcaagggttatgtggggcgtgggctctcc

SEQ ID NO: 850 - DNA, 50 bp, Artificial Sequence
  Synthetic primer
actctggacttcagccaacaggattcctgagctgaagtgcagatgacaat

SEQ ID NO: 851 - DNA, 50 bp, Artificial Sequence
  Synthetic primer
cctgacactggagggcagctgtcttgtgcattacttgtgttcccagcacc

SEQ ID NO: 852 - DNA, 50 bp, Artificial Sequence
  Synthetic primer
ataccatggctcgtacaaggtggactgcccgcaaatctaccggtggtata

SEQ ID NO: 853 - DNA, 50 bp, Artificial Sequence
  Synthetic primer
tgccagaatcccctacacaaagagtcagaccgaagctcattacagctgcc

SEQ ID NO: 854 - DNA, 50 bp, Artificial Sequence
  Synthetic primer
cccacaccgtagaacatatattaatctactggcgaatgggaaaggagacc

SEQ ID NO: 855 - DNA, 50 bp, Artificial Sequence
  Synthetic primer
gtatttcaaaatgagtgaagcatgactctcacagatgaaggcctagacgc

SEQ ID NO: 856 - DNA, 50 bp, Artificial Sequence
  Synthetic primer
gctttcatgtcccagaacttagcctttacctgtgaagtgttactacagcc

SEQ ID NO: 857 - DNA, 50 bp, Artificial Sequence
  Synthetic primer
aaaacaaaatcaaccgggaccccagcttttcagaactgcagggaaacagc

SEQ ID NO: 858 - DNA, 50 bp, Artificial Sequence
  Synthetic primer
gccccaacctgggattgctgagcagggaagctttgcatgttgctctaagg

SEQ ID NO: 859 - DNA, 50 bp, Artificial Sequence
  Synthetic primer
tggcccccaagaagggagacagttctgctgaagaactcgaactggatact

SEQ ID NO: 860 - DNA, 50 bp, Artificial Sequence
  Synthetic primer
ggcccctatggcggtggaggccaatactttgccaaactgtgaaaccaagg

SEQ ID NO: 861 - DNA, 50 bp, Artificial Sequence
  Synthetic primer
aaaacatggcaataaatggctcgtgggctctggctccctgggaccccctc

SEQ ID NO: 862 - DNA, 50 bp, Artificial Sequence
  Synthetic primer
gctatggacaccttagtgacagagctccaagacctaagcaaccagtttcg

SEQ ID NO: 863 - DNA, 50 bp, Artificial Sequence
  Synthetic primer
aattcccaggtgttgtggtaggtaattgaatcatgggggcagtttccctc

SEQ ID NO: 864 - DNA, 50 bp, Artificial Sequence
  Synthetic primer
ggtgcagaagctacgccactctttactatcccaccaggagttctgggctc

SEQ ID NO: 865 - DNA, 50 bp, Artificial Sequence
  Synthetic primer
ggcttcctgctcaagacaccgattccgctacattccacccaacacaccag

SEQ ID NO: 866 - DNA, 50 bp, Artificial Sequence
  Synthetic primer
ggtctgtgtatgcttacttccccatcatcgtcgttatcccggagaatggg

SEQ ID NO: 867 - DNA, 50 bp, Artificial Sequence
  Synthetic primer
gggacactaccacaagccccttctggaaaggatgcagaaaagaccccagc

SEQ ID NO: 868 - DNA, 50 bp, Artificial Sequence
  Synthetic primer
agaggttcacatgcctgtctgcacattaaaagctctgggaagacctgttg

SEQ ID NO: 869 - DNA, 50 bp, Artificial Sequence
  Synthetic primer
gaatgtgactcaagactcgaggccgatacgaggctgtgattctgcctttg

SEQ ID NO: 870 - DNA, 50 bp, Artificial Sequence
  Synthetic primer
cacacatgcttttctgcacgtggttgccttagtcatcttcctacagcacc

SEQ ID NO: 871 - DNA, 50 bp, Artificial Sequence
  Synthetic primer
gctgatttcctaccagcctttgcctcttccttcaatgtggtttccatggg

SEQ ID NO: 872 - DNA, 50 bp, Artificial Sequence
  Synthetic primer
agaagaccctgacccactgagggtttgttgtgagaattgctgaagccacg

SEQ ID NO: 873 - DNA, 50 bp, Artificial Sequence
  Synthetic primer
ggcccgattcctgcttctcaacaacctctttggactcagtgcctacccat

SEQ ID NO: 874 - DNA, 50 bp, Artificial Sequence
  Synthetic primer
ccctctgccacagttatcaagaatgggtcaggagaccgctgcttctgggc

SEQ ID NO: 875 - DNA, 50 bp, Artificial Sequence
  Synthetic primer
ggctcaccagagtacccagaagaatcagtatggaattagaggacagtggc

SEQ ID NO: 876 - DNA, 50 bp, Artificial Sequence
  Synthetic primer
ccaggtgccgcccactcttgacgtgatacttaccgtcaatgctccttacc

SEQ ID NO: 877 - DNA, 50 bp, Artificial Sequence
  Synthetic primer
ggagctggggctcaacccactgtttttaaggatttgcgcttacttgtggc

SEQ ID NO: 878 - DNA, 50 bp, Artificial Sequence
  Synthetic primer
ctgcctggctcctccttgaggctggaactctctccagggtggttaactct

SEQ ID NO: 879 - DNA, 50 bp, Artificial Sequence
  Synthetic primer
ggccctgagacacgtgaacacctccctcctatgcatcacaaaccttctcc

SEQ ID NO: 880 - DNA, 50 bp, Artificial Sequence
  Synthetic primer
accttcactcccagctccatcctcattgccccctctacctcctaaggtcc

SEQ ID NO: 881 - DNA, 50 bp, Artificial Sequence
  Synthetic primer
ggtgtcctccatcccccagtgccttcacatcttgaggatatgcttctgta

SEQ ID NO: 882 - DNA, 50 bp, Artificial Sequence
  Synthetic primer
cctgcctgcacgccttcagtaactccgagcagaaatcacatcttgcccac

SEQ ID NO: 883 - DNA, 50 bp, Artificial Sequence
  Synthetic primer
tctatgaagcccactcacttgccattccagggccaaaggaccggaggttt

SEQ ID NO: 884 - DNA, 50 bp, Artificial Sequence
  Synthetic primer
cgcctgtgtgaaagctctggtctccctgaggagctaccatctgcaaatcg

SEQ ID NO: 885 - DNA, 50 bp, Artificial Sequence
  Synthetic primer
ctgttctcagggaccatctcggctgcctcctgtacccagactctaacctg

SEQ ID NO: 886 - DNA, 50 bp, Artificial Sequence
  Synthetic primer
gcacagtcccaggtcccagctcccctcttatggtttctgtcataatgtgc

SEQ ID NO: 887 - DNA, 50 bp, Artificial Sequence
  Synthetic primer
gaaaaccctgggtctcacatcgagctgctggaggaggtggccgagaaggt

SEQ ID NO: 888 - DNA, 50 bp, Artificial Sequence
  Synthetic primer
cctgtttggatcacatggtcttgtcctgataacttggaagaggttgcttc

SEQ ID NO: 889 - DNA, 50 bp, Artificial Sequence
  Synthetic primer
ggataagtgggagctagccagataaagctgcaggaaatagcattccaggc

SEQ ID NO: 890 - DNA, 50 bp, Artificial Sequence
  Synthetic primer
cagaggaactttgctcaaggcgcagatccgtcaccagtcccttgacagtc

SEQ ID NO: 891 - DNA, 50 bp, Artificial Sequence
  Synthetic primer
ggacccctggatccttgccattcccctcagctaatgacggagtgctcctt

SEQ ID NO: 892 - DNA, 50 bp, Artificial Sequence
  Synthetic primer
tgggtgagtttcccccctccttattctgtcctgagaccacgggcaaagct

SEQ ID NO: 893 - DNA, 50 bp, Artificial Sequence
  Synthetic primer
ttggctgggcagagtggcttacacctataatcccagcaatttgggaggcc

SEQ ID NO: 894 - DNA, 50 bp, Artificial Sequence
  Synthetic primer
agacttttggtctgtgggccatttaacctggatgccaccattttatgggg

SEQ ID NO: 895 - DNA, 50 bp, Artificial Sequence
  Synthetic primer
ggaggtaagattgccacgtgcctgctggtactgtgatgcatttcaagtgg

SEQ ID NO: 896 - DNA, 50 bp, Artificial Sequence
  Synthetic primer
ctaccactatagatgagggtgaggagtagggagtgcagccctgagcctgc

SEQ ID NO: 897 - DNA, 50 bp, Artificial Sequence
  Synthetic primer
caaggaggagtccctgctggctctgacgagtatgaagatgaatgccctca

SEQ ID NO: 898 - DNA, 50 bp, Artificial Sequence
  Synthetic primer
ttgtgaaaacattggccacctagtagcagtggtgaggagtgggagggccc

SEQ ID NO: 899 - DNA, 50 bp, Artificial Sequence
  Synthetic primer
cgtggggagagggcgtaaattgtcttgtgaatgtgtgctaaccaaaaccc

SEQ ID NO: 900 - DNA, 50 bp, Artificial Sequence
  Synthetic primer
aaatggagcccaaggctgagaccgagaccaagacccagaccgagatggag

SEQ ID NO: 901 - DNA, 50 bp, Artificial Sequence
  Synthetic primer
gcagcgttactggcctatgtatcatccgtctatggcttccacccagtctc

SEQ ID NO: 902 - DNA, 50 bp, Artificial Sequence
  Synthetic primer
cggagtagattccgggtgcctttactccactgtgacctcataaatccagg

SEQ ID NO: 903 - DNA, 50 bp, Artificial Sequence
  Synthetic primer
cctccagcattcagtccagggggagccacggaaaccatgttcttgcttaa

SEQ ID NO: 904 - DNA, 50 bp, Artificial Sequence
  Synthetic primer
tcggtcctgctggaggccacgggtgccacacactcggtcccgacatgatg

SEQ ID NO: 905 - DNA, 50 bp, Artificial Sequence
  Synthetic primer
gagtagtcccacgctgttgttgagctgactctgtgcccgaatggtggtgc

SEQ ID NO: 906 - DNA, 50 bp, Artificial Sequence
  Synthetic primer
gagagaactcagccccctggtctaagctggacttacctctgtggattctg

SEQ ID NO: 907 - DNA, 50 bp, Artificial Sequence
  Synthetic primer
tccttcgacctccactgcgccccacctccctgcctgtgtgtgttatttca

SEQ ID NO: 908 - DNA, 50 bp, Artificial Sequence
  Synthetic primer
gggattacaggcgtgagtgaccatgcctagctcacttccaggtttaacag

SEQ ID NO: 909 - DNA, 50 bp, Artificial Sequence
  Synthetic primer
gcaggtttcaaggccaagctggtgaccctgctgagtcgtgagcggaggaa

SEQ ID NO: 910 - DNA, 50 bp, Artificial Sequence
  Synthetic primer
tgggacacagctggccaagagcggttcaagacaataactactgcctacta

SEQ ID NO: 911 - DNA, 50 bp, Artificial Sequence
  Synthetic primer
ggagaagaatccatcaccccggattgtgaaaacacatctaccgactgatc

SEQ ID NO: 912 - DNA, 50 bp, Artificial Sequence
  Synthetic primer
cagaggcatccgccttcccatccacactgcctttagtggtacgtccgtca

SEQ ID NO: 913 - DNA, 50 bp, Artificial Sequence
  Synthetic primer
tggggggaagcacataatcccgccccagggcccactagcatccactgatt

SEQ ID NO: 914 - DNA, 50 bp, Artificial Sequence
  Synthetic primer
gtctcagcgaggtgtaaagttgttcgccgcgtggaatgtgagtgtgtttg

SEQ ID NO: 915 - DNA, 50 bp, Artificial Sequence
  Synthetic primer
cgatgtagccaaggtcaacaccctgattaggcctgagggagagaaggcat

SEQ ID NO: 916 - DNA, 50 bp, Artificial Sequence
  Synthetic primer
tttggatgcacttgagcaggggtccccaacccctgagccatggagccgta

SEQ ID NO: 917 - DNA, 50 bp, Artificial Sequence
  Synthetic primer
ggcccgcgtctcgaaggggcacctgtgaactcaaaaggctcttttcagag

SEQ ID NO: 918 - DNA, 50 bp, Artificial Sequence
  Synthetic primer
cgactttcccgatcgccaggcaggagtttctctcggtgactactatcgct

SEQ ID NO: 919 - DNA, 50 bp, Artificial Sequence
  Synthetic primer
tctggaggtgtccaggctgtggggaccacattgctccaagccagatatgg

SEQ ID NO: 920 - DNA, 50 bp, Artificial Sequence
  Synthetic primer
catagggctgaggcaggatttggaatcaggccatgtcttctccagagccc

SEQ ID NO: 921 - DNA, 50 bp, Artificial Sequence
  Synthetic primer
cgctgggagacctttgggacgtggggtggaatttggggtatctgtgcctt

SEQ ID NO: 922 - DNA, 50 bp, Artificial Sequence
  Synthetic primer
ggcccttcactgcctttgctcctcccatgttatgcccagcgtttgatggg

SEQ ID NO: 923 - DNA, 50 bp, Artificial Sequence
  Synthetic primer
ctgcagagcctggttcaaaatcagtcactcccttcagaagcagacatggc

SEQ ID NO: 924 - DNA, 50 bp, Artificial Sequence
  Synthetic primer
gcctaaaccaggtatgccaatctgtcttgtgtccacatactaacagaggg

SEQ ID NO: 925 - DNA, 50 bp, Artificial Sequence
  Synthetic primer
cacacctgtagtactcttccccatttcctctgacactcatgcagaatgag

SEQ ID NO: 926 - DNA, 50 bp, Artificial Sequence
  Synthetic primer
gcccagccagggtcctctctcaaccactagacttggctctcaggaactct

SEQ ID NO: 927 - DNA, 50 bp, Artificial Sequence
  Synthetic primer
ccagctctaccagcccctcaaggatcgagaagatgaccagtacagccacc

SEQ ID NO: 928 - DNA, 50 bp, Artificial Sequence
  Synthetic primer
tagaagacctgggacagaggactgctgtctgccctctctggtcaccctgc

SEQ ID NO: 929 - DNA, 50 bp, Artificial Sequence
  Synthetic primer
ccttccacctgctggggcaccattcttagtatacagaggtggccttacac

SEQ ID NO: 930 - DNA, 50 bp, Artificial Sequence
  Synthetic primer
tcactgacccccgcctcttcagcaagaccacagccatgacaaacaccagg

SEQ ID NO: 931 - DNA, 50 bp, Artificial Sequence
  Synthetic primer
gggtgaggggccagagggaagtctgctgtgttctcatgtaggatgtcagc

SEQ ID NO: 932 - DNA, 50 bp, Artificial Sequence
  Synthetic primer
tctgccaaccccaccagtgatgaaaattaactgtgggccactcgctgcag

SEQ ID NO: 933 - DNA, 50 bp, Artificial Sequence
  Synthetic primer
gctactgcaggaccagaagcacaaagtgcggtttcccaagcctttgtcca

SEQ ID NO: 934 - DNA, 50 bp, Artificial Sequence
  Synthetic primer
gtgtcactgttacatttcccccaagaagttcaataaaccagcttagccac

SEQ ID NO: 935 - DNA, 50 bp, Artificial Sequence
  Synthetic primer
ctactctctgccatcacaaggcccacttttgtagcttccgcgtgagtgag

SEQ ID NO: 936 - DNA, 50 bp, Artificial Sequence
  Synthetic primer
acagggccgccctccacacctgcctcacacgccaaggattacacagtgga

SEQ ID NO: 937 - DNA, 50 bp, Artificial Sequence
  Synthetic primer
ccacccttgggagtggcttggtggagctggggacaaggttttgtttacta

SEQ ID NO: 938 - DNA, 50 bp, Artificial Sequence
  Synthetic primer
tgagtcagcacccctcctagcaacctgccttgggacttagaacaccgccg

SEQ ID NO: 939 - DNA, 50 bp, Artificial Sequence
  Synthetic primer
ctgctcctgtcaccctccacgattccacagaccaggacattccatgtgcc

SEQ ID NO: 940 - DNA, 50 bp, Artificial Sequence
  Synthetic primer
agtggataccagattggagaagacttccaagagagatgatgccaagaaat

SEQ ID NO: 941 - DNA, 50 bp, Artificial Sequence
  Synthetic primer
ggtgctgatcctaccacctactgctaccttccttagcttcaccctggcta

SEQ ID NO: 942 - DNA, 50 bp, Artificial Sequence
  Synthetic primer
ccagccactggtaaccattcttctactctgtatgcccatgaggtcaattg

SEQ ID NO: 943 - DNA, 50 bp, Artificial Sequence
  Synthetic primer
aacaactgcaagacccaaggctccagtgctgactctcattggggagggcg

SEQ ID NO: 944 - DNA, 50 bp, Artificial Sequence
  Synthetic primer
gtgtggaaattttgcctgaagtcctccacctaaaaacctgatgccattgg

SEQ ID NO: 945 - DNA, 50 bp, Artificial Sequence
  Synthetic primer
acgcagggaccgtttttaaatcacaggggcgtgtgtcagcctgccctagg

SEQ ID NO: 946 - DNA, 50 bp, Artificial Sequence
  Synthetic primer
gtccctagaccactttgtatgaccgtttgcagtctgagcaggccaggggc

SEQ ID NO: 947 - DNA, 50 bp, Artificial Sequence
  Synthetic primer
agccaagacagcagctctacatccttacctaggtaattcaggcatgcgcc

SEQ ID NO: 948 - DNA, 50 bp, Artificial Sequence
  Synthetic primer
gggcaccggctgcctttccccagggaatcctctccagatcttaaccaaga

SEQ ID NO: 949 - DNA, 50 bp, Artificial Sequence
  Synthetic primer
gttttgtacagtgcctggcactctgtgggtgctcaataaatggataggag

SEQ ID NO: 950 - DNA, 50 bp, Artificial Sequence
  Synthetic primer
aaaaacttacagcgatggccccaccaaggcctagacacggcactggcctc

SEQ ID NO: 951 - DNA, 50 bp, Artificial Sequence
  Synthetic primer
tcatgtgtcagttttcacacagcctgcacatcgttctgacatgccctttt

SEQ ID NO: 952 - DNA, 50 bp, Artificial Sequence
  Synthetic primer
acccggccatccatttttcttccatgcatcctggctgggccgacacccca

SEQ ID NO: 953 - DNA, 50 bp, Artificial Sequence
  Synthetic primer
gcagcagctcacacctctctgcggttccctctcacttttagatcggaagc

SEQ ID NO: 954 - DNA, 50 bp, Artificial Sequence
  Synthetic primer
cacatggcaaatgcctcctttcacaatagagcatggtgctgtttcctcac

SEQ ID NO: 955 - DNA, 50 bp, Artificial Sequence
  Synthetic primer
gggctcgcatccaaaagaatataacactccaagcatgaaagagtggcagc

SEQ ID NO: 956 - DNA, 50 bp, Artificial Sequence
  Synthetic primer
aaggccccagagagctaactcacccttccaccatatgaggacgtggcaag

SEQ ID NO: 957 - DNA, 50 bp, Artificial Sequence
  Synthetic primer
gcgcaggtgtgagtgtaagttcatgtaggagagtgtatgcgtgtgcgcct

SEQ ID NO: 958 - DNA, 50 bp, Artificial Sequence
  Synthetic primer
tccactgtcctgccaaggtacacctcagtactccccttcccaactgaagt

SEQ ID NO: 959 - DNA, 50 bp, Artificial Sequence
  Synthetic primer
agcttcttctgaaagaattatttgccttctgaaatgtgacccccaagtcc

SEQ ID NO: 960 - DNA, 50 bp, Artificial Sequence
  Synthetic primer
agaagctgccaccagagactaaagggaaggctgctatggaggaactacag

SEQ ID NO: 961 - DNA, 50 bp, Artificial Sequence
  Synthetic primer
ttgccagcctctgctgctcctgctcctgggtttgaagatgcaggccgatc

SEQ ID NO: 962 - DNA, 50 bp, Artificial Sequence
  Synthetic primer
gcagtgatcagggtcctgcaagcagtggggaagggggccaaggtattgga

SEQ ID NO: 963 - DNA, 50 bp, Artificial Sequence
  Synthetic primer
ggctgcaaagggaataggctgatggagctgttctgtatggtactgtgtgg

SEQ ID NO: 964 - DNA, 50 bp, Artificial Sequence
  Synthetic primer
gtgagctgagacagataagagaataccttggtctcacctatcctgccctc

SEQ ID NO: 965 - DNA, 50 bp, Artificial Sequence
  Synthetic primer
ttcctgagccctgagtggaagcctttctgtgccttgccctttgcccactc

SEQ ID NO: 966 - DNA, 50 bp, Artificial Sequence
  Synthetic primer
cctctgtgcggccctctgtggatgagcagaagcgcagacttaatgatgtg

SEQ ID NO: 967 - DNA, 50 bp, Artificial Sequence
  Synthetic primer
agaaaagcattacccattttgcggccaagttcccgacgagaggctggacc

SEQ ID NO: 968 - DNA, 50 bp, Artificial Sequence
  Synthetic primer
gagtgtagagcccatatccccatagagtctacctagagcaggggtcgcca

SEQ ID NO: 969 - DNA, 50 bp, Artificial Sequence
  Synthetic primer
atggcaagaaaagctggcaatttctatgtacctgcagaagccaaattggc

SEQ ID NO: 970 - DNA, 50 bp, Artificial Sequence
  Synthetic primer
cagggcagaacctctgaagacgccactcctccaagccttcaccctgtgga

SEQ ID NO: 971 - DNA, 50 bp, Artificial Sequence
  Synthetic primer
aggcagtagttctggaaaagccactgggagcaagacaggtgctaaagttg

SEQ ID NO: 972 - DNA, 50 bp, Artificial Sequence
  Synthetic primer
gctgaagattcccttgagaacaagtactgtccctagtttcccagtgctgg

SEQ ID NO: 973 - DNA, 50 bp, Artificial Sequence
  Synthetic primer
acaggaagcaaactaagcccccgctgtaatgaaacaccttctctggagcc

SEQ ID NO: 974 - DNA, 50 bp, Artificial Sequence
  Synthetic primer
cacaaatggtaaactcctccacgtgcttcctgcgttccgtgcaagccgcc

SEQ ID NO: 975 - DNA, 50 bp, Artificial Sequence
  Synthetic primer
agtgctgccctctggggacatgcggagtgggggtcttatccctgtgctga

SEQ ID NO: 976 - DNA, 50 bp, Artificial Sequence
  Synthetic primer
cagtgagctgcccccaaatcaagtttagtgccctcatccatttatgtctc

SEQ ID NO: 977 - DNA, 50 bp, Artificial Sequence
  Synthetic primer
acccagtgttttaggagcatgagtgccgtgtgtgtgcgtcctgtcggagc

SEQ ID NO: 978 - DNA, 50 bp, Artificial Sequence
  Synthetic primer
gttacggcgtctggagataaaaccattcgcatctgggatgtgaggactac

SEQ ID NO: 979 - DNA, 50 bp, Artificial Sequence
  Synthetic primer
cagagcccctggtgcaatgcggtcacaggttttatgggactttggtgagc

SEQ ID NO: 980 - DNA, 50 bp, Artificial Sequence
  Synthetic primer
gtgctgcactcaactatggaaagaggcagagtttgcttgcccaattgcca

SEQ ID NO: 981 - DNA, 50 bp, Artificial Sequence
  Synthetic primer
ctgcagaaggagtcggcgtatggagtctcgaacccgaggatttgtctgcc

SEQ ID NO: 982 - DNA, 50 bp, Artificial Sequence
  Synthetic primer
ttgtcatcctttgagctgctgactgccaataacattcttcgatctctggg

SEQ ID NO: 983 - DNA, 50 bp, Artificial Sequence
  Synthetic primer
cctgaagtgctggaggcagttatggaaggagctgcttagggaagaggggt

SEQ ID NO: 984 - DNA, 50 bp, Artificial Sequence
  Synthetic primer
cctcttccccaccctgtccctgtagaaaaccagtgaagcaggtacggagc

SEQ ID NO: 985 - DNA, 50 bp, Artificial Sequence
  Synthetic primer
gagatggaaaacaagcttctggtgggtgcattttctggcccggagttgcc

SEQ ID NO: 986 - DNA, 50 bp, Artificial Sequence
  Synthetic primer
gaggacagacattgcccggaaactcagtctatttattcttcagcttgccc

SEQ ID NO: 987 - DNA, 50 bp, Artificial Sequence
  Synthetic primer
cagcctctctgttgcctcagctctccaagttccaggcttccggtccttag

SEQ ID NO: 988 - DNA, 50 bp, Artificial Sequence
  Synthetic primer
atgcgggtagtattagccacccccctccatctgttcccagcaccggagaa

SEQ ID NO: 989 - DNA, 50 bp, Artificial Sequence
  Synthetic primer
gatcttccaggcctatggatagatcaggaggcatcactgaggccaggagc

SEQ ID NO: 990 - DNA, 50 bp, Artificial Sequence
  Synthetic primer
tcgacatgccattactgaagcaggcccagaacactgttacgcttgccacc

SEQ ID NO: 991 - DNA, 50 bp, Artificial Sequence
  Synthetic primer
agggtattgaagctgcagagggatcaacttgtgcttgccagaggacgcca

SEQ ID NO: 992 - DNA, 50 bp, Artificial Sequence
  Synthetic primer
attcttggtggcttcttcatagcaggtaagcctctccttctaaaaacttc

SEQ ID NO: 993 - DNA, 50 bp, Artificial Sequence
  Synthetic primer
gagtctgcctccatgctgcagtgttcgagtggattgtaggtgcaagatgg

SEQ ID NO: 994 - DNA, 50 bp, Artificial Sequence
  Synthetic primer
tgagccacaactgctggacacacacagagaggaggaagtctcccccgcca

SEQ ID NO: 995 - DNA, 50 bp, Artificial Sequence
  Synthetic primer
tactccaggccaaaggaagcaccagagccaatggaacttgatggccctaa

SEQ ID NO: 996 - DNA, 50 bp, Artificial Sequence
  Synthetic primer
gcacacagtgctttccgtggcactgcatacaatctgaggcctcctctctc

SEQ ID NO: 997 - DNA, 50 bp, Artificial Sequence
  Synthetic primer
aagctctgtgaactgcctgctgtcctgtgaactgcctgctgccctgtgag

SEQ ID NO: 998 - DNA, 50 bp, Artificial Sequence
  Synthetic primer
ctgattcctgcaagccagcagccaaaggagaataagacctctgaagaccc

SEQ ID NO: 999 - DNA, 50 bp, Artificial Sequence
  Synthetic primer
cctcgtatgcactctggcctgcttctccagttacttgcttgtgtaagaac

SEQ ID NO: 1000 - DNA, 50 bp, Artificial Sequence
  Synthetic primer
ccttccaggctgcaataaccagcaaggttttcagttaaagccctatctgc

SEQ ID NO: 1001 - DNA, 50 bp, Artificial Sequence
  Synthetic primer
aggaattcatcttcagtctaccagcccccgctgtgtcggatacacactcg

SEQ ID NO: 1002 - DNA, 50 bp, Artificial Sequence
  Synthetic primer
tgaggctgcgccctgagccggggctggttttctatgaacgattccggcct

SEQ ID NO: 1003 - DNA, 50 bp, Artificial Sequence
  Synthetic primer
gcaacaccctggaaaggacctgcattcctgatctgactgtcctgaaaacc

SEQ ID NO: 1004 - DNA, 50 bp, Artificial Sequence
  Synthetic primer
cagagccccagcccctcatgtcttgccgcccttcctccatgtgtttgtaa

SEQ ID NO: 1005 - DNA, 50 bp, Artificial Sequence
  Synthetic primer
gatcttcaaagctgctgcgggcattctatgtccccttcctgtcagatcag

SEQ ID NO: 1006 - DNA, 50 bp, Artificial Sequence
  Synthetic primer
gtcagatgctgttgggtcacatagaagaacaagataaggtcctccactgc

SEQ ID NO: 1007 - DNA, 50 bp, Artificial Sequence
  Synthetic primer
cccaagagggtataaatccctgctttactgctgagctcctgctggaggtg

SEQ ID NO: 1008 - DNA, 50 bp, Artificial Sequence
  Synthetic primer
gttgaacacagccagcacagtttcgacaggtcaaggaagagctgtttcag

SEQ ID NO: 1009 - DNA, 50 bp, Artificial Sequence
  Synthetic primer
ccgtgtgcggggctgagcccggctgataaagcactttctctctgaaaaaa

SEQ ID NO: 1010 - DNA, 50 bp, Artificial Sequence
  Synthetic primer
tattgcagccatccatcttgggggctcatccatcacacccgggttgctag

SEQ ID NO: 1011 - DNA, 50 bp, Artificial Sequence
  Synthetic primer
gcgtacactctctccaggcaaccagctctaccacctcatccagaattggc

SEQ ID NO: 1012 - DNA, 50 bp, Artificial Sequence
  Synthetic primer
ccagttcttcctgacaccggatgggtgcttgggaaccgtttgagccttat

SEQ ID NO: 1013 - DNA, 50 bp, Artificial Sequence
  Synthetic primer
gtgggctaagataggtcctactgcaaaccacccctccatatttccgtacc

SEQ ID NO: 1014 - DNA, 50 bp, Artificial Sequence
  Synthetic primer
gatggccataaggcggtgttcgtggcacgggtgctgactggcgactacgg

SEQ ID NO: 1015 - DNA, 50 bp, Artificial Sequence
  Synthetic primer
gccgatggccataaggcggtgttcgtggcacgggtgctgactggcgacta

SEQ ID NO: 1016 - DNA, 50 bp, Artificial Sequence
  Synthetic primer
agcagcgaagaggaacacgaaagcaaatggcagtagccctgagacggccg

SEQ ID NO: 1017 - DNA, 50 bp, Artificial Sequence
  Synthetic primer
ggagggtgacagtggccgcccaaggcccacgagcttgttctttgttcttt

SEQ ID NO: 1018 - DNA, 50 bp, Artificial Sequence
  Synthetic primer
ctggctcctaaaatgtgccaagaagctgctgtacccaaaatccttccttg

SEQ ID NO: 1019 - DNA, 50 bp, Artificial Sequence
  Synthetic primer
ggcagagaaggaggccaagaagccaaccatcaagaagcccctcaatgcct

SEQ ID NO: 1020 - DNA, 50 bp, Artificial Sequence
  Synthetic primer
cctggatgagagtgacaatgacttctgacccttttgccaagggaccctgg

SEQ ID NO: 1021 - DNA, 50 bp, Artificial Sequence
  Synthetic primer
cgtgaccctcacgggcaggctaaacaagtgtgggatgattagccccagag

SEQ ID NO: 1022 - DNA, 50 bp, Artificial Sequence
  Synthetic primer
aaaaaagccttttggctctgaccagcaccatggcagttggcaagaacaag

SEQ ID NO: 1023 - DNA, 50 bp, Artificial Sequence
  Synthetic primer
tggtggacgttaagttcggaggggccgccgtcttccccaaccaggagcag

SEQ ID NO: 1024 - DNA, 50 bp, Artificial Sequence
  Synthetic primer
ggctgctccttgctatagaagacctgggacagaggactgctgtctgccct

SEQ ID NO: 1025 - DNA, 50 bp, Artificial Sequence
  Synthetic primer
ccctgcaggcccaggctgagccactccaggcaagagctgatgaggttgct

SEQ ID NO: 1026 - DNA, 50 bp, Artificial Sequence
  Synthetic primer
ccttgctatagaagacctgggacagaggactgctgtctgccctctctggt

SEQ ID NO: 1027 - DNA, 50 bp, Artificial Sequence
  Synthetic primer
acagcagttaccccgacgaaccctggaggataacagaagaacagcgcgag

SEQ ID NO: 1028 - DNA, 50 bp, Artificial Sequence
  Synthetic primer
gtcaccttgtgcgagaacctcaaccgcacctatcttgggaaccgggtata

SEQ ID NO: 1029 - DNA, 50 bp, Artificial Sequence
  Synthetic primer
gcccttcctctcccataagatggacaaaagtgtttctgtatcactgtgtc

SEQ ID NO: 1030 - DNA, 50 bp, Artificial Sequence
  Synthetic primer
ccctacaatggagcacaggactggatttactctggcagctactgggacag

SEQ ID NO: 1031 - DNA, 50 bp, Artificial Sequence
  Synthetic primer
agcgagagcctcactggttcccaggacatcaacctgaactggatcctccc

SEQ ID NO: 1032 - DNA, 50 bp, Artificial Sequence
  Synthetic primer
ggacggtccccttcctttcatctctggcctgtttgattctggcagcagcc

SEQ ID NO: 1033 - DNA, 50 bp, Artificial Sequence
  Synthetic primer
gggatgcaggaagtaaaactgaactcttacgttgtgagcggcaccgtcgg

SEQ ID NO: 1034 - DNA, 50 bp, Artificial Sequence
  Synthetic primer
cagtggctaccacctgtaatctcagcagtttgggagaccaaagcaggacg

SEQ ID NO: 1035 - DNA, 50 bp, Artificial Sequence
  Synthetic primer
ctgggctgtggtatttgggtgatctttacattcttcagactcatgtgtgt

SEQ ID NO: 1036 - DNA, 50 bp, Artificial Sequence
  Synthetic primer
ccacggaggtgaatatgagaagcaggactctccaagaaccccttagcgac

SEQ ID NO: 1037 - DNA, 50 bp, Artificial Sequence
  Synthetic primer
ccctagagtggtttccatctcacagagaatcagacagggccacagccccc

SEQ ID NO: 1038 - DNA, 50 bp, Artificial Sequence
  Synthetic primer
attgggaccccataccgttagcccttgtatgtataccaacactgccaaag

SEQ ID NO: 1039 - DNA, 50 bp, Artificial Sequence
  Synthetic primer
aactctacagatggcatacctgtgcctgcttctggggttggaagtgtgac

SEQ ID NO: 1040 - DNA, 50 bp, Artificial Sequence
  Synthetic primer
ggcagggacgaggacaaatatggatgaggtggagagtgggaagcaggagc

SEQ ID NO: 1041 - DNA, 50 bp, Artificial Sequence
  Synthetic primer
aaagccgagctcagttaccatagggacaggtccacctctactgggccctc

SEQ ID NO: 1042 - DNA, 50 bp, Artificial Sequence
  Synthetic primer
gtggagaggttagagccagccacacttgaacctaataccctgcccttgac

SEQ ID NO: 1043 - DNA, 50 bp, Artificial Sequence
  Synthetic primer
aaacacaagaagttctcgaaatgggagtttgaagacctggaatgggggag

SEQ ID NO: 1044 - DNA, 50 bp, Artificial Sequence
  Synthetic primer
cgcccagggcggcgttttgcctaacatccaggccgttctgttaccaaaga

SEQ ID NO: 1045 - DNA, 50 bp, Artificial Sequence
  Synthetic primer
acaccaaggcagtcagttaaggcagctatggtttggaaaggcatacggac

SEQ ID NO: 1046 - DNA, 50 bp, Artificial Sequence
  Synthetic primer
cccgggagtggattctaaatgtgattttcctaggctactgcaggagcccc

SEQ ID NO: 1047 - DNA, 50 bp, Artificial Sequence
  Synthetic primer
ggtggaggttccagtgagccaaaatcatgccaatgtattccagtctgggt

SEQ ID NO: 1048 - DNA, 50 bp, Artificial Sequence
  Synthetic primer
tatttagggcaataagcagcattggcctggagtgccagagcgtcacctcc

SEQ ID NO: 1049 - DNA, 50 bp, Artificial Sequence
  Synthetic primer
ctcgggcatccttcccagggttgggtcttacacaaatagaaggctcttgc

SEQ ID NO: 1050 - DNA, 50 bp, Artificial Sequence
  Synthetic primer
gctaatcttcagcccgtaccaaaaagtagagtggagcctctttgcactac

SEQ ID NO: 1051 - DNA, 50 bp, Artificial Sequence
  Synthetic primer
ctctctcaagggtctcacgttgcagcattcttaccagaggccattgggcc

SEQ ID NO: 1052 - DNA, 50 bp, Artificial Sequence
  Synthetic primer
acgcttgtagggtttcaccaggtaagcggtgttgaactttctgcttgtgt

SEQ ID NO: 1053 - DNA, 50 bp, Artificial Sequence
  Synthetic primer
cagggatggctctttgccatgggtcttattttcaccctcttttctgtaag

SEQ ID NO: 1054 - DNA, 50 bp, Artificial Sequence
  Synthetic primer
tgtgccctttggggaatgaatacttttgttcatgtctagcccactgggaa

SEQ ID NO: 1055 - DNA, 50 bp, Artificial Sequence
  Synthetic primer
gctagcatttttccaacgagggaacttattccgcacgggcctactgtagg

SEQ ID NO: 1056 - DNA, 50 bp, Artificial Sequence
  Synthetic primer
catggacagaagaaggcagcaggtggcagaatggggtgcatgaaggtttc

SEQ ID NO: 1057 - DNA, 50 bp, Artificial Sequence
  Synthetic primer
tggaccactccattgccatctacctgctcaaccctgacggcctcttcacg

SEQ ID NO: 1058 - DNA, 50 bp, Artificial Sequence
  Synthetic primer
tgcattgggaccagtcatttcctaagctacatatggccatcttgacagtg

SEQ ID NO: 1059 - DNA, 50 bp, Artificial Sequence
  Synthetic primer
acacagaggaagttggctagaggccggtcccttccttgggcccctctcat

SEQ ID NO: 1060 - DNA, 50 bp, Artificial Sequence
  Synthetic primer
ggatgtgggagtttttaaatgctatagccctgggcttgccccatttgggg

SEQ ID NO: 1061 - DNA, 50 bp, Artificial Sequence
  Synthetic primer
gtgacggtgactgtggccttgaagcctggaggactgttgggcatgtagac

SEQ ID NO: 1062 - DNA, 50 bp, Artificial Sequence
  Synthetic primer
ccaggctccctccgcagactgactttcctctgtgtctgggtgttacagtc

SEQ ID NO: 1063 - DNA, 50 bp, Artificial Sequence
  Synthetic primer
ggtgatagtcagagagtggtgtttttgttcaggtgggaaggattggaaac

SEQ ID NO: 1064 - DNA, 50 bp, Artificial Sequence
  Synthetic primer
agccacctgagctgaagattcccttgagaacaagtactgtcctgtggttt

SEQ ID NO: 1065 - DNA, 50 bp, Artificial Sequence
  Synthetic primer
ctaggctttgcttagtatcatgtccatgtttccttcacctcagtggagct

SEQ ID NO: 1066 - DNA, 50 bp, Artificial Sequence
  Synthetic primer
atccaatcagagcccaccctcctggaagagacccccgtgttcagagtgct

SEQ ID NO: 1067 - DNA, 50 bp, Artificial Sequence
  Synthetic primer
ctcatcaccttcctctttggcatggtggtcctggccctggtggtctggaa

SEQ ID NO: 1068 - DNA, 50 bp, Artificial Sequence
  Synthetic primer
cgtccacggctacttcctcatcaccttcctctttggcatggtggtcctgg

SEQ ID NO: 1069 - DNA, 50 bp, Artificial Sequence
  Synthetic primer
tctcagcttaacatggaaatgaggatcccaccagccccagaaccctctgg

SEQ ID NO: 1070 - DNA, 50 bp, Artificial Sequence
  Synthetic primer
ggaacagactgagaagggcaaacattcctgggagctgggcaaggagatcc

SEQ ID NO: 1071 - DNA, 50 bp, Artificial Sequence
  Synthetic primer
ctggcccacctccagcctcagctagcattacccttctgacactctctatg

SEQ ID NO: 1072 - DNA, 50 bp, Artificial Sequence
  Synthetic primer
ccacctgattcccggacagacctccccaactccgcgtgagacagagaatt

SEQ ID NO: 1073 - DNA, 50 bp, Artificial Sequence
  Synthetic primer
ctatcaggtcgtgagtccaggggtctacaagtcccgggccccccagttca

SEQ ID NO: 1074 - DNA, 50 bp, Artificial Sequence
  Synthetic primer
tctacaggcacaactgctgcctcacaacagccttttttggcccagttcct

SEQ ID NO: 1075 - DNA, 50 bp, Artificial Sequence
  Synthetic primer
gtcccagtttcccactctgaagatcccaacatggtcctagcactgcacac

SEQ ID NO: 1076 - DNA, 50 bp, Artificial Sequence
  Synthetic primer
tcagctcctcaggaacgactggggaagctgtggcttcatggtccccgagg

SEQ ID NO: 1077 - DNA, 50 bp, Artificial Sequence
  Synthetic primer
gggctgggaaaaatattcaataactgtaagcccccttttggatgccaaac

SEQ ID NO: 1078 - DNA, 50 bp, Artificial Sequence
  Synthetic primer
atccatcacctgcaaagctacctcatgcccctttacattccctccttgcc

SEQ ID NO: 1079 - DNA, 50 bp, Artificial Sequence
  Synthetic primer
gaggtgtgctgcttgtcagcatgtattgtcacggacactcaataagtgcc

SEQ ID NO: 1080 - DNA, 50 bp, Artificial Sequence
  Synthetic primer
cagaagacagagggttcgaatcccagcactgccgtctactcactgtagta

SEQ ID NO: 1081 - DNA, 50 bp, Artificial Sequence
  Synthetic primer
gctacaaacaagctcatctttggaactggcactctgcttgctgtccagcc

SEQ ID NO: 1082 - DNA, 50 bp, Artificial Sequence
  Synthetic primer
gcaggagaaagagttcaaatacacgtgtccgcacagcgccgagatcctgg

SEQ ID NO: 1083 - DNA, 50 bp, Artificial Sequence
  Synthetic primer
ccaggaggtctggggtaccagtgggctcagatgaagctcttgtgctcatg

SEQ ID NO: 1084 - DNA, 50 bp, Artificial Sequence
  Synthetic primer
agctattaagaagcacgtgtgtgcaggaagcggaggcgcaggacctcacc

SEQ ID NO: 1085 - DNA, 50 bp, Artificial Sequence
  Synthetic primer
gtcaacaggggtgtgatggtgaaggcagagtgtgaaaaacgtgagagagg

SEQ ID NO: 1086 - DNA, 50 bp, Artificial Sequence
  Synthetic primer
gtagtcagaggagctatgatagaccacacccaagcaaggctgccctcaaa

SEQ ID NO: 1087 - DNA, 50 bp, Artificial Sequence
  Synthetic primer
gggtactttcagtacacaacacccctaagatttcccagtggtccgagcag

SEQ ID NO: 1088 - DNA, 50 bp, Artificial Sequence
  Synthetic primer
cttgctatagaagacctgggacagaggactgctgtctgccctctctggtc

SEQ ID NO: 1089 - DNA, 50 bp, Artificial Sequence
  Synthetic primer
tgctctccggaaggaattctggtttcgcgtgatcctgcctgcgtccgtgt

SEQ ID NO: 1090 - DNA, 50 bp, Artificial Sequence
  Synthetic primer
gccacaaatagaaaagttttctgcaaatgtaccccattcccctgtgcgag

SEQ ID NO: 1091 - DNA, 50 bp, Artificial Sequence
  Synthetic primer
caattctcccaatgagccttttgtctgtgggaaaagcaggagacgcttcg

SEQ ID NO: 1092 - DNA, 50 bp, Artificial Sequence
  Synthetic primer
ccagctaagcccagaccccgtggattctagatagattttagaggcagcag

SEQ ID NO: 1093 - DNA, 50 bp, Artificial Sequence
  Synthetic primer
ccttccagaatcccggataaagccaacgtgcagagagaccatgctagctg

SEQ ID NO: 1094 - DNA, 50 bp, Artificial Sequence
  Synthetic primer
acaggctttaccggccccacctggcatcggacaccatctaccagatcatg

SEQ ID NO: 1095 - DNA, 50 bp, Artificial Sequence
  Synthetic primer
cctcttcatgcaatctggtgagcagtggtgcaggcggggagccagagaaa

SEQ ID NO: 1096 - DNA, 50 bp, Artificial Sequence
  Synthetic primer
ctggcagtggaaacaccagatagaagatcttaggagaggcccagaaatgc

SEQ ID NO: 1097 - DNA, 50 bp, Artificial Sequence
  Synthetic primer
cacctaggcgctctggtggcccttgcagaagccctaagtacggttactta

SEQ ID NO: 1098 - DNA, 50 bp, Artificial Sequence
  Synthetic primer
ttggtcggcccacagcttcctgaagccaagctccccgggcccagctcagg

SEQ ID NO: 1099 - DNA, 50 bp, Artificial Sequence
  Synthetic primer
ccctcaaagtgtcacaatgctcctcctgatgactcctccccagaaaacca

SEQ ID NO: 1100 - DNA, 50 bp, Artificial Sequence
  Synthetic primer
ggccggttagaacccgtgactgtattctctcccttggatacccctatgcc

SEQ ID NO: 1101 - DNA, 50 bp, Artificial Sequence
  Synthetic primer
cgccatcacagctgaacttgttgagatccccgtgttactgcctatcagca

SEQ ID NO: 1102 - DNA, 50 bp, Artificial Sequence
  Synthetic primer
ggcgggggccgggcttggaacgatgtacgataccctcatagtgaccatta

SEQ ID NO: 1103 - DNA, 50 bp, Artificial Sequence
  Synthetic primer
accaattcacagggaaactagagactacggctgtggtggtaacctgcaga

SEQ ID NO: 1104 - DNA, 50 bp, Artificial Sequence
  Synthetic primer
ctccactttgctggttcagccttcgtgtggctcctggtaacgtggctcca

SEQ ID NO: 1105 - DNA, 50 bp, Artificial Sequence
  Synthetic primer
gactgtcaggaagggtcggagtctgtaaaaccagcatacagtttggcttt

SEQ ID NO: 1106 - DNA, 50 bp, Artificial Sequence
  Synthetic primer
gttcttacccttgcactgtcacccatacaggacgcacagggatggacgcg

SEQ ID NO: 1107 - DNA, 50 bp, Artificial Sequence
  Synthetic primer
ccaggatccctgagctttcccagtagcctccagtttcctttgtaagaccc

SEQ ID NO: 1108 - DNA, 50 bp, Artificial Sequence
  Synthetic primer
tgcagctgctccccggtgcacccgcaacaggcgttttgcaatgcagatgt

SEQ ID NO: 1109 - DNA, 50 bp, Artificial Sequence
  Synthetic primer
gcgaggctgccacagaggatggaaatgtccttaagcgcagccctgagttg

SEQ ID NO: 1110 - DNA, 50 bp, Artificial Sequence
  Synthetic primer
caaagactaatggggagagggagggccgggaagccaggaaagcttggtgt

SEQ ID NO: 1111 - DNA, 50 bp, Artificial Sequence
  Synthetic primer
gctctgagacgaatgctatgggctgcagatgacttcttagaggatttgcc

SEQ ID NO: 1112 - DNA, 50 bp, Artificial Sequence
  Synthetic primer
cactggctacttctgaagttctggcttccttctctctaggaggagtcgtg

SEQ ID NO: 1113 - DNA, 50 bp, Artificial Sequence
  Synthetic primer
tatgggagcatcggctactgctggtgtgtcttccccaacggcacggaggt

SEQ ID NO: 1114 - DNA, 50 bp, Artificial Sequence
  Synthetic primer
catttcctgagaccacagagagaggggagaagcctgggattgacagaagc

SEQ ID NO: 1115 - DNA, 50 bp, Artificial Sequence
  Synthetic primer
gctgccacatgtcaggccttatgctaagggtgagtaattccatggtgcac

SEQ ID NO: 1116 - DNA, 50 bp, Artificial Sequence
  Synthetic primer
cctactcctacagtgccttgcattccgtagctgctcagtacattaaccca

SEQ ID NO: 1117 - DNA, 50 bp, Artificial Sequence
  Synthetic primer
aatatgactattctaaaggctgtgaggccatggggtattggttaagttgc

SEQ ID NO: 1118 - DNA, 50 bp, Artificial Sequence
  Synthetic primer
gctgtattgctccatctcccccagcttctcattatgtacctcagggtatg

SEQ ID NO: 1119 - DNA, 50 bp, Artificial Sequence
  Synthetic primer
gtgcgtcctattgagtcactgctaatttcttgagcctggtatttgctgcc

SEQ ID NO: 1120 - DNA, 50 bp, Artificial Sequence
  Synthetic primer
ccaccgttttcacagggagaagcaggtctaggactcccctgcacctttgc

SEQ ID NO: 1121 - DNA, 50 bp, Artificial Sequence
  Synthetic primer
ccaggtcccccagagccaatctgccccctctctatgctcttgaacttcta

SEQ ID NO: 1122 - DNA, 50 bp, Artificial Sequence
  Synthetic primer
gggcaccaaggtgttaatggcaagcgtgcaaggttcaaagagaaggaagc

SEQ ID NO: 1123 - DNA, 50 bp, Artificial Sequence
  Synthetic primer
gtatcatgcagggtcctgtgatctagccaggagcagccattaacctagtg

SEQ ID NO: 1124 - DNA, 50 bp, Artificial Sequence
  Synthetic primer
ggtccctgccttaggcctacctgatggaagtaaagcctcaaccacaaaaa

SEQ ID NO: 1125 - DNA, 50 bp, Artificial Sequence
  Synthetic primer
tgttcaagtcctagtctataggattggcagtttaaatgctttactccccc

SEQ ID NO: 1126 - DNA, 50 bp, Artificial Sequence
  Synthetic primer
ctgggctggtcggatgagctgaggccattgacttctctacaaatcacttg

SEQ ID NO: 1127 - DNA, 50 bp, Artificial Sequence
  Synthetic primer
agaactttctccagtcagcctctacagaccaagctcatgactcacaatgg

SEQ ID NO: 1128 - DNA, 50 bp, Artificial Sequence
  Synthetic primer
cagaactttctccagtcagcctctacagaccaagctcatgactcacaatg

SEQ ID NO: 1129 - DNA, 50 bp, Artificial Sequence
  Synthetic primer
aggccgtgtcagcacgatggaccccaaacagaccaccctcctgtgtcttg

SEQ ID NO: 1130 - DNA, 50 bp, Artificial Sequence
  Synthetic primer
ccttagcccttctgtggttgtcatggcttaatgtgtgtctttccagaact

SEQ ID NO: 1131 - DNA, 50 bp, Artificial Sequence
  Synthetic primer
ctggacttacaaaatgccaagggggtgactggaagttgtggatatcaggg

SEQ ID NO: 1132 - DNA, 50 bp, Artificial Sequence
  Synthetic primer
gcagtggcttcactggaaaaatggtgtggctagcatttccctttgagtca

SEQ ID NO: 1133 - DNA, 50 bp, Artificial Sequence
  Synthetic primer
gcaaatgctatgagatgcatggagggctgccctgtaccccagcacttgtg

SEQ ID NO: 1134 - DNA, 50 bp, Artificial Sequence
  Synthetic primer
acaaggtgctaaaacaggttcaccccgatactggcatctcatccaaggcc

SEQ ID NO: 1135 - DNA, 50 bp, Artificial Sequence
  Synthetic primer
cctcagggaccgcttgtgctcggatggcttcttatttccccaatacccca

SEQ ID NO: 1136 - DNA, 50 bp, Artificial Sequence
  Synthetic primer
caccttcctggtctgttggatgccttatatcgtgatctgcttcttggtgg

SEQ ID NO: 1137 - DNA, 50 bp, Artificial Sequence
  Synthetic primer
ggaatggaagacccatctatctctttctccaaacccaagaaaaagaaatc

SEQ ID NO: 1138 - DNA, 50 bp, Artificial Sequence
  Synthetic primer
ccttggtcagagggactcaggtttgggacagcacaagctgaaggctggag

SEQ ID NO: 1139 - DNA, 50 bp, Artificial Sequence
  Synthetic primer
ccagagccagctagcccacacagaccccatcagagcaaaacagacaagag

SEQ ID NO: 1140 - DNA, 50 bp, Artificial Sequence
  Synthetic primer
tttcgcccctcggagaaccggtaggttggggtccctcggcgggggtctag

SEQ ID NO: 1141 - DNA, 50 bp, Artificial Sequence
  Synthetic primer
ctgctccccttgcataaaggatctctatatcctagaatcccaggtttagg

SEQ ID NO: 1142 - DNA, 50 bp, Artificial Sequence
  Synthetic primer
gtccgatacctggaaattagcttagtacacaagactcccaattactattt

SEQ ID NO: 1143 - DNA, 50 bp, Artificial Sequence
  Synthetic primer
ggctggtcacatctggaagcattgttcagcaggattaacctgatggccta

SEQ ID NO: 1144 - DNA, 50 bp, Artificial Sequence
  Synthetic primer
ttgtgccactgcacactccaatctgggtgaaagaccgagactccgcctca

SEQ ID NO: 1145 - DNA, 50 bp, Artificial Sequence
  Synthetic primer
ggctctcctagtgcccagagacaggcccagaggtttacaagttttctaag

SEQ ID NO: 1146 - DNA, 50 bp, Artificial Sequence
  Synthetic primer
tgtggcaacacccccagctttggctccaaaatcatccgaggtcctgagga

SEQ ID NO: 1147 - DNA, 50 bp, Artificial Sequence
  Synthetic primer
ttcccagggcaaggctgatctgttgccgtattagtccgttttcacacagc

SEQ ID NO: 1148 - DNA, 50 bp, Artificial Sequence
  Synthetic primer
tccaagtgcggcactgcaaagagacgcttcaagtggggagaagcggcgat

SEQ ID NO: 1149 - DNA, 50 bp, Artificial Sequence
  Synthetic primer
tggaggaagttctacactcgcctcaccaacagccgacacggtgagaccac

SEQ ID NO: 1150 - DNA, 50 bp, Artificial Sequence
  Synthetic primer
caggtcctgcagtctggctgagccctgcttggttgtctccacacacagct

SEQ ID NO: 1151 - DNA, 50 bp, Artificial Sequence
  Synthetic primer
ccctacctgaaagccatctcaagactcctcagatgccaccaacaacccca

SEQ ID NO: 1152 - DNA, 50 bp, Artificial Sequence
  Synthetic primer
ctgtgtgctggaaaatgatcccggggccaggacagacaaaccagagcctc

SEQ ID NO: 1153 - DNA, 50 bp, Artificial Sequence
  Synthetic primer
ccaagttcatcctgggctccattgggtctgccattgcggctgtcattgcg

SEQ ID NO: 1154 - DNA, 50 bp, Artificial Sequence
  Synthetic primer
caggactctgttacctaagatgtgataagctgggctgcaggcggttcagc

SEQ ID NO: 1155 - DNA, 50 bp, Artificial Sequence
  Synthetic primer
gccagtgtctttctttggtgcctttcctgttcagcattcttagcctgtgg

SEQ ID NO: 1156 - DNA, 50 bp, Artificial Sequence
  Synthetic primer
agtcctggcctccgcagatgcttcattttgacccttggctgcagtggaag

SEQ ID NO: 1157 - DNA, 50 bp, Artificial Sequence
  Synthetic primer
cagggtatgaaagtgcccatttctagccaacattagataccctcagtctc

SEQ ID NO: 1158 - DNA, 50 bp, Artificial Sequence
  Synthetic primer
tgagtggaggcgaaccactgagccaaggagctggtagttacgttgtccgc

SEQ ID NO: 1159 - DNA, 50 bp, Artificial Sequence
  Synthetic primer
cgaaaacccgtcggacattctgtaagaagtgtgacaagcaccaacctcac

SEQ ID NO: 1160 - DNA, 50 bp, Artificial Sequence
  Synthetic primer
gcttctactggcgcgtgagttcccggagtgagttgaaccaggtggaccaa

SEQ ID NO: 1161 - DNA, 50 bp, Artificial Sequence
  Synthetic primer
aaagaagggcccgagcttagtttccccaggactggcctaggaaggagcac

SEQ ID NO: 1162 - DNA, 50 bp, Artificial Sequence
  Synthetic primer
cttgaaaaggatgttggtgccttatgtgacacgagccagagcctgctggg

SEQ ID NO: 1163 - DNA, 50 bp, Artificial Sequence
  Synthetic primer
ggttttctcctacatggaccgacagccaaatctgccttcacctggcacgc

SEQ ID NO: 1164 - DNA, 50 bp, Artificial Sequence
  Synthetic primer
tccccacaggaagccccaagtcagccgcccgctacatactcactggtaaa

SEQ ID NO: 1165 - DNA, 50 bp, Artificial Sequence
  Synthetic primer
gtggagaccggcacccagacaggtggcgacggcagaggagcggaattctc

SEQ ID NO: 1166 - DNA, 50 bp, Artificial Sequence
  Synthetic primer
cgggacctgtgtgggacctgtgtcctgtggtggccgtttgcagtttctct

SEQ ID NO: 1167 - DNA, 50 bp, Artificial Sequence
  Synthetic primer
cttttccaagttcccaaggcctacagctgaagcccttaggtacctgtgtt

SEQ ID NO: 1168 - DNA, 50 bp, Artificial Sequence
  Synthetic primer
tgaggacagttcagaggaggattcagacgagtgtcgctgcgtgagtggcc

SEQ ID NO: 1169 - DNA, 50 bp, Artificial Sequence
  Synthetic primer
gcgcttaccagaaagccgcctgacaccatgggaggtgtggtttattggca

SEQ ID NO: 1170 - DNA, 50 bp, Artificial Sequence
  Synthetic primer
acgtcctgaggacagttctgtgaactgaatgcagcctggacactggcctc

SEQ ID NO: 1171 - DNA, 50 bp, Artificial Sequence
  Synthetic primer
cccttggacagaagactggcctacctagcagacctggatttttcttcctg

SEQ ID NO: 1172 - DNA, 50 bp, Artificial Sequence
  Synthetic primer
gctggagtatatcctgacccagacctacgacattgaagatttgcagccgg

SEQ ID NO: 1173 - DNA, 50 bp, Artificial Sequence
  Synthetic primer
gccaacattcagtctggtatgtgaggcgtgcgtgaagcaagaactcctgg

SEQ ID NO: 1174 - DNA, 50 bp, Artificial Sequence
  Synthetic primer
ggtgacctatcctggccctctactcagaaaccggcaatttgtcttcactc

SEQ ID NO: 1175 - DNA, 50 bp, Artificial Sequence
  Synthetic primer
cgggacctcaaggtgatgacagatgtagccggcaaccctgaagaggagcg

SEQ ID NO: 1176 - DNA, 50 bp, Artificial Sequence
  Synthetic primer
ccctgcctgagaggttagcttcctacatataagctccttttgtattggac

SEQ ID NO: 1177 - DNA, 50 bp, Artificial Sequence
  Synthetic primer
gtgccaggtgcatctgtggacaggggccatgctcctgggcttccaaagtt

SEQ ID NO: 1178 - DNA, 50 bp, Artificial Sequence
  Synthetic primer
cctaccgatcaagatgagttcagctagaagtcataccaccctcaggaatc

SEQ ID NO: 1179 - DNA, 50 bp, Artificial Sequence
  Synthetic primer
gctgagtcctgaagaccagagagtattcaactttgacgtgcgccagttga

SEQ ID NO: 1180 - DNA, 50 bp, Artificial Sequence
  Synthetic primer
ggcgtcccccaacttcttagagggacaagtggccttcagccacccgaaat

SEQ ID NO: 1181 - DNA, 50 bp, Artificial Sequence
  Synthetic primer
gcaggggatcgacattgagaccaagatgcacgtccgcttccttaacatgg

SEQ ID NO: 1182 - DNA, 50 bp, Artificial Sequence
  Synthetic primer
gcctgctcctaacaattcagcaagtcaggggcttcctctctgttagtccc

SEQ ID NO: 1183 - DNA, 50 bp, Artificial Sequence
  Synthetic primer
ttgcacgtgtggtaagcataggcttgaagaggtgggtaggcaggtacatg

SEQ ID NO: 1184 - DNA, 50 bp, Artificial Sequence
  Synthetic primer
tgcatgagatcacacaactaggcggtgactgagtccaacacaccaaagcc

SEQ ID NO: 1185 - DNA, 50 bp, Artificial Sequence
  Synthetic primer
ttctgacctgctggcctctctgaagggggtgtctgctctaactggatcaa

SEQ ID NO: 1186 - DNA, 50 bp, Artificial Sequence
  Synthetic primer
ctttcgagagagaagtttaagcttcaccaatgctgctcctgaccaatgga

SEQ ID NO: 1187 - DNA, 50 bp, Artificial Sequence
  Synthetic primer
attcatcctcactcccactcatacccgcctccctggacagttccctgctg

SEQ ID NO: 1188 - DNA, 50 bp, Artificial Sequence
  Synthetic primer
ctggtgaccgccacagccccgctttgtaaccagggaatacacagtcattt

SEQ ID NO: 1189 - DNA, 50 bp, Artificial Sequence
  Synthetic primer
tgaccccagcacacctctggctaacccataccccacacctgcccagctct

SEQ ID NO: 1190 - DNA, 50 bp, Artificial Sequence
  Synthetic primer
cggacagtgatggctcttggaaatgggtggatggcacagactataggcac

SEQ ID NO: 1191 - DNA, 50 bp, Artificial Sequence
  Synthetic primer
cacagaaatgcttgcagcctaaggcagggttttcagaccgtgggtcccag

SEQ ID NO: 1192 - DNA, 50 bp, Artificial Sequence
  Synthetic primer
aagacctcatgctcatgcagtggacatttcttggcctgttttcctgtggg

SEQ ID NO: 1193 - DNA, 50 bp, Artificial Sequence
  Synthetic primer
gctgacccagcatcagccacactctgggttggaaaatgtttgcctgttgg

SEQ ID NO: 1194 - DNA, 50 bp, Artificial Sequence
  Synthetic primer
cttcgaggctggccctgccaggcagttttcccggcgttttaggatctgta

SEQ ID NO: 1195 - DNA, 50 bp, Artificial Sequence
  Synthetic primer
ccttcatggaagtccttgctctgcgtggcatctgtcagcttttcaccttt

SEQ ID NO: 1196 - DNA, 50 bp, Artificial Sequence
  Synthetic primer
ccatgggccttttcacaggggacacaggcttcttaaaacaacccggcttc

SEQ ID NO: 1197 - DNA, 50 bp, Artificial Sequence
  Synthetic primer
gctggccgaaactgagtatgtgcagtaggatggatgtttgagcgacacac

SEQ ID NO: 1198 - DNA, 50 bp, Artificial Sequence
  Synthetic primer
cctagaggggcaaaagatggagtgagccacaggaagtttggggcgtggtg

SEQ ID NO: 1199 - DNA, 50 bp, Artificial Sequence
  Synthetic primer
cctcctgtaacgactggaagcaccaggctaagaacctgacccctcggact

SEQ ID NO: 1200 - DNA, 50 bp, Artificial Sequence
  Synthetic primer
cagacccagaagaagcaaaattactttcccctgactccatccgagcccag

SEQ ID NO: 1201 - DNA, 50 bp, Artificial Sequence
  Synthetic primer
tcatctgcaaatgtggccaggcttggggaacaatgatggtgcacaaaggc

SEQ ID NO: 1202 - DNA, 50 bp, Artificial Sequence
  Synthetic primer
ctgtggaggatgcaggattaccttcttatgaacaggcagtggcgctgacc

SEQ ID NO: 1203 - DNA, 50 bp, Artificial Sequence
  Synthetic primer
tgactcaagctaccacttactgttgattgtgacgaaatgccagctgaggc

SEQ ID NO: 1204 - DNA, 50 bp, Artificial Sequence
  Synthetic primer
ctgccacatcttcccactcccgggactcagcccagtacacctcgctgatt

SEQ ID NO: 1205 - DNA, 50 bp, Artificial Sequence
  Synthetic primer
cccagctacttgagagatgacgagggaagatcacttgagcccaggagtta

SEQ ID NO: 1206 - DNA, 50 bp, Artificial Sequence
  Synthetic primer
ctgccgccattcctgcagcatcaccataagcagtgcagggtgtctccatc

SEQ ID NO: 1207 - DNA, 50 bp, Artificial Sequence
  Synthetic primer
ccctctgtacctctcataactggtcaacgactgtaacaggttacatcagg

SEQ ID NO: 1208 - DNA, 50 bp, Artificial Sequence
  Synthetic primer
ccagaggccatattcaaaacagggtcttctcagtgtatgcaaggggctgc

SEQ ID NO: 1209 - DNA, 50 bp, Artificial Sequence
  Synthetic primer
cgctgagtagtctgaggagcaagaggagttggtctttctgtcccagtggc

SEQ ID NO: 1210 - DNA, 50 bp, Artificial Sequence
  Synthetic primer
catggcccgggtgtagagaaaccaagcggcagccaccaccagtgttgttt

SEQ ID NO: 1211 - DNA, 50 bp, Artificial Sequence
  Synthetic primer
gatgctcataggagaacgcaccctgttgtgaaccgtgcatgtgagggatc

SEQ ID NO: 1212 - DNA, 50 bp, Artificial Sequence
  Synthetic primer
gcatctgccgagcacctcctgtttgccaggcgctttctatacttaatccc

SEQ ID NO: 1213 - DNA, 50 bp, Artificial Sequence
  Synthetic primer
gagtgcctccggcctccccatccacctctgcctaagtaaatctgctctca

SEQ ID NO: 1214 - DNA, 50 bp, Artificial Sequence
  Synthetic primer
gaggagctgcagattagctaggggacagctggaattatgctggcttctga

SEQ ID NO: 1215 - DNA, 50 bp, Artificial Sequence
  Synthetic primer
gcaggcaggaacagtgtgggtgaattgctatggcgtggtaagtgcccagt

SEQ ID NO: 1216 - DNA, 50 bp, Artificial Sequence
  Synthetic primer
gaagaagatgatcgagacagccgacatcagcgtgacagcaggagatgccc

SEQ ID NO: 1217 - DNA, 50 bp, Artificial Sequence
  Synthetic primer
tcccatgagtcagctgatttcagtgctgctgaactaatttctgtgtctaa

SEQ ID NO: 1218 - DNA, 50 bp, Artificial Sequence
  Synthetic primer
cacccctatctaacaccacccttggctcccactccagctccctgtattga

SEQ ID NO: 1219 - DNA, 50 bp, Artificial Sequence
  Synthetic primer
attgaaaggagagcaacggggctgaggccgtgtcagcacgatggacccca

SEQ ID NO: 1220 - DNA, 50 bp, Artificial Sequence
  Synthetic primer
ctgcaggtctcccatgaaggccaccccatggtctgatgggcatgaagcat

SEQ ID NO: 1221 - DNA, 50 bp, Artificial Sequence
  Synthetic primer
catcaacagctaaactgcacagggaggaggatcgaacggatccctcccgc

SEQ ID NO: 1222 - DNA, 50 bp, Artificial Sequence
  Synthetic primer
gggctcagaagctgcactaggccccgagtccccatgtgtctccttgaatt

SEQ ID NO: 1223 - DNA, 50 bp, Artificial Sequence
  Synthetic primer
cttcaagggttctggaggagggaagggtctgcaggttccatgggtgacag

SEQ ID NO: 1224 - DNA, 50 bp, Artificial Sequence
  Synthetic primer
ccaggaggatcccttgatcccttgtggccaggagttgggagaccagcctg

SEQ ID NO: 1225 - DNA, 50 bp, Artificial Sequence
  Synthetic primer
atgtggaggtggctggttcccatgaacgtggttgtcagaggcgggggaca

SEQ ID NO: 1226 - DNA, 50 bp, Artificial Sequence
  Synthetic primer
ctatcgccgtccccttttgcagccctctctggggatggactgggtaaatg

SEQ ID NO: 1227 - DNA, 50 bp, Artificial Sequence
  Synthetic primer
tggccacatttgtctcaaactcaagtctacacatttctctctcttttccc

SEQ ID NO: 1228 - DNA, 50 bp, Artificial Sequence
  Synthetic primer
cccatctccaactcggaagtaagcccaagagaacaacataaagcaaacaa

SEQ ID NO: 1229 - DNA, 50 bp, Artificial Sequence
  Synthetic primer
gtctcggttcaaattccaaacctaccatcttcagttgtgcgaccttgggc

SEQ ID NO: 1230 - DNA, 50 bp, Artificial Sequence
  Synthetic primer
acccaaatctaggtttgaacggtgaggtgtcaggtcatcaaatattcatg

SEQ ID NO: 1231 - DNA, 50 bp, Artificial Sequence
  Synthetic primer
tgaatgaagccctggagtactatgagcgggccctgagactggctgctgac

SEQ ID NO: 1232 - DNA, 50 bp, Artificial Sequence
  Synthetic primer
ggggaaaagggtgggattgtccagcatgcttgtatgtatatttcagaacc

SEQ ID NO: 1233 - DNA, 50 bp, Artificial Sequence
  Synthetic primer
gactgtgactcctactacctgccagccttcttccttgctctgcgctggga

SEQ ID NO: 1234 - DNA, 50 bp, Artificial Sequence
  Synthetic primer
tgaggcccaaagcacttgcttacatcctctgatagctgtttcaaatgtgc

SEQ ID NO: 1235 - DNA, 50 bp, Artificial Sequence
  Synthetic primer
actctaggcacaatcggtgtcaatcgctctcaggctccagtgctgttgtc

SEQ ID NO: 1236 - DNA, 50 bp, Artificial Sequence
  Synthetic primer
gtcagtggtggatattctcagtcatgcatctgtgtggctgttccgagggg

SEQ ID NO: 1237 - DNA, 50 bp, Artificial Sequence
  Synthetic primer
cctcaaaaacctcttcatgaggatggtgaccccagccatgaggaccctcg

SEQ ID NO: 1238 - DNA, 50 bp, Artificial Sequence
  Synthetic primer
agaggaggctacatcctgccctggcaagagcctgctatgaacactcactc

SEQ ID NO: 1239 - DNA, 50 bp, Artificial Sequence
  Synthetic primer
tggatgccacggatcaggaaagcctggagctgaagccaacctcccgagca

SEQ ID NO: 1240 - DNA, 50 bp, Artificial Sequence
  Synthetic primer
ccactctgtggaggttaatttctgggaccttagggataatatgcctttcg

SEQ ID NO: 1241 - DNA, 50 bp, Artificial Sequence
  Synthetic primer
gacaccagtcatactggctcagggcccaccgctaatgccttaatgaaatc

SEQ ID NO: 1242 - DNA, 50 bp, Artificial Sequence
  Synthetic primer
agatgtgttttcagagctaggtacagaggaatgtttgctacctttagcgg

SEQ ID NO: 1243 - DNA, 50 bp, Artificial Sequence
  Synthetic primer
ggaaaccagctttctggttaaggttaacaccagatggtgcccctcattgg

SEQ ID NO: 1244 - DNA, 50 bp, Artificial Sequence
  Synthetic primer
catcctgctcttggtttttgtcacagaagttgttgtagtggttttgggat

SEQ ID NO: 1245 - DNA, 50 bp, Artificial Sequence
  Synthetic primer
tctccaggggcaaacctctgatgtcttctttgcagccagtagcttgactg

SEQ ID NO: 1246 - DNA, 50 bp, Artificial Sequence
  Synthetic primer
ttgacgacggagaagatcctttggatgcagagagccagcaaggaggcggc

SEQ ID NO: 1247 - DNA, 50 bp, Artificial Sequence
  Synthetic primer
ttcccaggctggaaccagggtctctctttacctcctaccccatggtggca

SEQ ID NO: 1248 - DNA, 50 bp, Artificial Sequence
  Synthetic primer
tgacgccattcctcctaagaaggctcaaagtaatggacctgaaaagcagg

SEQ ID NO: 1249 - DNA, 50 bp, Artificial Sequence
  Synthetic primer
atgtcactcctctgctgggctgttcattgtcattgctgtgttcagggacc

SEQ ID NO: 1250 - DNA, 50 bp, Artificial Sequence
  Synthetic primer
gccatcggtagagatgaggatcaggaagctggtgttcagagggatcatgg

SEQ ID NO: 1251 - DNA, 50 bp, Artificial Sequence
  Synthetic primer
gtacgacgtttgatccatgcccatccaaaaggatgatgaagttcaggttg

SEQ ID NO: 1252 - DNA, 50 bp, Artificial Sequence
  Synthetic primer
ggctgtgtgccccagtttgagaagtggttgcaggacaatttaaccatcgt

SEQ ID NO: 1253 - DNA, 50 bp, Artificial Sequence
  Synthetic primer
gcccttgaacttggtcgttaggaaacatccacactgaagaggaacctgac

SEQ ID NO: 1254 - DNA, 50 bp, Artificial Sequence
  Synthetic primer
agaaacccgagacaagctgcccagcacagaggtgaaggaagacagtgcgt

SEQ ID NO: 1255 - DNA, 50 bp, Artificial Sequence
  Synthetic primer
ttgagaagcgcttcgtacccagccagcactatgaagccttccctgactag

SEQ ID NO: 1256 - DNA, 50 bp, Artificial Sequence
  Synthetic primer
ggccaaacttggagcaggtgtccatcccagccctgtgtagttagagcagg

SEQ ID NO: 1257 - DNA, 50 bp, Artificial Sequence
  Synthetic primer
aggcaggcctcatttcatcacgcagcatgtgcaggcctggaagagcaaag

SEQ ID NO: 1258 - DNA, 50 bp, Artificial Sequence
  Synthetic primer
gcctgtccagctccctctccccaagaaacaacatgaatgagcaacttcag

SEQ ID NO: 1259 - DNA, 50 bp, Artificial Sequence
  Synthetic primer
gtgaagcaactgaatcttcagcatgttctcatcggcggagccttcttgtg

SEQ ID NO: 1260 - DNA, 50 bp, Artificial Sequence
  Synthetic primer
ggactggctttctggttggatttcaggtaagatgtgtttaaggccagagc

SEQ ID NO: 1261 - DNA, 50 bp, Artificial Sequence
  Synthetic primer
gcaggactggttgggtctggggatctatggcatccaagacagtgacactc

SEQ ID NO: 1262 - DNA, 50 bp, Artificial Sequence
  Synthetic primer
gggccatcatcctcttcatcatcctcttcatcctgctgctgttcctggcc

SEQ ID NO: 1263 - DNA, 50 bp, Artificial Sequence
  Synthetic primer
ccctcggagagcttgctttagagtcttggagagacggccatggtctgcgt

SEQ ID NO: 1264 - DNA, 50 bp, Artificial Sequence
  Synthetic primer
gtgaacacaggcatggcggcagaagtgccaaaagtgagccctctccagca

SEQ ID NO: 1265 - DNA, 50 bp, Artificial Sequence
  Synthetic primer
gggcaaacctcaggggacctgcaatcagaagacgtgaaggcagattttca

SEQ ID NO: 1266 - DNA, 50 bp, Artificial Sequence
  Synthetic primer
agcccccttactgggggagggtccttgcaattccagcgaatcggaggcca

SEQ ID NO: 1267 - DNA, 50 bp, Artificial Sequence
  Synthetic primer
cagcactggaacttcttgagcaggagcatacccagggcttcataatcacc

SEQ ID NO: 1268 - DNA, 50 bp, Artificial Sequence
  Synthetic primer
gtgtcccaggtagcattgactcccgtcattggagtgaaatggatcaaagt

SEQ ID NO: 1269 - DNA, 50 bp, Artificial Sequence
  Synthetic primer
gcggctatcaccgaagcaggagtggccaaaatgaagtttaatccctttgt

SEQ ID NO: 1270 - DNA, 50 bp, Artificial Sequence
  Synthetic primer
gagctgatttgatcgaggagcgcggttaccggacgggctgggtctatggt

SEQ ID NO: 1271 - DNA, 50 bp, Artificial Sequence
  Synthetic primer
gggcccagaagctcctcaagtcggcctctccagacccacttgcagcctcc

SEQ ID NO: 1272 - DNA, 50 bp, Artificial Sequence
  Synthetic primer
tagcggggatcctgaactggactgaataaaacgtggtctcccactgcgcc

SEQ ID NO: 1273 - DNA, 50 bp, Artificial Sequence
  Synthetic primer
agctgctgaatgccagaggcacgaagtgacgttggtctcccgaaagcccg

SEQ ID NO: 1274 - DNA, 50 bp, Artificial Sequence
  Synthetic primer
gctgctaaagttccagcaaaaaagatcaccgccgcgagtaaaaaggctcc

SEQ ID NO: 1275 - DNA, 50 bp, Artificial Sequence
  Synthetic primer
catgaatggattctgggtggagatgcccctgacccttgacaaatctatcg

SEQ ID NO: 1276 - DNA, 50 bp, Artificial Sequence
  Synthetic primer
gtaccgtcagcaacctggacagagcctgacactgatcgcaactgcaaatc

SEQ ID NO: 1277 - DNA, 50 bp, Artificial Sequence
  Synthetic primer
cttgacatgagcacctttagatcccttcccctccatgggctttgggccac

SEQ ID NO: 1278 - DNA, 50 bp, Artificial Sequence
  Synthetic primer
ccttcggtgctcctcagagaaaatcgcacccccagcatctgacttcctgg

SEQ ID NO: 1279 - DNA, 50 bp, Artificial Sequence
  Synthetic primer
acaaatcagcctggtcaccagcttttcggaacagcagagacacagagggc

SEQ ID NO: 1280 - DNA, 50 bp, Artificial Sequence
  Synthetic primer
gacaaccccctccaccgccagtaataaagtcagccctgaaaagcatgaag

SEQ ID NO: 1281 - DNA, 50 bp, Artificial Sequence
  Synthetic primer
cctgagttaccatcagtgctaatgttcctcttttgggtatagttctccag

SEQ ID NO: 1282 - DNA, 50 bp, Artificial Sequence
  Synthetic primer
ccctccaaggcagcaaagcagaatcgggagcagtggagcagaaatgtgca

SEQ ID NO: 1283 - DNA, 50 bp, Artificial Sequence
  Synthetic primer
tttaaggactgctgatgccccctcaggcctcccccaagtttgctgggctt

SEQ ID NO: 1284 - DNA, 50 bp, Artificial Sequence
  Synthetic primer
gaagacagaagctctggcccctatggtggtagaggccaatactctgccaa

SEQ ID NO: 1285 - DNA, 50 bp, Artificial Sequence
  Synthetic primer
gcacaactgctgcctcacaacagccttttttggcccagttcctgtccagc

SEQ ID NO: 1286 - DNA, 50 bp, Artificial Sequence
  Synthetic primer
catgccacatccatgttcttgggtcatcttttttgccctttcatggagtc

SEQ ID NO: 1287 - DNA, 50 bp, Artificial Sequence
  Synthetic primer
cctgggttccaatcctggctctgtggcttgctagctatgtgaccttgagc

SEQ ID NO: 1288 - DNA, 50 bp, Artificial Sequence
  Synthetic primer
acgaaggagaccggcacccagacaggtggcgacggcaaaggagtaagtga

SEQ ID NO: 1289 - DNA, 50 bp, Artificial Sequence
  Synthetic primer
attcagagcatagcaggactggctggaccaccccttgcgggtttgttggt

SEQ ID NO: 1290 - DNA, 50 bp, Artificial Sequence
  Synthetic primer
taggcataaggaaactcgtttgcaggctctctgtccagggctgcttcctg

SEQ ID NO: 1291 - DNA, 50 bp, Artificial Sequence
  Synthetic primer
ggacattaagttcagtggccatgttagctgggtcgggaagacatccatgg

SEQ ID NO: 1292 - DNA, 50 bp, Artificial Sequence
  Synthetic primer
ctgttcagctcaggcacaggggcacagcagaggtttgggaagcggtctcc

SEQ ID NO: 1293 - DNA, 50 bp, Artificial Sequence
  Synthetic primer
gggcctcccgtttctaaagcacccactatggatgcacagccggacaaact

SEQ ID NO: 1294 - DNA, 50 bp, Artificial Sequence
  Synthetic primer
aggcacatgggatggatgtccagtgctaccacccaagccccctccttctt

SEQ ID NO: 1295 - DNA, 50 bp, Artificial Sequence
  Synthetic primer
tccaaggcctggatgctaatcaagatgaacaggtcgactttcaagaattc

SEQ ID NO: 1296 - DNA, 50 bp, Artificial Sequence
  Synthetic primer
agctcccacctggacgcatccctcaccacgtccggatttccttctttgga

SEQ ID NO: 1297 - DNA, 50 bp, Artificial Sequence
  Synthetic primer
gcacacctgctatcgtggaacagcctagaaaccaagggactccaccacca

SEQ ID NO: 1298 - DNA, 50 bp, Artificial Sequence
  Synthetic primer
gacttccactgccatcctcccaaggggcccaaattctttcagtggctacc

SEQ ID NO: 1299 - DNA, 50 bp, Artificial Sequence
  Synthetic primer
gtcaggttcccgtgggtcacttgctactcctaagggaagtttcttctgcc

SEQ ID NO: 1300 - DNA, 50 bp, Artificial Sequence
  Synthetic primer
acgtgctccctctgccaggaggagaatgaagacgtggtgcgagatgcgct

SEQ ID NO: 1301 - DNA, 50 bp, Artificial Sequence
  Synthetic primer
gccttcagggctgctcggagtcacctgttggaatggactaaaaggaccct

SEQ ID NO: 1302 - DNA, 50 bp, Artificial Sequence
  Synthetic primer
tcctgcaggtatggtcagaacccagtgacctcctggagattccggtctca

SEQ ID NO: 1303 - DNA, 50 bp, Artificial Sequence
  Synthetic primer
ctgcctccctcatggtgtgcgtgtcgttctcttcctgacgcatctgtgat

SEQ ID NO: 1304 - DNA, 50 bp, Artificial Sequence
  Synthetic primer
ggccacagcgaatcttaacctaacagccttgacaaactgcaccataggtg

SEQ ID NO: 1305 - DNA, 50 bp, Artificial Sequence
  Synthetic primer
tgcatggacggcgcaggcccgccagacttaagggacctggtcaccacgct

SEQ ID NO: 1306 - DNA, 50 bp, Artificial Sequence
  Synthetic primer
catgaggataacttccttgcccctgctctgtagccacctccttggcaccg

SEQ ID NO: 1307 - DNA, 50 bp, Artificial Sequence
  Synthetic primer
tacctgacagtatgaccgatctctgcgcctttctgggggcgggcaagctg

SEQ ID NO: 1308 - DNA, 50 bp, Artificial Sequence
  Synthetic primer
ccctgtgcaaatcagagtatgtgagtggaagaaggggtgagtcctaactg

SEQ ID NO: 1309 - DNA, 50 bp, Artificial Sequence
  Synthetic primer
ctcacagcagattcttcaggcccagcatctgcctcactgtggacccccca

SEQ ID NO: 1310 - DNA, 50 bp, Artificial Sequence
  Synthetic primer
gaagaaggacagcgtagtggcacacaaggcaaaaagccaccctgaggtgc

SEQ ID NO: 1311 - DNA, 50 bp, Artificial Sequence
  Synthetic primer
cccacccctcctccagggcaaccccttggtcctacagcaagaagccagaa

SEQ ID NO: 1312 - DNA, 50 bp, Artificial Sequence
  Synthetic primer
cactcacatgcacacactaaccttggagccgatgggattgagtgactggc

SEQ ID NO: 1313 - DNA, 50 bp, Artificial Sequence
  Synthetic primer
ccgacatgactaagtatctggaaggctcactgtaccccagcacccagcag

SEQ ID NO: 1314 - DNA, 50 bp, Artificial Sequence
  Synthetic primer
gttgagctgtggacgcgggtctctgttctgcaggatgggggtaaaactga

SEQ ID NO: 1315 - DNA, 50 bp, Artificial Sequence
  Synthetic primer
catgaacagccgtgagtatggggcctggaagcagcaggtggaatccaaga

SEQ ID NO: 1316 - DNA, 50 bp, Artificial Sequence
  Synthetic primer
cgtatggagtctcgaacccgaggatttgtctgccgtcacgatggagcggt

SEQ ID NO: 1317 - DNA, 50 bp, Artificial Sequence
  Synthetic primer
atgctaactagttacgcgacccccgagcgggtctgcgcctgccacctgtg

SEQ ID NO: 1318 - DNA, 50 bp, Artificial Sequence
  Synthetic primer
ccacctttgattggccgaaactcagtgattggcacaagagtaggttgcag

SEQ ID NO: 1319 - DNA, 50 bp, Artificial Sequence
  Synthetic primer
cactcacgagggcttccactgtgctgccgccctcttaatattcaccaata

SEQ ID NO: 1320 - DNA, 50 bp, Artificial Sequence
  Synthetic primer
ggaagagctatcaaggagtaccaatcaggtcggggcactcaggtgtttgt

SEQ ID NO: 1321 - DNA, 50 bp, Artificial Sequence
  Synthetic primer
ctggatcagagaccctgcctctgtttgaccccgcactgactgaataaagc

SEQ ID NO: 1322 - DNA, 50 bp, Artificial Sequence
  Synthetic primer
caggggaggcactgtctgaggagactacagcaggtgaacagtctacaagg

SEQ ID NO: 1323 - DNA, 50 bp, Artificial Sequence
  Synthetic primer
tcgccaccctcaccatccaaggcatccggtttgaggacaatggcatctac

SEQ ID NO: 1324 - DNA, 50 bp, Artificial Sequence
  Synthetic primer
gattgtggagccagttccaagtgccagattagagcagggctatgcagccc

SEQ ID NO: 1325 - DNA, 50 bp, Artificial Sequence
  Synthetic primer
atgtgtcacctggagctgggcactaaccattccaagccgccgcatcgctt

SEQ ID NO: 1326 - DNA, 50 bp, Artificial Sequence
  Synthetic primer
caggtaactttgaagagatgagcagtgagtgaccaggcagtttttctgcc

SEQ ID NO: 1327 - DNA, 50 bp, Artificial Sequence
  Synthetic primer
gcctgtacccgcgactaactagacttttcttgctgttgtgcaaagttcag

SEQ ID NO: 1328 - DNA, 50 bp, Artificial Sequence
  Synthetic primer
ccacagaggatgctcaggccgcagtggattattacacaaccacaccagcg

SEQ ID NO: 1329 - DNA, 50 bp, Artificial Sequence
  Synthetic primer
caggacttgcaacaccctgattgctcctgtctgattctttctgacgatca

SEQ ID NO: 1330 - DNA, 50 bp, Artificial Sequence
  Synthetic primer
ccctcttcctcactgacttccccttgcacaggggcttctgttttatccca

SEQ ID NO: 1331 - DNA, 50 bp, Artificial Sequence
  Synthetic primer
caccgtgtactttgatgggaagtatgccagccccgacgtcttcaaatcct

SEQ ID NO: 1332 - DNA, 50 bp, Artificial Sequence
  Synthetic primer
tggggagtgggcagtgtccccgttaccagtgccagcatctctgtgagtgg

SEQ ID NO: 1333 - DNA, 50 bp, Artificial Sequence
  Synthetic primer
cctccgacggcgtctccaggccccaaacggcctccggtcggtgggcttct

SEQ ID NO: 1334 - DNA, 50 bp, Artificial Sequence
  Synthetic primer
gctctctcagtggcctccttcttggtcccgtttcgttgggtcattgcctg

SEQ ID NO: 1335 - DNA, 50 bp, Artificial Sequence
  Synthetic primer
tctgctcaggccccttcagctactgccacttcgccttacatccctgctga

SEQ ID NO: 1336 - DNA, 50 bp, Artificial Sequence
  Synthetic primer
agtcagattcccacccctctgacgctctgaccaggaaaaaaccggccaag

SEQ ID NO: 1337 - DNA, 50 bp, Artificial Sequence
  Synthetic primer
ggtggagatgactgaaccaagaagataatctccaaaggtccacgatgggc

SEQ ID NO: 1338 - DNA, 50 bp, Artificial Sequence
  Synthetic primer
ggctctgtcagccatgtgactgtagtccacatggagctctcagcataccg

SEQ ID NO: 1339 - DNA, 50 bp, Artificial Sequence
  Synthetic primer
gcagccctagaaagtaagcccagggcttcagatctaagttagtccaaaag

SEQ ID NO: 1340 - DNA, 50 bp, Artificial Sequence
  Synthetic primer
acctaattgtggatgatgattgcgcagggaggggtacgtggcacctcttc

SEQ ID NO: 1341 - DNA, 50 bp, Artificial Sequence
  Synthetic primer
cagatacaactacggggttgtggagagcttcacagtgcagcggcgagtcc

SEQ ID NO: 1342 - DNA, 50 bp, Artificial Sequence
  Synthetic primer
aagaggccaaagaggccccagccgacaagtgatcgcccacaagccttact

SEQ ID NO: 1343 - DNA, 50 bp, Artificial Sequence
  Synthetic primer
taggattcacaccccacctgcgcttcacttgggtccaggcctactcctgt

SEQ ID NO: 1344 - DNA, 50 bp, Artificial Sequence
  Synthetic primer
gggctggctgtggtgacgctgctcaatttcctggtgtgcttcggacctta

SEQ ID NO: 1345 - DNA, 50 bp, Artificial Sequence
  Synthetic primer
ggctaaatagtcaaggggtaatatgggcctgttgtttagtgtctccttcc

SEQ ID NO: 1346 - DNA, 50 bp, Artificial Sequence
  Synthetic primer
aacgtggatggagggtgtgtcagggaggttgggacagaggcagagaagag

SEQ ID NO: 1347 - DNA, 50 bp, Artificial Sequence
  Synthetic primer
gtcttgtggacaacatctttcctcctgtggtcaacatcacatggctgagc

SEQ ID NO: 1348 - DNA, 50 bp, Artificial Sequence
  Synthetic primer
cgtgtcaaccccaaatttttacgtatgagatcctttagtccacccaatgg

SEQ ID NO: 1349 - DNA, 50 bp, Artificial Sequence
  Synthetic primer
tacttcacagaatggaggtttcagcagccaaagccccaagtgccgcagac

SEQ ID NO: 1350 - DNA, 50 bp, Artificial Sequence
  Synthetic primer
aggaggggaacgtggtaaaacccaagacattaaatctgccatctcaggcc

SEQ ID NO: 1351 - DNA, 50 bp, Artificial Sequence
  Synthetic primer
tggagaaggccgccagccgggaggaacctatcgaatgggagttctgaggg

SEQ ID NO: 1352 - DNA, 50 bp, Artificial Sequence
  Synthetic primer
ggcattaccccggaccagcagaggctcatctttgcagtcaagcaggtgga

SEQ ID NO: 1353 - DNA, 50 bp, Artificial Sequence
  Synthetic primer
ggactgaatctggttctcctcttgtacacccctcaaccctatgcagcctg

SEQ ID NO: 1354 - DNA, 50 bp, Artificial Sequence
  Synthetic primer
gagtgggcagacatcgaagccaaacagcagtatcccggaagcactcatgc

SEQ ID NO: 1355 - DNA, 50 bp, Artificial Sequence
  Synthetic primer
agttcagaaggtgtgtccttgaactgaggccctgtgatatggcggcctgc

SEQ ID NO: 1356 - DNA, 50 bp, Artificial Sequence
  Synthetic primer
cctgcccactaacaatctcagtccgtgaacaaaaccaacatgaacattcc

SEQ ID NO: 1357 - DNA, 50 bp, Artificial Sequence
  Synthetic primer
cctgaggcatccgtgtcttaacccatctgccgttcaccagttggcaagct

SEQ ID NO: 1358 - DNA, 50 bp, Artificial Sequence
  Synthetic primer
gggagctgattgtagcagcctcgttagtgtcaccccctcctccctgatct

SEQ ID NO: 1359 - DNA, 50 bp, Artificial Sequence
  Synthetic primer
acactgctcacctgattggcctggtgtacatgggtcatcgggtaccctct

SEQ ID NO: 1360 - DNA, 50 bp, Artificial Sequence
  Synthetic primer
gcctccgtggcctccagcatccgacaagaagcttcagccatgcaggcccc

SEQ ID NO: 1361 - DNA, 50 bp, Artificial Sequence
  Synthetic primer
tgagcccaaggaagaatgagtcaccccagagccctagagggtcagatggg

SEQ ID NO: 1362 - DNA, 50 bp, Artificial Sequence
  Synthetic primer
tggaacacccaagaaaccatgcaaagcagtggacaacacagaacacgccc

SEQ ID NO: 1363 - DNA, 50 bp, Artificial Sequence
  Synthetic primer
tgcataggtaagatcctcgatctggtgttctctgcgtggctgttagggac

SEQ ID NO: 1364 - DNA, 50 bp, Artificial Sequence
  Synthetic primer
gctgcatgacaaagccgtccaaatccctactcccccacctaaagactggg

SEQ ID NO: 1365 - DNA, 50 bp, Artificial Sequence
  Synthetic primer
ggggtggcccctgacagcaagacaactggcagctcatacccttttcagct

SEQ ID NO: 1366 - DNA, 50 bp, Artificial Sequence
  Synthetic primer
cagtctggtcagtgagaaagggccgagggtagacaggcagcttctgtggt

SEQ ID NO: 1367 - DNA, 50 bp, Artificial Sequence
  Synthetic primer
gactttccagttggccctgattttcaaccatgtgattgtttcactcctgg

SEQ ID NO: 1368 - DNA, 50 bp, Artificial Sequence
  Synthetic primer
ccccatgaatgatcagaactgacatttaattcatgtttgtctcgccatgc

SEQ ID NO: 1369 - DNA, 50 bp, Artificial Sequence
  Synthetic primer
cagtgatgtcgactctttgcccaccgccatctccagctgttgccaagaca

SEQ ID NO: 1370 - DNA, 50 bp, Artificial Sequence
  Synthetic primer
tatatttataagaaagggataaaggtaatagagcccttctgccccccacc

SEQ ID NO: 1371 - DNA, 50 bp, Artificial Sequence
  Synthetic primer
atgtggactgccctacatttggcctgtgccactggccaaccggaaatggt

SEQ ID NO: 1372 - DNA, 50 bp, Artificial Sequence
  Synthetic primer
ctttaattcttgggcctccaataagtgtcccataggtgtctggccaggcc

SEQ ID NO: 1373 - DNA, 50 bp, Artificial Sequence
  Synthetic primer
tctttgatatcgtactgaggtaacttccacgtagccccttgccacgcggc

SEQ ID NO: 1374 - DNA, 50 bp, Artificial Sequence
  Synthetic primer
ctgagcagactggacacaatcatctctcccttcctctatgtcaagcactg

SEQ ID NO: 1375 - DNA, 50 bp, Artificial Sequence
  Synthetic primer
cccaagtgccaggtgtgtgtagctcagagttccttttacagtgaggtgtc

SEQ ID NO: 1376 - DNA, 50 bp, Artificial Sequence
  Synthetic primer
ggggagggtggaagaaatggtggactgtatctctcacgttctgaagcagc

SEQ ID NO: 1377 - DNA, 50 bp, Artificial Sequence
  Synthetic primer
tgaaacaagtcaaccgaagctttgtggtgcaggagctgagggtgccccag

SEQ ID NO: 1378 - DNA, 50 bp, Artificial Sequence
  Synthetic primer
agggccgaggccgtggagctcatggaggcaaagctgaggataaggagtgg

SEQ ID NO: 1379 - DNA, 50 bp, Artificial Sequence
  Synthetic primer
gccccctaattgactgaatggaacccctcttgaccaaagtgaccccagaa

SEQ ID NO: 1380 - DNA, 50 bp, Artificial Sequence
  Synthetic primer
ctgagagacaaagaccaacgagccaccacagccaccagtcccagaacccg

SEQ ID NO: 1381 - DNA, 50 bp, Artificial Sequence
  Synthetic primer
gctgctccccctggtggtggatcctggaattttctcacgcaggagccatt

SEQ ID NO: 1382 - DNA, 50 bp, Artificial Sequence
  Synthetic primer
cgtgactaccgcctgcgccacctcgtgcctttctttatctacagcggctt

SEQ ID NO: 1383 - DNA, 50 bp, Artificial Sequence
  Synthetic primer
tgagtcactggacgttggcagggagttggctgtgagattgtaggtgctca

SEQ ID NO: 1384 - DNA, 50 bp, Artificial Sequence
  Synthetic primer
gaggccatccctgaagagccttggaacgtggcgggtctgtgctcctttta

SEQ ID NO: 1385 - DNA, 50 bp, Artificial Sequence
  Synthetic primer
gactccctggattaatgctgtgtacccgtgggcaaggtgcccgaattctc

SEQ ID NO: 1386 - DNA, 50 bp, Artificial Sequence
  Synthetic primer
aacaggctggagtcgttgtgcctcagtatgaccgaacacgccctgggaga

SEQ ID NO: 1387 - DNA, 50 bp, Artificial Sequence
  Synthetic primer
gtgtcactaaagttggtatacaaccccccactgctaaatttgactggctt

SEQ ID NO: 1388 - DNA, 50 bp, Artificial Sequence
  Synthetic primer
tcttggtggggagattactgggaacccttccgtgcccgtctgaatgccca

SEQ ID NO: 1389 - DNA, 50 bp, Artificial Sequence
  Synthetic primer
catcgaatggtggcgggttaaactgtactgcttaagtggagcggctaccg

SEQ ID NO: 1390 - DNA, 50 bp, Artificial Sequence
  Synthetic primer
gtttggtcagctaaaactcagctgcagcctggacagtagagcgagacccc

SEQ ID NO: 1391 - DNA, 50 bp, Artificial Sequence
  Synthetic primer
gctggggacacagcatcagctcaagacgtcaccctccattctgcactcag

SEQ ID NO: 1392 - DNA, 50 bp, Artificial Sequence
  Synthetic primer
tggaaaaagagttaccacgtgttgcagtggttcctgacgctgctgcccgc

SEQ ID NO: 1393 - DNA, 50 bp, Artificial Sequence
  Synthetic primer
agaaactcgtggaggggctgtccgctctggtggtggacgttaagttcgga

SEQ ID NO: 1394 - DNA, 50 bp, Artificial Sequence
  Synthetic primer
catctataaaaggaaaagtgatgagggctgtgctgaagtttgctgctgcc

SEQ ID NO: 1395 - DNA, 50 bp, Artificial Sequence
  Synthetic primer
ggtcagtgtccaggctggaacaaagcgccagtgaactctgactgtatgag

SEQ ID NO: 1396 - DNA, 50 bp, Artificial Sequence
  Synthetic primer
cactgcacactcccacttcctcagcgaccgggcccttctccacctcctgc

SEQ ID NO: 1397 - DNA, 50 bp, Artificial Sequence
  Synthetic primer
aaagaagagcaacacgattctgggatcccaggaggggaacaccatgaaga

SEQ ID NO: 1398 - DNA, 50 bp, Artificial Sequence
  Synthetic primer
catggggaaggggaaaggggaatggctgcttttgatatgttccctgacac

SEQ ID NO: 1399 - DNA, 50 bp, Artificial Sequence
  Synthetic primer
cagcaaatgcctggttctgaagggtcacggggctgtcaacaggtgttcct

SEQ ID NO: 1400 - DNA, 50 bp, Artificial Sequence
  Synthetic primer
gcaggagttggacatctccaggcatttcacagagtgtatcaaagctcagg

SEQ ID NO: 1401 - DNA, 50 bp, Artificial Sequence
  Synthetic primer
ggagcttgaaaatctgccgcagaatggccgccagtgttacagctgcaagg

SEQ ID NO: 1402 - DNA, 50 bp, Artificial Sequence
  Synthetic primer
gtgggaccatccagttgcaggaaaacaagcttaacacgcccactgattct

SEQ ID NO: 1403 - DNA, 50 bp, Artificial Sequence
  Synthetic primer
gtatggaaactaatgctgccactctcatccccgtcccaaccatctctgtc

SEQ ID NO: 1404 - DNA, 50 bp, Artificial Sequence
  Synthetic primer
actgaggccttgcgatattttgtgtgaatagatatgccctaggagttcag

SEQ ID NO: 1405 - DNA, 50 bp, Artificial Sequence
  Synthetic primer
acttgtttcaaatgcatgatcaaatgcaacctcacaaccttggctgagtc

SEQ ID NO: 1406 - DNA, 50 bp, Artificial Sequence
  Synthetic primer
cgcactctttacggcttcggtggctaaggctcctgcttgctgcactctta

SEQ ID NO: 1407 - DNA, 50 bp, Artificial Sequence
  Synthetic primer
tcaatgttcagtgctcaggtatgtagtaagtactgtagtcctgtgggggc

SEQ ID NO: 1408 - DNA, 50 bp, Artificial Sequence
  Synthetic primer
gcacccgcactgtggctccttcgcactgagtatgttggactctgccatag

SEQ ID NO: 1409 - DNA, 50 bp, Artificial Sequence
  Synthetic primer
ggatatcagtgcctggcccacatgagagaacagatccatacacactttga

SEQ ID NO: 1410 - DNA, 50 bp, Artificial Sequence
  Synthetic primer
gttgttagctgcagattaaccccagcccctctgtcttctgttaagtacag

SEQ ID NO: 1411 - DNA, 50 bp, Artificial Sequence
  Synthetic primer
agggtggtttgcccttaagaagaaagctgttgggacaaagacaccgagcc

SEQ ID NO: 1412 - DNA, 50 bp, Artificial Sequence
  Synthetic primer
actcgtctgacccatcagagacgccacagcagagaaacacctctcaaatg

SEQ ID NO: 1413 - DNA, 50 bp, Artificial Sequence
  Synthetic primer
ccatggagagggacccagcataggaaagccacatactcagcctggatggg

SEQ ID NO: 1414 - DNA, 50 bp, Artificial Sequence
  Synthetic primer
ctgaaagagggcagatattgggtccttgtgacttcaactgactcttccga

SEQ ID NO: 1415 - DNA, 50 bp, Artificial Sequence
  Synthetic primer
atgaacgttctcattaacacgcaggagtaccgggagccctgaaccgcccg

SEQ ID NO: 1416 - DNA, 50 bp, Artificial Sequence
  Synthetic primer
atgccatgcatacctcctgccccgcgggaccacaataaaaaccttggcag

SEQ ID NO: 1417 - DNA, 50 bp, Artificial Sequence
  Synthetic primer
agtccggtggtttcggaatgaccaggaggagacagctggcgttgtgtcca

SEQ ID NO: 1418 - DNA, 50 bp, Artificial Sequence
  Synthetic primer
gctaagctggaagggtgcttatccctgcgtagaaacgcctgccaatgctt

SEQ ID NO: 1419 - DNA, 50 bp, Artificial Sequence
  Synthetic primer
aatgccactgacccttctgcccgcacgctgacccactacaagtcagctgt

SEQ ID NO: 1420 - DNA, 50 bp, Artificial Sequence
  Synthetic primer
gcccgatctttctaaaatacagttctgaatatgctgctcatccccacctg

SEQ ID NO: 1421 - DNA, 50 bp, Artificial Sequence
  Synthetic primer
cctccgactctgccgttccatccagtgaaactcttcattctggggtaaag

SEQ ID NO: 1422 - DNA, 50 bp, Artificial Sequence
  Synthetic primer
gcagcatggtccctgccttaggcctacctgatggaagtaaagcctcaacc

SEQ ID NO: 1423 - DNA, 50 bp, Artificial Sequence
  Synthetic primer
gagctctgggcccttccagtgcttgccaccctacatactctttgtctggc

SEQ ID NO: 1424 - DNA, 50 bp, Artificial Sequence
  Synthetic primer
gaacccgaacagttccccctggtgcagggagtggccataggtggctccta

SEQ ID NO: 1425 - DNA, 50 bp, Artificial Sequence
  Synthetic primer
cctgagcttatgctaaaaggtgggatgactcagagctggggctggtcacc

SEQ ID NO: 1426 - DNA, 50 bp, Artificial Sequence
  Synthetic primer
tgccttccttgaccatctaccttgaactaacccactcccagctcccagcc

SEQ ID NO: 1427 - DNA, 50 bp, Artificial Sequence
  Synthetic primer
cacttaacatagtgacctccagttccatccatgctgtcgcaagtgacagg

SEQ ID NO: 1428 - DNA, 50 bp, Artificial Sequence
  Synthetic primer
gtagattgctggcctgttgtaggtggtagggacacagatgaccgacctgg

SEQ ID NO: 1429 - DNA, 50 bp, Artificial Sequence
  Synthetic primer
ggctgtgctcgccccctggacagttgagctttggattatcttcctcttca

SEQ ID NO: 1430 - DNA, 50 bp, Artificial Sequence
  Synthetic primer
cacgtgtccttaacacctgatctggcccaagctgcgtgccctttaagcca

SEQ ID NO: 1431 - DNA, 50 bp, Artificial Sequence
  Synthetic primer
cccaggcaatgctgtcgctgcttgagaacatacacaccaagcccagcttc

SEQ ID NO: 1432 - DNA, 50 bp, Artificial Sequence
  Synthetic primer
ccgagtattggttgaaaggagtgctgctgaaactgtaaccaaaggaggca

SEQ ID NO: 1433 - DNA, 50 bp, Artificial Sequence
  Synthetic primer
ttgctccgggacgatgagagtatttcccatgctggagtcggcgagaaggg

SEQ ID NO: 1434 - DNA, 50 bp, Artificial Sequence
  Synthetic primer
caaactgaaaggcaataaatcctatgtgaacatggacctctctccggtgg

SEQ ID NO: 1435 - DNA, 50 bp, Artificial Sequence
  Synthetic primer
ggccttccctgtcagaagggggttgtggcaaaagccacattacaagctgc

SEQ ID NO: 1436 - DNA, 50 bp, Artificial Sequence
  Synthetic primer
ccacagcttggggtgttcagcacttgaggacgggtggagcttgttcaacc

SEQ ID NO: 1437 - DNA, 50 bp, Artificial Sequence
  Synthetic primer
tcaccaactggactcccatctgtggtgaatatgagtcccttgcccccaac

SEQ ID NO: 1438 - DNA, 50 bp, Artificial Sequence
  Synthetic primer
gcacacgttctcgggacctcctgaagctgcgtcacaggcactaatcaaag

SEQ ID NO: 1439 - DNA, 50 bp, Artificial Sequence
  Synthetic primer
ctctctgccagcatcaaccactcacaccccagggggatgagctctccaag

SEQ ID NO: 1440 - DNA, 50 bp, Artificial Sequence
  Synthetic primer
gcccatggacagtcctgtgtctggatgaggaggactcagtgctggcagat

SEQ ID NO: 1441 - DNA, 50 bp, Artificial Sequence
  Synthetic primer
cgatgatgtccatggctttgtgggaagatactgtggagatgagcttccag

SEQ ID NO: 1442 - DNA, 50 bp, Artificial Sequence
  Synthetic primer
ggcggcagaatgccatcaagtgtgggtggctgaggaagcaaggaggcttt

SEQ ID NO: 1443 - DNA, 50 bp, Artificial Sequence
  Synthetic primer
actgcatccaaatcaaccatctacaggataccctcagcatgtcggtgctc

SEQ ID NO: 1444 - DNA, 50 bp, Artificial Sequence
  Synthetic primer
tagaactattattgaccacgcctcctccaagtcccagcgagcccgtgtac

SEQ ID NO: 1445 - DNA, 50 bp, Artificial Sequence
  Synthetic primer
gcctcatgactgtgtttgatgtcctttattgatacaaagtgagcctgtgc

SEQ ID NO: 1446 - DNA, 50 bp, Artificial Sequence
  Synthetic primer
gagcgcgccattcatcatcagacagcccacactggtgagaaatgatggaa

SEQ ID NO: 1447 - DNA, 50 bp, Artificial Sequence; identical to SEQ ID NO: 441
  Synthetic primer

SEQ ID NO: 1448 - DNA, 50 bp, Artificial Sequence; identical to SEQ ID NO: 827
  Synthetic primer

SEQ ID NO: 1449 - DNA, 50 bp, Artificial Sequence; identical to SEQ ID NO: 1184
  Synthetic primer

SEQ ID NO: 1450 - DNA, 50 bp, Artificial Sequence
  Synthetic primer
ccaggaggccgaacacttctttctgctttcttgacatccgctcaccaggc

SEQ ID NO: 1451 - DNA, 50 bp, Artificial Sequence
  Synthetic primer
ttccagggcacgagttcgaggccagcctggtccacatgggtcggaaaaaa

(19) United States
(12) Patent Application Publication
(10) Pub. No.: US 2015/0315643 A1
(43) Pub. Date: Nov. 5, 2015

(54) BLOOD TRANSCRIPTIONAL SIGNATURES OF ACTIVE PULMONARY TUBERCULOSIS AND SARCOIDOSIS

(71) Applicants: BAYLOR RESEARCH INSTITUTE, Dallas, TX (US); MEDICAL RESEARCH COUNCIL, Swindon (GB); IMPERIAL COLLEGE HEALTHCARE NHS TRUST, London (GB)

(72) Inventors: Anne O'Garra, London (GB); Chloe Bloom, London (GB); Matthew Paul Reddoch Berry, London (GB); Jacques F. Banchereau, Montclair, NJ (US); Damien Chaussabel, Bainbridge Island, WA (US); Viginia Maria Pascual, Dallas, TX (US)

(21) Appl. No.: 14/651,989

(22) PCT Filed: Dec. 13, 2013
(86) PCT No.: PCT/US2013/075097

§ 371 Date: Jun. 12, 2015

Related U.S. Application Data
(60) Provisional application No. 61/736,908, filed on Dec. 13, 2012.

(51) Int. Cl. C12Q 1/68

(52) U.S. Cl. CPC C12Q 1/6883; C12Q 1/6886; C12Q 2600/158; C12Q 2600/16; C12Q 2600/118; C12Q 2600/112; C12Q 2600/106

(57) ABSTRACT

The present invention includes a method of determining a lung disease from a patient suspected of sarcoidosis, tuberculosis, lung cancer or pneumonia comprising: obtaining a sample from whole blood of the patient suspected of sarcoidosis, tuberculosis, lung cancer or pneumonia; detecting expression of (although not exclusive) six or more disease genes, markers, or probes selected from SEQ ID NOS.: 1 to 1446, wherein increased expression of mRNA of upregulated sarcoidosis, tuberculosis, lung cancer and pneumonia markers of SEQ ID NOS.: 1 to 1446 and/or decreased expression of mRNA of downregulated sarcoidosis, tuberculosis, lung cancer or pneumonia markers of SEQ ID NOS.: 1 to 1446 relative to the expression of the mRNAs from a normal sample; and determining the lung disease based on the expression level of the six or more disease markers of SEQ ID NOS.: 1 to 1446 based on a comparison of the expression level of sarcoidosis, tuberculosis, lung cancer, and pneumonia.

### CROSS-REFERENCE TO RELATED APPLICATIONS

None.

### TECHNICAL FIELD OF THE INVENTION

The present invention relates in general to the field of medical diagnosis and medical treatment, and more particularly, to a novel blood transcriptional signatures to distinguish between active pulmonary tuberculosis, sarcoidosis, lung cancer and pneumonia.

### STATEMENT OF FEDERALLY FUNDED RESEARCH

None.

### INCORPORATION-BY-REFERENCE OF MATERIALS

A number of lengthy tables are included herewith and the content incorporated herein by reference. The text file Symbol-Regulation-ID.txt is 47 Kb, Symbol-Sequence-ID.txt is 92 Kb, and 1359-List.txt is 88 Kb and are filed herewith and incorporated by reference in their entirety.

LENGTHY TABLES
The patent application contains a lengthy table section. A copy of the table is available in electronic form from the USPTO web site (http://seqdata.uspto.gov/?pageRequest=docDetail&DocID=US20150315643A1). An electronic copy of the table will also be available from the USPTO upon request and payment of the fee set forth in 37 CFR 1.19(b)(3).

### BACKGROUND OF THE INVENTION

Without limiting the scope of the invention, its background is described in connection with transcriptional signatures. Over nine million new cases of active tuberculosis (TB), and 1.4 million deaths from TB, are estimated to occur around the world every year (1). One of the difficulties of curing pulmonary TB is the ability to diagnose the disease from other similar pulmonary diseases such as pulmonary sarcoidosis, community acquired pneumonia and lung cancer. TB and sarcoidosis are widespread multisystem diseases that preferentially involve the lung and present in a very similar clinical, radiological and histological manner. Distinguishing these diseases therefore often requires an invasive biopsy.

Granuloma formation is fundamental to both these diseases and although the aetiology of TB is well-recognised as the pathogen Mycobacterium tuberculosis, the predominant cause of sarcoidosis remains unknown (2). The underlying pathways of granulomatous inflammation are also poorly understood and there is little understanding of disease-specific differences. Both sarcoidosis and TB can affect adults within the same age group, who then present with similar pulmonary symptoms and radiological thoracic abnormalities (3, 4). TB can also display a similar presentation to other pulmonary infectious diseases such as community acquired pneumonia and other lung inflammatory disorders such as primary lung cancer. Due to the complexity of these diseases a systems biology approach offers the ability to help unravel the principal host immune responses. Peripheral blood has the capacity to reflect pathological and immunological changes in the body, and identification of disease-associated alterations can be determined by a blood transcriptional signature (5). In addition the applicants have published a IFN-inducible neutrophil blood transcriptional signature in active TB patients that is absent in the majority of latent individuals and healthy controls, that correlates significantly with the extent of lung radiographic disease (5) and is diminished upon treatment (5, 12).

Blood gene expression profiling has been successfully applied to other infectious and inflammatory disorders, such as systemic lupus erythematosus (SLE), to help understand disease mechanisms and improve diagnosis and treatment (5). Two recent studies have used blood transcriptional profiling for the comparison of pulmonary TB and sarcoidosis; both studies found the diseases had similar transcriptional responses, which involved the overexpression of IFN-inducible genes (9, 10). However, these studies did not differentiate signatures from other pulmonary diseases leaving to question if the transcriptional signatures were non-specific for pulmonary disorders.

### SUMMARY OF THE INVENTION

In one embodiment, the present invention includes a method of determining if a human subject is afflicted with pulmonary disease comprising: obtaining a sample from a subject suspected of having a pulmonary disease; determining the expression level of six or more genes from each of the following genes expressed in one or more of the following expression pathways: EIF2 signaling; mTOR signaling; regulation of eIF4 and p70s6K signaling; interferon signaling; antigen presentation pathways; T cell signaling pathways; and other signaling pathways; comparing the expression level of the six or more genes with the expression level of the same genes from individuals not afflicted with a pulmonary disease, and determining the level of expression of the six or more genes in the sample from the subject relative to the samples from individuals not afflicted with a pulmonary disease for the genes expressed in the one or more expression pathways, wherein co-expression of genes in the EIF2 signaling and mTOR signaling pathways are indicative of active sarcoidosis; co-expression of genes in the regulation of eIF4 and p70s6K signaling pathways is indicative of pneumonia; co-expression of genes in the interferon signaling and antigen presentation pathways are indicative of tuberculosis; and co-expression of genes in the T cell signaling pathways; and other signaling pathways is indicative of lung cancer. In one aspect, the genes associated with tuberculosis are selected from at least 3, 4, 5 or 6 genes selected from ANKRD22; FCGR1A; SERPING1; BATF2; FCGR1C; FCGR1B; LOC728744; IFITM3; EPSTI1; GBP5; IF144L; GBP6; GBP1; LOC400759; IFIT3; AIM2; SEPT4; C1QB; GBP1; RSAD2; RTP4; CARD17; IFIT3; CASP5; CEACAM1; CARD17; ISG15; IF127; TIMM10; WARS; IF16; TNFAIP6; PSTPIP2; IF144; SCO2; FBXO6; FER1L3; CXCL10; DHRS9; OAS1; STAT1; HP; DHRS9; CEACAM1; SLC26A8; CACNA1E; OLFM4; and APOL6, wherein the genes are evaluated at least one of: in aggregate, in the order listed, aggregated into pathways, or selected from 7, 8, 9, 10, 11, 12, 13, 15, 20, 25, 35, 40, 45, or 49 genes. In another aspect, the genes associated with tuberculosis and not active sarcoidosis, pneumonia or lung cancer are selected from C1QB; IF127; SMARCD3; SOCS1; KCNJ15; LPCAT2; ZDHHC19; FYB; SP140; IFITM1; ALAS2; CEACAM6; OAS2; C1QC; LOC100133565; ITGA2B; LY6E; SP140; CASP7; GADD45G; FRMD3; CMPK2; AQP10; CXCL14; ITPRIPL2; FAS; XK; CARD16; SLAMF8; SELP; NDN; OAS2; TAPBP; BPI; DHX58; GAS6; CPT1B; CD300C; LILRA6; USF1; C2; 38231.0; NFXL1; GCH1; CCR1; OAS2; CCR2; F2RL1; SNX20; and ARAP2, wherein the genes are evaluated at least one of: in aggregate, in the order listed, aggregated into pathways, or selected from 7, 8, 9, 10, 11, 12, 13, 15, 20, 25, 35, 40, 45, or 49 genes. In another aspect, the genes associated with active sarcoidosis are selected from FCGR1A; ANKRD22; FCGR1C; FCGR1B; SERPING1; FCGR1B; BATF2; GBP5; GBP1; IFIT3; ANKRD22; LOC728744; GBP1; EPSTI1; IF144L; INDO; IFITM3; GBP6; RSAD2; DHRS9; TNFAIP6; IFIT3; P2RY14; DHRS9; IDO1; STAT1; WARS; TIMM10; P2RY14; LOC389386; FER1L3; IFIT3; RTP4; SCO2; GBP4; IFIT1; LAP3; OASL; CEACAM1; LIMK2; CASP5; STAT1; CCL23; WARS; ATF3; IF16; PSTPIP2; ASPRV1; FBXO6; and CXCL10, wherein the genes are evaluated at least one of: in aggregate, in the order listed, aggregated into pathways, or selected from 7, 8, 9, 10, 11, 12, 13, 15, 20, 25, 35, 40, 45, or 49 genes. In another aspect, the genes associated with active sarcoidosis and not tuberculosis, pneumonia or lung cancer are selected from CCL23; PIK3R6; EMR4; CCDC146; KLF4; GRINA; SLC4A1; PLA2G7; GRAMD1B; RAPGEF1; NXNL1; TRIM58; GABBR1; TAGLN; KLF4; MFAP3L; LOC641798; RIPK2; LOC650840; FLJ43093; ASAP2; C15orf26; REC8; KIAA0319L; GRINA; FLJ30092; BTN2A1; HIF1A; LOC440313; HOXA1; LOC645153; ST3GAL6; LONRF1; PPP1R3B; MPPE1; LOC652699; LOC646144; SGMS1; BMP2K; SLC31A1; ARSB; CAMK1D; ICAM4; HIF1A; LOC641996; RNASE10; PI15; SLC30A1; LOC389124; and ATP1A3, wherein the genes are evaluated at least one of: in aggregate, in the order listed, aggregated into pathways, or selected from 7, 8, 9, 10, 11, 12, 13, 15, 20, 25, 35, 40, 45, or 49 genes. In another aspect, the genes associated with pneumonia are selected from OLFM4; LTF; VNN1; HP; DEFA4; OPLAH; CEACAM8; DEFA1B; ELANE; C19orf59; ARG1; CDK5RAP2; DEFA1B; DEFA3; DEFA1B; FCGR1A; MMP8; FCGR1B; SLPI; SLC26A8; MAPK14; CAMP; NLRC4; FCAR; RNASE3; FCGR1B; NAIP; OLR1; FCGR1C; ANXA3; DEFA1; PGLYRP1; TCN1; ANKDD1A; COL17A1; SLC26A8; TMEM144; SAMD14; MAPK14; RETN; NAIP; GPR84; CASP5; MPO; MMP9; CR1; MYL9; CLEC4D; ITGAX; and ANKRD22, wherein the genes are evaluated at least one of: in aggregate, in the order listed, aggregated into pathways, or selected from 7, 8, 9, 10, 11, 12, 13, 15, 20, 25, 35, 40, 45, or 49 genes. In another aspect, the genes associated with pneumonia and not tuberculosis, active sarcoidosis, or lung cancer are selected from DEFA4; ELANE; MMP8; OLR1; COL17A1; RETN; GPR84; LOC100134379; TACSTD2; SLC2A11; LOC100130904; MCTP2; AZU1; DACH1; GADD45A; NSUN7; CR1; CDK5RAP2; LOC284648; GPR177; CLEC5A; UPB1; SLC2A5; GPR177; APP; LAMC1; REPS2; PIK3CB; SMPDL3A; UBE2C; NDUFAF3; CDC20; CTSK; RAB13; LOC651524; TMEM176A; PDGFC; ATP9A; SV2A; SPOCD1; MARCO; CCDC109A; NUSAP1; SLCO4C1; CYP27A1; LOC644615; PKM2; BMX; PADI4; and NAMPT, wherein the genes are evaluated at least one of: in aggregate, in the order listed, aggregated into pathways, or selected from 7, 8, 9, 10, 11, 12, 13, 15, 20, 25, 35, 40, 45, or 49 genes. In another aspect, the genes associated with lung cancer are selected from ARG1; TPST1; FCGR1A; C19orf59; SLPI; FCGR1B; IL1R1; FCGR1C; TDRD9; SLC26A8; FCGR1B; CLEC4D; LOC100132858; SLC22A4; LOC100133177; SIPA1L2; ANXA3; LIMK2; TMEM88; MMP9; ASPRV1; MANSC1; TLR5; CD163; CAMP; LOC642816; DPRXP4; LOC643313; NTN3; MRVI1; F5; SOCS3; TncRNA; MIR21; LOC100170939; LOC100129904; GRB10; ASGR2; LOC642780; LOC400499; FCAR; KREMEN1; SLC22A4; CR1; LOC730234; SLC26A8; C7orf53; VNN1; NLRC4; and LOC400499, wherein the genes are evaluated at least one of: in aggregate, in the order listed, aggregated into pathways, or selected from 7, 8, 9, 10, 11, 12, 13, 15, 20, 25, 35, 40, 45, or 49 genes. In another aspect, the genes associated with lung cancer and not tuberculosis, active sarcoidosis, or pneumonia are selected from TPST1; MRVI1; C7orf53; ECHDC3; LOC651612; LOC100134660; TIAM2; KIAA1026; HECW2; TLE3; TBC1D24; LOC441193; CD163; RFX2; LOC100134688; LOC642342; FKBP9L; PHF20L1; LOC402176; CD163; OSBPL1A; PRMT5; UBTD1; ADORA3; SH2D3C; RBP7; ERGIC1; TMEM45B; CUX1; TREM1; C1GALT1C1; MAML3; C15orf29; DSC2; RRP12; LRP3; HDAC7A; FOS; C14orf4; LIPN; MAP1LC3B2; LOC400793; LOC647834; PHF20L1; CCNJL; SLC12A6; FLJ42957; CCDC147; SLC25A40; and LOC649270, wherein the genes are evaluated at least one of: in aggregate, in the order listed, aggregated into pathways, or selected from 7, 8, 9, 10, 11, 12, 13, 15, 20, 25, 35, 40, 45, or 49 genes. In another aspect, the genes associated with lung cancer and not tuberculosis, active sarcoidosis, or pneumonia are selected from wherein the genes associated with lung cancer and not tuberculosis, active sarcoidosis, or pneumonia are selected from Table 1 by: parsing the genes into the expression pathways, and determining that the subject is afflicted with a pulmonary disease selected from tuberculosis, sarcoidosis, cancer or pneumonia based on the gene expression from a sample obtained from the subject when compared to the level of expression of the genes in each of the expression pathways. In another aspect, the specificity is 90 percent or greater and sensitivity is 80 percent or greater for a diagnosis of tuberculosis or sarcoidosis. In another aspect, the method further comprises a method for displaying if the patient has tuberculosis or sarcoidosis aggregating the expression data from the 3, 4, 5, 6 or more genes into a single visual display of a vector of expression for tuberculosis, sarcoidosis, cancer or an infectious pulmonary disease. In another aspect, the method further comprises the step of detecting and evaluating 7, 8, 9, 10, 12, 15, 20, 25, 35, 50, 75, 90, 100, 125, or 144 genes for the analysis. In another aspect, the method further comprises the step of detecting and evaluating the EIF2 signaling; mTOR signaling; regulation of eIF4 and p70s6K signaling; interferon signaling; antigen presentation pathways; T cell signaling pathways; and other signaling pathways from 7, 8, 9, 10, 12, 15, 20, 25, 35, 50, 75, 90, 100, 125, or 144 genes that are upregulated or downregulated and are selected from UBE2J2; ALPL; JMJD6; FCER1G; LILRA5; LY96; FCGR1C; C10orf33; GPR109B; PROK2; PIM3; SH3GLB1; DUSP3; PPAP2C; SLPI; MCTP1; KIF1B; FLJ32255; BAGE5; IFITM1; GPR109A; IF135; LOC653591; KREMEN1; IL18R1; CACNA1E; ABCA2; CEACAM1; MXD4; TncRNA; LMNB1; H2AFJ; HP; ZNF438; FCER1A; SLC22A4; DISC1; MEFV; ABCA1; ITPRIPL2; KCNJ15; LOC728519; ERLIN1; NLRC4; B4GALT5; LOC653610; HIST2H2BE; AIM2; P2RY10; CCR3; EMR4P; NTN3; C1QB; TAOK1; FCGR1B; GATA2; FKBP5; DGAT2; TLR5; CARD17; INCA; MSL3L1; ESPN; LOC645159; C19orf59; CDK5RAP2; PLSCR1; RGL4; IFI30; LOC641710; GAGGCTTTCAGGTAGGAGGACAATGGTAGCACTGTAGGTCCCCAGTGTCG (SEQ ID NO.: 754); LOC100008589; LOC100008589; SMARCD3; NGFRAP1; LOC100132394; OPLAH; CACNG6; LILRB4; HIST2H2AA4; CYP1B1; PGS1; SPATA13; PFKFB3; HIST1H3D; SNORA73B; SLC26A8; SULT1B1; ADM; HIST2H2AA3; HIST2H2AA3; GYG1; CST7; EMR4; LILRA6; MEF2D; IFITM3; MSL3; DHRS13; EMR4; C16orf57; HIST2H2AC; EEF1D; TDRD9; GPR97; ZNF792; LOC100134364; SRGAP3; FCGR1A; HPSE; LOC728417; LOC728417; MIR21; HIST1H2BG; COP1; SMARCD3; LOC441763; ZSCAN18; GNG8; MTRF1L; ANKRD33; PLAC8; PLAC8; SLC26A8; AGTRAP; FLJ43093; LPCAT2; AGTRAP; S100A12; SVIL; LILRA5; LILRA5; ZFP91; CLC; LOC100133565; LTB4R; SEPT04; ANXA3; BHLHB2; IL4R; IFNAR1; MAZ; GCCCCCTAATTGACTGAATGGAACCCCTCTTGACCAAAGTGACCCCAGAA (SEQ ID NO.: 1379); OSM; and optionally excluding at least one of ADM, SEPT4, IFITM1, FCER1G, MED2F, CDK5RAP2 or CARD16. In another aspect, the genes that are downregulated are selected from MEF2D; BHLHB2; CLC; FCER1A; SRGAP3; FLJ43093; CCR3; EMR4; ZNF792; C10orf33; CACNG6; P2RY10; GATA2; EMR4P; ESPN; EMR4; MXD4; and ZSCAN18. In another aspect, the interferon inducible genes are selected from CD274; CXCL10; GBP1; GBP2; GBP5; IF116; IF135; IF144; IF144L; IF16; IFIH1; IFIT2; IFIT3; IFIT5; IFITM1; IFITM3; IRF7; OAS1; OAS2; OAS3; SOCS1; STAT1; STAT2; TAP1; and TAP2. In another aspect, the sample is a blood, peripheral blood mononuclear cells, sputum, or lung biopsy. In another aspect, the expression level comprises a mRNA expression level and is quantitated by a method selected from the group consisting of polymerase chain reaction, real time polymerase chain reaction, reverse transcriptase polymerase chain reaction, hybridization, probe hybridization and gene expression array. In another aspect, the expression level is determined using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing. In another aspect, the expression level is determined by microarray analysis that comprises use of oligonucleotides that hybridize to mRNA transcripts or cDNAs for the six or more genes, and wherein the oligonucleotides are disposed or directly synthesized on the surface of a chip or wafer. In another aspect, the oligonucleotides are about 10 to about 50 nucleotides in length. In another aspect, the method further comprises the step of using the determined comparative gene product information to formulate at least one of diagnosis, a prognosis or a treatment plan. In another aspect, the patient's disease state is further determined by radiological analysis of the patient's lungs. In another aspect, the method further comprises the step of determining a treated patient gene expression dataset after the patient has been treated and determining if the treated patient gene expression dataset has returned to a normal gene expression dataset thereby determining if the patient has been treated.

Another embodiment of the present invention includes a method of determining a lung disease from a patient suspected of sarcoidosis, tuberculosis, lung cancer or pneumonia comprising: obtaining a sample from the patient suspected of sarcoidosis, tuberculosis, lung cancer or pneumonia; detecting expression of 3, 4, 5, 6 or more disease genes, markers, or probes of Table 1 (SEQ ID NOS.: 1 to 1446), wherein increased expression of mRNA of upregulated sarcoidosis, tuberculosis, lung cancer and pneumonia markers of Table 1 and/or decreased expression of mRNA of downregulated sarcoidosis, tuberculosis, lung cancer or pneumonia markers of Table 1 relative to the expression of the mRNAs from a normal sample; and determining the lung disease based on the expression level of the six or more disease markers of Table 1 based on a comparison of the expression level of sarcoidosis, tuberculosis, lung cancer, and pneumonia. In one aspect, the method further comprises the step of selecting 3, 4, 5, 6 or more genes that are differentially expressed between sarcoidosis, tuberculosis, lung cancer, and pneumonia. In another aspect, the method further comprises the step of differentiating between sarcoidosis that is active sarcoidosis and inactive sarcoidosis by determining the expression levels of six or more genes, markers, or probes selected from: TMEM144; FBLN5; FBLN5; ERI1; CXCR3; GLUL; LOC728728; KLHDC8B; KCNJ15; RNF125; CCNB1IP1; PSG9; LOC100170939; QPCT; CD177; LOC400499; LOC400499; LOC100134634; TMEM88; LOC729028; EPSTI1; INSC; LOC728484; ERP27; CCDC109A; LOC729580; C2; TTRAP; ALPL; MAEA; COX10; GPR84; TRMT11; ANKRD22; MATK; TBC1D24; LILRA5; TMEM176B; CAMP; PKIA; PFTK1; TPM2; TPM2; PRKCQ; PSTPIP2; LOC129607; APRT; VAMPS; FCGR1C; SHKBP1; CD79B; SIGIRR; FKBP9L; LOC729660; WDR74; LOC646434; LOC647834; RECK; MGST1; PIWIL4; LILRB1; FCGR1B; NOC3L; ZNF83; FCGBP; SNORD13; LOC642267; GBP5; EOMES; BST1; C5; CHMP7; ETV7; ILVBL; LOC728262; GNLY; LOC388572; GATA1; MYBL1; LOC441124; LOC441124; IL12RB1; BRIX1; GAS6; GAS6; LOC100133740; GPSM1; C6orf129; IER3; MAPK14; PROK1; GPR109B; SASP; LOC728093; PROK2; CTSW; ABHD2; LOC100130775; SLITRK4; FBXW2; RTTN; TAF15; FUT7; DUSP3; LOC399715; LOC642161; LOC100129541; TCTN1; SLAMF8; TGM2; ECE1; CD38; INPP4B; ID3; CR1; CR1; TAPBP; PPAP2C; MBOAT2; MS4A2; FAM176B; LOC390183; SERPING1; LOC441743; H1F0; SOD2; LOC642828; POLB; TSPAN9; ORMDL3; FER1L3; LBH; PNKD; SLPI; SIRPB1; LOC389386; REC8; GNLY; GNLY; FOLR3; LOC730286; SKAP1; SELP; DHX30; KIAA1618; NQO2; ANKRD46; LOC646301; LOC400464; LOC100134703; C20orf106; SLC25A38; YPEL1; IL1R1; EPHA1; CHD6; LIMK2; LOC643733; LOC441550; MGC3020; ANKRD9; NOD2; MCTP1; BANK1; ZNF30; FBXO7; FBXO7; ABLIM1; LAMP3; CEBPE; LOC646909; BCL11B; TRIM58; SAMD3; SAMD3; MYOF; TTPAL; LOC642934; FLJ32255; LOC642073; CAMKK2; OAS2; RASGRP1; CAPG; LOC648343; CETP; CETP; CXCR7; UBASH3A; LOC284648; IL1R2; AGK; GTPBP8; LEF1; LEF1; GPR109A; IF135; IRF7; IRF7; SP4; IL2RB; ABLIM1; TAPBP; MAL; TCEA3; KREMEN1; KREMEN1; VNN1; GBP1; GBP1; UBE2C; DET1; ANKRD36; DEFA4; GCH1; IL7R; TMCO3; FBXO6; LACTB; LOC730953; LOC285296; IL18R1; PRR5; LOC400061; TSEN2; MGC15763; SH3YL1; ZNF337; AFF3; TYMS; ZCCHC14; SLC6A12; LY6E; KLF12; LOC100132317; TYW3; BTLA; SLC24A4; NCALD; ORAI2; ITGB3BP; GYPE; DOCKS; RASGRP4; LOC339290; PRF1; TGFBR3; LGALS9; LGALS9; BATF2; MGC57346; TXK; DHX58; EPB41L3; LOC100132499; LOC100129674; GDPD5; ACP2; C3AR1; APOB48R; UTRN; SLC2A14; CLEC4D; PKM2; CDCA5; CACNA1E; OSBPL3; SLC22A15; VPREB3; LOC642780; MEGF6; LOC93622; PFAS; LOC729389; CREBZF; IMPDH1; DHRS3; AXIN2; DDX60L; TMTC1; ABCA2; CEACAM1; CEACAM1; FLJ42957; SIAH2; DDAH2; C13orf18; TAGLN; LCN2; RELB; NR1I2; BEND7; PIK3C2B; IF16; DUT; SETD6; LOC100131572; TNRC6A; LOC399744; MAPK13; TAP2; CCDC15; TncRNA; SIPA1L2; HIST1H4E; PTPRE; ELANE; TGM2; ARSD; LOC651451; CYFIP1; CYFIP1; LOC642255; ASCC2; ZNF827; STAB1; LMNB1; MAP4K1; PSMB9; ATF3; CPEB4; ATP5S; CD5; SYTL2; H2AFJ; HP; SORT1; KLHL18; HIST1H2BK; KRTAP19-6; RNASE2; LOC100134393; C11orf82; BLK; CD160; LOC100128460; CD19; ZNF438; MBNL3; MBNL3; LOC729010; NAGA; FCER1A; C6orf25; SLC22A4; LOC729686; CTSL1; BCL11A; ACTA2; KIAA1632; UBE2C; CASP4; SLC22A4; SFT2D2; TLR2; C10orf105; EIF2AK2; TATDN1; RAB24; FAH; DISC1; LOC641848; ARG1; LCK; WDFY3; RNF165; MLKL; LOC100132673; ANKDD1A; MSRB3; LOC100134379; MEFV; C12orf57; CCDC102A; LOC731777; LOC729040; TBC1D8; KLRF1; KLRF1; ABCA1; LOC650761; LOC653867; LOC648710; SLC2A11; LOC652578; GPR114; MANSC1; MANSC1; DGKA; LIN7A; ITPRIPL2; ANO9; KCNJ15; KCNJ15; LOC389386; LOC100132960; LOC643332; SF11; ABCE1; ABCE1; SERPINA1; OR2W3; ABI3; LOC400759; LOC728519; LOC654053; LOC649553; HSD17B8; C16orf30; GADD45G; TPST1; GNG7; SV2A; LOC649946; LOC100129697; RARRES3; C8orf83; TNFSF13B; SNRPD3; LOC645232; PI3; WDFY1; LOC100133678; BAMBI; POPS; TARBP1; IRAK3; ZNF7; NLRC4; SKAP1; GAS7; C12orf29; KLRD1; ABHD15; CCDC146; CASP5; AARS2; LOC642103; LOC730385; GAR1; MAF; ARAP2; C16orf7; HLA-C; FLJ22662; DACH1; CRY1; CRY1; LRRC25; KIAA0564; UPF3A; MARCO; SRPRB; MAD1L1; LOC653610; P4HTM; CCL4L1; LAPTM4B; MAPK14; CD96; TLR7; KCNMB1; P2RX7; LOC650140; LOC791120; LTF; C3orf75; GPX7; SPRYD5; MOV10; EEF1B2; CTDSPL; HIST2H2BE; SLC38A1; AIM2; LOC100130904; LOC650546; P2RY10; ILSRA; MMP8; LOC100128485; RPS23; HDAC7; GUCY1A3; TGFA; NAIP; NAIP; NELL2; SIDT1; SLAMF1; MAPK14; CCR3; MKNK1; D4S234E; NBN; LOC654346; FGFBP2; BTLA; LRRN3; MT2A; LOC728790; LOC646672; NTN3; CD8A; CD8A; ZBP1; LDOC1L; CHM; LOC440731; LOC100131787; TNFRSF10C; LOC651612; STX11; LOC100128060; C1QB; PVRL2; ZMYND15; TRAPPC2P1; SECTM1; TRAT1; CAMKK2; CXCR5; CD163; FAS; RPL12P6; LOC100134734; CD36; FCGR1B; NR3C2; CSGALNACT2; GATA2; EBI2; EBI2; FKBP5; CRISPLD2; LOC152195; LOC100132199; DGAT2; SCML1; LSS; CIITA; SAP30; TLR5; NAMPT; GZMK; CARD17; INCA; MSL3L1; CD8A; MIIP; SRPK1; SLC6A6; C10orf119; C17orf60; LOC642816; AKR1C3; LHFPL2; CR1; KIAA1026; CCDC91; FAM102A; FAM102A; UPRT; PLEKHA1; CACNA2D3; DDX10; RPL23A; C2orf44; LSP1; C7orf53; DNAJC5; SLAIN1; CDKN1C; HIATL1; CRELD1; ZNHIT6; TIFA; ARL4C; PIGU; MEF2A; PIK3CB; CDK5RAP2; FLNB; GRAP; BATF; CYP4F3; KIR2DL3; C19orf59; NRG1; PPP2R2B; CDK5RAP2; PLSCR1; UBL7; HES4; ZNF256; DKFZp761E198; SAMD14; BAG3; PARP14; MS4A7; ECHDC3; OCIAD2; LOC90925; RGL4; PARP9; PARP9; CD151; SAAL1; LOC388076; SIGLEC5; LRIG1; PTGDR; PTGDR; NBPF8; NHS; ACSL1; HK3; SNX20; F2RL1; F2RL1; PARP12; LOC441506; MFGE8; SERPINA10; FAM69A; IL4R; KIAA1671; OAS3; PRR5; TMEM194; MS4A1; MTHFD2; LOC400793; CEACAM1; APP; RRBP1; SLCO4C1; XAF1; XAF1; SLC2A6; ZNF831; ZNF831; POLR1C; GLT1D1; VDR; IFIT5; SNHG8; TOP1MT; UPP1; SYTL2; LOC440359; KLRB1; MTMR3; S1PR1; FYB; CDC20; MEX3C; FAM168B; SLC4A7; CD79B; FAM84B; LOC100134688; LOC651738; PLAGL1; TIMM10; LOC641710; TRAF5; TAP1; FCRL2; SRC; RALGAPA1; OCIAD2; PON2; LOC730029; LOC100134768; LOC100134241; LOC26010; PLA2G12A; BACH1; DSC1; NOB1; LOC645693; LOC643313; BTBD11; REPS2; ZNF23; C18orf55; APOL2; APOL2; PASK; FER1L3; U2AF1; LOC285359; SIGLEC14; ARL1; C19orf62; NCR3; HOXB2; RNF135; IFIT1; KLF12; LILRB2; LOC728835; GSN; LOC100008589; LOC100008589; FLJ14213; SH2D3C; LOC100133177; HIST2H2AB; KIAA1618; C21orf2; CREB5; FAS; MTF1; RSAD2; ANPEP; C14orf179; TXNL4B; MYL9; MYL9; LOC100130828; LOC391019; ITGA2B; KLRC3; RASGRP2; NDST1; LOC388344; IF16; OAS1; OAS1; TRIM10; LIMK2; LIMK2; ATP5S; SMARCD3; PHC2; SOX8; LCK; SAMD9L; EHBP1; E2F2; CEACAM6; LOC100132394; LOC728014; LOC728014; SIRPG; OPLAH; FTHL2; CXorf21; CACNG6; C11orf75; LY9; LILRB4; STAT2; RAB20; SOCS1; PLOD2; UGDH; MAK16; ITGB3; DHRS9; PLEKHF1; ASAP1IT1; PSME2; LOC100128269; ALX1; BAK1; XPO4; CD247; FAM43A; ICOS; ISG15; HIST2H2AA4; CD79A; SLC25A4; TMEM158; GPR18; LAP3; TNFSF13B; TC2N; HSF2; CD7; C20orf3; HLA-DRB3; SESN1; LOC347376; P2RY14; P2RY14; P2RY14; CYP1B1; IFIT3; IFIT3; RPL13L; LOC729423; DBN1; TTC27; DPH5; GPR141; RBBP8; LOC654350; SLC30A1; PRSS23; JAM3; GNPDA2; IL7R; ACAD11; LOC642788; ALPK1; LOC439949; BCAT1; ATPGD1; TREML1; PECR; SPATA13; MAN1C1; ID01; TSEN54; SCRN1; LOC441193; LOC202134; KIAA0319L; MOSC1; PFKFB3; GNB4; ANKRD22; PROS1; CD40LG; RIOK2; AFF1; HIST1H3D; SLC26A8; SLC26A8; RNASE3; UBE2L6; UBE2L6; SSH1; KRBA1; SLC25A23; DTX3L; DOK3; SULT1B1; RASGRP4; ALOX15B; ADM; LOC391825; LOC730234; HIST2H2AA3; HIST2H2AA3; LIMK2; MMRN1; FKBP1A; GYG1; ASF1A; CD248; CD3G; DEFA1; EPHX2; CST7; ABLIM3; ANKRD55; SLC45A3; RAB33B; LILRA6; LILRA6; SPTLC2; CDA; PGD; LOC100130769; ECHDC2; KIF20B; B3GNT8; PYHIN1; LBH; LBH; BPI; GAR1; ST3GAL4; TMEM19; DHRS12; DHRS12; FAM26F; FCRLA; OSBPL7; CTSB; ALDH1A1; SRRD; TOLLIP; ICAM1; LAX1; CASP7; ZDHHC19; LOC732371; DENND1A; EMR2; LOC643308; ADA; LOC646527; LOC643313; GZMB; OLIG2; HLA-DPB1; MX1; THOC3; TRPM6; GK; JAK2; ARHGEF11; ARHGEF11; HOMER2; TACSTD2; CA4; GAA; IFITM3; CLYBL; CLYBL; MME; ZNF408; STAT1; STAT1; PNPLA7; INDO; PDZD8; PDGFD; CTSL1; HOMER3; CEP78; SBK1; ALG9; IL1R2; RAB40B; MMP23B; PGLYRP1; UHRF1; IF144L; PARP10; PARP10; GOLGA8A; CCR7; HEMGN; TCF7; CLUAP1; LOC390735; LOC641849; TYMP; DEFA1B; DEFA1B; DEFA1B; REPS2; REPS2; OSBPL1A; C11orf1; MCTP2; EMR4; LOC653316; FCRL6; MRPS26; RHOBTB3; DIRC2; CD27; PLEKHG4; CDH6; C4orf23; HIST2H2AC; SLC7A6; SLC7A6; SLAMF6; RETN; FAIM3; TMEM99; LOC728411; TMEM194A; NAPEPLD; ACOX1; CTLA4; SCO2; STK3; FLT3LG; VASP; FBXO31; TDRD9; TDRD9; LOC646144; NUSAP1; GPR97; GPR97; GPR97; EMR1; SLAMF6; CCDC106; ODF3B; LOC100129904; PADI4; LOC100132858; PIK3AP1; ZNF792; DIP2A; OSCAR; CLIC3; FANCE; TECPR2; P2RY10; ADORA3; IL18RAP; DEFA3; BRSK1; LOC647691; S1PR5; CPA3; BMX; DDX58; RHOBTB1; TNFRSF25; LOC730387; OLR1; HERC5; STAT1; NELF; STAP1; ZNF516; ARHGAP26; TIMP2; FCGR1A; RHOH; IF144; MTX3; CD74; LCK; TLR4; DSC2; CXorf45; ENPP4; CD300C; OASL; HPSE; MTHFD2; GSTM2; OLFM4; ABHD12B; LOC728417; LOC728417; FCAR; GTPBP3; KLF4; HOPX; THBD; HIST1H2BG; LOC730995; NOP56; ZBTB9; NLRC3; LOC100134083; COP1; CARD16; SP140; CD96; POLD2; IL32; LOC728744; FZD2; ZAP70; PYHIN1; SCARF1; IF127; PFKFB2; PAM; WARS; TCN1; LOC649839; MMP9; TMEM194A; TAP2; C17orf87; LOC728650; PNMA3; CPT1B; LTBP3; CCDC34; PRAGMIN; C9orf91; SMPDL3A; GPR56; C14orf147; SMARCD3; FAM119A; LOC642334; ENOSF1; FAR2; LOC441763; TESC; CECR6; KIAA1598; GPR109B; LRRN3; RNF213; LRP3; ASGR2; ASGR2; ZSCAN18; MCOLN2; IFIT2; PLCH2; MAP7; GBP4; MGMT; GAL3ST4; C2orf89; TXNDC3; IFIH1; PRRG4; LOC641693; LOC728093; TNFAIP8L1; AP3M2; BACH2; BACH2; C9orf123; CACNA1I; LOC100132287; CAMK1D; ANKRD33; CCR6; ALDH1A1; LOC100132797; CD163; ESAM; FCAR; TCN2; CD6; CD3E; CCDC76; MS4A1; IFIT1; MED13L; SLC26A8; NOV; FLJ20035; UGT1A3; LOC653600; LOC642684; KIAA0319L; KLRD1; TRIM22; C4orf18; TSPAN3; TSPAN3; DNAJC3; AGTRAP; LOC646786; NCALD; TTC25; TSPAN5; ZNF559; NFKB2; LOC652616; HLA-DOA; WARS; GBP2; AUTS2; IGF2BP3; OASL; DYSF; FLJ43093; MS4A14; TGFB1I1; RAD51C; CALD1; LOC730281; MUC1; C14orf124; RPL14; APOL6; KCTD12; ITGAX; IFIT3; LPCAT2; ZNF529; AGTRAP; LOC402112; LOC100134822; SH2D1B; MPO; LOC100131967; LOC440459; FAM44B; ACOT9; LOC729915; PDZK1IP1; S100A12; RAB3IL1; TMEM204; CXCL10; TSR1; MXD3; LILRA5; CKAP4; C6orf190; ECGF1; LDLRAP1; GRB10; FCRL3; LOC731275; ZFP91; CTRL; BCL6; SAMD3; LOC647436; CLC; GK; LOC100133565; OAS2; LOC644937; SIRPD; GPBAR1; GNL3; CD79B; ELF2; GAA; CD47; NMT2; MATR3; TMEM107; GCM1; RORA; MGAM; LOC100132491; KRT72; SEPT04; ACADVL; ANXA3; MEGF9; MEGF9; PTPRJ; HLA-DRB4; FFAR2; PML; HLA-DQA1; CEACAM8; SH3KBP1; TRPM2; CUX1; LOC648390; SUV39H1; USF1; VAPA; ALOX15; CD79A; DPRXP4; LOC652750; ECM1; ST6GAL1; KLHL3; RTP4; FAM179A; HDC; SACS; C9orf72; C9orf72; LOC652726; PVRIG; PPP1R16B; NSUN7; NSUN7; ZNF783; LOC441013; LOC100129343; OSM; UNC93B1; DNAJC30; FLJ14166; C9orf72; SAMD4A; F5; PARP15; PAFAH2; COL17A1; TYMP; LOC389672; ABCB1; LOC644852; TARP; SLAMF7; FRMD3; LOC648984; PLAUR; LOC100132119; KLRG1; INTS2; MYC; HIST1H4H; C9orf45; GBP6; KIFAP3; HSPC159; SOCS3; GOLGA8B; LOC100133583; ARL4A; ASNS; ITGAX; LOC153561; GSTM1; OAS2; OAS2; TRIM25; ABHD14A; LOC642342; GPR56; C4orf18; AK1; PIK3R6; HSPE1; ASPHD2; DHRS9; GRN; BOAT; LOC100134300; SDSL; TNFAIP6; LOC402176; LOC441019; FAM134B; ZNF573, GGGGTAACACAGAGTGCCCTTATGAAGGAGTTGGAGATCCTgcaaggaag (SEQ ID NO.:69); AAACCCGTCACCCAGATCGTCAGCGCCGAGGCCTGGGGTAGAGCAGGTGA (SEQ ID NO.:87); TGTTCTTCCCCATGTCCTGGATGCCACTGGAAGTGCACACTGCTTGTATG (SEQ ID NO.:93); CCCTGGAAAGCTCCCCGACAACCTCCACTGCCATTACCCACTAGGCAAGT (SEQ ID NO.:95); CCTCCAGTGGTTTAGGCAGGACCCTGGGAAAGGTCTCACATCTCTGTTGC (SEQ ID NO.:174); GCACCATGCATGGAGTCAGCCATTTCTCTAGGAACCTTGATTCCTGTCTG (SEQ ID NO.:193); CCCCACGCCTGTTTGTATTGGGAGCTCTGGACCAATAGTGTCTCTCCTAG (SEQ ID NO.:196); CCAGCCACTCTACTCAAGGGGCATATATTTTGGCATGAGGTGGGATAGAG (SEQ ID NO.:240); gcatgtgtatgatgtgtgtgcgtcggaccgcttctaggctactaagtgtc (SEQ ID NO.:257); AGGGGCAGTATACTCTTATCAGTGCGAGGTAGCTGGGGCCTGTGATAGTT (SEQ ID NO.:299); CAAGCCTGGCAGTAAATCCGAATATCCAGAACCCTGACCCTGCCGTGTAC (SEQ ID NO.:319); CAGCATGTAGGGCAGTGCTTGCACGTAGCATCTGGTGCCTAACCAGTGTT (SEQ ID NO.:336); CTGAGGTTATGTACAACCAACTCTCAGAATTCAGACTTCCTGCAGCTGCC (SEQ ID NO.:370); GTAGGCCCCCAAAGTGCCGTCTTTCCCTAGCATTTTACTCAATGTTTGCC (SEQ ID NO.:392); GAATCAAGGAGGTCAAGTAAGGTCACAGGGGCACTTGGGTTGAGCCAGGG (SEQ ID NO.:437); CCCCAGATGGTTCCAAATATTCCTTACCTCGTTTGGTTCCCAAGTCACAG (SEQ ID NO.:450); GAATAGAAACCAGACAGCAATTCTTTAGTTCCAGCCACCATTCGCCCCAC (SEQ ID NO.:454); TCAACAAAGAGGTGCTGACCTGAGAGTAGGGCACATAACCTCAGCCACTG (SEQ ID NO.:471); ATGTAGATGGGGAGTGACCACCGCCAACAGAAGTGTGGCCATCTTGCCCG (SEQ ID NO.:535); CTTTGGGCACCATTTGGATATAGTTAGTGGTGGTTTAGCTATGGCGTTCC (SEQ ID NO.:609); GGCAAATTCCGGGTATGCACTCAACTTCGGCAAAGGCACCTCGCTGTTGG (SEQ ID NO.:637); GAGGCTTTCAGGTAGGAGGACAATGGTAGCACTGTAGGTCCCCAGTGTCG (SEQ ID NO.:754); AGTAAACCCATATATCCAGAACCCTGACCCTGCCGTGTACCAGCTGAGAG (SEQ ID NO.:800); CCTGTGGCAAGCCAGCAAGATGGCCCTGGTGACAGCAAAAGAAACTGCAC (SEQ ID NO.:837); CCAGGTGCCGCCCACTCTTGACGTGATACTTACCGTCAATGCTCCTTACC (SEQ ID NO.:876); GCCTAAACCAGGTATGCCAATCTGTCTTGTGTCCACATACTAACAGAGGG (SEQ ID NO.:924); AGCCAAGACAGCAGCTCTACATCCTTACCTAGGTAATTCAGGCATGCGCC (SEQ ID NO.:947); CACATGGCAAATGCCTCCTTTCACAATAGAGCATGGTGCTGTTTCCTCAC (SEQ ID NO.:954); TATTGCAGCCATCCATCTTGGGGGCTCATCCATCACACCCGGGTTGCTAG (SEQ ID NO.:1010); CTGGGCTGTGGTATTTGGGTGATCTTTACATTCTTCAGACTCATGTGTGT (SEQ ID NO.:1035); GCTACAAACAAGCTCATCTTTGGAACTGGCACTCTGCTTGCTGTCCAGCC (SEQ ID NO.:1081); CCTACTCCTACAGTGCCTTGCATTCCGTAGCTGCTCAGTACATTAACCCA (SEQ ID NO.:1116); CAGGGTATGAAAGTGCCCATTTCTAGCCAACATTAGATACCCTCAGTCTC (SEQ ID NO.:1157); TGGCCACATTTGTCTCAAACTCAAGTCTACACATTTCTCTCTCTTTTCCC (SEQ ID NO.:1227); GTACCGTCAGCAACCTGGACAGAGCCTGACACTGATCGCAACTGCAAATC (SEQ ID NO.:1276); and Gccccctaattgactgaatggaacccctcttgaccaaagtgaccccagaa (SEQ ID NO.:1379). In another aspect, the method further comprises the step of differentiating between sarcoidosis and tuberculosis, lung cancer or pneumonia by determining the expression levels of the following genes, markers, or probes: PHF20L1; LOC400304; SELM; DPM2; RPLP1; SF1; ZNF683; CTTN; PTCRA; SNORA28; RPGRIP1; GPR160; PPIA; DNASE1L1; HEMGN; RAB13; NFIA; LOC728843; LOC100134660; LOC100132564; HIP1; PRMT1; PDGFC; NCRNA00085; NFATC3; GIMAP7; LOC100130905; AKAP7; TLE3; NRSN2; RPL37; CSTA; C20orf107; TMEM169; GCAT; TMEM176A; CMTM5; C3orf26; FANCD2; C9orf114; TIAM2; LOC644615; PADI2; GRINA; CHST13; ANGPT1; KIF27; ZNF550; PIK3C2A; NR1H3; ALG8; SLC2A5; ITGB5; OPN3; UBE2O; RIN3; LOC100129203; B3GNT1; NEK8; SLC38A5; GPR183; LOC728748; LOC646966; FAM159A; LOC441073; CCNC; MRPL9; SLC37A1; NSUN5; GHRL; ALAS2; MPZL2; RNF13; SUMO1P1; UHRF2; RNY4; LOC651524; KBTBD8; ZNF224; OLIG1; TNFRSF4; BEND7; LOC728323; ARHGAP24; CCCTGCCCTCATGTTGCTTTGGGTCTAGTGGAGGAGAGAGACAGATAAGC (SEQ ID NO.:1447); CAAGTTCTTAACCATCCCGGGTTCCAGTGGTTACAGAGTTCTGCCCTGGG; (SEQ ID NO.:1448) and TGCATGAGATCACACAACTAGGCGGTGACTGAGTCCAACACACCAAAGCC (SEQ ID NO.:1449). In another aspect, the method further comprises the step of differentiating between sarcoidosis that is active and sarcoidosis that is inactive by determining the expression levels of the following genes, markers, or probes: LOC442132; HOXA1; LOC652102; PPIE; C22orf27; TEX10; LMTK2; LOC283663; SUCNR1; COLQ; HLA-DOB; SAMSN1; INPP5E; CYP4F3; CRYZ; CDC14A; LOC653061; KIR2DL4; PCYOX1L; TCEAL3; FRRS1; PHF17; PDK4; LOC440313; ZNF260; SLFN13; VASH1; GM2A; ASAP2; VARS2; RPL14; KIR2DL1; SBDSP; S1PR3; and METTL1; CCAGGAGGCCGAACACTTCTTTCTGCTTTCTTGACATCCGCTCACCAGGC (SEQ ID NO.:1450), and TTCCAGGGCACGAGTTCGAGGCCAGCCTGGTCCACATGGGTCGGaaaaaa (SEQ ID NO.:1451). In another aspect, the method further comprises the step of using 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 144, 150, 200, 250, 300, 400, 500, 600, 700, 800, 900, 1,000, 1,100, 1,200, 1,300, 1,400, or 1,446 genes selected from SEQ ID NOS.: 1 to 1446 to determine if the patient has at least one of tuberculosis, sarcoidosis, cancer or pneumonia.

Yet another embodiment of the present invention includes a method for determining the effectiveness of a treating a sarcoidosis patient comprising: obtaining a sample from a subject suspected of having a pulmonary disease; determining the expression level of 3, 4, 5, 6 or more genes selected from IL1R2; GRB10; CEACAM4; SIPA1L2; BMX; IL1RAP; REPS2; ANXA3; MMP9; PHC2; HAUS4; DUSP1; CA4; SAMSN1; KLHL2; ACSL1; NSUN7; IL18RAP; GNG10; SMAP2; MGAM; LIN7A; IRAK3; USP10; CEBPD; TGFA; FOS; MANSC1; SLC26A8; ROPN1L; GPR97; NAMPT; MRVI1; KCNJ15; KLHL8; GNG10; MEGF9; GPR160; B4GALT5; STEAP4; LRG1; F5; PHTF1; HMGB2; DGAT2; SLC11A1; QPCT; PANX2; GPR141; or LMNB1; wherein overexpression of the genes is indicative of a reduction in sarcoidosis.

Another embodiment of the present invention includes a method of identifying a subject with a pulmonary disease comprising: obtaining a sample from a subject suspected of having a pulmonary disease; determining the expression level of six or more genes from each of the following genes selected from: UBE2J2; ALPL; JMJD6; FCER1G; LILRA5; LY96; FCGR1C; C10orf33; GPR109B; PROK2; PIM3; SH3GLB1; DUSP3; PPAP2C; SLPI; MCTP1; KIF1B; FLJ32255; BAGE5; IFITM1; GPR109A; IF135; LOC653591; KREMEN1; IL18R1; CACNA1E; ABCA2; CEACAM1; MXD4; TncRNA; LMNB1; H2AFJ; HP; ZNF438; FCER1A; SLC22A4; DISC1; MEFV; ABCA1; ITPRIPL2; KCNJ15; LOC728519; ERLIN1; NLRC4; B4GALT5; LOC653610; HIST2H2BE; AIM2; P2RY10; CCR3; EMR4P; NTN3; C1QB; TAOK1; FCGR1B; GATA2; FKBP5; DGAT2; TLR5; CARD17; INCA; MSL3L1; ESPN; LOC645159; C19orf59; CDK5RAP2; PLSCR1; RGL4; IFI30; LOC641710; GAGGCTTTCAGGTAGGAGGACAATGGTAGCACTGTAGGTCCCCAGTGTCG (SEQ ID NO.: 754); LOC100008589; LOC100008589; SMARCD3; NGFRAP1; LOC100132394; OPLAH; CACNG6; LILRB4; HIST2H2AA4; CYP1B1; PGS1; SPATA13; PFKFB3; HIST1H3D; SNORA73B; SLC26A8; SULT1B1; ADM; HIST2H2AA3; HIST2H2AA3; GYG1; CST7; EMR4; LILRA6; MEF2D; IFITM3; MSL3; DHRS13; EMR4; C16orf57; HIST2H2AC; EEF1D; TDRD9; GPR97; ZNF792; LOC100134364; SRGAP3; FCGR1A; HPSE; LOC728417; LOC728417; MIR21; HIST1H2BG; COP1; SMARCD3; LOC441763; ZSCAN18; GNG8; MTRF1L; ANKRD33; PLAC8; PLAC8; SLC26A8; AGTRAP; FLJ43093; LPCAT2; AGTRAP; S100A12; SVIL; LILRA5; LILRA5; ZFP91; CLC; LOC100133565; LTB4R; SEPT04; ANXA3; BHLHB2; IL4R; IFNAR1; MAZ; gccccctaattgactgaatggaacccctcttgaccaaagtgaccccagaa (SEQ ID NO.: 1379); comparing the expression level of the 3, 4, 5, 6 or more genes with the expression level of the same genes from individuals not afflicted with a pulmonary disease, and determining the level of expression of the six or more genes in the sample from the subject relative to the samples from individuals not afflicted with a pulmonary disease for the genes expressed in the one or more expression pathways, selected from: EIF2 signaling and mTOR signaling pathways are indicative of active sarcoidosis; co-expression of genes in the regulation of eIF4 and p70s6K signaling pathways is indicative of pneumonia; co-expression of genes in the interferon signaling and antigen presentation pathways are indicative of tuberculosis; and co-expression of genes in the T cell signaling pathways; and other signaling pathways is indicative of lung cancer. In one aspect, the genes that are downregulated are selected from MEF2D; BHLHB2; CLC; FCER1A; SRGAP3; FLJ43093; CCR3; EMR4; ZNF792; C10orf33; CACNG6; P2RY10; GATA2; EMR4P; ESPN; EMR4; MXD4; and ZSCAN18. In another aspect, the method further comprises a method for displaying if the patient has tuberculosis, sarcoidosis, cancer or pneumonia by aggregating the expression data from the six or more genes into a single visual display of a vector of expression for tuberculosis, sarcoidosis, cancer or pneumonia. In another aspect, the method further comprises the step of detecting and evaluating 7, 8, 9, 10, 12, 15, 20, 25, 35, 50, 75, 90, 100, 125, or 144 genes for the analysis. In another aspect, the sample is a blood, peripheral blood mononuclear cells, sputum, or lung biopsy. In another aspect, the expression level comprises an mRNA expression level and is quantitated by a method selected from the group consisting of polymerase chain reaction, real time polymerase chain reaction, reverse transcriptase polymerase chain reaction, hybridization, probe hybridization and gene expression array. In another aspect, the expression level is determined using at least one technique selected from polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing. In another aspect, the expression level is determined by microarray analysis that comprises use of oligonucleotides that hybridize to mRNA transcripts or cDNAs for the six or more genes, and wherein the oligonucleotides are disposed or directly synthesized on the surface of a chip or wafer. In another aspect, the oligonucleotides are about 10 to about 50 nucleotides in length. In another aspect, the method further comprises the step of using the determined comparative gene product information to formulate at least one of diagnosis, a prognosis or a treatment plan. In another aspect, the patient's disease state is further determined by radiological analysis of the patient's lungs. In another aspect, the method further comprises step of determining a treated patient gene expression dataset after the patient has been treated and determining if the treated patient gene expression dataset has returned to a normal gene or a changed gene expression dataset thereby determining if the patient has been treated. In another aspect, a non-overlapping set of genes is used to distinguish between Tb, sarcoidosis, pneumonia and lung cancer, versus, Tb, active sarcoidosis, non-active sarcoidosis, pneumonia and lung cancer are selected from Table 11, 12 or both. Yet another embodiment of the present invention includes a computer readable medium comprising computer-executable instructions for performing the methods of the present invention.

### BRIEF DESCRIPTION OF THE DRAWINGS

For a more complete understanding of the features and advantages of the present invention, reference is now made to the detailed description of the invention along with the accompanying figures and in which:

FIG. 1 shows a heatmap of pulmonary granulomatous diseases, TB and sarcoidosis, display similar transcriptional signatures (of 1446 transcripts) to each other but distinct from pneumonia and lung cancer.

FIG. 2 shows a heat map with three dominant clusters of transcripts in the unsupervised clustering of the 1446 transcripts are associated with distinct Ingenuity Pathway Analysis canonical pathways.

FIGS. 3A and 3B (quantitative) show that sarcoidosis patients clinically classified as active sarcoidosis display similar transcriptional signatures to the TB patients but are very distinct from the transcriptional signatures of the clinically classified non-active sarcoidosis patients, which in turn resemble the healthy controls.

FIGS. 4A to 4E show a modular analysis of the Training Set shows the similarity of the biological pathways associated with TB and sarcoidosis (which show particularly overexpression of the IFN modules), differing from pneumonia and lung cancer (particularly overexpression of the inflammation modules). All are quantitated in FIGS. 4D and 4E

FIGS. 5A to 5E show a Comparison Ingenuity Pathway Analysis of the four disease groups compared to their matched controls reveals the four most significant pathways.

FIGS. 6A to 6D shows both modular analysis and molecular distance to health reveal that the blood transcriptome of the pneumonia and TB patients after successfully completing treatment are no different from the healthy controls, however the sarcoidosis patients show an overexpression of inflammation genes during a clinically successful response to glucocorticoids.

FIGS. 7A to 7E shows that the Interferon-inducible gene expression is most abundant in the neutrophils in both TB and sarcoidosis.

FIGS. 8A and 8B are graphs with the results for the pulmonary diseases using the genes in the neutrophil module.

FIG. 9 is a 4-set Venn diagram comparing the differentially expressed genes for each disease group compared to their ethnicity and gender matched controls.

FIG. 10A is a Venn diagram comparing the gene lists used in the class prediction. FIG. 10B is a Venn diagram comparing the genes that distinguish between Tb, sarcoidosis, pneumonia and lung cancer, versus, Tb, active sarcoidosis, non-active sarcoidosis, pneumonia and lung cancer.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides methods, compositions, biomarkers and tests for evaluating the immunopathogenesis underlying TB and other pulmonary diseases, by comparing the blood transcriptional responses in pulmonary TB patients to that found in pulmonary sarcoidosis, pneumonia and lung cancer patients. It also provides for the first time a complete, reproducible comparison of blood transcriptional responses before and after treatment in each disease, and examining the transcriptional responses seen in the different leucocyte populations of the granulomatous diseases. In addition the present inventors investigated the association between the clinical heterogeneity of sarcoidosis and the observed blood transcriptional heterogeneity.

As used herein, the term “array” refers to a solid support or substrate with one or more peptides or nucleic acid probes attached to the support. Arrays typically have one or more different nucleic acid or peptide probes that are coupled to a surface of a substrate in different, known locations. These arrays, also described as “microarrays” or “gene-chips” that may have 10,000; 20,000, 30,000; or 40,000 different identifiable genes based on the known genome, e.g., the human genome. These pan-arrays are used to detect the entire “transcriptome” or transcriptional pool of genes that are expressed or found in a sample, e.g., nucleic acids that are expressed as RNA, mRNA and the like that may be subjected to RT and/or RT-PCR to made a complementary set of DNA replicons. The microarray is well known in the art, for example, U.S. Pat. Nos. 5,445,934 and 5,744,305. The term also includes all the devices so called in Schena (ed.), DNA Microarrays: A Practical Approach (Practical Approach Series), Oxford University Press (1999) (ISBN: 0199637768); Nature Genet. 21(1)(suppl):1-60 (1999); and Schena (ed.), Microarray Biochip: Tools and Technology, Eaton Publishing Company/BioTechniques Books Division (2000) (ISBN: 1881299376)(relevant portions incorporated herein by reference), the disclosures of which are incorporated herein by reference in their entirety. Arrays may be produced using mechanical synthesis methods, light directed synthesis methods and the like that incorporate a combination of non-lithographic and/or photolithographic methods and solid phase synthesis methods. In one embodiment, the present invention includes simplified arrays that can include a limited number of probes, e.g., 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 144, 150, 200, 250, 300, 400, 500, 600, 700, 800, 900, 1,000, 1,100, 1,200, 1,300, 1,400, or even 1,446 genes or probes in a customized or customizable microarray adapted for pulmonary disease detection, diagnosis and evaluation.

As used herein the term “biomarker” refers to a specific biochemical in the body that has a particular molecular feature to make it useful for diagnosing and measuring the progress of disease or the effects of treatment. Certain biomarkers form part of the present invention and are attached to this application as Lengthy Tables, that are included herewith and the content incorporated herein by reference. The text file Symbol-Regulation-ID.txt is 47Kb and Symbol-Sequence-ID.txt provide the list of 1446 probe sequences and genes that are associated with the majority of the same. Also included herewith is a list of 1359 genes that overlay in certain conditions as described hereinbelow.

Various techniques for the synthesis of these nucleic acid arrays have been described, e.g., fabricated on a surface of virtually any shape or even a multiplicity of surfaces. Arrays may be peptides or nucleic acids on beads, gels, polymeric surfaces, fibers such as fiber optics, glass or any other appropriate substrate. Arrays may be packaged in such a manner as to allow for diagnostics or other manipulation of an all inclusive device, see for example, U.S. Pat. No. 6,955,788, relevant portions incorporated herein by reference.

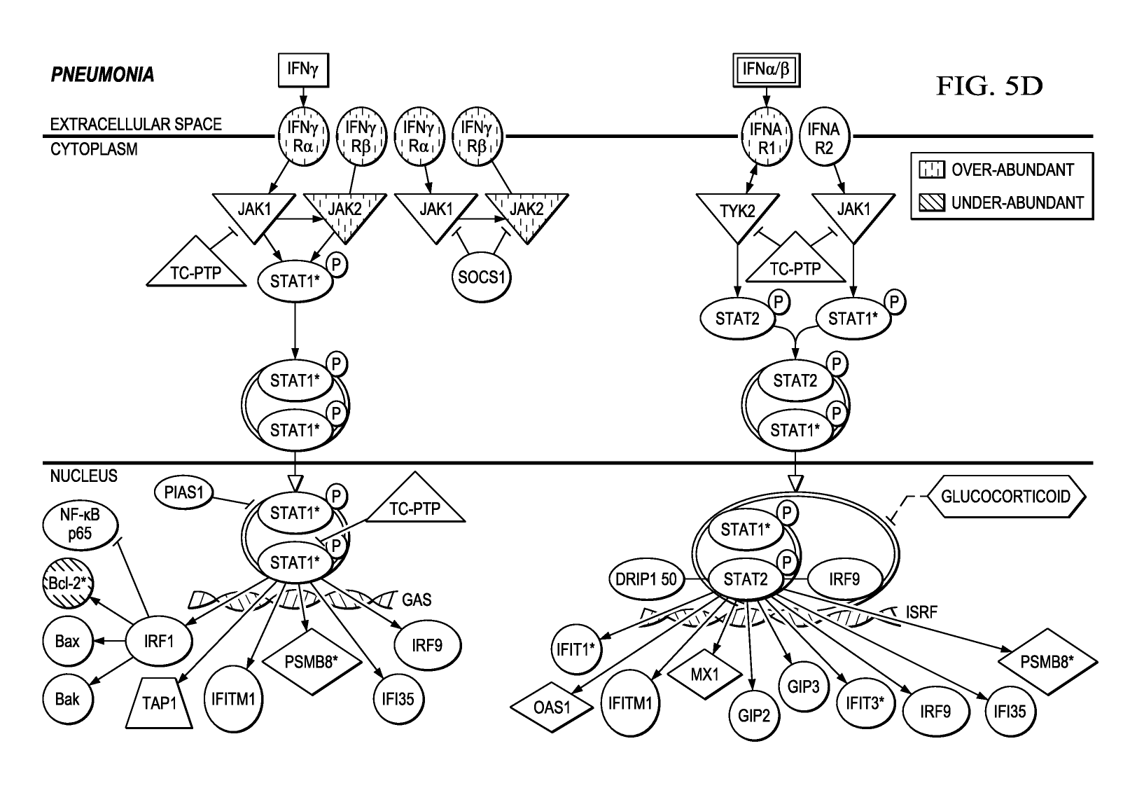

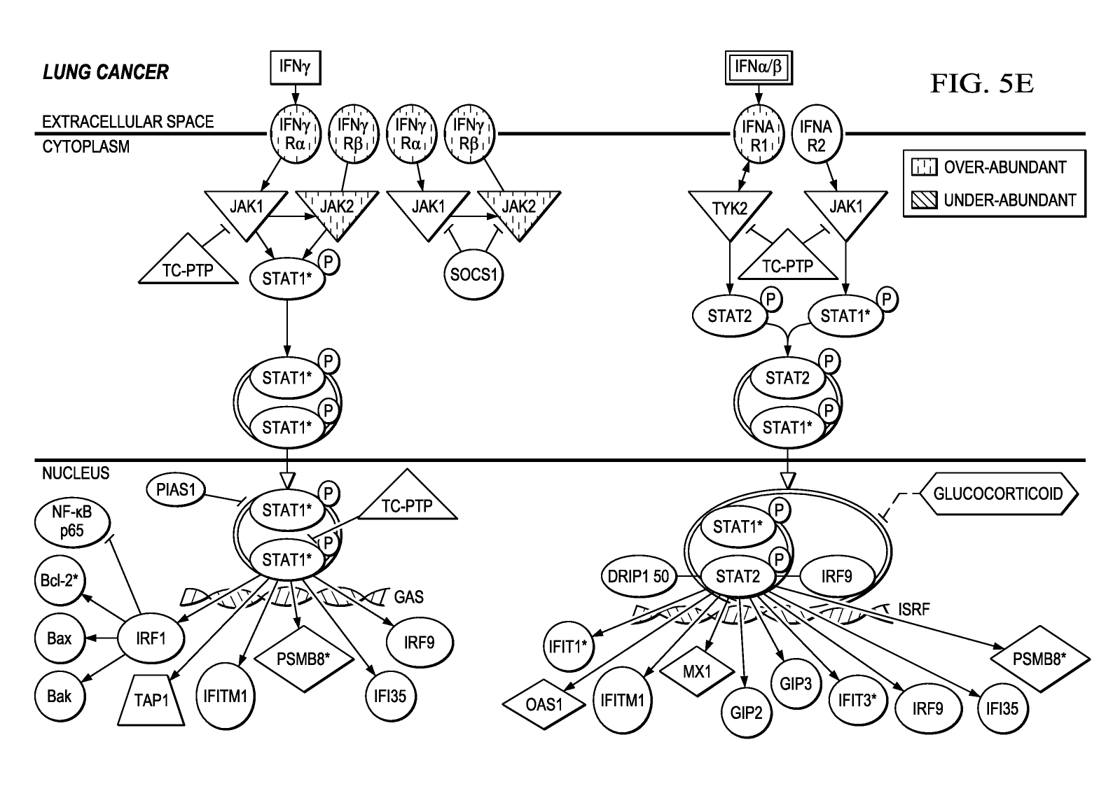

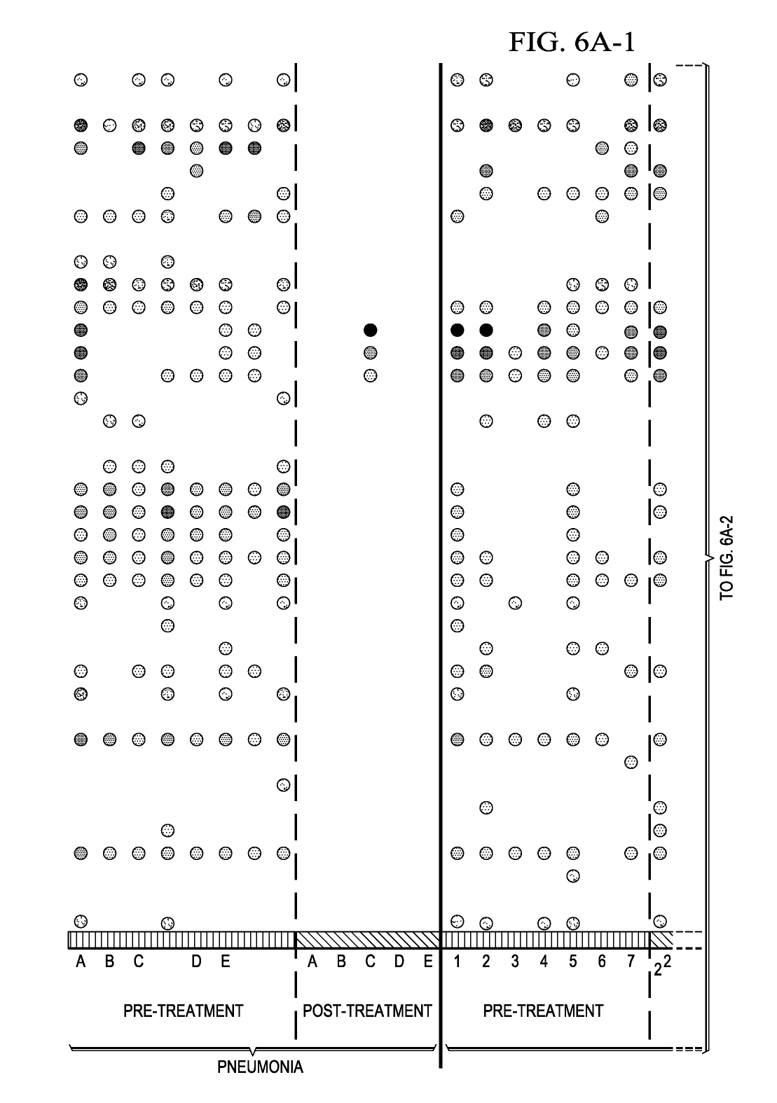

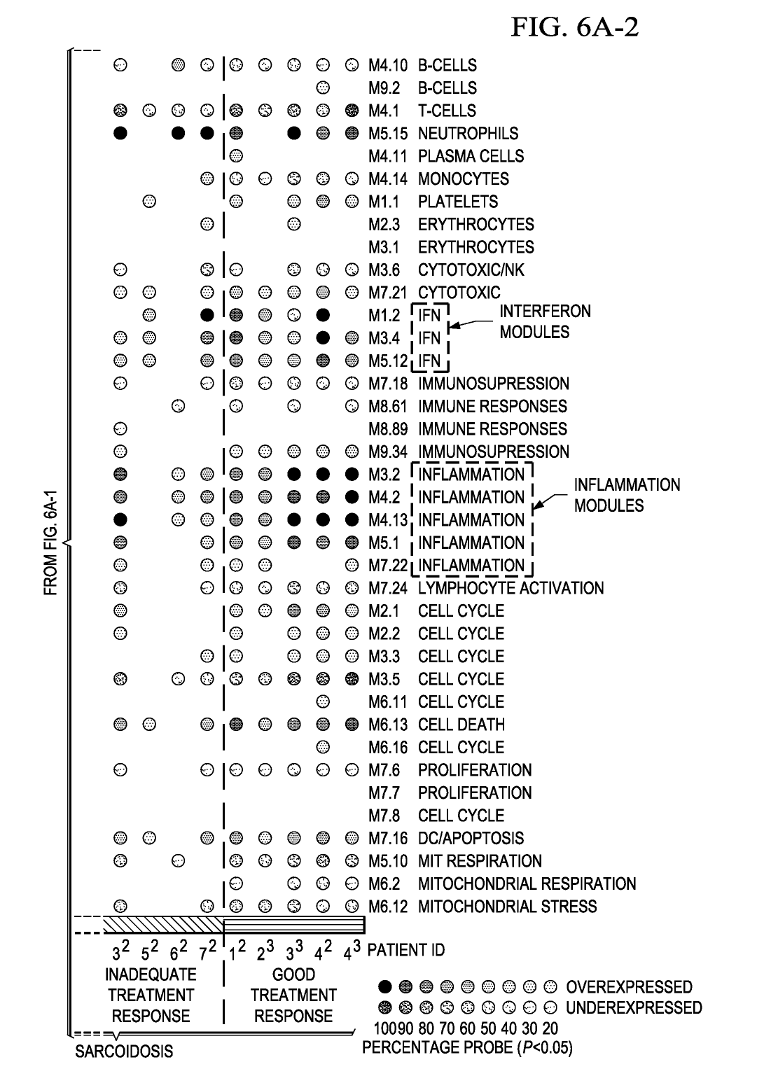

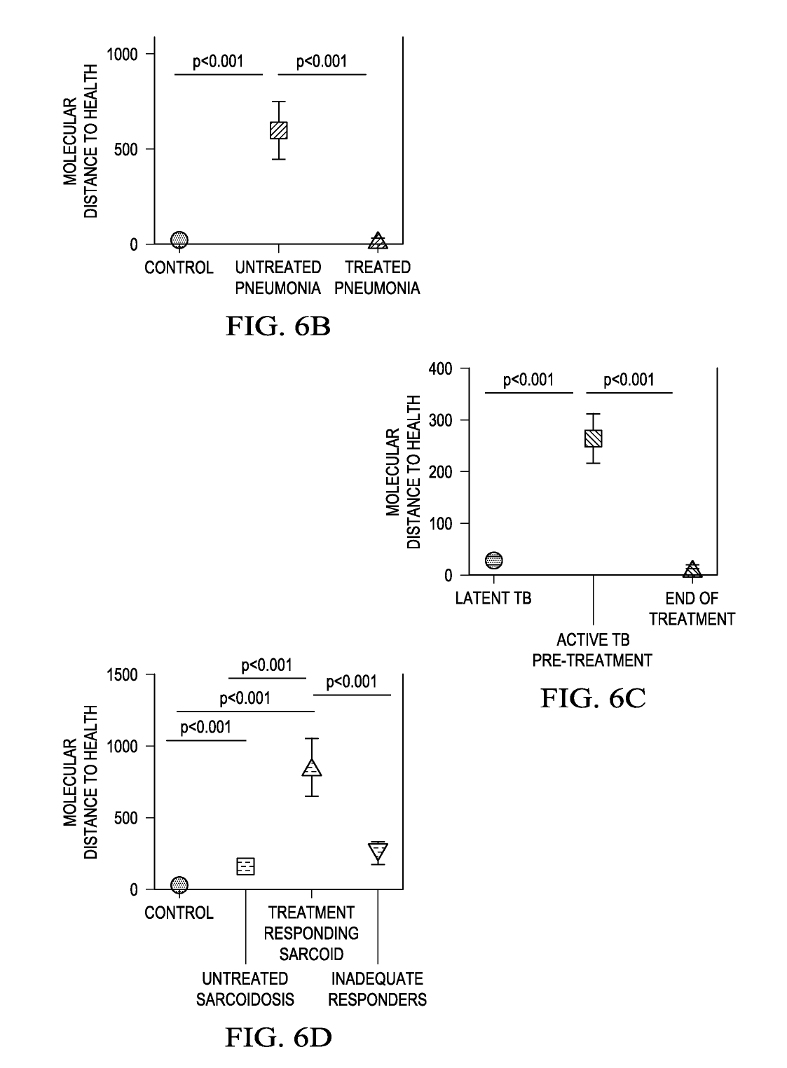

As used herein, the term “disease” refers to a physiological state of an organism with any abnormal biological state of a cell. Disease includes, but is not limited to, an interruption, cessation or disorder of cells, tissues, body functions, systems or organs that may be inherent, inherited, caused by an infection, caused by abnormal cell function, abnormal cell division and the like. A disease that leads to a “disease state” is generally detrimental to the biological system, that is, the host of the disease. With respect to the present invention, any biological state, such as an infection (e.g., viral, bacterial, fungal, helminthic, etc.), inflammation, autoinflammation, autoimmunity, anaphylaxis, allergies, premalignancy, malignancy, surgical, transplantation, physiological, and the like that is associated with a disease or disorder is considered to be a disease state. A pathological state is generally the equivalent of a disease state. Disease states may also be categorized into different levels of disease state. As used herein, the level of a disease or disease state is an arbitrary measure reflecting the progression of a disease or disease state as well as the physiological response upon, during and after treatment. Generally, a disease or disease state will progress through levels or stages, wherein the affects of the disease become increasingly severe. The level of a disease state may be impacted by the physiological state of cells in the sample. As used herein, the terms “module”, “modular transcriptional vectors”, or “vectors of gene expression” refer to transcriptional expression data that reflects a proportion of differentially expressed genes having a common gene expression pathway (e.g., interferon inducible genes), are typically expressed only or predominantly in a certain cell type (e.g., genes expressed by neutrophils), or are grouped into a module of genes to yield, in the aggregate a single vector of gene expression, such that the overall expression is expressed as a single vector that includes both a direction (under expressed or over expressed) and intensity of the under or over expression. For example, for each module the proportion of transcripts differentially expressed between at least two groups (e.g., healthy subjects versus patients, or certain patients of a first disease versus a group of patients with a second disease). The vector of expression is derived from the comparison of two or more groups of samples. The first analytical step is used for the selection of disease-specific sets of transcripts within each module. Next, there is the “expression level.” The group comparison for a given disease provides the list of differentially expressed transcripts for each module. It was found that different diseases yield different subsets of modular transcripts. With this expression level it is then possible to calculate a vector of expression for each of the module(s) for a single sample by averaging expression values of disease-specific subsets of genes identified as being differentially expressed. This approach permits the generation of maps of modular expression vectors for a single sample, e.g., those described in the module maps disclosed herein. These vector of expression or module maps represent an averaged expression level for each module (instead of a proportion of differentially expressed genes) that can be derived for each sample. An example of the vector of gene expression is shown in, e.g., FIG. 6A.

Using the present invention it is possible to identify and distinguish pulmonary diseases not only at the module-level, but also at the gene-level; i.e., two, three or four diseases can have for certain modules the same vector (identical proportion of differentially expressed transcripts, identical “polarity”), but the gene composition of the vector can still be disease-specific, and vice versa. Gene-level expression provides the distinct advantage of greatly increasing the resolution of the analysis.

Gene expression monitoring systems for use with the present invention may include customized gene arrays with a limited and/or basic number of genes that are specific and/or customized for the one or more target diseases. Unlike the general, pan-genome arrays that are in customary use, the present invention provides for not only the use of these general pan-arrays for retrospective gene and genome analysis without the need to use a specific platform, but more importantly, it provides for the development of customized arrays that provide an optimal gene set for analysis without the need for the thousands of other, non-relevant genes. One distinct advantage of the optimized arrays and modules of the present invention over the existing art is a reduction in the financial costs (e.g., cost per assay, materials, equipment, time, personnel, training, etc.), and more importantly, the environmental cost of manufacturing pan-arrays where the vast majority of the data is irrelevant. The modules of the present invention allow for the first time the design of simple, custom arrays that provide optimal data with the least number of probes while maximizing the signal to noise ratio. By eliminating the total number of genes for analysis, it is possible to, e.g., eliminate the need to manufacture thousands of expensive platinum masks for photolithography during the manufacture of pan-genetic chips that provide vast amounts of irrelevant data. Using the present invention it is possible to completely avoid the need for microarrays if the limited probe set(s) of the present invention are used with, e.g., digital optical chemistry arrays, ball bead arrays, beads (e.g., Luminex), multiplex PCR, quantitiative PCR, run-on assays, Northern blot analysis, or even, for protein analysis, e.g., Western blot analysis, 2-D and 3-D gel protein expression, MALDI, MALDI-TOF, fluorescence activated cell sorting (FACS) (cell surface or intracellular), enzyme linked immunosorbent assays (ELISA), chemiluminescence studies, enzymatic assays, proliferation studies or any other method, apparatus and system for the determination and/or analysis of gene expression that are readily commercially available.

As used herein, the term “differentially expressed” refers to the measurement of a cellular constituent (e.g., nucleic acid, protein, enzymatic activity and the like) that varies in two or more samples, e.g., between a disease sample and a normal sample. The cellular constituent may be on or off (present or absent), upregulated relative to a reference or downregulated relative to the reference. For use with gene-chips or gene-arrays, differential gene expression of nucleic acids, e.g., mRNA or other RNAs (miRNA, siRNA, hnRNA, rRNA, tRNA, etc.) may be used to distinguish between cell types or nucleic acids. Most commonly, the measurement of the transcriptional state of a cell is accomplished by quantitative reverse transcriptase (RT) and/or quantitative reverse transcriptase-polymerase chain reaction (RT-PCR), genomic expression analysis, post-translational analysis, modifications to genomic DNA, translocations, in situ hybridization and the like.

As used herein, the terms “therapy” or “therapeutic regimen” refer to those medical steps taken to alleviate or alter a disease state, e.g., a course of treatment intended to reduce or eliminate the affects or symptoms of a disease using pharmacological, surgical, dietary and/or other techniques. A therapeutic regimen may include a prescribed dosage of one or more drugs or surgery. Therapies will most often be beneficial and reduce the disease state but in many instances the effect of a therapy will have non-desirable or side-effects. The effect of therapy will also be impacted by the physiological state of the host, e.g., age, gender, genetics, weight, other disease conditions, etc.

As used herein, the term “pharmacological state” or “pharmacological status” refers to those samples from diseased individuals that will be, are and/or were treated with one or more drugs, surgery and the like that may affect the pharmacological state of one or more nucleic acids in a sample, e.g., newly transcribed, stabilized and/or destabilized as a result of the pharmacological intervention. The pharmacological state of a sample relates to changes in the biological status before, during and/or after drug treatment and may serve as a diagnostic or prognostic function, as taught herein. Some changes following drug treatment or surgery may be relevant to the disease state and/or may be unrelated side-effects of the therapy. Changes in the pharmacological state are the likely results of the duration of therapy, types and doses of drugs prescribed, degree of compliance with a given course of therapy, and/or un-prescribed drugs ingested.

As used herein, the term “biological state” refers to the state of the transcriptome (that is the entire collection of RNA transcripts) of the cellular sample isolated and purified for the analysis of changes in expression. The biological state reflects the physiological state of the cells in the blood sample by measuring the abundance and/or activity of cellular constituents, characterizing according to morphological phenotype or a combination of the methods for the detection of transcripts. As used herein, the term “expression profile” refers to the relative abundance of RNA, DNA abundances or activity levels. The expression profile can be a measurement for example of the transcriptional state or the translational state by any number of methods and using any of a number of gene-chips, gene arrays, beads, multiplex PCR, quantitiative PCR, run-on assays, Northern blot analysis, or using RNA-seq, nanostring, nanopore RNA sequencing etc. Apparatus and system for the determination and/or analysis of gene expression that are readily commercially available.

As used herein the term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “transcriptional state” of a sample includes the identities and relative abundances of the RNA species, especially mRNAs present in the sample. The entire transcriptional state of a sample, that is the combination of identity and abundance of RNA, is also referred to herein as the transcriptome. Generally, a substantial fraction of all the relative constituents of the entire set of RNA species in the sample are measured.

Regarding the “expression level,” the group comparison for a given disease provides the list of differentially expressed transcripts. It was found that different diseases yield different subsets of gene transcripts as demonstrated herein.

Gene expression monitoring systems for use with the present invention may include customized gene arrays with a limited and/or basic number of genes that are specific and/or customized for the one or more target diseases. Unlike the general, pan-genome arrays that are in customary use, the present invention provides for not only the use of these general pan-arrays for retrospective gene and genome analysis without the need to use a specific platform, but more importantly, it provides for the development of customized arrays that provide an optimal gene set for analysis without the need for the thousands of other, non-relevant genes. One distinct advantage of the optimized arrays and gene sets of the present invention over the existing art is a reduction in the financial costs (e.g., cost per assay, materials, equipment, time, personnel, training, etc.), and more importantly, the environmental cost of manufacturing pan-arrays where the vast majority of the data is irrelevant. By eliminating the total number of genes for analysis, it is possible to, e.g., eliminate the need to manufacture thousands of expensive platinum masks for photolithography during the manufacture of pan-genetic chips that provide vast amounts of irrelevant data. Using the present invention it is possible to completely avoid the need for microarrays if the limited probe set(s) of the present invention are used with, e.g., digital optical chemistry arrays, ball bead arrays, multiplex PCR, quantitiative PCR, “RNA-seq” for measuring mRNA levels using next-generation sequencing technologies, nanostring-type technologies or any other method, apparatus and system for the determination and/or analysis of gene expression that are readily commercially available.

The “molecular fingerprinting system” of the present invention may be used to facilitate and conduct a comparative analysis of expression in different cells or tissues, different subpopulations of the same cells or tissues, different physiological states of the same cells or tissue, different developmental stages of the same cells or tissue, or different cell populations of the same tissue against other diseases and/or normal cell controls. In some cases, the normal or wild-type expression data may be from samples analyzed at or about the same time or it may be expression data obtained or culled from existing gene array expression databases, e.g., public databases such as the NCBI Gene Expression Omnibus database.

As used herein, the term “differentially expressed” refers to the measurement of a cellular constituent (e.g., nucleic acid, protein, enzymatic activity and the like) that varies in two or more samples, e.g., between a disease sample and a normal sample. The cellular constituent may be on or off (present or absent), upregulated relative to a reference or downregulated relative to the reference. For use with gene-chips or gene-arrays, differential gene expression of nucleic acids, e.g., mRNA or other RNAs (miRNA, siRNA, hnRNA, rRNA, tRNA, etc.) may be used to distinguish between cell types or nucleic acids. Most commonly, the measurement of the transcriptional state of a cell is accomplished by quantitative reverse transcriptase (RT) and/or quantitative reverse transcriptase-polymerase chain reaction (RT-PCR), genomic expression analysis, post-translational analysis, modifications to genomic DNA, translocations, in situ hybridization and the like.

The skilled artisan will appreciate readily that samples may be obtained from a variety of sources including, e.g., single cells, a collection of cells, tissue, cell culture and the like. In certain cases, it may even be possible to isolate sufficient RNA from cells found in, e.g., urine, blood, saliva, tissue or biopsy samples and the like. In certain circumstances, enough cells and/or RNA may be obtained from: mucosal secretion, feces, tears, blood plasma, peritoneal fluid, interstitial fluid, intradural, cerebrospinal fluid, sweat or other bodily fluids. The nucleic acid source, e.g., from tissue or cell sources, may include a tissue biopsy sample, one or more sorted cell populations, cell culture, cell clones, transformed cells, biopies or a single cell. The tissue source may include, e.g., brain, liver, heart, kidney, lung, spleen, retina, bone, neural, lymph node, endocrine gland, reproductive organ, blood, nerve, vascular tissue, and olfactory epithelium.

The present invention includes the following basic components, which may be used alone or in combination, namely, one or more data mining algorithms, one novel algorithm specifically developed for this TB treatment monitoring, the Temporal Molecular Response; the characterization of blood leukocyte transcriptional gene sets; the use of aggregated gene transcripts in multivariate analyses for the molecular diagnostic/prognostic of human diseases; and/or visualization of transcriptional gene set-level data and results. Using the present invention it is also possible to develop and analyze composite transcriptional markers. The composite transcriptional markers for individual patients in the absence of control sample analysis may be further aggregated into a reduced multivariate score.

An explosion in data acquisition rates has spurred the development of mining tools and algorithms for the exploitation of microarray data and biomedical knowledge. Approaches aimed at uncovering the function of transcriptional systems constitute promising methods for the identification of robust molecular signatures of disease. Indeed, such analyses can transform the perception of large-scale transcriptional studies by taking the conceptualization of microarray data past the level of individual genes or lists of genes.

The present inventors have recognized that current microarray-based research is facing significant challenges with the analysis of data that are notoriously “noisy,” that is, data that is difficult to interpret and does not compare well across laboratories and platforms. A widely accepted approach for the analysis of microarray data begins with the identification of subsets of genes differentially expressed between study groups. Next, the users try subsequently to “make sense” out of resulting gene lists using the novel Temporal Molecular Response discovery algorithms and existing scientific knowledge and by validating in independent sample sets and in different microarray analyses.

Pulmonary tuberculosis (PTB) is a major and increasing cause of morbidity and mortality worldwide caused by Mycobacterium tuberculosis (M. tuberculosis). However, the majority of individuals infected with M. tuberculosis remain asymptomatic, retaining the infection in a latent form and it is thought that this latent state is maintained by an active immune response. Blood is the pipeline of the immune system, and as such is the ideal biologic material from which the health and immune status of an individual can be established.

Blood represents a reservoir and a migration compartment for cells of the innate and the adaptive immune systems, including neutrophils, dendritic cells and monocytes, or B and T lymphocytes, respectively, which during infection will have been exposed to infectious agents in the tissue. For this reason whole blood from infected individuals provides an accessible source of clinically relevant material where an unbiased molecular phenotype can be obtained using gene expression microarrays for the study of cancer in tissues autoimmunity), and inflammation, infectious disease, or in blood or tissue. Microarray analyses of gene expression in blood leucocytes have identified diagnostic and prognostic gene expression signatures, which have led to a better understanding of mechanisms of disease onset and responses to treatment. These microarray approaches have been attempted for the study of active and latent TB but as yet have yielded small numbers of differentially expressed genes only, and in relatively small numbers of patients, therefore not reaching statistical significance, which may not be robust enough to distinguish between other inflammatory and infectious diseases. The present inventors recognized that a neutrophil driven blood transcriptional signature in active TB patients was missing in the majority of Latent TB individuals and in healthy controls. For this description see, also, the study of Berry et al., 2010 (5), by the present inventors. This signature of active TB was reflective of lung radiographic disease and was diminished after 2 months of treatment (5) and more recently the present inventors have shown that the blood transcriptional signature of TB was diminished as early as 2 weeks after commencement of treatment (12). The signature was dominated by interferon-inducible genes, and at a modular level the active TB signature (5, 12) was distinct from other infectious or autoimmune diseases (5).

In the present findings and the basis of this application the blood transcriptional profiles of the pulmonary granulomatous diseases (TB and sarcoidosis) clustered together but distinctly from the similar pulmonary diseases pneumonia and lung cancer.

It has previously been shown that TB and sarcoidosis have similar transcriptional profiles however no published studies have determined if this similar blood gene expression profile is due to generalized transcriptional activity associated with pulmonary diseases or due to specific host responses associated with TB and sarcoidosis. Therefore, we recruited three cohorts of TB and sarcoidosis patients (Training, Test and Validation Sets) alongside patients with similar pulmonary diseases community acquired pneumonia and lung cancer. On average the sarcoidosis patients presented with a milder and more chronic presentation than the TB and pneumonia patients. There was little difference in the demographics and clinical characteristics of the participants in the Training and Test Sets.

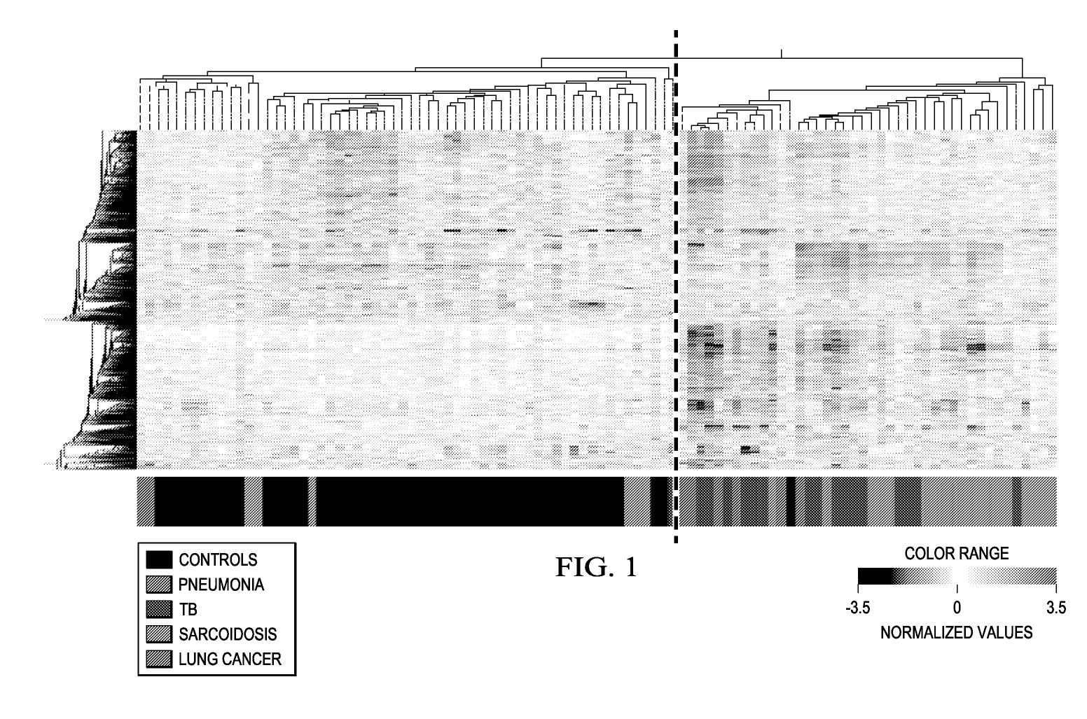

Unbiased analysis followed by unsupervised hierarchical clustering of the blood transcriptional profiles from all the Training Set participants clearly demonstrated that the TB and sarcoidosis patients transcriptional profiles clustered together but distinctly from the pneumonia and cancer patients transcriptional profiles which themselves clustered together (3422 transcripts). Adding a statistical filter generated 1446 differentially expressed transcripts. Applying unsupervised hierarchical clustering of the 1446-transcripts and the Training Set samples again showed the same clustering pattern. This finding was verified in an independent cohort, the Test Set, which likewise showed the TB and most sarcoidosis patients clustered together while the pneumonia and lung cancer patients also clustered together but separately from the granulomatous diseases (FIG. 1). Clustering was not influenced by ethnicity or gender (data not shown).

FIG. 1. The pulmonary granulomatous diseases, TB and sarcoidosis, display similar transcriptional signatures to each other but distinct from pneumonia and lung cancer. 1446-transcripts were differentially expressed in the whole blood of the Training Set healthy controls, pulmonary TB patients, pulmonary sarcoidosis patients, pneumonia patients and lung cancer patients. The clustering of the 1446-transcripts were tested in an independent cohort from which they were derived from, the Test Set. The heatmap shows the transcripts and patients' profiles as organised by the unbiased algorithm of unsupervised hierarchical clustering. A dotted line is added to the heatmap to help visualisation of the main clusters generated by the clustering algorithm. Transcript intensity values are normalised to the median of all transcripts. Red transcripts are relatively over-abundant and blue transcripts under-abundant. The coloured bar at the bottom of the heatmap indicates which group the profile belongs to.

TABLE 1
List of 1446 genes that differentiate between lung
cancer, pneumonia, TB and sarcodiosis.

| | Cancer vs | Pneumonia vs | Sarcoidosis | Tb vs | SEQ |
|---|---|---|---|---|---|
| Symbol | Control | Control | vs Control | Control | ID NO: |
| TMEM144 | UP | UP | UP | UP | 1 |
| FBLN5 | DOWN | DOWN | DOWN | DOWN | 2 |
| FBLN5 | DOWN | DOWN | DOWN | DOWN | 3 |
| ERI1 | UP | UP | UP | UP | 4 |
| CXCR3 | DOWN | DOWN | DOWN | DOWN | 5 |
| GLUL | UP | UP | UP | UP | 6 |
| LOC728728 | UP | UP | UP | UP | 7 |
| KLHDC8B | UP | UP | UP | UP | 8 |
| KCNJ15 | UP | UP | UP | UP | 9 |
| RNF125 | DOWN | DOWN | DOWN | DOWN | 10 |
| CCNB1IP1 | DOWN | DOWN | DOWN | DOWN | 11 |
| PSG9 | UP | UP | UP | UP | 12 |
| LOC100170939 | UP | UP | UP | UP | 13 |
| QPCT | UP | UP | UP | UP | 14 |
| CD177 | UP | UP | UP | UP | 15 |
| LOC400499 | UP | UP | UP | UP | 16 |
| LOC400499 | UP | UP | UP | UP | 17 |
| LOC100134634 | UP | UP | UP | UP | 18 |
| TMEM88 | UP | UP | UP | UP | 19 |
| LOC729028 | UP | UP | DOWN | UP | 20 |
| EPSTI1 | UP | UP | UP | UP | 21 |
| INSC | UP | UP | UP | UP | 22 |
| LOC728484 | DOWN | DOWN | DOWN | DOWN | 23 |
| ERP27 | DOWN | UP | DOWN | DOWN | 24 |
| CCDC109A | UP | UP | UP | UP | 25 |
| LOC729580 | UP | UP | UP | UP | 26 |
| C2 | DOWN | UP | UP | UP | 27 |
| TTRAP | UP | UP | DOWN | UP | 28 |
| ALPL | UP | UP | DOWN | UP | 29 |
| MAEA | UP | UP | UP | UP | 30 |
| COX10 | DOWN | DOWN | DOWN | DOWN | 31 |
| GPR84 | UP | UP | UP | UP | 32 |
| PHF20L1 | UP | UP | UP | UP | 33 |
| TRMT11 | DOWN | DOWN | DOWN | DOWN | 34 |
| ANKRD22 | UP | UP | UP | UP | 35 |
| MATK | DOWN | DOWN | DOWN | DOWN | 36 |
| TBC1D24 | UP | UP | UP | UP | 37 |
| LILRA5 | UP | UP | UP | UP | 38 |
| TMEM176B | UP | UP | UP | UP | 39 |
| CAMP | UP | UP | UP | UP | 40 |
| PKIA | DOWN | DOWN | DOWN | DOWN | 41 |
| PFTK1 | UP | UP | UP | UP | 42 |
| TPM2 | DOWN | DOWN | DOWN | DOWN | 43 |
| TPM2 | DOWN | DOWN | DOWN | DOWN | 44 |
| PRKCQ | DOWN | DOWN | DOWN | DOWN | 45 |
| PSTPIP2 | UP | UP | UP | UP | 46 |
| LOC129607 | UP | UP | UP | UP | 47 |
| APRT | DOWN | DOWN | DOWN | DOWN | 48 |
| VAMPS | UP | UP | UP | UP | 49 |
| FCGR1C | UP | UP | UP | UP | 50 |
| SHKBP1 | UP | UP | UP | UP | 51 |
| CD79B | DOWN | DOWN | DOWN | DOWN | 52 |
| SIGIRR | DOWN | DOWN | DOWN | DOWN | 53 |
| FKBP9L | UP | UP | UP | UP | 54 |
| LOC729660 | UP | UP | UP | UP | 55 |
| WDR74 | DOWN | DOWN | DOWN | DOWN | 56 |
| LOC646434 | UP | UP | UP | UP | 57 |
| LOC647834 | UP | UP | DOWN | UP | 58 |
| RECK | DOWN | DOWN | DOWN | DOWN | 59 |
| MGST1 | UP | UP | UP | UP | 60 |
| PIWIL4 | UP | UP | UP | UP | 61 |
| LILRB1 | UP | UP | UP | UP | 62 |
| FCGR1B | UP | UP | UP | UP | 63 |
| NOC3L | DOWN | DOWN | DOWN | DOWN | 64 |
| ZNF83 | DOWN | DOWN | DOWN | DOWN | 65 |
| FCGBP | DOWN | DOWN | DOWN | DOWN | 66 |
| SNORD13 | DOWN | DOWN | DOWN | DOWN | 67 |
| LOC642267 | UP | UP | UP | UP | 68 |
| | UP | UP | UP | UP | 69 |
| GBP5 | DOWN | UP | UP | UP | 70 |
| EOMES | DOWN | DOWN | DOWN | DOWN | 71 |
| BST1 | UP | UP | UP | UP | 72 |
| C5 | UP | UP | UP | UP | 73 |
| CHMP7 | DOWN | DOWN | DOWN | DOWN | 74 |
| ETV7 | UP | UP | UP | UP | 75 |
| LOC400304 | DOWN | DOWN | DOWN | DOWN | 76 |
| ILVBL | DOWN | DOWN | DOWN | DOWN | 77 |
| LOC728262 | UP | UP | UP | UP | 78 |
| GNLY | DOWN | DOWN | DOWN | DOWN | 79 |
| LOC388572 | UP | UP | UP | UP | 80 |
| GATA1 | DOWN | DOWN | UP | UP | 81 |
| MYBL1 | DOWN | DOWN | DOWN | DOWN | 82 |
| SELM | DOWN | DOWN | DOWN | DOWN | 83 |
| LOC441124 | UP | UP | UP | UP | 84 |
| LOC441124 | UP | UP | UP | UP | 85 |
| IL12RB1 | DOWN | DOWN | UP | UP | 86 |
| | DOWN | DOWN | DOWN | DOWN | 87 |
| BRIX1 | DOWN | DOWN | DOWN | DOWN | 88 |
| GAS6 | DOWN | UP | UP | UP | 89 |
| GAS6 | UP | UP | UP | UP | 90 |
| LOC100133740 | UP | UP | UP | UP | 91 |
| GPSM1 | DOWN | DOWN | DOWN | DOWN | 92 |
| | DOWN | UP | UP | UP | 93 |
| C6ORF129 | DOWN | DOWN | DOWN | DOWN | 94 |
| | UP | UP | UP | UP | 95 |
| IER3 | UP | UP | UP | UP | 96 |
| MAPK14 | UP | UP | UP | UP | 97 |
| PROK1 | UP | UP | UP | UP | 98 |
| GPR109B | UP | UP | UP | UP | 99 |
| SASP | UP | UP | UP | UP | 100 |
| LOC728093 | UP | UP | UP | UP | 101 |
| PROK2 | UP | UP | DOWN | UP | 102 |
| CTSW | DOWN | DOWN | DOWN | DOWN | 103 |
| ABHD2 | UP | UP | UP | UP | 104 |
| LOC100130775 | DOWN | DOWN | DOWN | DOWN | 105 |
| SLITRK4 | UP | UP | UP | UP | 106 |
| FBXW2 | UP | UP | UP | UP | 107 |
| RTTN | DOWN | DOWN | DOWN | DOWN | 108 |
| TAF15 | UP | UP | DOWN | DOWN | 109 |
| FUT7 | UP | UP | UP | UP | 110 |
| DUSP3 | UP | UP | UP | UP | 111 |
| LOC399715 | UP | UP | DOWN | UP | 112 |
| LOC642161 | DOWN | DOWN | DOWN | DOWN | 113 |
| LOC100129541 | UP | UP | UP | UP | 114 |
| TCTN1 | DOWN | DOWN | DOWN | DOWN | 115 |
| SLAMF8 | DOWN | UP | UP | UP | 116 |
| TGM2 | DOWN | DOWN | DOWN | DOWN | 117 |
| ECE1 | UP | UP | UP | UP | 118 |
| CD38 | UP | UP | UP | UP | 119 |
| INPP4B | DOWN | DOWN | DOWN | DOWN | 120 |
| ID3 | DOWN | DOWN | DOWN | DOWN | 121 |
| DPM2 | DOWN | DOWN | UP | DOWN | 122 |
| CR1 | UP | UP | UP | UP | 123 |
| CR1 | UP | UP | UP | UP | 124 |
| TAPBP | DOWN | UP | UP | UP | 125 |
| PPAP2C | UP | UP | DOWN | UP | 126 |
| MBOAT2 | UP | UP | UP | UP | 127 |
| MS4A2 | DOWN | DOWN | UP | DOWN | 128 |
| FAM176B | UP | UP | UP | UP | 129 |
| LOC390183 | DOWN | DOWN | DOWN | DOWN | 130 |
| RPLP1 | DOWN | DOWN | DOWN | DOWN | 131 |
| SERPING1 | UP | UP | UP | UP | 132 |
| LOC441743 | DOWN | DOWN | DOWN | DOWN | 133 |
| H1F0 | UP | UP | UP | UP | 134 |
| SOD2 | UP | UP | DOWN | UP | 135 |
| LOC642828 | DOWN | DOWN | DOWN | DOWN | 136 |
| POLB | UP | UP | UP | UP | 137 |
| TSPAN9 | UP | UP | UP | UP | 138 |
| ORMDL3 | DOWN | DOWN | UP | DOWN | 139 |
| FER1L3 | UP | UP | UP | UP | 140 |
| LBH | DOWN | DOWN | DOWN | DOWN | 141 |
| PNKD | UP | UP | UP | UP | 142 |
| SLPI | UP | UP | DOWN | UP | 143 |
| SIRPB1 | UP | UP | UP | UP | 144 |
| LOC389386 | UP | UP | UP | UP | 145 |
| REC8 | UP | UP | UP | UP | 146 |
| GNLY | DOWN | DOWN | DOWN | DOWN | 147 |
| GNLY | DOWN | DOWN | DOWN | DOWN | 148 |
| FOLR3 | UP | UP | UP | UP | 149 |
| LOC730286 | UP | UP | UP | UP | 150 |
| SKAP1 | DOWN | DOWN | DOWN | DOWN | 151 |
| SELP | UP | UP | UP | UP | 152 |
| DHX30 | DOWN | DOWN | DOWN | DOWN | 153 |
| KIAA1618 | DOWN | UP | UP | UP | 154 |
| NQO2 | UP | UP | DOWN | UP | 155 |
| SF1 | UP | DOWN | DOWN | UP | 156 |
| ANKRD46 | DOWN | DOWN | DOWN | DOWN | 157 |
| LOC646301 | UP | UP | UP | UP | 158 |
| LOC400464 | DOWN | DOWN | DOWN | DOWN | 159 |
| LOC100134703 | UP | UP | UP | UP | 160 |
| C20ORF106 | UP | UP | UP | UP | 161 |
| ZNF683 | DOWN | DOWN | DOWN | DOWN | 162 |
| SLC25A38 | DOWN | DOWN | UP | DOWN | 163 |
| YPEL1 | DOWN | DOWN | DOWN | DOWN | 164 |
| IL1R1 | UP | UP | UP | UP | 165 |
| EPHAl | DOWN | DOWN | DOWN | DOWN | 166 |
| CHD6 | DOWN | DOWN | DOWN | DOWN | 167 |
| LIMK2 | UP | UP | UP | UP | 168 |
| LOC643733 | DOWN | DOWN | DOWN | DOWN | 169 |
| LOC441550 | DOWN | DOWN | DOWN | DOWN | 170 |
| MGC3020 | DOWN | DOWN | DOWN | DOWN | 171 |
| ANKRD9 | UP | UP | UP | UP | 172 |
| NOD2 | UP | UP | UP | UP | 173 |
| | DOWN | DOWN | DOWN | DOWN | 174 |
| MCTP1 | UP | UP | UP | UP | 175 |
| BANK1 | DOWN | DOWN | DOWN | DOWN | 176 |
| ZNF30 | DOWN | DOWN | DOWN | DOWN | 177 |
| CTTN | UP | UP | UP | UP | 178 |
| PTCRA | UP | UP | UP | UP | 179 |
| FBXO7 | DOWN | DOWN | UP | DOWN | 180 |
| FBXO7 | DOWN | DOWN | UP | DOWN | 181 |
| ABLIM1 | DOWN | DOWN | DOWN | DOWN | 182 |
| LAMP3 | DOWN | UP | UP | UP | 183 |
| CEBPE | UP | UP | UP | UP | 184 |
| LOC646909 | DOWN | DOWN | DOWN | DOWN | 185 |
| BCL11B | DOWN | DOWN | DOWN | DOWN | 186 |
| TRIM58 | DOWN | DOWN | UP | UP | 187 |
| SAMD3 | DOWN | DOWN | DOWN | DOWN | 188 |
| SAMD3 | DOWN | DOWN | DOWN | DOWN | 189 |
| MYOF | UP | UP | UP | UP | 190 |
| TTPAL | UP | UP | UP | DOWN | 191 |
| LOC642934 | DOWN | DOWN | DOWN | DOWN | 192 |
| | UP | UP | UP | UP | 193 |
| SNORA28 | UP | UP | UP | UP | 194 |
| FLJ32255 | UP | DOWN | UP | UP | 195 |
| | DOWN | DOWN | DOWN | DOWN | 196 |
| LOC642073 | DOWN | DOWN | UP | UP | 197 |
| CAMKK2 | UP | UP | UP | UP | 198 |
| OAS2 | UP | UP | UP | UP | 199 |
| RASGRP1 | DOWN | DOWN | DOWN | DOWN | 200 |
| CAPG | UP | UP | UP | UP | 201 |
| LOC648343 | DOWN | DOWN | DOWN | DOWN | 202 |
| CETP | UP | UP | UP | UP | 203 |
| CETP | UP | UP | UP | UP | 204 |
| CXCR7 | DOWN | DOWN | DOWN | DOWN | 205 |
| UBASH3A | DOWN | DOWN | DOWN | DOWN | 206 |
| LOC284648 | DOWN | UP | UP | UP | 207 |
| IL1R2 | UP | UP | UP | UP | 208 |
| AGK | DOWN | DOWN | DOWN | DOWN | 209 |
| GTPBP8 | DOWN | DOWN | DOWN | DOWN | 210 |
| LEF1 | DOWN | DOWN | DOWN | DOWN | 211 |
| LEF1 | DOWN | DOWN | DOWN | DOWN | 212 |
| GPR109A | UP | UP | UP | UP | 213 |
| IFI35 | UP | UP | UP | UP | 214 |
| IRF7 | UP | UP | UP | UP | 215 |
| IRF7 | UP | UP | UP | UP | 216 |
| SP4 | DOWN | DOWN | DOWN | DOWN | 217 |
| IL2RB | DOWN | DOWN | DOWN | DOWN | 218 |
| ABLIM1 | DOWN | DOWN | DOWN | DOWN | 219 |
| TAPBP | UP | UP | UP | UP | 220 |
| MAL | DOWN | DOWN | DOWN | DOWN | 221 |
| TCEA3 | DOWN | DOWN | DOWN | DOWN | 222 |
| KREMEN1 | UP | UP | UP | UP | 223 |
| KREMEN1 | UP | UP | UP | UP | 224 |
| VNN1 | UP | UP | UP | UP | 225 |
| GBP1 | DOWN | UP | UP | UP | 226 |
| GBP1 | DOWN | UP | UP | UP | 227 |
| UBE2C | UP | UP | UP | UP | 228 |
| DET1 | DOWN | DOWN | UP | DOWN | 229 |
| ANKRD36 | DOWN | DOWN | DOWN | DOWN | 230 |
| DEFA4 | UP | UP | UP | UP | 231 |
| GCH1 | UP | UP | UP | UP | 232 |
| IL7R | DOWN | DOWN | DOWN | DOWN | 233 |
| TMCO3 | UP | UP | DOWN | UP | 234 |
| FBXO6 | UP | UP | UP | UP | 235 |
| LACTB | UP | UP | UP | UP | 236 |
| LOC730953 | UP | UP | UP | UP | 237 |
| LOC285296 | UP | UP | UP | UP | 238 |
| IL18R1 | UP | UP | UP | UP | 239 |
| | UP | UP | UP | UP | 240 |
| PRR5 | DOWN | DOWN | UP | DOWN | 241 |
| LOC400061 | DOWN | DOWN | DOWN | DOWN | 242 |
| TSEN2 | DOWN | DOWN | DOWN | DOWN | 243 |
| MGC15763 | DOWN | DOWN | DOWN | DOWN | 244 |
| SH3YL1 | DOWN | DOWN | DOWN | DOWN | 245 |
| ZNF337 | DOWN | DOWN | DOWN | DOWN | 246 |
| AFF3 | DOWN | DOWN | DOWN | DOWN | 247 |
| TYMS | UP | UP | UP | UP | 248 |
| ZCCHC14 | DOWN | DOWN | DOWN | DOWN | 249 |
| SLC6A12 | UP | UP | UP | UP | 250 |
| LY6E | DOWN | UP | UP | UP | 251 |
| KLF12 | DOWN | DOWN | DOWN | DOWN | 252 |
| LOC100132317 | UP | UP | UP | UP | 253 |
| TYW3 | DOWN | DOWN | DOWN | DOWN | 254 |
| BTLA | DOWN | DOWN | DOWN | DOWN | 255 |
| SLC24A4 | UP | UP | UP | UP | 256 |
| | DOWN | DOWN | DOWN | DOWN | 257 |
| NCALD | DOWN | DOWN | DOWN | DOWN | 258 |
| ORAI2 | UP | UP | UP | UP | 259 |
| ITGB3BP | DOWN | DOWN | DOWN | DOWN | 260 |
| GYPE | UP | UP | UP | UP | 261 |
| DOCKS | UP | UP | UP | UP | 262 |
| RASGRP4 | UP | UP | UP | UP | 263 |
| LOC339290 | DOWN | DOWN | DOWN | DOWN | 264 |
| PRF1 | DOWN | DOWN | DOWN | DOWN | 265 |
| TGFBR3 | DOWN | DOWN | DOWN | DOWN | 266 |
| LGALS9 | UP | UP | UP | UP | 267 |
| LGALS9 | UP | UP | UP | UP | 268 |
| BATF2 | UP | UP | UP | UP | 269 |
| MGC57346 | DOWN | DOWN | DOWN | DOWN | 270 |
| TXK | DOWN | DOWN | DOWN | DOWN | 271 |
| DHX58 | UP | DOWN | UP | UP | 272 |
| EPB41L3 | UP | UP | UP | UP | 273 |
| LOC100132499 | UP | DOWN | DOWN | DOWN | 274 |
| LOC100129674 | UP | UP | UP | UP | 275 |
| GDPD5 | DOWN | DOWN | UP | UP | 276 |
| ACP2 | UP | UP | UP | UP | 277 |
| C3AR1 | UP | UP | UP | UP | 278 |
| APOB48R | UP | UP | UP | UP | 279 |
| UTRN | DOWN | DOWN | UP | DOWN | 280 |
| SLC2A14 | UP | UP | UP | UP | 281 |
| CLEC4D | UP | UP | UP | UP | 282 |
| PKM2 | UP | UP | UP | UP | 283 |
| CDCA5 | UP | UP | UP | UP | 284 |
| CACNA1E | UP | UP | UP | UP | 285 |
| OSBPL3 | DOWN | DOWN | DOWN | DOWN | 286 |
| SLC22A15 | UP | UP | UP | UP | 287 |
| VPREB3 | DOWN | DOWN | DOWN | DOWN | 288 |
| LOC642780 | UP | UP | UP | UP | 289 |
| MEGF6 | DOWN | DOWN | DOWN | DOWN | 290 |
| LOC93622 | DOWN | DOWN | DOWN | DOWN | 291 |
| PFAS | DOWN | DOWN | DOWN | DOWN | 292 |
| LOC729389 | DOWN | DOWN | DOWN | DOWN | 293 |
| CREBZF | UP | DOWN | DOWN | DOWN | 294 |
| IMPDH1 | UP | UP | UP | UP | 295 |
| DHRS3 | DOWN | DOWN | DOWN | DOWN | 296 |
| AXIN2 | DOWN | DOWN | DOWN | DOWN | 297 |
| DDX60L | UP | UP | UP | UP | 298 |
| | UP | UP | UP | UP | 299 |
| RPGRIP1 | UP | DOWN | UP | DOWN | 300 |
| GPR160 | UP | UP | UP | UP | 301 |
| TMTC1 | UP | UP | UP | UP | 302 |
| ABCA2 | UP | UP | DOWN | UP | 303 |
| CEACAM1 | UP | UP | UP | UP | 304 |
| CEACAM1 | UP | UP | UP | UP | 305 |
| FLJ42957 | UP | UP | UP | UP | 306 |
| SIAH2 | UP | UP | UP | UP | 307 |
| DDAH2 | UP | UP | UP | UP | 308 |
| C13ORF18 | UP | UP | DOWN | DOWN | 309 |
| TAGLN | UP | UP | UP | UP | 310 |
| LCN2 | UP | UP | UP | UP | 311 |
| RELB | UP | UP | UP | UP | 312 |
| NR1I2 | UP | UP | UP | UP | 313 |
| BEND7 | UP | UP | UP | UP | 314 |
| PIK3C2B | DOWN | DOWN | DOWN | DOWN | 315 |
| IFI6 | UP | UP | UP | UP | 316 |
| DUT | DOWN | DOWN | DOWN | DOWN | 317 |
| SETD6 | DOWN | DOWN | DOWN | DOWN | 318 |
| | DOWN | DOWN | DOWN | DOWN | 319 |
| LOC100131572 | DOWN | DOWN | DOWN | DOWN | 320 |
| TNRC6A | DOWN | DOWN | UP | DOWN | 321 |
| LOC399744 | UP | UP | UP | UP | 322 |
| MAPK13 | UP | UP | DOWN | UP | 323 |
| TAP2 | UP | UP | UP | UP | 324 |
| CCDC15 | DOWN | DOWN | UP | DOWN | 325 |
| TNCRNA | UP | UP | UP | UP | 326 |
| SIPA1L2 | UP | UP | UP | UP | 327 |
| HIST1H4E | DOWN | UP | UP | UP | 328 |
| PTPRE | UP | UP | UP | UP | 329 |
| ELANE | UP | UP | UP | UP | 330 |
| TGM2 | UP | UP | UP | UP | 331 |
| ARSD | UP | UP | UP | UP | 332 |
| LOC651451 | DOWN | DOWN | DOWN | DOWN | 333 |
| CYFIP1 | UP | UP | UP | UP | 334 |
| CYFIP1 | UP | UP | UP | UP | 335 |
| | UP | UP | UP | UP | 336 |
| PPIA | DOWN | DOWN | DOWN | DOWN | 337 |
| LOC642255 | UP | UP | DOWN | UP | 338 |
| ASCC2 | DOWN | DOWN | UP | DOWN | 339 |
| ZNF827 | DOWN | DOWN | DOWN | DOWN | 340 |
| STAB1 | UP | UP | UP | UP | 341 |
| DNASE1L1 | UP | UP | UP | UP | 342 |
| LMNB1 | UP | UP | UP | UP | 343 |
| MAP4K1 | DOWN | DOWN | DOWN | DOWN | 344 |
| PSMB9 | UP | UP | UP | UP | 345 |
| ATF3 | UP | UP | UP | UP | 346 |
| CPEB4 | UP | UP | UP | UP | 347 |
| ATP5S | DOWN | DOWN | UP | DOWN | 348 |
| CD5 | DOWN | DOWN | DOWN | DOWN | 349 |
| SYTL2 | DOWN | DOWN | DOWN | DOWN | 350 |
| H2AFJ | UP | UP | UP | UP | 351 |
| HP | UP | UP | UP | UP | 352 |
| SORT1 | UP | UP | UP | UP | 353 |
| KLHL18 | UP | UP | UP | UP | 354 |
| HIST1H2BK | UP | UP | UP | UP | 355 |
| HEMGN | DOWN | DOWN | UP | DOWN | 356 |
| KRTAP19-6 | UP | UP | UP | UP | 357 |
| RNASE2 | UP | UP | UP | UP | 358 |
| RAB13 | UP | UP | UP | UP | 359 |
| LOC100134393 | DOWN | DOWN | DOWN | DOWN | 360 |
| C11ORF82 | UP | UP | UP | UP | 361 |
| BLK | DOWN | DOWN | DOWN | DOWN | 362 |
| CD160 | DOWN | DOWN | DOWN | DOWN | 363 |
| NFIA | DOWN | DOWN | UP | UP | 364 |
| LOC100128460 | UP | UP | UP | UP | 365 |
| CD19 | DOWN | DOWN | DOWN | DOWN | 366 |
| ZNF438 | UP | UP | UP | UP | 367 |
| MBNL3 | DOWN | DOWN | UP | DOWN | 368 |
| MBNL3 | DOWN | DOWN | UP | DOWN | 369 |
| | UP | UP | UP | UP | 370 |
| LOC729010 | UP | UP | UP | UP | 371 |
| NAGA | UP | UP | UP | UP | 372 |
| FCER1A | DOWN | DOWN | DOWN | DOWN | 373 |
| C6ORF25 | UP | UP | UP | UP | 374 |
| SLC22A4 | UP | UP | UP | UP | 375 |
| LOC729686 | DOWN | DOWN | DOWN | DOWN | 376 |
| LOC728843 | DOWN | DOWN | DOWN | DOWN | 377 |
| CTSL1 | DOWN | UP | UP | UP | 378 |
| BCL11A | DOWN | DOWN | DOWN | DOWN | 379 |
| ACTA2 | UP | UP | UP | UP | 380 |
| KIAA1632 | UP | UP | UP | UP | 381 |
| UBE2C | UP | UP | UP | UP | 382 |
| CASP4 | UP | UP | UP | UP | 383 |
| SLC22A4 | UP | UP | UP | UP | 384 |
| SFT2D2 | UP | UP | UP | UP | 385 |
| TLR2 | UP | UP | UP | UP | 386 |
| C10ORF105 | UP | UP | UP | UP | 387 |
| EIF2AK2 | UP | UP | UP | UP | 388 |
| TATDN1 | DOWN | DOWN | DOWN | DOWN | 389 |
| RAB24 | UP | UP | UP | UP | 390 |
| FAH | UP | UP | UP | UP | 391 |
| | DOWN | DOWN | DOWN | DOWN | 392 |
| DISC1 | UP | UP | UP | UP | 393 |
| LOC641848 | DOWN | DOWN | DOWN | DOWN | 394 |
| ARG1 | UP | UP | UP | UP | 395 |
| LCK | DOWN | DOWN | DOWN | DOWN | 396 |
| WDFY3 | UP | UP | UP | UP | 397 |
| RNF165 | DOWN | DOWN | DOWN | DOWN | 398 |
| MLKL | UP | UP | UP | UP | 399 |
| LOC100132673 | DOWN | DOWN | DOWN | DOWN | 400 |
| ANKDD1A | UP | UP | UP | UP | 401 |
| MSRB3 | UP | UP | UP | UP | 402 |
| LOC100134379 | UP | UP | UP | UP | 403 |
| MEFV | UP | UP | UP | UP | 404 |
| C12ORF57 | DOWN | DOWN | DOWN | DOWN | 405 |
| CCDC102A | DOWN | DOWN | DOWN | DOWN | 406 |
| LOC731777 | DOWN | DOWN | UP | DOWN | 407 |
| LOC729040 | UP | UP | UP | UP | 408 |
| TBC1D8 | UP | UP | UP | UP | 409 |
| KLRF1 | DOWN | DOWN | DOWN | DOWN | 410 |
| KLRF1 | DOWN | DOWN | DOWN | DOWN | 411 |
| ABCA1 | UP | UP | UP | UP | 412 |
| LOC650761 | DOWN | DOWN | DOWN | DOWN | 413 |
| LOC653867 | UP | UP | DOWN | UP | 414 |
| LOC648710 | UP | UP | UP | UP | 415 |
| SLC2A11 | UP | UP | UP | UP | 416 |
| LOC652578 | UP | UP | UP | UP | 417 |
| GPR114 | DOWN | DOWN | UP | DOWN | 418 |
| MANSC1 | UP | UP | DOWN | UP | 419 |
| MANSC1 | UP | UP | DOWN | UP | 420 |
| DGKA | DOWN | DOWN | DOWN | DOWN | 421 |
| LIN7A | UP | UP | UP | UP | 422 |
| ITPRIPL2 | UP | UP | UP | UP | 423 |
| ANO9 | DOWN | DOWN | DOWN | DOWN | 424 |
| KCNJ15 | UP | UP | UP | UP | 425 |
| KCNJ15 | UP | UP | UP | UP | 426 |
| LOC389386 | UP | UP | UP | UP | 427 |
| LOC100132960 | UP | UP | UP | UP | 428 |
| LOC643332 | UP | UP | UP | UP | 429 |
| SFI1 | DOWN | DOWN | DOWN | DOWN | 430 |
| ABCE1 | DOWN | DOWN | DOWN | DOWN | 431 |
| ABCE1 | DOWN | DOWN | DOWN | DOWN | 432 |
| SERPINA1 | UP | UP | UP | UP | 433 |
| OR2W3 | DOWN | DOWN | UP | DOWN | 434 |
| ABI3 | DOWN | DOWN | UP | DOWN | 435 |
| LOC400759 | UP | UP | UP | UP | 436 |
| | UP | UP | DOWN | UP | 437 |
| LOC728519 | UP | UP | UP | UP | 438 |
| LOC654053 | UP | UP | UP | UP | 439 |
| LOC649553 | DOWN | DOWN | DOWN | DOWN | 440 |
| | UP | UP | UP | UP | 441 |
| HSD17B8 | DOWN | DOWN | DOWN | DOWN | 442 |
| C16ORF30 | DOWN | DOWN | DOWN | DOWN | 443 |
| GADD45G | UP | UP | UP | UP | 444 |
| TPST1 | UP | UP | UP | UP | 445 |
| GNG7 | DOWN | DOWN | DOWN | DOWN | 446 |
| SV2A | UP | UP | UP | UP | 447 |
| LOC649946 | DOWN | DOWN | DOWN | DOWN | 448 |
| LOC100129697 | UP | UP | UP | UP | 449 |
| | DOWN | DOWN | DOWN | DOWN | 450 |
| RARRES3 | DOWN | DOWN | UP | UP | 451 |
| C8ORF83 | UP | UP | UP | UP | 452 |
| TNFSF13B | UP | UP | UP | UP | 453 |
| | DOWN | DOWN | DOWN | DOWN | 454 |
| SNRPD3 | UP | DOWN | DOWN | DOWN | 455 |
| LOC645232 | UP | UP | UP | UP | 456 |
| PI3 | UP | UP | UP | DOWN | 457 |
| WDFY1 | UP | UP | UP | UP | 458 |
| LOC100134660 | UP | UP | UP | UP | 459 |
| LOC100133678 | DOWN | DOWN | UP | UP | 460 |
| BAMBI | UP | UP | UP | UP | 461 |
| POP5 | DOWN | DOWN | DOWN | DOWN | 462 |
| TARBP1 | DOWN | DOWN | DOWN | DOWN | 463 |
| IRAK3 | UP | UP | UP | UP | 464 |
| ZNF7 | DOWN | DOWN | DOWN | DOWN | 465 |
| NLRC4 | UP | UP | UP | UP | 466 |
| SKAP1 | DOWN | DOWN | DOWN | DOWN | 467 |
| GAS7 | UP | UP | UP | UP | 468 |
| C12ORF29 | DOWN | DOWN | DOWN | DOWN | 469 |
| KLRD1 | DOWN | DOWN | DOWN | DOWN | 470 |
| | DOWN | DOWN | DOWN | DOWN | 471 |
| ABHD15 | DOWN | DOWN | DOWN | DOWN | 472 |
| CCDC146 | UP | DOWN | UP | UP | 473 |
| CASP5 | UP | UP | UP | UP | 474 |
| AARS2 | DOWN | DOWN | DOWN | DOWN | 475 |
| LOC642103 | UP | UP | UP | UP | 476 |
| LOC730385 | UP | UP | UP | UP | 477 |
| GAR1 | DOWN | DOWN | DOWN | DOWN | 478 |
| MAF | DOWN | DOWN | DOWN | DOWN | 479 |
| ARAP2 | UP | UP | UP | UP | 480 |
| C16ORF7 | UP | UP | UP | UP | 481 |
| HLA-C | UP | DOWN | DOWN | UP | 482 |
| FLJ22662 | UP | UP | UP | UP | 483 |
| DACH1 | UP | UP | UP | UP | 484 |
| CRY1 | DOWN | DOWN | DOWN | DOWN | 485 |
| CRY1 | DOWN | DOWN | DOWN | DOWN | 486 |
| LRRC25 | UP | UP | UP | UP | 487 |
| KIAA0564 | DOWN | DOWN | DOWN | DOWN | 488 |
| UPF3A | DOWN | DOWN | DOWN | DOWN | 489 |
| MARCO | UP | UP | UP | UP | 490 |
| LOC100132564 | UP | UP | DOWN | UP | 491 |
| SRPRB | DOWN | DOWN | DOWN | DOWN | 492 |
| MAD1L1 | DOWN | DOWN | DOWN | DOWN | 493 |
| LOC653610 | UP | UP | UP | UP | 494 |
| P4HTM | DOWN | DOWN | DOWN | DOWN | 495 |
| CCL4L1 | DOWN | DOWN | DOWN | DOWN | 496 |
| LAPTM4B | UP | UP | DOWN | UP | 497 |
| MAPK14 | UP | UP | UP | UP | 498 |
| CD96 | DOWN | DOWN | DOWN | DOWN | 499 |
| TLR7 | UP | UP | UP | UP | 500 |
| KCNMB1 | UP | UP | UP | UP | 501 |
| HIP1 | UP | UP | UP | UP | 502 |
| P2RX7 | UP | UP | UP | UP | 503 |
| LOC650140 | UP | UP | UP | UP | 504 |
| LOC791120 | DOWN | DOWN | DOWN | DOWN | 505 |
| LTF | UP | UP | UP | UP | 506 |
| C3ORF75 | DOWN | DOWN | DOWN | DOWN | 507 |
| GPX7 | DOWN | DOWN | DOWN | DOWN | 508 |
| SPRYD5 | DOWN | DOWN | UP | DOWN | 509 |
| MOV10 | DOWN | UP | UP | UP | 510 |
| EEF1B2 | DOWN | DOWN | DOWN | DOWN | 511 |
| CTDSPL | UP | UP | UP | UP | 512 |
| HIST2H2BE | UP | UP | UP | UP | 513 |
| SLC38A1 | DOWN | DOWN | DOWN | DOWN | 514 |
| AIM2 | UP | UP | UP | UP | 515 |
| LOC100130904 | UP | UP | DOWN | UP | 516 |
| LOC650546 | UP | UP | UP | UP | 517 |
| P2RY10 | DOWN | DOWN | DOWN | DOWN | 518 |
| IL5RA | DOWN | DOWN | UP | DOWN | 519 |
| MMP8 | UP | UP | UP | UP | 520 |
| LOC100128485 | UP | UP | UP | UP | 521 |
| RPS23 | DOWN | DOWN | DOWN | DOWN | 522 |
| HDAC7 | UP | UP | UP | UP | 523 |
| GUCY1A3 | UP | UP | UP | UP | 524 |
| TGFA | UP | UP | UP | UP | 525 |
| NAIP | UP | UP | UP | UP | 526 |
| NAIP | UP | UP | UP | UP | 527 |
| NELL2 | DOWN | DOWN | DOWN | DOWN | 528 |
| SIDT1 | DOWN | DOWN | DOWN | DOWN | 529 |
| SLAMF1 | DOWN | DOWN | DOWN | DOWN | 530 |
| MAPK14 | UP | UP | UP | UP | 531 |
| CCR3 | DOWN | DOWN | UP | DOWN | 532 |
| MKNK1 | UP | UP | UP | UP | 533 |
| D4S234E | DOWN | DOWN | DOWN | DOWN | 534 |
| | DOWN | DOWN | DOWN | DOWN | 535 |
| NBN | UP | UP | UP | UP | 536 |
| LOC654346 | DOWN | UP | UP | UP | 537 |
| FGFBP2 | DOWN | DOWN | DOWN | DOWN | 538 |
| BTLA | DOWN | DOWN | DOWN | DOWN | 539 |
| PRMT1 | DOWN | DOWN | DOWN | DOWN | 540 |
| PDGFC | UP | UP | UP | UP | 541 |
| LRRN3 | DOWN | DOWN | DOWN | DOWN | 542 |
| MT2A | DOWN | DOWN | UP | UP | 543 |
| LOC728790 | UP | UP | UP | UP | 544 |
| LOC646672 | DOWN | DOWN | DOWN | DOWN | 545 |
| NTN3 | UP | UP | UP | UP | 546 |
| CD8A | DOWN | DOWN | DOWN | DOWN | 547 |
| CD8A | DOWN | DOWN | DOWN | DOWN | 548 |
| ZBP1 | UP | UP | UP | UP | 549 |
| LDOC1L | DOWN | DOWN | DOWN | DOWN | 550 |
| CHM | DOWN | DOWN | DOWN | DOWN | 551 |
| LOC440731 | UP | UP | UP | UP | 552 |
| LOC100131787 | DOWN | DOWN | DOWN | DOWN | 553 |
| TNFRSF10C | UP | UP | UP | UP | 554 |
| LOC651612 | UP | UP | DOWN | UP | 555 |
| STX11 | UP | UP | UP | UP | 556 |
| LOC100128060 | DOWN | DOWN | DOWN | DOWN | 557 |
| C1QB | UP | UP | UP | UP | 558 |
| PVRL2 | UP | UP | UP | UP | 559 |
| ZMYND15 | UP | UP | UP | UP | 560 |
| TRAPPC2P1 | DOWN | DOWN | DOWN | DOWN | 561 |
| SECTM1 | UP | UP | UP | UP | 562 |
| TRAT1 | DOWN | DOWN | DOWN | DOWN | 563 |
| CAMKK2 | UP | UP | UP | UP | 564 |
| CXCR5 | DOWN | DOWN | DOWN | DOWN | 565 |
| CD163 | UP | UP | UP | UP | 566 |
| FAS | UP | UP | UP | UP | 567 |
| RPL12P6 | DOWN | DOWN | DOWN | DOWN | 568 |
| LOC100134734 | UP | UP | UP | UP | 569 |
| CD36 | UP | UP | UP | UP | 570 |
| FCGR1B | UP | UP | UP | UP | 571 |
| NR3C2 | DOWN | DOWN | DOWN | DOWN | 572 |
| CSGALNACT2 | UP | UP | UP | UP | 573 |
| NCRNA00085 | UP | UP | UP | UP | 574 |
| GATA2 | DOWN | DOWN | UP | DOWN | 575 |
| EBI2 | DOWN | DOWN | DOWN | DOWN | 576 |
| EBI2 | DOWN | DOWN | DOWN | DOWN | 577 |
| FKBP5 | UP | UP | UP | UP | 578 |
| CRISPLD2 | UP | UP | UP | UP | 579 |
| LOC152195 | UP | UP | UP | UP | 580 |
| LOC100132199 | DOWN | DOWN | DOWN | DOWN | 581 |
| DGAT2 | UP | UP | UP | UP | 582 |
| SCML1 | DOWN | DOWN | DOWN | DOWN | 583 |
| LSS | DOWN | DOWN | DOWN | DOWN | 584 |
| CIITA | DOWN | DOWN | UP | UP | 585 |
| SAP30 | UP | UP | UP | UP | 586 |
| TLR5 | UP | UP | UP | UP | 587 |
| NFATC3 | DOWN | DOWN | DOWN | DOWN | 588 |
| NAMPT | UP | UP | UP | UP | 589 |
| GZMK | DOWN | DOWN | DOWN | DOWN | 590 |
| CARD17 | UP | UP | UP | UP | 591 |
| INCA | UP | UP | UP | UP | 592 |
| MSL3L1 | UP | UP | UP | UP | 593 |
| CD8A | DOWN | DOWN | DOWN | DOWN | 594 |
| MIIP | UP | UP | UP | UP | 595 |
| SRPK1 | UP | UP | UP | UP | 596 |
| SLC6A6 | UP | UP | UP | UP | 597 |
| C10ORF119 | UP | UP | UP | UP | 598 |
| C17ORF60 | UP | UP | UP | UP | 599 |
| LOC642816 | UP | UP | UP | UP | 600 |
| AKR1C3 | DOWN | DOWN | DOWN | DOWN | 601 |
| LHFPL2 | UP | UP | UP | UP | 602 |
| CR1 | UP | UP | UP | UP | 603 |
| KIAA1026 | UP | UP | UP | UP | 604 |
| CCDC91 | DOWN | DOWN | DOWN | DOWN | 605 |
| FAM102A | DOWN | DOWN | DOWN | DOWN | 606 |
| FAM102A | DOWN | DOWN | DOWN | DOWN | 607 |
| UPRT | DOWN | DOWN | DOWN | DOWN | 608 |
| | UP | UP | DOWN | UP | 609 |
| PLEKHA1 | DOWN | DOWN | DOWN | DOWN | 610 |
| GIMAP7 | DOWN | DOWN | DOWN | DOWN | 611 |
| CACNA2D3 | DOWN | DOWN | DOWN | DOWN | 612 |
| DDX10 | DOWN | DOWN | DOWN | DOWN | 613 |
| RPL23A | DOWN | DOWN | DOWN | DOWN | 614 |
| C2ORF44 | DOWN | DOWN | DOWN | DOWN | 615 |
| LSP1 | UP | UP | UP | UP | 616 |
| C7ORF53 | UP | UP | UP | UP | 617 |
| LOC100130905 | DOWN | DOWN | UP | DOWN | 618 |
| DNAJC5 | UP | UP | UP | UP | 619 |
| SLAIN1 | DOWN | DOWN | DOWN | DOWN | 620 |
| CDKN1C | DOWN | DOWN | UP | UP | 621 |
| AKAP7 | DOWN | DOWN | DOWN | DOWN | 622 |
| HIATL1 | UP | UP | UP | UP | 623 |
| CRELD1 | DOWN | DOWN | DOWN | DOWN | 624 |
| ZNHIT6 | DOWN | DOWN | DOWN | DOWN | 625 |
| TIFA | DOWN | UP | UP | UP | 626 |
| ARL4C | DOWN | DOWN | DOWN | DOWN | 627 |
| PIGU | DOWN | DOWN | DOWN | DOWN | 628 |
| MEF2A | UP | UP | UP | UP | 629 |
| PIK3CB | UP | UP | UP | UP | 630 |
| CDK5RAP2 | UP | UP | UP | UP | 631 |
| FLNB | DOWN | DOWN | DOWN | DOWN | 632 |
| GRAP | DOWN | DOWN | DOWN | DOWN | 633 |
| TLE3 | UP | UP | UP | UP | 634 |
| BATF | UP | UP | UP | UP | 635 |
| CYP4F3 | UP | UP | UP | UP | 636 |
| | DOWN | DOWN | DOWN | DOWN | 637 |
| KIR2DL3 | DOWN | DOWN | DOWN | DOWN | 638 |
| C19ORF59 | UP | UP | UP | UP | 639 |
| NRG1 | UP | UP | UP | UP | 640 |
| PPP2R2B | DOWN | DOWN | DOWN | DOWN | 641 |
| CDK5RAP2 | UP | UP | UP | UP | 642 |
| PLSCR1 | UP | UP | UP | UP | 643 |
| UBL7 | DOWN | DOWN | UP | DOWN | 644 |
| HES4 | DOWN | DOWN | UP | UP | 645 |
| ZNF256 | DOWN | DOWN | DOWN | DOWN | 646 |
| DKFZP761E198 | UP | UP | UP | UP | 647 |
| SAMD14 | UP | UP | UP | UP | 648 |
| BAG3 | DOWN | DOWN | DOWN | DOWN | 649 |
| PARP14 | UP | UP | UP | UP | 650 |
| MS4A7 | UP | DOWN | UP | UP | 651 |
| ECHDC3 | UP | UP | UP | UP | 652 |
| OCIAD2 | DOWN | DOWN | DOWN | DOWN | 653 |
| LOC90925 | DOWN | DOWN | DOWN | DOWN | 654 |
| RGL4 | UP | UP | DOWN | UP | 655 |
| PARP9 | UP | UP | UP | UP | 656 |
| PARP9 | UP | UP | UP | UP | 657 |
| CD151 | UP | UP | UP | UP | 658 |
| SAAL1 | DOWN | DOWN | DOWN | DOWN | 659 |
| LOC388076 | DOWN | DOWN | DOWN | DOWN | 660 |
| SIGLEC5 | UP | UP | UP | UP | 661 |
| LRIG1 | DOWN | DOWN | DOWN | DOWN | 662 |
| PTGDR | DOWN | DOWN | DOWN | DOWN | 663 |
| PTGDR | DOWN | DOWN | DOWN | DOWN | 664 |
| NBPF8 | UP | UP | DOWN | DOWN | 665 |
| NHS | UP | DOWN | DOWN | DOWN | 666 |
| ACSL1 | UP | UP | UP | UP | 667 |
| HK3 | UP | UP | UP | UP | 668 |
| SNX20 | UP | UP | UP | UP | 669 |
| F2RL1 | UP | UP | UP | UP | 670 |
| F2RL1 | UP | UP | UP | UP | 671 |
| PARP12 | DOWN | DOWN | UP | UP | 672 |
| LOC441506 | DOWN | DOWN | DOWN | DOWN | 673 |
| MFGE8 | DOWN | DOWN | DOWN | DOWN | 674 |
| SERPINA10 | DOWN | DOWN | DOWN | DOWN | 675 |
| FAM69A | DOWN | DOWN | DOWN | DOWN | 676 |
| IL4R | UP | UP | DOWN | UP | 677 |
| KIAA1671 | DOWN | DOWN | DOWN | DOWN | 678 |
| OAS3 | DOWN | UP | UP | UP | 679 |
| PRR5 | DOWN | DOWN | UP | DOWN | 680 |
| TMEM194 | DOWN | DOWN | DOWN | DOWN | 681 |
| MS4A1 | DOWN | DOWN | DOWN | DOWN | 682 |
| NRSN2 | UP | UP | UP | UP | 683 |
| MTHFD2 | UP | UP | UP | UP | 684 |
| LOC400793 | UP | UP | DOWN | UP | 685 |
| CEACAM1 | UP | UP | UP | UP | 686 |
| RPL37 | DOWN | DOWN | DOWN | DOWN | 687 |
| APP | UP | UP | DOWN | DOWN | 688 |
| RRBP1 | UP | UP | UP | UP | 689 |
| SLCO4C1 | UP | UP | DOWN | DOWN | 690 |
| XAF1 | DOWN | DOWN | UP | UP | 691 |
| XAF1 | DOWN | UP | UP | UP | 692 |
| SLC2A6 | DOWN | UP | UP | UP | 693 |
| ZNF831 | DOWN | DOWN | DOWN | DOWN | 694 |
| ZNF831 | DOWN | DOWN | DOWN | DOWN | 695 |
| POLR1C | DOWN | DOWN | DOWN | DOWN | 696 |
| GLT1D1 | UP | UP | UP | UP | 697 |
| VDR | UP | UP | UP | UP | 698 |
| IFIT5 | UP | UP | UP | UP | 699 |
| CSTA | UP | UP | UP | UP | 700 |
| SNHG8 | DOWN | DOWN | DOWN | DOWN | 701 |
| TOP1MT | DOWN | DOWN | DOWN | DOWN | 702 |
| UPP1 | UP | UP | UP | UP | 703 |
| SYTL2 | DOWN | DOWN | DOWN | DOWN | 704 |
| LOC440359 | DOWN | DOWN | UP | UP | 705 |
| KLRB1 | DOWN | DOWN | DOWN | DOWN | 706 |
| MTMR3 | UP | UP | UP | UP | 707 |
| S1PR1 | DOWN | DOWN | DOWN | DOWN | 708 |
| FYB | UP | UP | UP | UP | 709 |
| CDC20 | UP | UP | UP | UP | 710 |
| MEX3C | DOWN | DOWN | DOWN | DOWN | 711 |
| FAM168B | DOWN | DOWN | DOWN | DOWN | 712 |
| C20ORF107 | UP | UP | UP | UP | 713 |
| SLC4A7 | DOWN | DOWN | DOWN | DOWN | 714 |
| CD79B | DOWN | DOWN | DOWN | DOWN | 715 |
| FAM84B | DOWN | DOWN | DOWN | DOWN | 716 |
| LOC100134688 | UP | UP | UP | UP | 717 |
| LOC651738 | UP | UP | UP | UP | 718 |
| PLAGL1 | UP | UP | UP | UP | 719 |
| TIMM10 | DOWN | UP | UP | UP | 720 |
| LOC641710 | UP | UP | UP | UP | 721 |
| TRAF5 | DOWN | DOWN | DOWN | DOWN | 722 |
| TAP1 | UP | UP | UP | UP | 723 |
| FCRL2 | DOWN | DOWN | DOWN | DOWN | 724 |
| SRC | UP | UP | UP | UP | 725 |
| RALGAPA1 | DOWN | DOWN | DOWN | DOWN | 726 |
| OCIAD2 | DOWN | DOWN | DOWN | DOWN | 727 |
| PON2 | DOWN | DOWN | DOWN | DOWN | 728 |
| LOC730029 | DOWN | DOWN | DOWN | DOWN | 729 |
| LOC100134768 | UP | UP | UP | UP | 730 |
| LOC100134241 | DOWN | DOWN | DOWN | DOWN | 731 |
| LOC26010 | DOWN | DOWN | UP | UP | 732 |
| PLA2G12A | UP | UP | DOWN | UP | 733 |
| BACH1 | UP | UP | UP | UP | 734 |
| DSC1 | DOWN | DOWN | DOWN | DOWN | 735 |
| NOB1 | UP | DOWN | DOWN | DOWN | 736 |
| LOC645693 | DOWN | DOWN | DOWN | DOWN | 737 |
| LOC643313 | UP | UP | DOWN | UP | 738 |
| BTBD11 | DOWN | DOWN | DOWN | DOWN | 739 |
| TMEM169 | UP | UP | UP | UP | 740 |
| REPS2 | UP | UP | UP | UP | 741 |
| ZNF23 | DOWN | DOWN | DOWN | DOWN | 742 |
| C18ORF55 | DOWN | DOWN | DOWN | DOWN | 743 |
| APOL2 | UP | UP | UP | UP | 744 |
| APOL2 | UP | UP | UP | UP | 745 |
| PASK | DOWN | DOWN | DOWN | DOWN | 746 |
| FER1L3 | UP | UP | UP | UP | 747 |
| U2AF1 | UP | UP | DOWN | DOWN | 748 |
| LOC285359 | DOWN | DOWN | DOWN | DOWN | 749 |
| SIGLEC14 | UP | UP | UP | DOWN | 750 |
| ARL1 | DOWN | DOWN | DOWN | DOWN | 751 |
| C19ORF62 | DOWN | DOWN | UP | DOWN | 752 |
| NCR3 | DOWN | DOWN | DOWN | DOWN | 753 |
| | UP | UP | UP | UP | 754 |
| HOXB2 | DOWN | DOWN | DOWN | DOWN | 755 |
| RNF135 | UP | UP | UP | UP | 756 |
| IFIT1 | UP | UP | UP | UP | 757 |
| GCAT | UP | DOWN | UP | UP | 758 |
| KLF12 | DOWN | DOWN | DOWN | DOWN | 759 |
| LILRB2 | DOWN | UP | UP | UP | 760 |
| LOC728835 | DOWN | DOWN | DOWN | DOWN | 761 |
| GSN | UP | UP | UP | UP | 762 |
| LOC100008589 | UP | DOWN | DOWN | UP | 763 |
| LOC100008589 | UP | UP | DOWN | UP | 764 |
| FLJ14213 | DOWN | DOWN | UP | UP | 765 |
| SH2D3C | UP | UP | UP | UP | 766 |
| LOC100133177 | UP | UP | UP | UP | 767 |
| TMEM176A | UP | UP | UP | UP | 768 |
| HIST2H2AB | UP | UP | UP | UP | 769 |
| KIAA1618 | UP | UP | UP | UP | 770 |
| CMTM5 | UP | UP | UP | UP | 771 |
| C21ORF2 | DOWN | DOWN | DOWN | DOWN | 772 |
| CREB5 | UP | UP | UP | UP | 773 |
| FAS | UP | UP | UP | UP | 774 |
| MTF1 | UP | UP | UP | UP | 775 |
| RSAD2 | UP | UP | UP | UP | 776 |
| ANPEP | UP | UP | UP | UP | 777 |
| C14ORF179 | DOWN | DOWN | DOWN | DOWN | 778 |
| TXNL4B | UP | UP | UP | UP | 779 |
| MYL9 | UP | UP | UP | UP | 780 |
| MYL9 | UP | UP | UP | UP | 781 |
| LOC100130828 | UP | UP | UP | UP | 782 |
| LOC391019 | DOWN | DOWN | DOWN | DOWN | 783 |
| ITGA2B | UP | UP | UP | UP | 784 |
| KLRC3 | DOWN | DOWN | DOWN | DOWN | 785 |
| RASGRP2 | DOWN | DOWN | DOWN | DOWN | 786 |
| NDST1 | UP | UP | UP | UP | 787 |
| LOC388344 | DOWN | DOWN | DOWN | DOWN | 788 |
| IFI6 | DOWN | UP | UP | UP | 789 |
| OAS1 | UP | UP | UP | UP | 790 |
| OAS1 | UP | UP | UP | UP | 791 |
| TRIM10 | DOWN | DOWN | UP | DOWN | 792 |
| LIMK2 | UP | UP | UP | UP | 793 |
| LIMK2 | UP | UP | UP | UP | 794 |
| ATP5S | DOWN | DOWN | DOWN | DOWN | 795 |
| SMARCD3 | UP | UP | UP | UP | 796 |
| PHC2 | UP | UP | UP | UP | 797 |
| SOX8 | DOWN | DOWN | DOWN | DOWN | 798 |
| LCK | DOWN | DOWN | DOWN | DOWN | 799 |
| | DOWN | DOWN | DOWN | DOWN | 800 |
| SAMD9L | UP | UP | UP | UP | 801 |
| EHBP1 | DOWN | DOWN | DOWN | DOWN | 802 |
| E2F2 | DOWN | DOWN | UP | DOWN | 803 |
| CEACAM6 | UP | UP | UP | UP | 804 |
| LOC100132394 | UP | DOWN | DOWN | UP | 805 |
| LOC728014 | DOWN | DOWN | DOWN | DOWN | 806 |
| LOC728014 | DOWN | DOWN | DOWN | DOWN | 807 |
| SIRPG | DOWN | DOWN | DOWN | DOWN | 808 |
| OPLAH | UP | UP | UP | UP | 809 |
| FTHL2 | UP | UP | UP | UP | 810 |
| CXORF21 | UP | UP | UP | UP | 811 |
| CACNG6 | DOWN | DOWN | UP | DOWN | 812 |
| C11ORF75 | UP | UP | UP | UP | 813 |
| LY9 | DOWN | DOWN | DOWN | DOWN | 814 |
| LILRB4 | UP | UP | UP | UP | 815 |
| STAT2 | UP | UP | UP | UP | 816 |
| RAB20 | UP | UP | UP | UP | 817 |
| SOCS1 | DOWN | UP | UP | UP | 818 |
| PLOD2 | UP | UP | UP | UP | 819 |
| UGDH | DOWN | DOWN | DOWN | DOWN | 820 |
| MAK16 | DOWN | DOWN | DOWN | DOWN | 821 |
| ITGB3 | UP | UP | UP | UP | 822 |
| DHRS9 | UP | UP | UP | UP | 823 |
| PLEKHF1 | DOWN | DOWN | DOWN | DOWN | 824 |
| ASAP1IT1 | UP | UP | UP | UP | 825 |
| PSME2 | DOWN | UP | UP | UP | 826 |
| | UP | UP | UP | UP | 827 |
| LOC100128269 | UP | UP | DOWN | UP | 828 |
| ALX1 | UP | UP | UP | UP | 829 |
| BAK1 | DOWN | UP | UP | UP | 830 |
| XPO4 | DOWN | DOWN | DOWN | DOWN | 831 |
| CD247 | DOWN | DOWN | DOWN | DOWN | 832 |
| C3ORF26 | DOWN | DOWN | DOWN | DOWN | 833 |
| FAM43A | DOWN | DOWN | DOWN | DOWN | 834 |
| ICOS | DOWN | DOWN | DOWN | DOWN | 835 |
| ISG15 | UP | UP | UP | UP | 836 |
| | UP | UP | UP | UP | 837 |
| HIST2H2AA4 | UP | UP | UP | UP | 838 |
| CD79A | DOWN | DOWN | DOWN | DOWN | 839 |
| SLC25A4 | DOWN | DOWN | DOWN | DOWN | 840 |
| TMEM158 | UP | UP | UP | UP | 841 |
| FANCD2 | DOWN | DOWN | DOWN | DOWN | 842 |
| GPR18 | DOWN | DOWN | DOWN | DOWN | 843 |
| LAP3 | UP | UP | UP | UP | 844 |
| TNFSF13B | UP | UP | UP | UP | 845 |
| TC2N | DOWN | DOWN | DOWN | DOWN | 846 |
| HSF2 | DOWN | DOWN | DOWN | DOWN | 847 |
| CD7 | DOWN | DOWN | DOWN | DOWN | 848 |
| C20ORF3 | UP | UP | UP | UP | 849 |
| HLA-DRB3 | DOWN | DOWN | UP | UP | 850 |
| SESN1 | DOWN | DOWN | DOWN | DOWN | 851 |
| LOC347376 | UP | UP | UP | UP | 852 |
| P2RY14 | DOWN | UP | UP | UP | 853 |
| P2RY14 | UP | UP | UP | UP | 854 |
| P2RY14 | DOWN | UP | UP | UP | 855 |
| CYP1B1 | UP | UP | DOWN | UP | 856 |
| IFIT3 | DOWN | UP | UP | UP | 857 |
| IFIT3 | UP | UP | UP | UP | 858 |
| RPL13L | DOWN | DOWN | DOWN | DOWN | 859 |
| LOC729423 | DOWN | DOWN | DOWN | DOWN | 860 |
| DBN1 | UP | UP | UP | UP | 861 |
| TTC27 | DOWN | DOWN | DOWN | DOWN | 862 |
| DPH5 | DOWN | DOWN | DOWN | DOWN | 863 |
| GPR141 | UP | UP | UP | UP | 864 |
| RBBP8 | UP | UP | UP | UP | 865 |
| LOC654350 | DOWN | DOWN | DOWN | DOWN | 866 |
| SLC30A1 | UP | UP | UP | UP | 867 |
| PRSS23 | DOWN | DOWN | DOWN | DOWN | 868 |
| JAM3 | UP | UP | UP | UP | 869 |
| GNPDA2 | DOWN | DOWN | DOWN | DOWN | 870 |
| IL7R | DOWN | DOWN | DOWN | DOWN | 871 |
| ACAD11 | DOWN | DOWN | DOWN | DOWN | 872 |
| LOC642788 | UP | UP | UP | UP | 873 |
| ALPK1 | UP | UP | UP | UP | 874 |
| LOC439949 | DOWN | DOWN | DOWN | DOWN | 875 |
| | UP | UP | UP | UP | 876 |
| BCAT1 | UP | UP | UP | UP | 877 |
| C9ORF114 | DOWN | DOWN | DOWN | DOWN | 878 |
| ATPGD1 | DOWN | DOWN | DOWN | DOWN | 879 |
| TREML1 | UP | UP | UP | UP | 880 |
| PECR | UP | UP | DOWN | DOWN | 881 |
| SPATA13 | UP | DOWN | DOWN | UP | 882 |
| MAN1C1 | DOWN | DOWN | DOWN | DOWN | 883 |
| IDO1 | DOWN | DOWN | UP | UP | 884 |
| TSEN54 | DOWN | DOWN | DOWN | DOWN | 885 |
| SCRN1 | DOWN | DOWN | UP | DOWN | 886 |
| LOC441193 | UP | UP | UP | UP | 887 |
| LOC202134 | DOWN | DOWN | DOWN | DOWN | 888 |
| KIAA0319L | UP | UP | UP | UP | 889 |
| TIAM2 | UP | UP | DOWN | DOWN | 890 |
| MOSC1 | UP | UP | UP | UP | 891 |
| PFKFB3 | UP | UP | UP | UP | 892 |
| GNB4 | UP | UP | UP | UP | 893 |
| ANKRD22 | UP | UP | UP | UP | 894 |
| PROS1 | UP | UP | UP | UP | 895 |
| CD40LG | DOWN | DOWN | DOWN | DOWN | 896 |
| RIOK2 | DOWN | DOWN | DOWN | DOWN | 897 |
| AFF1 | UP | UP | UP | UP | 898 |
| HIST1H3D | UP | UP | UP | UP | 899 |
| SLC26A8 | UP | UP | UP | UP | 900 |
| SLC26A8 | UP | UP | UP | UP | 901 |
| RNASE3 | UP | UP | UP | UP | 902 |
| UBE2L6 | DOWN | UP | UP | UP | 903 |
| UBE2L6 | DOWN | UP | UP | UP | 904 |
| SSH1 | UP | UP | DOWN | UP | 905 |
| KRBA1 | DOWN | DOWN | DOWN | DOWN | 906 |
| SLC25A23 | DOWN | DOWN | DOWN | DOWN | 907 |
| DTX3L | UP | UP | UP | UP | 908 |
| DOK3 | UP | UP | UP | UP | 909 |
| LOC644615 | UP | UP | UP | UP | 910 |
| SULT1B1 | UP | UP | DOWN | UP | 911 |
| RASGRP4 | UP | UP | UP | UP | 912 |
| ALOX15B | UP | UP | UP | UP | 913 |
| ADM | UP | UP | UP | UP | 914 |
| LOC391825 | DOWN | DOWN | DOWN | DOWN | 915 |
| LOC730234 | UP | UP | UP | UP | 916 |
| HIST2H2AA3 | UP | UP | UP | UP | 917 |
| HIST2H2AA3 | UP | UP | UP | UP | 918 |
| LIMK2 | UP | UP | UP | UP | 919 |
| MMRN1 | UP | UP | UP | UP | 920 |
| PADI2 | UP | UP | DOWN | UP | 921 |
| FKBP1A | UP | UP | UP | UP | 922 |
| GYG1 | UP | UP | UP | UP | 923 |
| | UP | UP | DOWN | UP | 924 |
| ASF1A | DOWN | DOWN | DOWN | DOWN | 925 |
| CD248 | DOWN | DOWN | DOWN | DOWN | 926 |
| CD3G | DOWN | DOWN | DOWN | DOWN | 927 |
| DEFA1 | UP | UP | UP | UP | 928 |
| EPHX2 | DOWN | DOWN | DOWN | DOWN | 929 |
| CST7 | UP | UP | DOWN | UP | 930 |
| ABLIM3 | UP | UP | UP | UP | 931 |
| ANKRD55 | DOWN | UP | DOWN | DOWN | 932 |
| SLC45A3 | DOWN | DOWN | UP | DOWN | 933 |
| RAB33B | UP | UP | UP | UP | 934 |
| LILRA6 | UP | UP | UP | UP | 935 |
| LILRA6 | UP | UP | UP | UP | 936 |
| SPTLC2 | UP | UP | UP | UP | 937 |
| CDA | UP | UP | UP | UP | 938 |
| PGD | UP | UP | UP | UP | 939 |
| LOC100130769 | DOWN | DOWN | UP | UP | 940 |
| ECHDC2 | DOWN | DOWN | DOWN | DOWN | 941 |
| KIF20B | DOWN | DOWN | DOWN | DOWN | 942 |
| B3GNT8 | UP | UP | UP | UP | 943 |
| PYHIN1 | DOWN | DOWN | DOWN | DOWN | 944 |
| LBH | DOWN | DOWN | DOWN | DOWN | 945 |
| LBH | DOWN | DOWN | DOWN | DOWN | 946 |
| | UP | UP | UP | UP | 947 |
| BPI | UP | UP | UP | UP | 948 |
| GAR1 | DOWN | DOWN | DOWN | DOWN | 949 |
| ST3GAL4 | UP | UP | DOWN | UP | 950 |
| TMEM19 | DOWN | DOWN | DOWN | DOWN | 951 |
| DHRS12 | UP | UP | UP | UP | 952 |
| DHRS12 | UP | UP | UP | UP | 953 |
| | UP | UP | UP | UP | 954 |
| FAM26F | DOWN | UP | UP | UP | 955 |
| FCRLA | DOWN | DOWN | DOWN | DOWN | 956 |
| OSBPL7 | DOWN | DOWN | DOWN | DOWN | 957 |
| CTSB | UP | DOWN | UP | UP | 958 |
| ALDH1A1 | UP | DOWN | UP | UP | 959 |
| SRRD | DOWN | DOWN | UP | DOWN | 960 |
| TOLLIP | UP | UP | UP | UP | 961 |
| ICAM1 | UP | UP | UP | UP | 962 |
| LAX1 | DOWN | DOWN | DOWN | DOWN | 963 |
| CASP7 | UP | UP | UP | UP | 964 |
| ZDHHC19 | UP | UP | UP | UP | 965 |
| LOC732371 | UP | UP | UP | UP | 966 |
| DENND1A | UP | UP | UP | UP | 967 |
| EMR2 | UP | UP | UP | UP | 968 |
| LOC643308 | DOWN | DOWN | DOWN | DOWN | 969 |
| ADA | DOWN | DOWN | UP | DOWN | 970 |
| LOC646527 | DOWN | DOWN | DOWN | DOWN | 971 |
| LOC643313 | UP | UP | UP | UP | 972 |
| GZMB | DOWN | DOWN | DOWN | DOWN | 973 |
| OLIG2 | DOWN | UP | UP | DOWN | 974 |
| GRINA | DOWN | UP | UP | UP | 975 |
| HLA-DPB1 | DOWN | DOWN | UP | UP | 976 |
| MX1 | DOWN | UP | UP | UP | 977 |
| THOC3 | DOWN | DOWN | DOWN | DOWN | 978 |
| CHST13 | UP | UP | UP | DOWN | 979 |
| TRPM6 | UP | UP | UP | UP | 980 |
| GK | UP | UP | UP | UP | 981 |
| JAK2 | UP | UP | UP | UP | 982 |
| ARHGEF11 | UP | UP | UP | UP | 983 |
| ARHGEF11 | UP | UP | UP | UP | 984 |
| HOMER2 | UP | UP | UP | UP | 985 |
| TACSTD2 | UP | UP | UP | UP | 986 |
| CA4 | UP | UP | UP | UP | 987 |
| GAA | UP | UP | UP | UP | 988 |
| IFITM3 | UP | UP | UP | UP | 989 |
| CLYBL | DOWN | DOWN | DOWN | DOWN | 990 |
| CLYBL | DOWN | DOWN | DOWN | DOWN | 991 |
| ANGPT1 | UP | DOWN | UP | DOWN | 992 |
| MME | UP | UP | UP | UP | 993 |
| ZNF408 | UP | UP | UP | UP | 994 |
| STAT1 | UP | UP | UP | UP | 995 |
| STAT1 | UP | UP | UP | UP | 996 |
| PNPLA7 | DOWN | DOWN | DOWN | DOWN | 997 |
| INDO | DOWN | UP | UP | UP | 998 |
| PDZD8 | UP | UP | UP | UP | 999 |
| PDGFD | DOWN | DOWN | DOWN | DOWN | 1000 |
| CTSL1 | UP | UP | UP | UP | 1001 |
| HOMER3 | UP | UP | UP | UP | 1002 |
| CEP78 | DOWN | DOWN | DOWN | DOWN | 1003 |
| SBK1 | DOWN | DOWN | DOWN | DOWN | 1004 |
| ALG9 | DOWN | DOWN | DOWN | DOWN | 1005 |
| KIF27 | UP | DOWN | UP | UP | 1006 |
| IL1R2 | UP | UP | UP | UP | 1007 |
| RAB40B | DOWN | DOWN | DOWN | DOWN | 1008 |
| MMP23B | DOWN | DOWN | DOWN | DOWN | 1009 |
| | UP | UP | UP | UP | 1010 |
| PGLYRP1 | UP | UP | UP | UP | 1011 |
| UHRF1 | UP | UP | UP | UP | 1012 |
| IFI44L | DOWN | UP | UP | UP | 1013 |
| PARP10 | DOWN | UP | UP | UP | 1014 |
| PARP10 | UP | UP | UP | UP | 1015 |
| GOLGA8A | DOWN | DOWN | DOWN | DOWN | 1016 |
| CCR7 | DOWN | DOWN | DOWN | DOWN | 1017 |
| HEMGN | DOWN | DOWN | DOWN | DOWN | 1018 |
| TCF7 | DOWN | DOWN | DOWN | DOWN | 1019 |
| CLUAP1 | DOWN | DOWN | DOWN | DOWN | 1020 |
| LOC390735 | DOWN | DOWN | DOWN | DOWN | 1021 |
| LOC641849 | DOWN | DOWN | DOWN | DOWN | 1022 |
| TYMP | UP | UP | UP | UP | 1023 |
| DEFA1B | UP | UP | UP | UP | 1024 |
| DEFA1B | UP | UP | UP | UP | 1025 |
| DEFA1B | UP | UP | UP | UP | 1026 |
| REPS2 | UP | UP | UP | UP | 1027 |
| REPS2 | UP | UP | UP | UP | 1028 |
| ZNF550 | DOWN | DOWN | DOWN | DOWN | 1029 |
| OSBPL1A | UP | UP | DOWN | DOWN | 1030 |
| C11ORF1 | DOWN | DOWN | DOWN | DOWN | 1031 |
| MCTP2 | UP | UP | UP | UP | 1032 |
| EMR4 | DOWN | DOWN | UP | UP | 1033 |
| LOC653316 | DOWN | DOWN | DOWN | DOWN | 1034 |
| | UP | UP | UP | UP | 1035 |
| FCRL6 | DOWN | DOWN | DOWN | DOWN | 1036 |
| MRPS26 | DOWN | DOWN | DOWN | DOWN | 1037 |
| RHOBTB3 | DOWN | DOWN | UP | UP | 1038 |
| DIRC2 | UP | UP | UP | UP | 1039 |
| CD27 | DOWN | DOWN | DOWN | DOWN | 1040 |
| PLEKHG4 | DOWN | DOWN | DOWN | DOWN | 1041 |
| CDH6 | UP | UP | UP | UP | 1042 |
| C4ORF23 | UP | UP | UP | UP | 1043 |
| HIST2H2AC | UP | UP | UP | UP | 1044 |
| SLC7A6 | DOWN | DOWN | DOWN | DOWN | 1045 |
| SLC7A6 | DOWN | DOWN | DOWN | DOWN | 1046 |
| SLAMF6 | DOWN | DOWN | DOWN | DOWN | 1047 |
| RETN | UP | UP | DOWN | UP | 1048 |
| FAIM3 | DOWN | DOWN | DOWN | DOWN | 1049 |
| PIK3C2A | DOWN | DOWN | DOWN | DOWN | 1050 |
| TMEM99 | DOWN | DOWN | DOWN | DOWN | 1051 |
| LOC728411 | DOWN | DOWN | DOWN | DOWN | 1052 |
| TMEM194A | DOWN | DOWN | DOWN | DOWN | 1053 |
| NAPEPLD | DOWN | DOWN | DOWN | DOWN | 1054 |
| ACOX1 | UP | UP | UP | UP | 1055 |
| CTLA4 | DOWN | DOWN | DOWN | DOWN | 1056 |
| SCO2 | UP | UP | UP | UP | 1057 |
| STK3 | UP | UP | UP | UP | 1058 |
| FLT3LG | DOWN | DOWN | DOWN | DOWN | 1059 |
| VASP | UP | UP | UP | UP | 1060 |
| FBXO31 | DOWN | DOWN | DOWN | DOWN | 1061 |
| TDRD9 | UP | UP | DOWN | UP | 1062 |
| TDRD9 | UP | UP | UP | UP | 1063 |
| LOC646144 | UP | UP | UP | UP | 1064 |
| NUSAP1 | UP | UP | UP | UP | 1065 |
| GPR97 | UP | UP | UP | UP | 1066 |
| GPR97 | UP | UP | UP | UP | 1067 |
| GPR97 | UP | UP | UP | UP | 1068 |
| EMR1 | DOWN | UP | UP | UP | 1069 |
| NR1H3 | DOWN | UP | UP | UP | 1070 |
| SLAMF6 | DOWN | DOWN | DOWN | DOWN | 1071 |
| CCDC106 | DOWN | DOWN | DOWN | DOWN | 1072 |
| ODF3B | UP | UP | UP | UP | 1073 |
| LOC100129904 | UP | UP | UP | UP | 1074 |
| PADI4 | UP | UP | UP | UP | 1075 |
| LOC100132858 | UP | UP | UP | UP | 1076 |
| PIK3AP1 | UP | UP | UP | UP | 1077 |
| ZNF792 | DOWN | DOWN | DOWN | DOWN | 1078 |
| DIP2A | DOWN | DOWN | DOWN | DOWN | 1079 |
| OSCAR | UP | UP | UP | UP | 1080 |
| | DOWN | DOWN | DOWN | DOWN | 1081 |
| CLIC3 | DOWN | DOWN | DOWN | DOWN | 1082 |
| FANCE | DOWN | DOWN | DOWN | DOWN | 1083 |
| TECPR2 | UP | UP | UP | UP | 1084 |
| P2RY10 | DOWN | DOWN | DOWN | DOWN | 1085 |
| ADORA3 | UP | UP | UP | UP | 1086 |
| IL18RAP | UP | UP | DOWN | UP | 1087 |
| DEFA3 | UP | UP | UP | UP | 1088 |
| BRSK1 | UP | UP | UP | UP | 1089 |
| LOC647691 | UP | UP | UP | UP | 1090 |
| ALG8 | DOWN | DOWN | DOWN | DOWN | 1091 |
| S1PR5 | DOWN | DOWN | DOWN | DOWN | 1092 |
| CPA3 | DOWN | DOWN | UP | DOWN | 1093 |
| BMX | UP | UP | UP | UP | 1094 |
| DDX58 | UP | UP | UP | UP | 1095 |
| RHOBTB1 | UP | UP | UP | UP | 1096 |
| TNFRSF25 | DOWN | DOWN | DOWN | DOWN | 1097 |
| LOC730387 | UP | UP | UP | UP | 1098 |
| OLR1 | UP | UP | UP | UP | 1099 |
| HERC5 | UP | UP | UP | UP | 1100 |
| STAT1 | UP | UP | UP | UP | 1101 |
| NELF | DOWN | DOWN | DOWN | DOWN | 1102 |
| STAP1 | DOWN | DOWN | DOWN | DOWN | 1103 |
| SLC2A5 | UP | UP | UP | UP | 1104 |
| ITGB5 | UP | UP | UP | UP | 1105 |
| ZNF516 | UP | UP | UP | UP | 1106 |
| ARHGAP26 | UP | UP | UP | UP | 1107 |
| TIMP2 | UP | UP | UP | UP | 1108 |
| FCGR1A | UP | UP | UP | UP | 1109 |
| RHOH | DOWN | DOWN | DOWN | DOWN | 1110 |
| IFI44 | UP | UP | UP | UP | 1111 |
| MTX3 | DOWN | DOWN | DOWN | DOWN | 1112 |
| CD74 | UP | DOWN | UP | UP | 1113 |
| LCK | DOWN | DOWN | DOWN | DOWN | 1114 |
| TLR4 | UP | UP | UP | UP | 1115 |
| | DOWN | DOWN | DOWN | DOWN | 1116 |
| DSC2 | UP | UP | UP | UP | 1117 |
| CXORF45 | DOWN | DOWN | DOWN | DOWN | 1118 |
| ENPP4 | DOWN | DOWN | DOWN | DOWN | 1119 |
| CD300C | UP | UP | UP | UP | 1120 |
| OASL | DOWN | UP | UP | UP | 1121 |
| HPSE | UP | UP | UP | UP | 1122 |
| MTHFD2 | UP | UP | UP | UP | 1123 |
| GSTM2 | DOWN | DOWN | DOWN | DOWN | 1124 |
| OLFM4 | UP | UP | UP | UP | 1125 |
| ABHD12B | UP | UP | UP | UP | 1126 |
| LOC728417 | UP | UP | UP | UP | 1127 |
| LOC728417 | UP | UP | UP | UP | 1128 |
| FCAR | UP | UP | UP | UP | 1129 |
| GTPBP3 | DOWN | DOWN | DOWN | DOWN | 1130 |
| KLF4 | UP | DOWN | UP | UP | 1131 |
| HOPX | DOWN | DOWN | DOWN | DOWN | 1132 |
| THBD | UP | UP | DOWN | UP | 1133 |
| HIST1H2BG | DOWN | UP | DOWN | UP | 1134 |
| LOC730995 | DOWN | DOWN | DOWN | DOWN | 1135 |
| OPN3 | DOWN | DOWN | DOWN | DOWN | 1136 |
| NOP56 | DOWN | DOWN | DOWN | DOWN | 1137 |
| ZBTB9 | DOWN | DOWN | DOWN | DOWN | 1138 |
| NLRC3 | DOWN | DOWN | DOWN | DOWN | 1139 |
| LOC100134083 | UP | UP | UP | UP | 1140 |
| COP1 | UP | UP | UP | UP | 1141 |
| CARD16 | UP | UP | UP | UP | 1142 |
| SP140 | DOWN | UP | UP | UP | 1143 |
| CD96 | DOWN | DOWN | DOWN | DOWN | 1144 |
| UBE2O | DOWN | DOWN | UP | DOWN | 1145 |
| POLD2 | DOWN | DOWN | DOWN | DOWN | 1146 |
| IL32 | DOWN | DOWN | DOWN | DOWN | 1147 |
| LOC728744 | UP | UP | UP | UP | 1148 |
| FZD2 | UP | UP | UP | UP | 1149 |
| ZAP70 | DOWN | DOWN | DOWN | DOWN | 1150 |
| PYHIN1 | DOWN | DOWN | DOWN | DOWN | 1151 |
| SCARF1 | UP | UP | UP | UP | 1152 |
| IFI27 | UP | UP | UP | UP | 1153 |
| PFKFB2 | UP | UP | UP | UP | 1154 |
| PAM | UP | UP | DOWN | DOWN | 1155 |
| WARS | DOWN | UP | UP | UP | 1156 |
| | DOWN | DOWN | DOWN | DOWN | 1157 |
| TCN1 | UP | UP | UP | UP | 1158 |
| LOC649839 | DOWN | DOWN | DOWN | DOWN | 1159 |
| MMP9 | UP | UP | UP | UP | 1160 |
| RIN3 | UP | UP | UP | UP | 1161 |
| TMEM194A | DOWN | DOWN | DOWN | DOWN | 1162 |
| TAP2 | UP | UP | UP | UP | 1163 |
| C17ORF87 | DOWN | DOWN | UP | UP | 1164 |
| LOC728650 | UP | UP | UP | UP | 1165 |
| PNMA3 | DOWN | DOWN | DOWN | DOWN | 1166 |
| CPT1B | UP | UP | UP | UP | 1167 |
| LTBP3 | DOWN | DOWN | DOWN | DOWN | 1168 |
| CCDC34 | DOWN | DOWN | UP | DOWN | 1169 |
| PRAGMIN | DOWN | DOWN | DOWN | DOWN | 1170 |
| C9ORF91 | DOWN | DOWN | UP | UP | 1171 |
| SMPDL3A | UP | UP | UP | UP | 1172 |
| GPR56 | DOWN | DOWN | DOWN | DOWN | 1173 |
| C14ORF147 | UP | UP | UP | UP | 1174 |
| SMARCD3 | UP | UP | UP | UP | 1175 |
| FAM119A | DOWN | DOWN | DOWN | DOWN | 1176 |
| LOC642334 | UP | UP | UP | UP | 1177 |
| ENOSF1 | DOWN | DOWN | DOWN | DOWN | 1178 |
| FAR2 | UP | UP | UP | UP | 1179 |
| LOC441763 | UP | UP | DOWN | UP | 1180 |
| TESC | DOWN | DOWN | UP | DOWN | 1181 |
| CECR6 | UP | UP | UP | UP | 1182 |
| KIAA1598 | UP | UP | UP | UP | 1183 |
| | UP | UP | UP | UP | 1184 |
| GPR109B | UP | UP | UP | UP | 1185 |
| LRRN3 | DOWN | DOWN | DOWN | DOWN | 1186 |
| RNF213 | DOWN | DOWN | UP | UP | 1187 |
| LRP3 | UP | UP | UP | UP | 1188 |
| ASGR2 | UP | UP | UP | UP | 1189 |
| ASGR2 | UP | UP | UP | UP | 1190 |
| ZSCAN18 | DOWN | DOWN | DOWN | DOWN | 1191 |
| MCOLN2 | DOWN | DOWN | DOWN | DOWN | 1192 |
| IFIT2 | UP | UP | UP | UP | 1193 |
| PLCH2 | DOWN | DOWN | DOWN | DOWN | 1194 |
| MAP7 | DOWN | DOWN | DOWN | DOWN | 1195 |
| GBP4 | DOWN | DOWN | UP | UP | 1196 |
| MGMT | DOWN | DOWN | DOWN | DOWN | 1197 |
| GAL3ST4 | DOWN | DOWN | DOWN | DOWN | 1198 |
| C2ORF89 | DOWN | DOWN | DOWN | DOWN | 1199 |
| TXNDC3 | UP | UP | UP | UP | 1200 |
| IFIH1 | DOWN | UP | UP | UP | 1201 |
| PRRG4 | UP | UP | UP | UP | 1202 |
| LOC641693 | UP | UP | UP | UP | 1203 |
| LOC728093 | UP | UP | UP | UP | 1204 |
| TNFAIP8L1 | DOWN | DOWN | UP | DOWN | 1205 |
| AP3M2 | DOWN | DOWN | DOWN | DOWN | 1206 |
| BACH2 | DOWN | DOWN | DOWN | DOWN | 1207 |
| BACH2 | DOWN | DOWN | DOWN | DOWN | 1208 |
| C9ORF123 | DOWN | DOWN | DOWN | DOWN | 1209 |
| CACNA1I | DOWN | DOWN | DOWN | DOWN | 1210 |
| LOC100132287 | UP | UP | UP | UP | 1211 |
| CAMK1D | UP | UP | UP | DOWN | 1212 |
| ANKRD33 | UP | UP | UP | UP | 1213 |
| CCR6 | DOWN | DOWN | DOWN | DOWN | 1214 |
| ALDH1A1 | DOWN | DOWN | UP | UP | 1215 |
| LOC100132797 | DOWN | UP | DOWN | DOWN | 1216 |
| CD163 | UP | UP | UP | UP | 1217 |
| ESAM | UP | UP | UP | UP | 1218 |
| FCAR | UP | UP | UP | UP | 1219 |
| TCN2 | UP | UP | UP | UP | 1220 |
| LOC100129203 | DOWN | DOWN | DOWN | UP | 1221 |
| CD6 | DOWN | DOWN | DOWN | DOWN | 1222 |
| B3GNT1 | DOWN | DOWN | DOWN | DOWN | 1223 |
| NEK8 | DOWN | DOWN | DOWN | DOWN | 1224 |
| SLC38A5 | UP | UP | UP | UP | 1225 |
| CD3E | DOWN | DOWN | DOWN | DOWN | 1226 |
| | DOWN | DOWN | DOWN | DOWN | 1227 |
| GPR183 | DOWN | DOWN | DOWN | DOWN | 1228 |
| CCDC76 | DOWN | DOWN | DOWN | DOWN | 1229 |
| MS4A1 | DOWN | DOWN | DOWN | DOWN | 1230 |
| IFIT1 | DOWN | UP | UP | UP | 1231 |
| MED13L | UP | UP | DOWN | DOWN | 1232 |
| SLC26A8 | UP | UP | UP | UP | 1233 |
| NOV | DOWN | DOWN | DOWN | DOWN | 1234 |
| FLJ20035 | DOWN | UP | UP | UP | 1235 |
| UGT1A3 | UP | UP | UP | UP | 1236 |
| LOC653600 | UP | UP | UP | UP | 1237 |
| LOC642684 | UP | UP | UP | UP | 1238 |
| KIAA0319L | UP | UP | UP | UP | 1239 |
| KLRD1 | DOWN | DOWN | DOWN | DOWN | 1240 |
| TRIM22 | UP | UP | UP | UP | 1241 |
| C4ORF18 | UP | UP | UP | UP | 1242 |
| TSPAN3 | DOWN | DOWN | DOWN | DOWN | 1243 |
| TSPAN3 | DOWN | DOWN | DOWN | DOWN | 1244 |
| LOC728748 | DOWN | DOWN | DOWN | DOWN | 1245 |
| DNAJC3 | UP | UP | UP | UP | 1246 |
| AGTRAP | UP | UP | UP | UP | 1247 |
| LOC646786 | UP | UP | DOWN | DOWN | 1248 |
| NCALD | DOWN | DOWN | DOWN | DOWN | 1249 |
| TTC25 | DOWN | DOWN | UP | DOWN | 1250 |
| LOC646966 | DOWN | DOWN | DOWN | DOWN | 1251 |
| TSPAN5 | DOWN | DOWN | UP | DOWN | 1252 |
| ZNF559 | DOWN | DOWN | DOWN | DOWN | 1253 |
| NFKB2 | UP | UP | UP | UP | 1254 |
| LOC652616 | UP | UP | UP | UP | 1255 |
| HLA-DOA | DOWN | DOWN | UP | DOWN | 1256 |
| WARS | DOWN | UP | UP | UP | 1257 |
| GBP2 | UP | UP | UP | UP | 1258 |
| AUTS2 | DOWN | DOWN | DOWN | DOWN | 1259 |
| IGF2BP3 | UP | UP | UP | UP | 1260 |
| OASL | UP | UP | UP | UP | 1261 |
| DYSF | UP | UP | UP | UP | 1262 |
| FLJ43093 | DOWN | DOWN | UP | DOWN | 1263 |
| FAM159A | DOWN | DOWN | DOWN | DOWN | 1264 |
| MS4A14 | UP | DOWN | UP | UP | 1265 |
| TGFB1I1 | UP | UP | UP | UP | 1266 |
| RAD51C | DOWN | DOWN | DOWN | DOWN | 1267 |
| CALD1 | UP | UP | UP | UP | 1268 |
| LOC441073 | DOWN | DOWN | DOWN | DOWN | 1269 |
| CCNC | DOWN | DOWN | DOWN | DOWN | 1270 |
| LOC730281 | UP | UP | UP | UP | 1271 |
| MUC1 | UP | UP | UP | UP | 1272 |
| C14ORF124 | DOWN | DOWN | DOWN | DOWN | 1273 |
| RPL14 | DOWN | DOWN | DOWN | DOWN | 1274 |
| APOL6 | UP | UP | UP | UP | 1275 |
| | DOWN | DOWN | DOWN | DOWN | 1276 |
| KCTD12 | UP | UP | UP | UP | 1277 |
| ITGAX | UP | UP | UP | UP | 1278 |
| IFIT3 | UP | UP | UP | UP | 1279 |
| LPCAT2 | DOWN | UP | UP | UP | 1280 |
| ZNF529 | DOWN | DOWN | DOWN | DOWN | 1281 |
| MRPL9 | DOWN | DOWN | DOWN | DOWN | 1282 |
| AGTRAP | UP | UP | UP | UP | 1283 |
| LOC402112 | DOWN | DOWN | DOWN | DOWN | 1284 |
| LOC100134822 | UP | UP | UP | UP | 1285 |
| SH2D1B | DOWN | DOWN | DOWN | DOWN | 1286 |
| MPO | UP | UP | UP | UP | 1287 |
| LOC100131967 | UP | UP | UP | UP | 1288 |
| LOC440459 | UP | UP | UP | UP | 1289 |
| FAM44B | DOWN | DOWN | DOWN | DOWN | 1290 |
| ACOT9 | UP | UP | UP | UP | 1291 |
| SLC37A1 | DOWN | UP | UP | UP | 1292 |
| LOC729915 | UP | UP | UP | UP | 1293 |
| PDZK1IP1 | DOWN | DOWN | UP | DOWN | 1294 |
| S100A12 | UP | UP | UP | UP | 1295 |
| RAB3IL1 | DOWN | DOWN | UP | UP | 1296 |
| TMEM204 | DOWN | DOWN | DOWN | DOWN | 1297 |
| CXCL10 | UP | UP | UP | UP | 1298 |
| TSR1 | DOWN | DOWN | DOWN | DOWN | 1299 |
| NSUN5 | DOWN | UP | DOWN | DOWN | 1300 |
| MXD3 | UP | UP | UP | UP | 1301 |
| LILRA5 | UP | UP | UP | UP | 1302 |
| CKAP4 | UP | UP | UP | UP | 1303 |
| C6ORF190 | DOWN | DOWN | DOWN | DOWN | 1304 |
| ECGF1 | UP | UP | UP | UP | 1305 |
| LDLRAP1 | DOWN | DOWN | DOWN | DOWN | 1306 |
| GRB10 | UP | UP | UP | UP | 1307 |
| FCRL3 | DOWN | DOWN | DOWN | DOWN | 1308 |
| LOC731275 | UP | UP | UP | UP | 1309 |
| ZFP91 | UP | UP | DOWN | UP | 1310 |
| CTRL | UP | UP | UP | UP | 1311 |
| BCL6 | UP | UP | UP | UP | 1312 |
| SAMD3 | DOWN | DOWN | DOWN | DOWN | 1313 |
| LOC647436 | DOWN | DOWN | DOWN | DOWN | 1314 |
| CLC | DOWN | DOWN | UP | DOWN | 1315 |
| GK | UP | UP | UP | UP | 1316 |
| LOC100133565 | UP | UP | DOWN | UP | 1317 |
| OAS2 | UP | DOWN | UP | UP | 1318 |
| LOC644937 | DOWN | DOWN | DOWN | DOWN | 1319 |
| SIRPD | UP | UP | UP | UP | 1320 |
| GPBAR1 | UP | DOWN | UP | UP | 1321 |
| GNL3 | DOWN | DOWN | DOWN | DOWN | 1322 |
| CD79B | DOWN | DOWN | DOWN | DOWN | 1323 |
| ELF2 | UP | UP | UP | UP | 1324 |
| GAA | UP | UP | UP | UP | 1325 |
| CD47 | DOWN | DOWN | DOWN | DOWN | 1326 |
| NMT2 | DOWN | DOWN | DOWN | DOWN | 1327 |
| MATR3 | DOWN | DOWN | DOWN | DOWN | 1328 |
| TMEM107 | UP | DOWN | DOWN | DOWN | 1329 |
| GCM1 | UP | UP | UP | UP | 1330 |
| RORA | DOWN | DOWN | DOWN | DOWN | 1331 |
| MGAM | UP | UP | UP | UP | 1332 |
| LOC100132491 | UP | UP | UP | UP | 1333 |
| KRT72 | DOWN | DOWN | DOWN | DOWN | 1334 |
| SEPT4 | UP | UP | UP | UP | 1335 |
| ACADVL | UP | UP | UP | UP | 1336 |
| ANXA3 | UP | UP | UP | UP | 1337 |
| MEGF9 | UP | UP | UP | UP | 1338 |
| MEGF9 | UP | UP | UP | UP | 1339 |
| PTPRJ | UP | UP | UP | UP | 1340 |
| HLA-DRB4 | DOWN | DOWN | UP | UP | 1341 |
| GHRL | DOWN | UP | UP | UP | 1342 |
| ALAS2 | DOWN | UP | UP | UP | 1343 |
| FFAR2 | UP | UP | UP | UP | 1344 |
| MPZL2 | DOWN | UP | UP | UP | 1345 |
| PML | DOWN | UP | UP | UP | 1346 |
| HLA-DQA1 | DOWN | DOWN | UP | UP | 1347 |
| CEACAM8 | UP | UP | UP | UP | 1348 |
| SH3KBP1 | DOWN | DOWN | DOWN | DOWN | 1349 |
| TRPM2 | UP | UP | UP | UP | 1350 |
| CUX1 | UP | UP | UP | UP | 1351 |
| LOC648390 | DOWN | DOWN | UP | DOWN | 1352 |
| SUV39H1 | DOWN | DOWN | DOWN | DOWN | 1353 |
| RNF13 | UP | UP | UP | UP | 1354 |
| USF1 | UP | UP | UP | UP | 1355 |
| VAPA | UP | UP | UP | UP | 1356 |
| ALOX15 | DOWN | DOWN | UP | DOWN | 1357 |
| CD79A | DOWN | DOWN | DOWN | DOWN | 1358 |
| DPRXP4 | UP | UP | UP | UP | 1359 |
| LOC652750 | DOWN | UP | UP | UP | 1360 |
| ECM1 | UP | UP | DOWN | UP | 1361 |
| ST6GAL1 | DOWN | DOWN | DOWN | DOWN | 1362 |
| KLHL3 | DOWN | DOWN | DOWN | DOWN | 1363 |
| RTP4 | DOWN | UP | UP | UP | 1364 |
| FAM179A | DOWN | DOWN | UP | DOWN | 1365 |
| HDC | DOWN | DOWN | UP | DOWN | 1366 |
| SUMO1P1 | UP | UP | DOWN | UP | 1367 |
| SACS | DOWN | DOWN | DOWN | DOWN | 1368 |
| C9ORF72 | UP | UP | UP | UP | 1369 |
| C9ORF72 | UP | UP | UP | UP | 1370 |
| LOC652726 | DOWN | DOWN | DOWN | DOWN | 1371 |
| PVRIG | DOWN | DOWN | DOWN | DOWN | 1372 |
| PPP1R16B | DOWN | DOWN | DOWN | DOWN | 1373 |
| NSUN7 | UP | UP | DOWN | DOWN | 1374 |
| NSUN7 | UP | UP | DOWN | UP | 1375 |
| UHRF2 | DOWN | DOWN | DOWN | DOWN | 1376 |
| ZNF783 | DOWN | DOWN | DOWN | DOWN | 1377 |
| LOC441013 | DOWN | DOWN | DOWN | DOWN | 1378 |
| | UP | UP | UP | UP | 1379 |
| LOC100129343 | UP | UP | UP | UP | 1380 |
| OSM | UP | UP | UP | UP | 1381 |
| UNC93B1 | UP | UP | UP | UP | 1382 |
| DNAJC30 | DOWN | DOWN | DOWN | DOWN | 1383 |
| FLJ14166 | UP | UP | DOWN | DOWN | 1384 |
| C9ORF72 | UP | UP | DOWN | UP | 1385 |
| SAMD4A | UP | UP | UP | UP | 1386 |
| RNY4 | DOWN | DOWN | DOWN | DOWN | 1387 |
| F5 | UP | UP | UP | UP | 1388 |
| PARP15 | DOWN | DOWN | DOWN | DOWN | 1389 |
| PAFAH2 | DOWN | DOWN | DOWN | DOWN | 1390 |
| COL17A1 | UP | UP | UP | UP | 1391 |
| LOC651524 | UP | UP | UP | UP | 1392 |
| TYMP | UP | UP | UP | UP | 1393 |
| LOC389672 | DOWN | DOWN | DOWN | DOWN | 1394 |
| ABCB1 | DOWN | DOWN | DOWN | DOWN | 1395 |
| LOC644852 | DOWN | DOWN | UP | UP | 1396 |
| TARP | DOWN | DOWN | DOWN | DOWN | 1397 |
| SLAMF7 | UP | UP | UP | UP | 1398 |
| FRMD3 | UP | UP | UP | UP | 1399 |
| LOC648984 | UP | UP | UP | UP | 1400 |
| PLAUR | UP | UP | UP | UP | 1401 |
| LOC100132119 | UP | UP | UP | UP | 1402 |
| KLRG1 | DOWN | DOWN | DOWN | DOWN | 1403 |
| INTS2 | DOWN | DOWN | DOWN | DOWN | 1404 |
| MYC | DOWN | DOWN | DOWN | DOWN | 1405 |
| HIST1H4H | UP | UP | UP | UP | 1406 |
| KBTBD8 | DOWN | DOWN | DOWN | DOWN | 1407 |
| C9ORF45 | DOWN | DOWN | DOWN | DOWN | 1408 |
| GBP6 | UP | UP | UP | UP | 1409 |
| KIFAP3 | DOWN | DOWN | DOWN | DOWN | 1410 |
| HSPC159 | UP | UP | UP | UP | 1411 |
| ZNF224 | DOWN | DOWN | DOWN | DOWN | 1412 |
| SOCS3 | UP | UP | UP | UP | 1413 |
| GOLGA8B | DOWN | DOWN | DOWN | DOWN | 1414 |
| OLIG1 | DOWN | DOWN | UP | DOWN | 1415 |
| TNFRSF4 | DOWN | DOWN | UP | DOWN | 1416 |
| LOC100133583 | DOWN | DOWN | UP | UP | 1417 |
| ARL4A | DOWN | DOWN | DOWN | DOWN | 1418 |
| ASNS | DOWN | DOWN | DOWN | DOWN | 1419 |
| ITGAX | UP | UP | UP | UP | 1420 |
| LOC153561 | UP | UP | UP | UP | 1421 |
| GSTM1 | DOWN | DOWN | DOWN | DOWN | 1422 |
| OAS2 | DOWN | DOWN | UP | UP | 1423 |
| OAS2 | UP | UP | UP | UP | 1424 |
| TRIM25 | UP | UP | UP | UP | 1425 |
| ABHD14A | DOWN | DOWN | DOWN | DOWN | 1426 |
| LOC642342 | UP | UP | DOWN | DOWN | 1427 |
| GPR56 | DOWN | DOWN | DOWN | DOWN | 1428 |
| C4ORF18 | UP | UP | UP | UP | 1429 |
| AK1 | DOWN | DOWN | DOWN | DOWN | 1430 |
| PIK3R6 | DOWN | UP | UP | UP | 1431 |
| HSPE1 | DOWN | DOWN | DOWN | DOWN | 1432 |
| ASPHD2 | DOWN | UP | UP | UP | 1433 |
| DHRS9 | UP | UP | UP | UP | 1434 |
| GRN | UP | UP | UP | UP | 1435 |
| BEND7 | UP | UP | UP | UP | 1436 |
| BOAT | DOWN | DOWN | DOWN | DOWN | 1437 |
| LOC728323 | UP | UP | DOWN | UP | 1438 |
| LOC100134300 | UP | UP | UP | UP | 1439 |
| SDSL | UP | UP | UP | UP | 1440 |
| TNFAIP6 | UP | UP | UP | UP | 1441 |
| ARHGAP24 | UP | UP | UP | UP | 1442 |
| LOC402176 | UP | UP | UP | DOWN | 1443 |
| LOC441019 | DOWN | DOWN | UP | UP | 1444 |
| FAM134B | DOWN | DOWN | DOWN | DOWN | 1445 |
| ZNF573 | DOWN | DOWN | DOWN | DOWN | 1446 |

Distinct biological pathways were found to be associated with the pulmonary granulomatous diseases differing from those associated with the acute pulmonary diseases, pneumonias and chronic lung diseases, lung cancers.

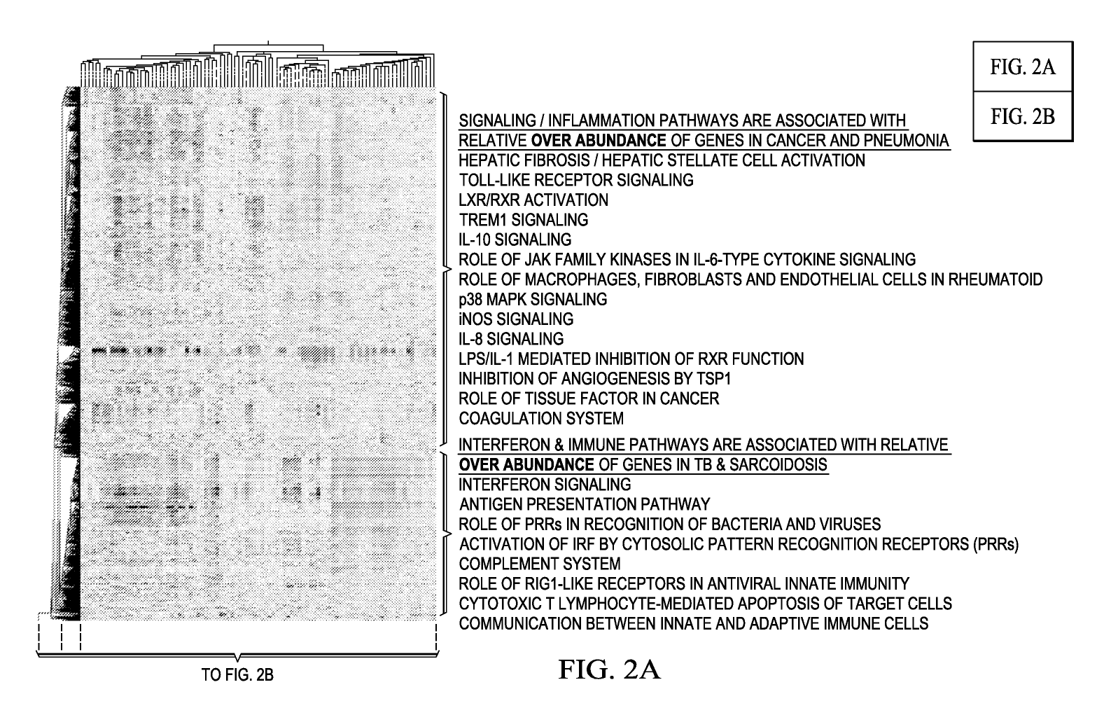

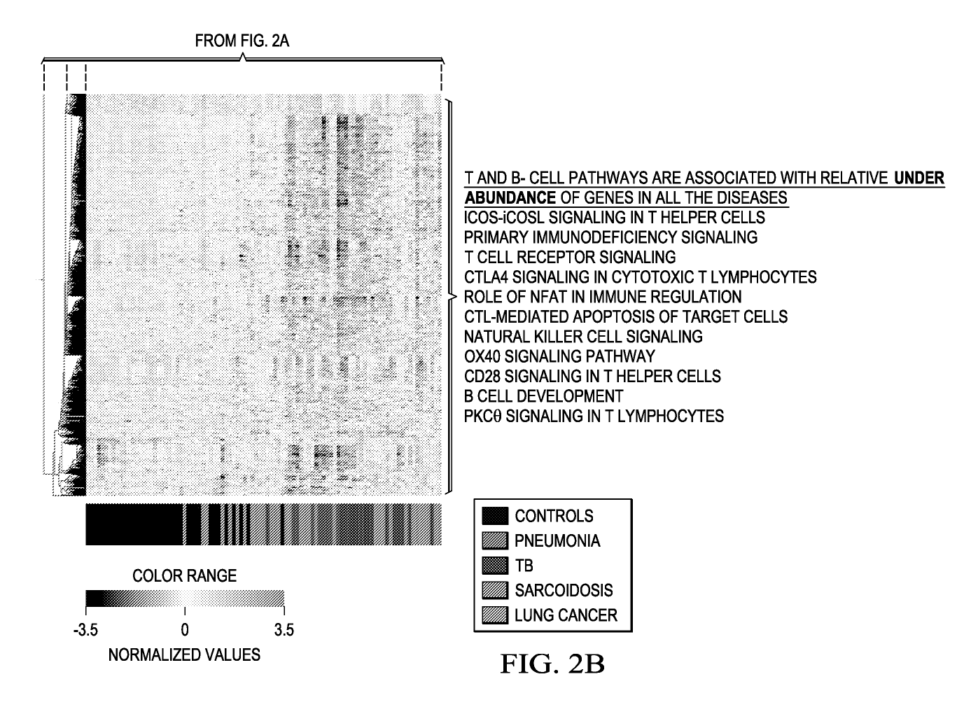

Having established by the derived 1446-transcript signature that the pulmonary granulomatous diseases had similar transcriptional profiles to each other but different to those of the pneumonia and lung cancer patients we wished to determine the main biological pathways associated with the 1446-transcripts in relation to each disease (SEQ ID NOS.:1 to 1,446). The 1446 unsupervised clustering revealed three main clusters of transcripts as can be seen from the vertical dendrogram (FIG. 2). Ingenuity Pathway Analysis (IPA) of the main clusters of transcripts revealed that the TB and sarcoidosis samples were associated with over-abundance of the interferon signalling pathway and other immune response pathways (FIG. 2). However the pneumonia and lung cancer samples were associated with over-abundance of pathways linked with inflammation. All four diseases associated with under-abundance of T and B cell pathways. Using the 1,446 genes or probes, the skilled artisan can select subsets of genes that will best differentiate between two, three or four pulmonary diseases by taking advantage of both the level of expression but also whether the gene is over- or under-expressed. As taught herein, certain subsets are demonstrated to be unique to certain pulmonary diseases, but can also be used to identify if a patient or subject has one, two, three or four of the pulmonary diseases.

FIG. 2. Three dominant clusters of transcripts in the unsupervised clustering of the 1446 transcripts are associated with distinct Ingenuity Pathway Analysis canonical pathways. Each of the three dominant clusters of transcripts is associated with different study groups in the Training Set. The top transcript cluster is over-abundant in the pneumonia and lung cancer patients and significantly associated with IPA pathways relating to inflammation (Fisher's exact p<0.05 Benjamini Hochberg). The middle transcript cluster is over-abundant in the TB and sarcoidosis patients and significantly associated with interferon signalling and other immune response IPA pathways (Fisher's exact p<0.05 Benjamini Hochberg). The bottom transcript cluster is under-abundant in all the patients and significantly associated with T and B cell IPA pathways (Fisher's exact p<0.05 Benjamini Hochberg).

The sarcoidosis patients' heterogeneous transcriptional profiles were explained by their clinical phenotype.

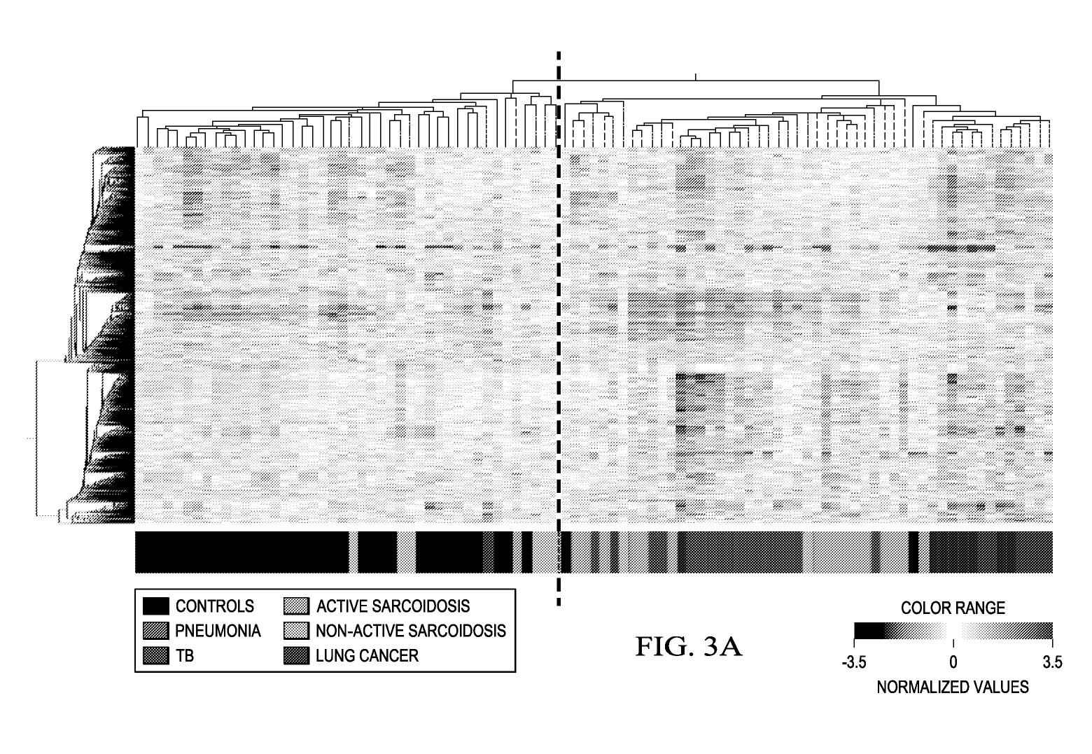

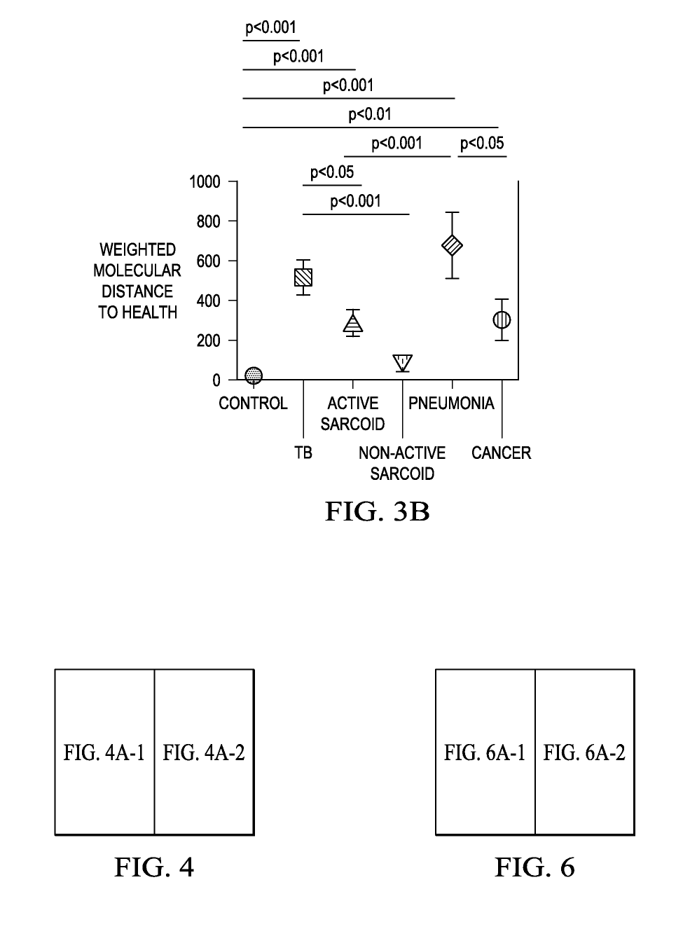

From the unsupervised clustering of the 1446-transcripts it can be seen that the sarcoidosis patients fell into two groups, those that clustered with the TB patients and those that clustered with the healthy controls (FIG. 1). As the blood transcriptional profile is a snap shot view of the host's immune response we applied the same approach to clinically phenotyping the patients to understand if their clinical classification correlates with their transcriptional profile. However there is no consensus on how to reliably assess disease activity and current classification systems all require continuous follow-up of the patient over a prolonged period of time before their activity status can be stated (1). Therefore a clinical classification was devised decision tree based on clinical variables that are both routinely measure in sarcoidosis patients and have been shown to be associated with disease activity (data not shown). Using exactly the same analysis strategy as for the 1446-transcripts, but this time with the sarcoidosis patients classified as either active or non-active, 1396-transcripts were found to be differentially expressed across all the disease groups. FIGS. 3A and 3B shows the results from the sarcoidosis patients clinically classified as active sarcoidosis display similar transcriptional signatures to the TB patients but are very distinct from the transcriptional signatures of the clinically classified non-active sarcoidosis patients which in turn resemble the healthy controls. 1396-transcripts are differentially expressed in the whole blood of healthy controls, pulmonary TB patients, active sarcoidosis patients, non-active sarcoidosis patients, pneumonia patients and lung cancer patients. FIG. 3A shows the 1396 transcripts and Training Set patients' profiles are organised by unsupervised hierarchical clustering. A dotted line is added to the heatmap to clarify the main clusters generated by the clustering algorithm. Transcript intensity values are normalised to the median of all transcripts. FIG. 3B shows the molecular distance to health of the 1396 transcripts in the Training and Test sets demonstrates the quantification of transcriptional change relative to the controls. The mean and SEM was compared between each disease group (ANOVA with Tukey's multiple comparison test).

Unsupervised hierarchical clustering again showed the same clustering pattern as seen with the 1446-transcripts (FIG. 3A). Applying the clinical classification decision tree it could be seen that those sarcoidosis patients clustering with the TB patients had been classified as active and those with the healthy controls as non-active. This was further validated in two independent cohorts, the Test and Validation Sets (data not shown). In addition, it was found that the applied clinical classification decision tree was able to predict if the sarcoidosis patients' transcriptional profiles clustered with the TB patients or the healthy controls better than any routinely measured single clinical variable (data not shown). Furthermore the clinical classification decision tree was still superior in its clustering predictive ability even if the single clinical variables with the highest predictive values were used in conjunction with each other or even when used together with the clinical classification criteria (data not shown). Molecular distance to health (MDTH) demonstrates the quantification of transcriptional change relative to the controls (FIG. 3B) (2). By applying this algorithm to all the disease groups for the 1396-transcripts it could be seen that the non-active sarcoidosis MDTH score was not significantly different from the controls, however the active sarcoidosis MDTH score was significantly different from the controls. In addition the TB patients' MDTH score was significantly higher than active sarcoidosis patients' score. Lung cancer and pneumonia both had significantly higher scores than the controls with pneumonia significantly higher than cancer. Pneumonia and TB had the highest MDTH scores. The significant differences in the MDTH scores between the patient groups suggest there is a quantitative as well as qualitative difference in blood transcriptional signatures between these similar pulmonary diseases.

Three different data mining strategies showed the same findings that both TB and active sarcoidosis were dominated by IFN-inducible genes, in contrast to pneumonia and lung cancer, which were dominated by inflammatory genes.

To further understand the biological pathways associated with each disease group we undertook three different data mining strategies to ensure our findings were robust and consistent. The three approaches applied were: modular analysis, Ingenuity Pathway Analysis and annotation of the top differentially expressed genes for each disease group.

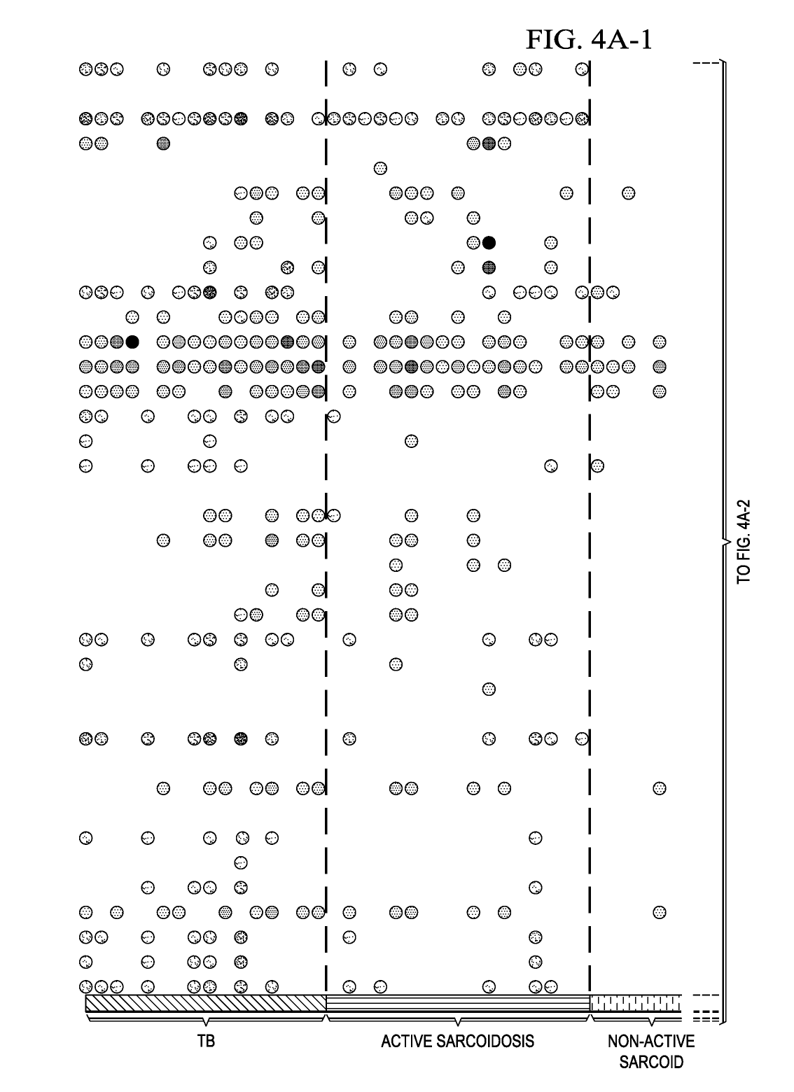

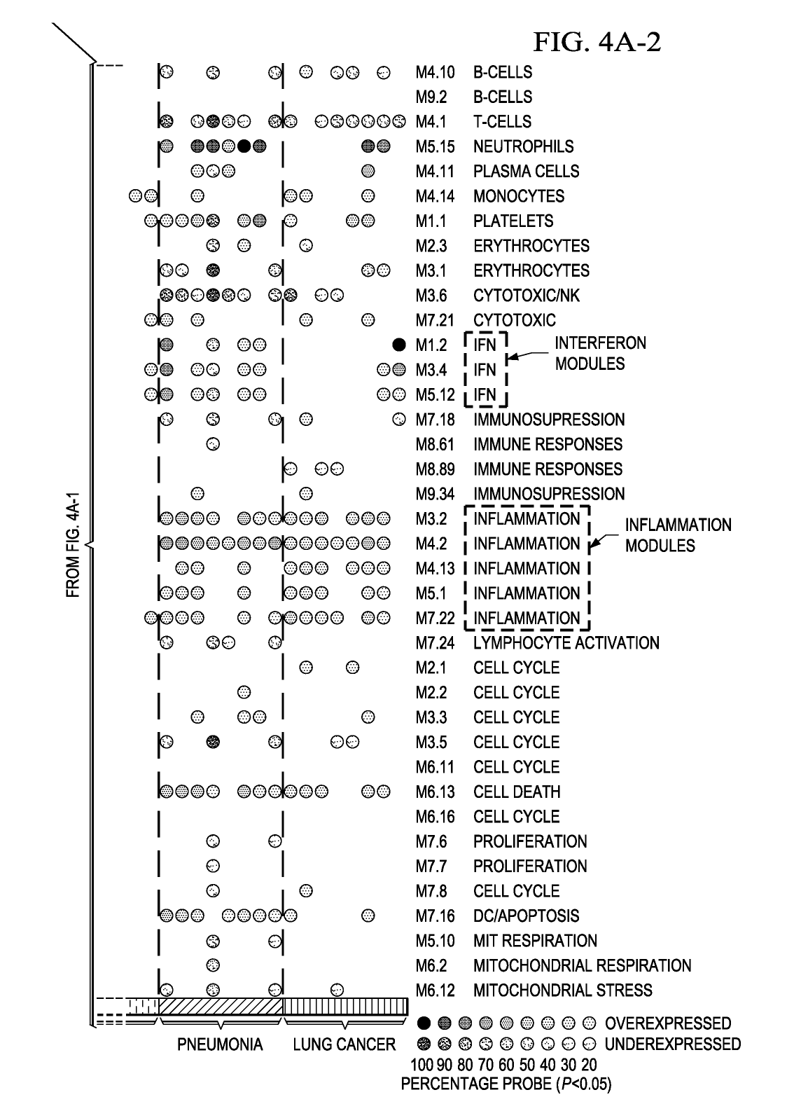

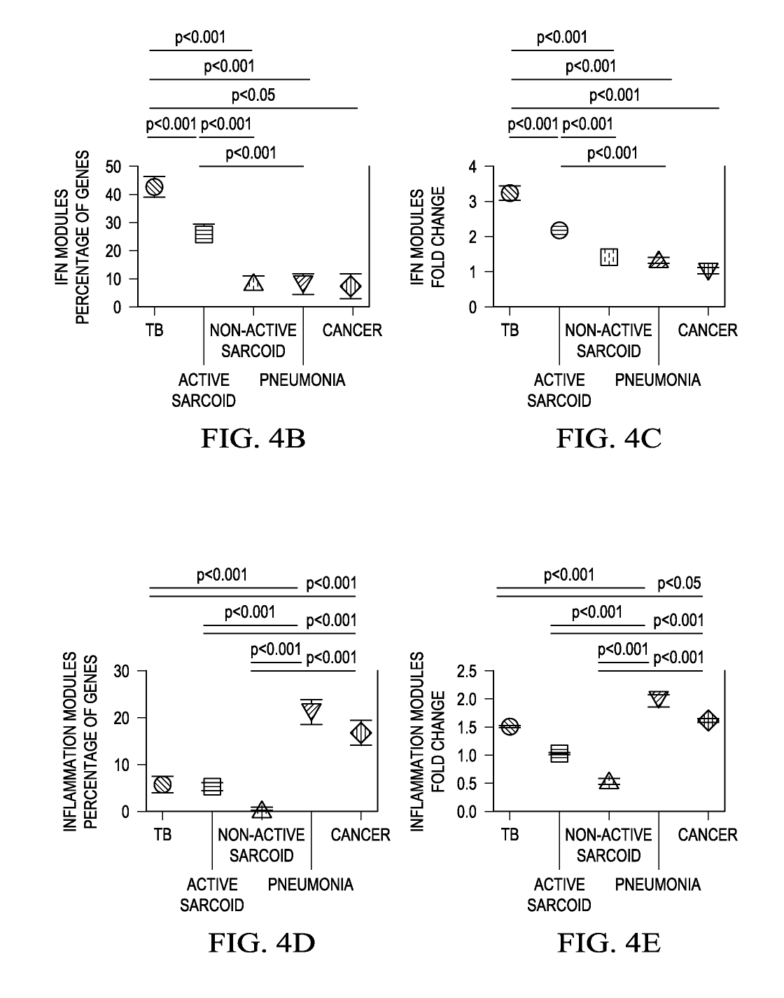

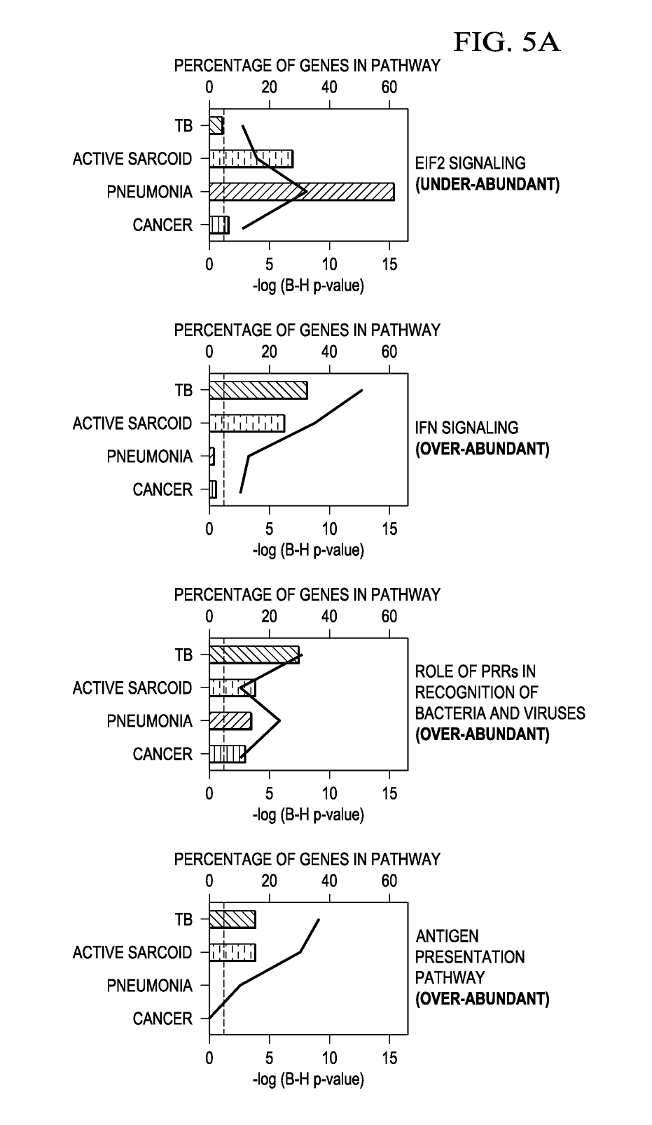

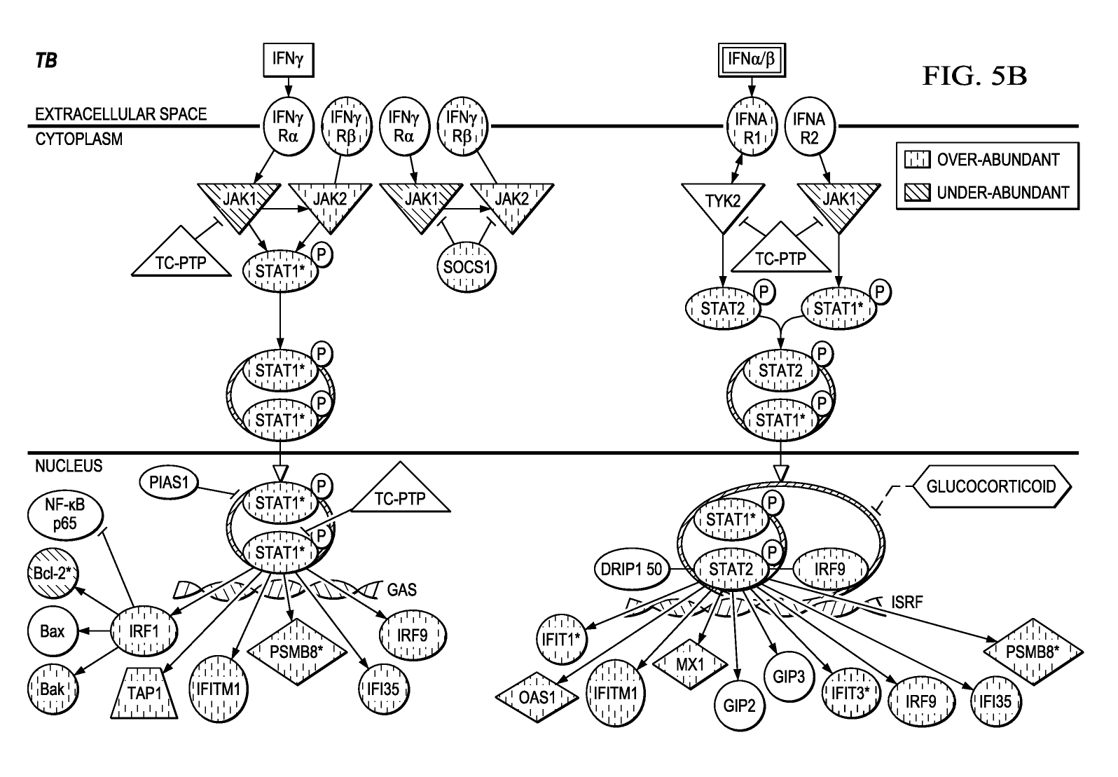

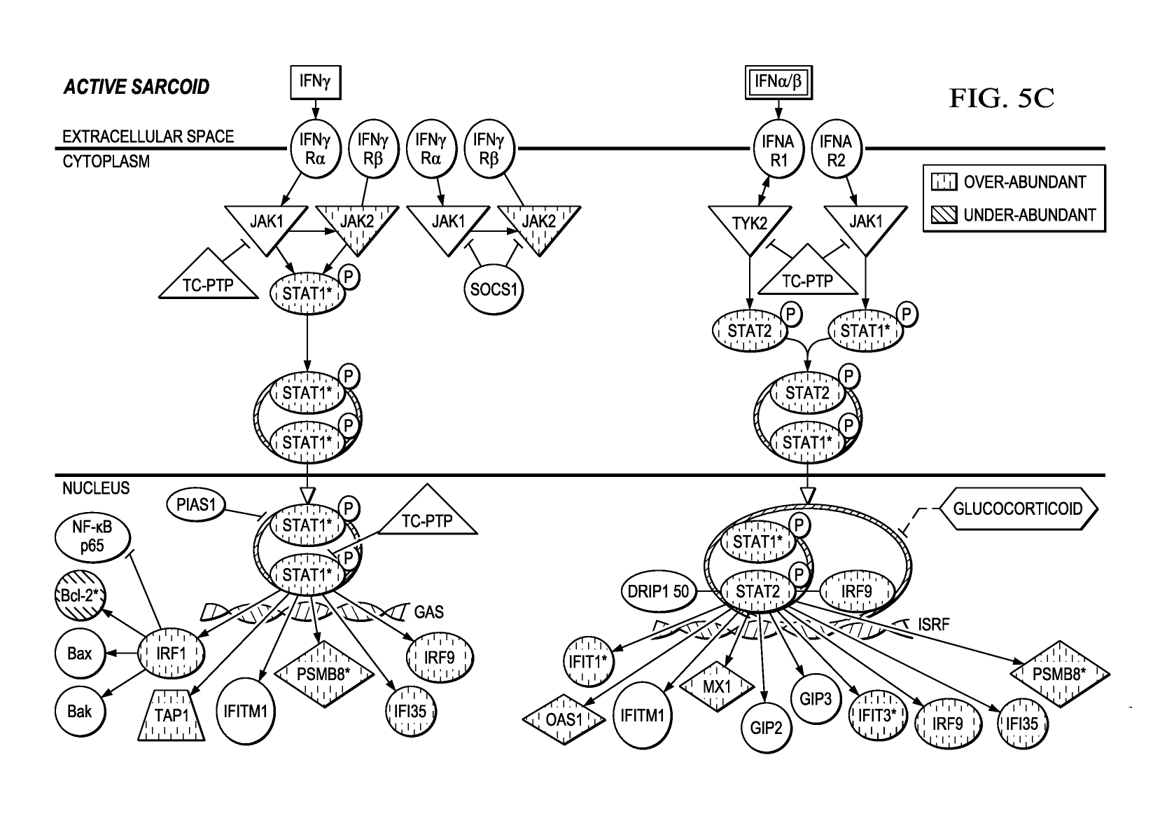

To carry out modular analysis all detectable genes (15,212 transcripts) in the whole Training set dataset were analysed. Each module corresponds to a set of co-regulated genes that were assigned biological functions by unbiased literature profiling (3). FIGS. 4A to 4E shows modular analysis of the Training Set shows the similarity of the biological pathways associated with TB and sarcoidosis (particularly overexpression of the IFN modules), differing from pneumonia and lung cancer (particularly overexpression of the inflammation modules). FIG. 4A shows gene expression levels of all transcripts that were significantly detected compared to background hybridisation (15,212 transcripts, p<0.01) were compared in the Training Set between each patient group: TB, active sarcoidosis, non-active sarcoidosis, pneumonia, lung cancer, to the healthy controls. Each module corresponds to a set of co-regulated genes that were assigned biological functions by unbiased literature profiling. A red dot indicates significant over-abundance of transcripts and a blue dot indicates significant under-abundance (p<0.05). The colour intensity correlates with the percentage of genes in that module that are significantly differentially expressed. The modular analysis can also be represented in graphical form as shown in 4B-E, including both the Training and Test Set samples. FIG. 4B shows the percentage of genes significantly overexpressed in the 3 IFN modules for each disease. FIG. 4C shows the fold change of the expression of the genes present in the IFN modules compared to the controls. FIG. 4D shows the percentage of genes significantly overexpressed in the 5 inflammation modules for each disease. FIG. 4E shows the fold change of the expression of the genes present in the inflammation modules compared to the controls. TB and active sarcoidosis show significant overexpression of the IFN modules compared to the other pulmonary disease groups (FIG. 4A). In contrast the pneumonia and cancer patients showed significant overexpression of the inflammation modules compared to TB and active sarcoidosis. These findings were then verified by modular analysis of the Test Set (Figure E7). The modular analysis therefore also substantiates our results determined from pathways linked to the 1446-transcripts signature described earlier (FIG. 2). TB patients showed a significant increase in the number of IFN genes (FIG. 4B), and their degree of expression (FIG. 4C), compared to the active sarcoidosis patients, demonstrating a quantitative difference in the IFN-inducible signature between TB and active sarcoidosis (FIG. 4B-C) The same genes in the IFN module that were overexpressed in the active sarcoidosis patients were also overexpressed in the TB patients (data not shown). Pneumonia and lung cancer showed a significant increase in the number of genes present in the inflammation modules (FIG. 4D), and their degree of expression (FIG. 4E), in comparison to TB and active sarcoidosis (FIG. 4A, D-E). Pneumonia patients also showed a significant overexpression of the number of genes present in the neutrophil module compared to all the other pulmonary diseases (Figure E8). Whole blood gene expression may correlate with the blood's cell composition or with the gene expression in particular cellular populations. For the neutrophil genes there was a significant correlation between the neutrophil module and the neutrophil count for all the pneumonia patients versus controls (Pearson's correlation, p<0.0001). The second data mining approach, comparison IPA, only used those genes that were differentially expressed between each disease group and a set of controls matched by ethnicity and gender (≧1.5 fold change from the mean of the controls, Mann Whitney Benjamini Hochberg p<0.01; TB=2524, active sarcoidosis=1391, pneumonia=2801 and lung cancer=1626 differentially expressed transcripts). FIG. 5A shows a comparison Ingenuity Pathway Analysis of the four disease groups compared to their matched controls reveals the four most significant pathways. Differentially expressed genes were derived from the Training Set by comparing each disease to healthy controls matched for ethnicity and gender: TB=2524, active sarcoidosis=1391, pneumonia=2801 and lung cancer=1626 transcripts (≧1.5 fold change from the mean of the controls, Mann Whitney Benjamini Hochberg p<0.01). FIG. 5A shows the IPA canonical pathways was used to determined the most significant pathways (i-iv) associated with each disease relative to the other diseases (Fisher's exact Benjamini Hochberg). The bottom x-axis and bars of each graph indicates the log(p-value) and the top x-axis and line indicates the percentage of genes present in the pathway. The genes in the EIF2 signalling pathway are predominately under-abundant genes however the genes in the other three pathways are predominantly over-abundant relative to the controls. Pathways above the blue dotted line are significant (p<0.05). FIGS. 5B, 5C and 5D show the interferon signalling IPA pathway is overlaid onto each disease group. Coloured genes are differentially expressed in that disease group compared to their matched controls (Fisher's exact p<0.05). Red genes represent over-abundance and green under-abundance.

The Comparison IPA reveals the most significant pathways when comparing across the diseases. The top four significant pathways were related to protein synthesis (EIF2 signalling) and immune response pathways (interferon signalling, role of pattern recognition receptors in recognition of bacteria and viruses and antigen presentation pathway)(FIG. 5A). The prominence of the EIF2 signalling pathway was driven by the pneumonia patients. The genes were significantly under-abundant in the pneumonia patients compared to the other pulmonary diseases. Many other genes related to protein synthesis (including eukaryotic initiation factors and ribosomal proteins) and the unfolded protein response (a stress response to excessive protein synthesis), were also significantly under-abundant in the pneumonia patients compared to the other pulmonary diseases, e.g. PERK, CHOP, ABCE1 (data not shown). The significance of the three immune response pathways was driven predominantly by the TB patients, but also by the sarcoidosis patients. The pathways were more significant (bottom x-axis bar graph in FIG. 5A) and contained a higher number of genes (top x-axis line graph in FIG. 5A) in both TB and active sarcoidosis than compared to the other pulmonary diseases, again demonstrating the similarity of the biological pathways underlying these pulmonary granulomatous diseases. However the interferon signalling pathway was more significant (bottom x-axis bar graph FIG. 5A) and contained a higher number of genes in the TB than the active sarcoidosis patients and were not represented in pneumonia and lung cancer (top x-axis line graph FIG. 5A, FIG. 5B and FIG. 5C).

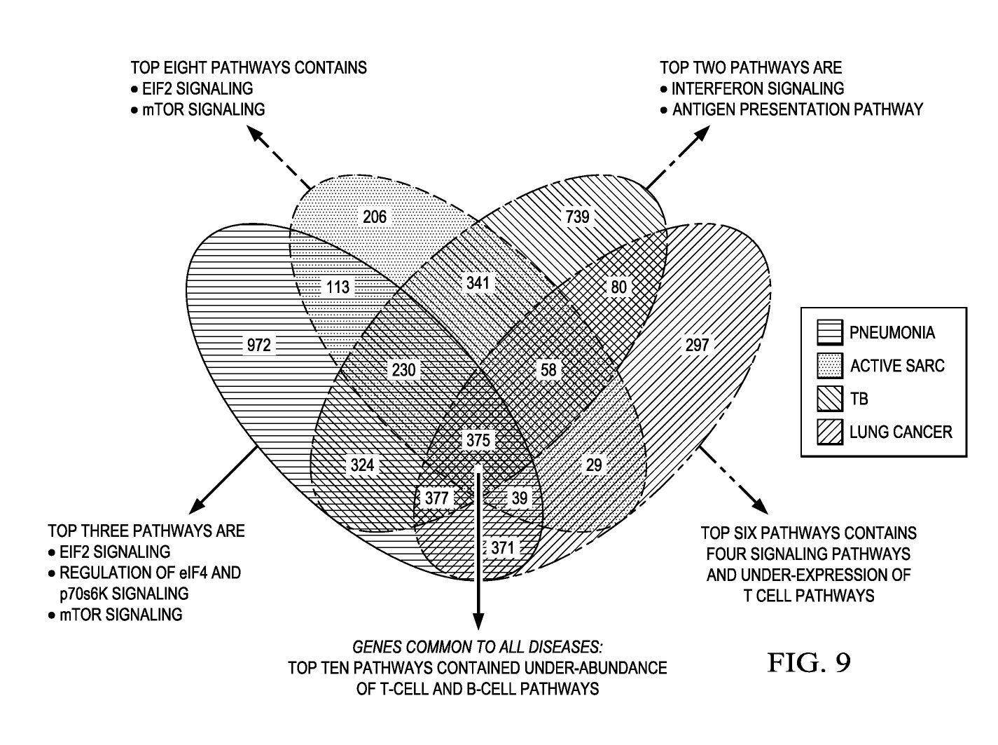

The third data mining strategy just examined the top 50 over-abundant differentially expressed transcripts for each disease. It could be seen that the transcripts correlate well with the findings from the modular and IPA analysis as both the TB and active sarcoidosis top 50 over-abundant transcripts were dominated by IFN-inducible genes e.g. IFITM3 (SEQ ID NO.:989), IFIT3 (SEQ ID NO.:1279), GBP1 (SEQ ID NO.:226), GBP6 (SEQ ID NO.:1409), CXCL10 (SEQ ID NO.:1298), OAS1 (SEQ ID NO.:790), STAT1 (SEQ ID NO.:995), IFI44L (SEQ ID NO.:1013), FCGR1B (SEQ ID NO.:63) (Table 6). However the expression fold change was much higher in the TB patients than the active sarcoidosis patients. In addition the pneumonia top 50 over-abundant transcripts were dominated by antimicrobial neutrophil-related genes e.g., ELANE (SEQ ID NO.:330), DEFA1B (SEQ ID NO.:1024), MMP8 (SEQ ID NO.:521), CAMP (SEQ ID NO.:40), DEFA3 (SEQ ID NO.:1088), DEFA4 (SEQ ID NO.:231), MPO (SEQ ID NO.:1287), LTF (SEQ ID NO.:506). The genes FCGR1A, B and C ((SEQ ID NO.:1109, 63, 50, respectively)) were over-abundant in the top 50 transcripts of all four pulmonary diseases. A 4-set Venn diagram of the differentially expressed genes was able to demonstrate the unique genes for each disease group (FIG. 9 and Table 7). There were over three times the number of unique TB genes than unique active sarcoidosis genes of which only the TB unique genes were significantly associated with the IPA IFN-signalling pathway. The unique pneumonia genes were associated with an under-abundance of pathways related to protein synthesis. The unique lung cancer genes were associated with over-abundance of inflammation related pathways. The overlapping genes common to all four disease groups were significantly associated with under-abundance of T and B cell pathways.

TB and pneumonia patients after treatment showed a diminishment of their transcriptional profiles to resemble the controls however the sarcoidosis patients who respond to glucocorticoids showed a significant increase in their transcriptional activity.

FIGS. 6A to 6D show both modular analysis and molecular distance to health reveal that the blood transcriptome of the pneumonia and TB patients after successfully completing treatment are no different from the healthy controls however the sarcoidosis patients show an overexpression of inflammation genes during a clinically successful response to glucocorticoids. FIG. 6A shows a modular analysis for gene expression levels of all transcripts that were significantly detected compared to background hybridisation (p<0.01) were compared between the healthy controls and each of the following the patient groups: pre-treatment pneumonia, post-treatment pneumonia patients and pre-treatment sarcoidosis, inadequate treatment response sarcoidosis and good treatment response sarcoidosis patients. A red dot indicates significant over-abundance of transcripts and a blue dot indicates under-abundance (p<0.05). The colour intensity correlates with the percentage of genes in that module that are significantly differentially expressed. MDTH demonstrates the quantification of transcriptional change after treatment in the 1446-transcripts relative to controls for pre-treatment pneumonia, post-treatment pneumonia patients, pre-treatment TB and post-treatment TB and and pre-treatment sarcoidosis, inadequate treatment response sarcoidosis and good treatment response sarcoidosis patients. The mean and SEM was compared between each disease group (ANOVA with Tukey's multiple comparison test). FIG. 6B, Pneumonia patients; FIG. 6C, TB patients from the Bloom et al, 2012 (12), study carried out in South Africa, the controls in this study were participants with latent TB; FIG. 6D Sarcoidosis patients.

More specifically, having determined the blood transcriptional signatures of untreated patients with the pulmonary granulomatous diseases TB and sarcoidosis and the infectious disease community and acute lung diseases of acquired pneumonia we next sought to examine their transcriptional response to treatment. The pneumonia patients were all followed-up at least 6 weeks after their hospital discharge and showed a good clinical response to their treatment with standard antibiotics (clinical data not shown but available). Using two completely different data mining strategies, modular analysis (all detectable transcripts were analysed) and MDTH (only the 1446-transcripts were analysed), it could be seen that the pneumonia patients after successful treatment showed a reversal of their transcriptional profiles such that there was no significant difference between the pneumonia post-treatment transcriptional profiles and the healthy controls (FIGS. 6A & B). We have previously studied the blood transcriptional response of a cohort of active TB patients from South Africa before and after successful anti-TB treatment (4). Therefore we used the same 1446-transcripts that were derived from this present study to assess the transcriptional response of these South African TB patients before and after treatment, compared to their latent TB controls. The MDTH score of the untreated active TB patients were significantly different from the latent TB controls however the transcriptional response after treatment again reversed with no significant difference between the treated active TB patients and the latent TB controls (FIG. 6C).

The treated sarcoidosis patients showed a variable clinical response after immunosuppressive treatment initiation as determined by their practising physician (clinical data not shown but available). If the physician increased their treatment at their clinic follow-up the patient was categorised as having an ‘inadequate treatment response’ but if the physician continued the same treatment or reduced their treatment this was categorised as having a ‘good treatment response’. Applying the same two data mining strategies as used for the pneumonia patients it could clearly be seen that the sarcoidosis patients who had a good clinical response to glucocorticoids had a significant overexpression of inflammatory genes that was not seen when the same or the different sarcoidosis patients had an inadequate response to immunosuppressive treatment (FIGS. 6A & D). The majority of the inflammatory genes that were overexpressed in the untreated pneumonia and lung cancer patients were also overexpressed in the good-treatment response sarcoidosis patients (Table 8), but many more transcripts were overexpressed in the good-treatment response sarcoidosis patients (clinical data not shown but available). The term inflammation comprises many forms and therefore there is a diversity of genes that are called inflammatory. Interestingly many of the top 50 overexpressed inflammatory genes in the good-treatment response sarcoidosis patients are known to be anti-inflammatory genes which are invariably induced alongside proinflammatory genes in what is termed an inflammatory response, e.g., IL1R2 (SEQ ID NO.:1007), DUSP1, IL18R (SEQ ID NO.:239), C-FOS, IκBα and MAPK1, as well as pro-inflammatory genes (Table 8).

The interferon-inducible genes were most abundant in the neutrophils in both TB and sarcoidosis. It was previously shown in the Berry, et al., 2010 publication (5) that the active TB signature was dominated by a neutrophil-driven IFN-inducible gene profile, consisting of both IFN-γ and type I IFN-αβ signalling (5). Therefore the inventors identified the main cell populations driving the IFN-inducible signature in the active sarcoidosis patients. A new cohort of patients (TB and active sarcoidosis) were recruited and controls to test the same IFN-inducible genes as used in the Berry, et al., 2010 publication (5) in the purified leucocyte populations of TB and sarcoidosis patients who had an IFN-inducible signature present in whole blood (Table 9).

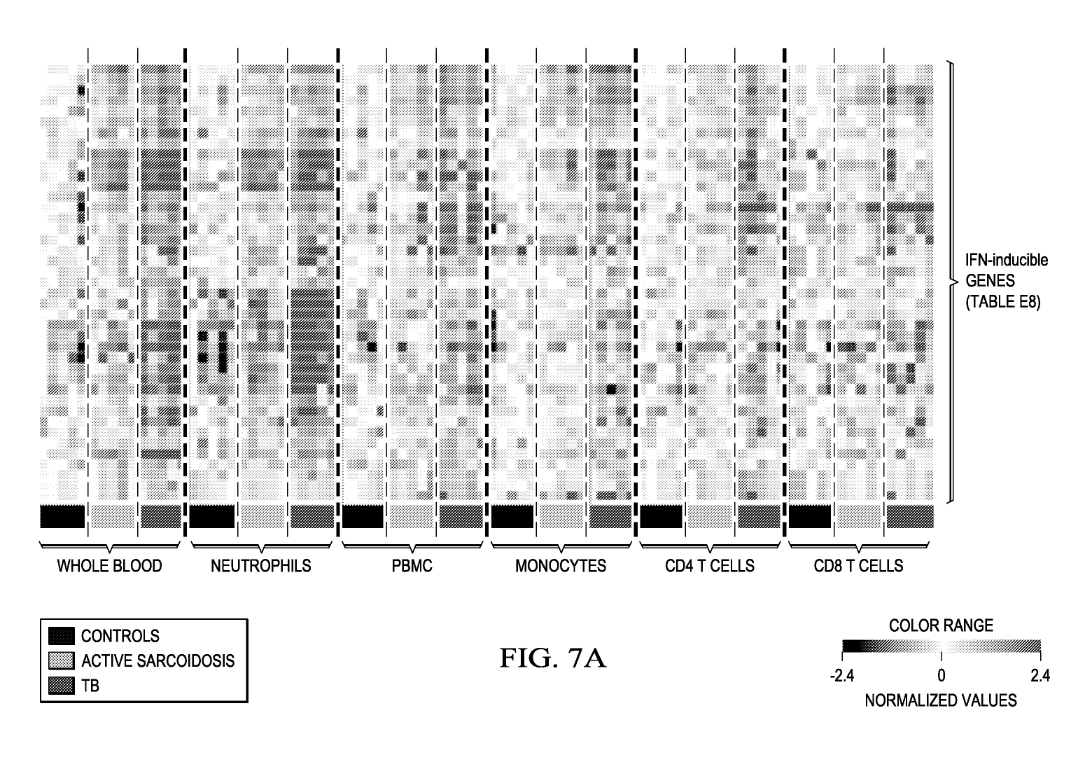

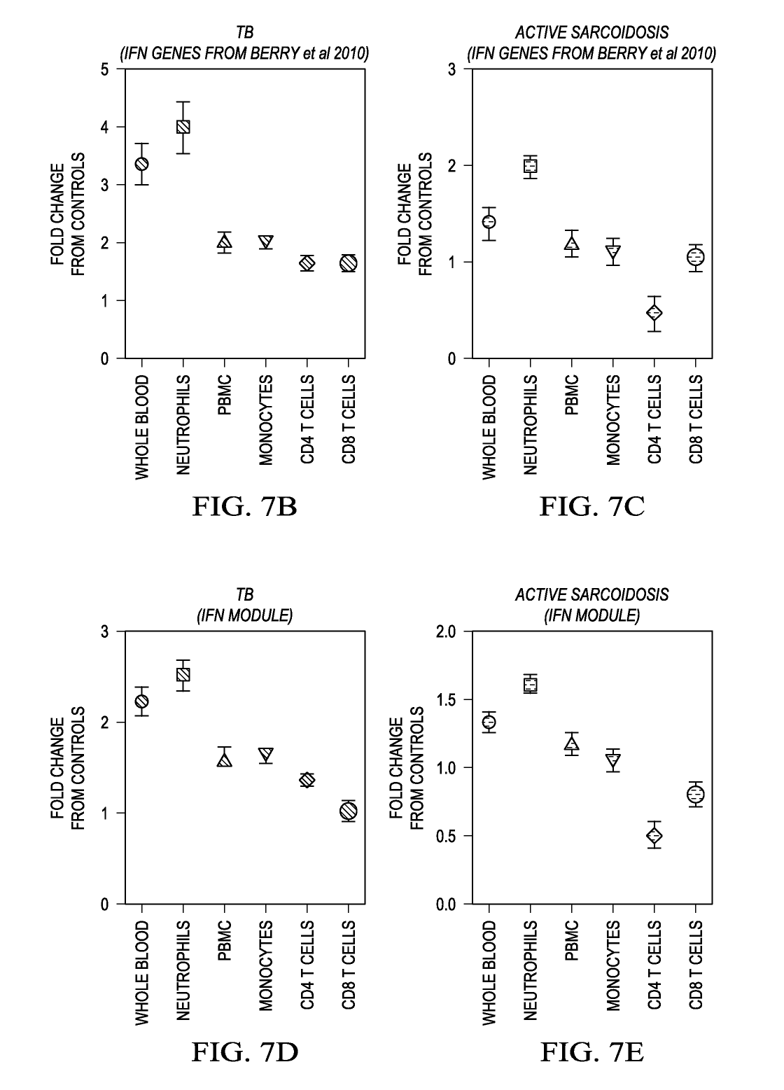

FIGS. 7A to 7E show that interferon-inducible gene expression is most abundant in the neutrophils in both TB and sarcoidosis. The expression of interferon-inducible genes was measured in purified leucocyte populations from whole blood. FIG. 7A is a heatmap that shows the expression of IFN-inducible transcripts, from the Berry, et al., 2010 study (5), for each disease group normalised to the controls for that cell type. FIG. 7B shows the expression fold change in the TB samples of the same IFN-inducible transcripts. FIG. 7C shows the expression fold change in the sarcoidosis samples of the same IFN-inducible transcripts. FIG. 7D shows the expression fold change in the TB samples of all the genes present in the three interferon modules compared to the controls. FIG. 7E shows the expression fold change in the sarcoidosis samples of all the genes present in the three interferon modules compared to the controls.

Again the neutrophils displayed the highest relative abundance of IFN-inducible genes in active TB (FIGS. 7A, 7B & 7D). The neutrophils also had the highest abundance of IFN-inducible genes in the sarcoidosis patients, although to a lesser extent than was seen in the TB patients (FIGS. 7A, 7C & 7E). The monocytes showed a higher abundance of IFN-inducible genes than the lymphocytes in both the TB and sarcoidosis patients (FIG. 7A-E), as previously shown (5).

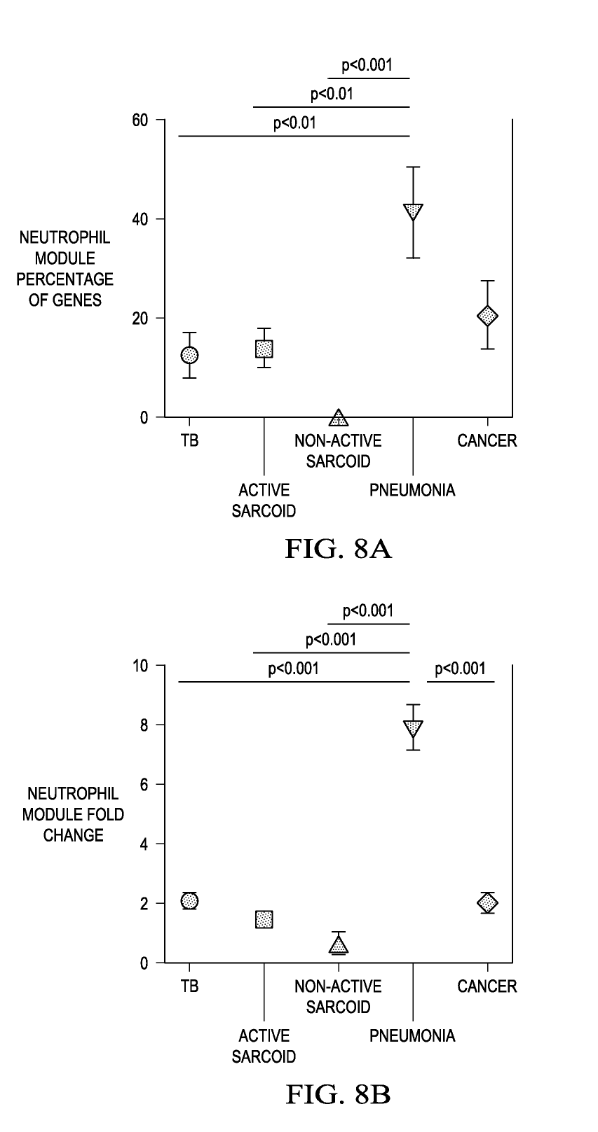

FIG. 8 shows the results for each of the pulmonary diseases using the genes expressed in a neutrophil module. FIG. 8A shows the percentage of genes significantly overexpressed in the neutrophil module for each disease in both the Training and Test set. FIG. 8B shows the fold change of the expression of the genes present in the neutrophil module compared to the controls.

FIG. 9 is a 4-set Venn diagram comparing the differentially expressed genes for each disease group compared to their ethnicity and gender matched controls. Differentially expressed genes were derived from the Training Set by comparing each disease to healthy controls matched for ethnicity and gender: TB=2524, active sarcoidosis=1391, pneumonia=2801 and lung cancer=1626 transcripts (≧1.5 fold change from the mean of the controls, Mann Whitney Benjamini Hochberg p<0.01). The 4-set Venn diagram was created using Venny (13). IPA canonical pathways was used to determined the most significant pathways associated with the unique transcripts for each disease (Fisher's exact p<0.05). Active Sarc=active sarcoidosis.

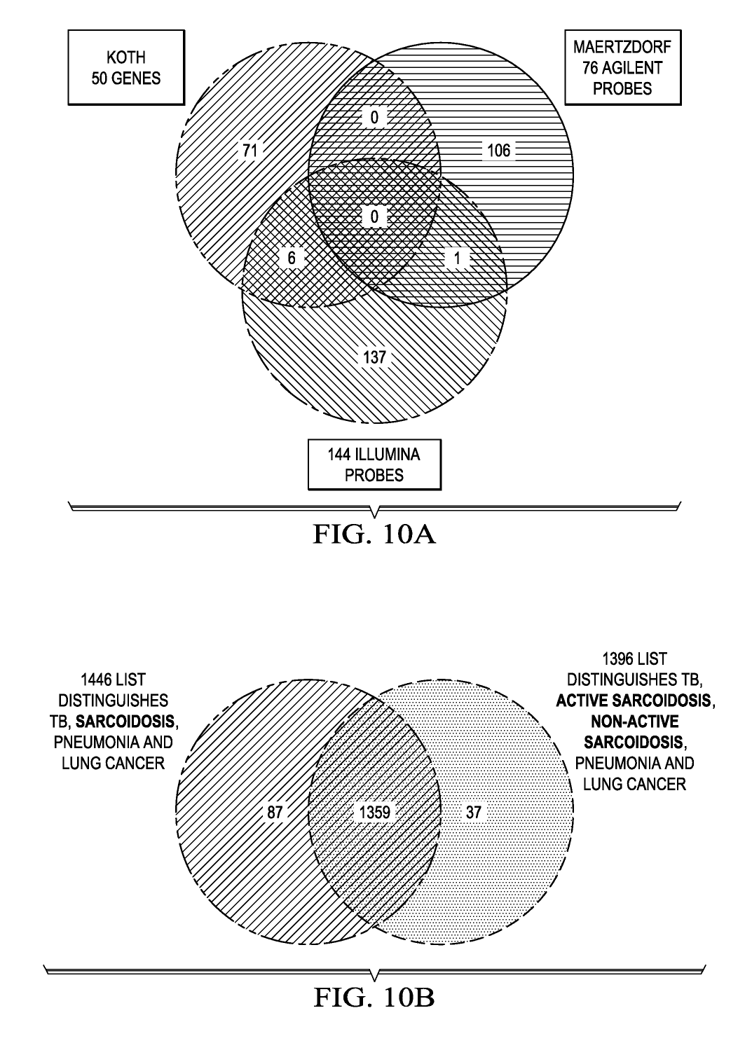

FIG. 10A is a Venn diagram comparing the gene lists used in the class prediction. The gene lists were obtained from this study (144 Illumina probes), Maertzdorf, et al., study (8) (100 Agilent probes of which only 76 probes were recognised as genes using NIH DAVID Gene ID Conversion Tool) and Koth, et al., study (7) (50 genes obtained from a Affymetrix platform although analysis also included data obtained from alternative studies from GEO databases which used other microarray platforms the majority from the Berry et al, 2010 (5) by current applicants). In the Illumina platform used to compare these lists some genes are represented by more than one transcript for example the 50 genes in Koth et al study (7) translate to 77 Illumina probes/transcripts.

144-transcripts were able to distinguish with good sensitivity and specificity the TB patients from the other pulmonary diseases and healthy controls.

Although the transcriptional profiles of the TB and active sarcoidosis patients appeared very similar we wished to determine if a gene list could distinguish the TB samples, from all the other patient and control samples. Therefore we compared the TB transcriptional profiles to the most similar group, active sarcoidosis, to derive a set of differentially expressed genes. 144 transcripts were differentially expressed between the TB and active sarcoidosis transcriptional profiles from the Training Set (significance analysis of microarray q<0.05, fold change≧1.5). Many of the transcripts were IFN-inducible genes and were all over-abundant in the TB profiles compared to the active sarcoidosis profiles (Table 2). Two recent publications also described gene lists that could distinguish TB from all sarcoidosis patients (7, 8). These previously published gene lists were derived from different cohorts and used different microarray platforms. We used a class prediction machine learned algorithm, support vector machines (SVM), to test our gene list and the two previously published gene lists for their ability to predict whether a transcriptional profile belonged to a TB patient or not. The prediction model is built using the transcriptional signature from samples with known disease-types to predict the classification of a new collection of samples. The SVM model should therefore be built in one study cohort and run in an independent cohort to prevent over-fitting the predictive signature. This was possible for all our cohorts. Where the study cohorts used a different microarray platform the SVM model had to be re-built in that cohort. However to reduce the effects of over-fitting the same parameters were used every time the SVM model was built.

TABLE 2
144 transcripts. The 144 transcripts are differentially
expressed genes between the TB and active sarcoidosis
profiles in the Training Set
(significance analysis of microarray q < 0.05, fold change ≧ 1.5).

| | Fold Change | |
|---|---|---|
| | TB vs Active | |
| Symbol | Sarcold | Regulation |
| C1QB | 10.6 | UP |
| LOC100133565 | 6.4 | UP |
| TDRD9 | 5.3 | UP |
| ABCA2 | 5.3 | UP |
| SMARCD3 | 5.3 | UP |
| CACNA1E | 5.1 | UP |
| HP | 4.2 | UP |
| NTN3 | 4.2 | UP |
| LOC100008589 | 3.3 | UP |
| CARD17 | 3.3 | UP |
| LOC441763 | 3.2 | UP |
| ERLIN1 | 3.1 | UP |
| SLPI | 3.1 | UP |
| SLC26AB | 2.9 | UP |
| AIM2 | 2.8 | UP |
| INCA | 2.8 | UP |
| OPLAH | 2.7 | UP |
| LPCAT2 | 2.6 | UP |
| SEPT4 | 2.5 | UP |
| DISC1 | 2.5 | UP |
| 2FP91 | 2.5 | UP |
| UBE2J2 | 2.4 | UP |
| KREMEN1 | 2.4 | UP |
| ALPL | 2.3 | UP |
| LOC100008589 | 2.3 | UP |
| KCN816 | 2.2 | UP |
| C19orf59 | 2.2 | UP |
| FCGR1A | 2.2 | UP |
| SPATA13 | 2.2 | UP |
| ADM | 2.2 | UP |
| CDKSRAP2 | 2.2 | UP |
| SNORA73B | 2.2 | UP |
| TncRNA | 2.1 | UP |
| PPAP2C | 2.1 | UP |
| IFITM3 | 2.1 | UP |
| FCGR1B | 2.1 | UP |
| JMJD6 | 2.1 | UP |
| HIST2H3D | 2.1 | UP |
| LMNB1 | 2.0 | UP |
| S100A12 | 2.0 | UP |
| FCGR1C | 2.0 | UP |
| LOC653591 | 2.0 | UP |
| LOC100132394 | 2.0 | UP |
| SLC26A8 | 2.0 | UP |
| ANXA3 | 2.0 | UP |
| NLRC4 | 1.9 | UP |
| LOC100134364 | 1.9 | UP |
| LILRA6 | 1.9 | UP |
| LOC653610 | 1.9 | UP |
| CST7 | 1.9 | UP |
| LILRB4 | 1.9 | UP |
| MSL3L1 | 1.9 | UP |
| HIST2H2BG | 1.9 | UP |
| OSM | 1.9 | UP |
| LILRAS | 1.9 | UP |
| GPR97 | 1.9 | UP |
| HIST2H2AC | 1.9 | UP |
| LILRAS | 1.8 | UP |
| TLR5 | 1.8 | UP |
| LOC728417 | 1.8 | UP |
| MSL3 | 1.8 | UP |
| HPSE | 1.8 | UP |
| RGL4 | 1.8 | UP |
| CYP1B1 | 1.8 | UP |
| HIST2H2AA3 | 1.8 | UP |
| AGTRAP | 1.8 | UP |
| PFKB3 | 1.8 | UP |
| GNG8 | 1.8 | UP |
| LIB4R | 1.8 | UP |
| H2AFJ | 1.8 | UP |
| LILRA5 | 1.8 | UP |
| ABCA1 | 1.8 | UP |
| SULT1B1 | 1.8 | UP |
| GYG1 | 1.7 | UP |
| IFITM1 | 1.7 | UP |
| SVIL | 1.7 | UP |
| DGAT2 | 1.7 | UP |
| MEFV | 1.7 | UP |
| PIM3 | 1.7 | UP |
| MTRF1L | 1.7 | UP |
| MAZ | 1.7 | UP |
| HIST2H2AA4 | 1.7 | UP |
| LOC728519 | 1.7 | UP |
| SMARCD3 | 1.7 | UP |
| LOC641710 | 1.7 | UP |
| HIST2H2BE | 1.7 | UP |
| ITPRIPL2 | 1.7 | UP |
| FXBP5 | 1.7 | UP |
| IFNAR1 | 1.6 | UP |
| LY96 | 1.6 | UP |
| LOC728417 | 1.6 | UP |
| DHRS13 | 1.6 | UP |
| IL18R1 | 1.6 | UP |
| GPR109B | 1.6 | UP |
| AGTRAP | 1.6 | UP |
| GPR1D9A | 1.6 | UP |
| PLAC8 | 1.6 | UP |
| BAGE5 | 1.6 | UP |
| DUSP3 | 1.6 | UP |
| SLC22A4 | 1.6 | UP |
| LOC645159 | 1.6 | UP |
| 1L4R | 1.6 | UP |
| FLI32255 | 1.6 | UP |
| HIST2H2AA3 | 1.6 | UP |
| PLAC8 | 2.6 | UP |
| SH3GLB1 | 1.6 | UP |
| PLSCR1 | 1.6 | UP |
| IFI35 | 1.6 | UP |
| TAOK1 | 1.6 | UP |
| MCTP1 | 1.6 | UP |
| CEACAM1 | 1.6 | UP |
| B4GALT5 | 1.6 | UP |
| COP1 | 1.6 | UP |
| PROK2 | 1.6 | UP |
| IFI30 | 1.6 | UP |
| FCER1G | 1.5 | UP |
| 2NF438 | 1.5 | UP |
| EEF1D | 1.5 | UP |
| MIR21 | 1.5 | UP |
| NGFRAP1 | 1.5 | UP |
| PGS1 | 1.5 | UP |
| KIF1B | 1.5 | UP |
| C16orf57 | 1.5 | UP |
| ANKRD33 | 1.5 | UP |
| MXD4 | −1.5 | DOWN |
| 2SCAN18 | −1.6 | DOWN |
| MEF2D | −1.6 | DOWN |
| BHLHB2 | −1.7 | DOWN |
| CLC | −2.3 | DOWN |
| FCER1A | −2.5 | DOWN |
| SRGAP3 | −2.6 | DOWN |
| FLI43093 | −2.8 | DOWN |
| CCR3 | −2.9 | DOWN |
| EMR4 | −3.0 | DOWN |
| ZNf792 | −3.1 | DOWN |
| C10orf33 | −3.5 | DOWN |
| CACNG6 | −3.8 | DOWN |
| P2RY10 | −4.2 | DOWN |
| GATA2 | −4.6 | DOWN |
| EMR4P | −6.6 | DOWN |
| ESPN | −7.0 | DOWN |
| EMR4 | −9.3 | DOWN |

The 144 Illumina transcripts showed good sensitivity (above 80%) and specificity (above 90%) in all three independent cohorts from our study (Training, Test and Validation Sets) and when using an external cohort from the Maertzdorf et al study. The 100 Agilent transcripts from the Maertzdorf et al 2012 study were also tested (7). Only 76 of these transcripts were recognised as genes by NIH DAVID Gene ID Conversion Tool. The same SVM parameters as used earlier were then applied using the Maertzdorf et al transcripts in our three independent cohorts (Training, Test and Validation Sets). The sensitivity however was much lower (45-56%), with similar specificity (above 90%). The 50 genes from the Koth et al 2011 (7) study run using an Affymetrix platform were also tested. The same SVM parameters were again applied to all our independent cohorts (Training, Test and Validation Sets). The sensitivity of this gene list was also lower (75-45%), with similar specificity (above 87%), than for our 144-transcripts. Neither the Koth et al 2011 (7) or the Maertzdorf et al 2012 (8) studies reported testing their derived gene lists in independent cohorts. As these study tested the 144-transcripts list from the present applicants (Bloom, O'Garra et al., to be submitted), in both internal and external independent cohorts this is likely to have improved the validity of the transcript list as a discriminative marker, and may explain why there was little overlap between their gene lists or overlap with the present applicants' 144 gene list (Figure E10). Tables 3, 4 and 5. Class prediction. Class prediction was performed using support vector machines (SVM).

Table 2 (above) shows the 144 transcripts derived from the Training Set which were then used to build the SVM model, the model was then run in the other four cohorts Table 3 (just below).

The 144 transcripts derived from the Training Set in this present
study, Bloom et al (Illumina), were tested in the cohorts below:

| | Present study | Present study | | |
|---|---|---|---|---|
| | Training Set | Test Set | | Maertzdorf |
| | (controls, TB, | (controls, TB, | Present study | et al |
| | sarcoid, | sarcoid, | Validation Set | controls, |
| | cancer, | cancer, | (controls, TB, | TB, |
| | pneumonia) | pneumonia) | sarcoid) | (sarcoid) |
| Sensitivity | 88% | 82% | 88% | 88% |
| Specificity | 94% | 91% | 92% | 97% |

Table 4 (below). The 100 Agilent transcripts from the Maertzdorf et al study (8) translated to 76 recognised genes using the DAVID gene converter. The SVM model was built in the Training Set and run in the Test and Validation Sets.

The 76 recognised genes out of the 100 probes from the Maertzdorf
et al study (Agilent) were tested in the cohorts below:

| | Present study | Present study | | Maertzdorf |
|---|---|---|---|---|
| | Training Set | Test Set | Present study | et al |
| | (controls, TB, | (controls, TB, | Validation Set | (controls, |
| | sarcoid, cancer, | sarcoid, cancer, | (controls, TB, | TB, |
| | pneumonia) | pneumonia) | sarcoid) | sarcoid) |
| Sensitivity | 56% | 45% | 75% | 88% |
| | | | | (as stated |
| | | | | in their |
| | | | | publication) |
| Specificity | 96% | 92% | 92% | 97% |
| | | | | (as stated |
| | | | | in their |
| | | | | publication) |

Table 5 (below) shows the 50 genes from the Koth et al study (7) were used to build the last SVM model in the Training Set and run in the Test and Validation Sets. N/A=not applicable.

The 50 genes from the Koth et al study (Affymetrix) were
tested in the cohorts below:

| | Present study | Present study | | Koth et al |
|---|---|---|---|---|
| | Training Set | Test Set | Present study | (sarcoid and |
| | (controls, TB, | (controls, TB, | Validation Set | all cohorts |
| | sarcoid, cancer, | sarcoid, cancer, | (controls, TB, | from Berry |
| | pneumonia) | pneumonia) | sarcoid) | et al study) |
| Sensitivity | 75% | 45% | 50% | Not shown |
| | | | | in their |
| | | | | publication |
| Specificity | 92% | 87% | 92% | Not shown |
| | | | | in their |
| | | | | publication |

Table 6 (below). The top 50 differentially expressed transcripts for each disease compared to matched controls (from the present applicants' study). Differentially expressed genes were derived from the Training Set by comparing each disease to healthy controls matched for ethnicity and gender: TB=2524, active sarcoidosis=1391, pneumonia=2801 and lung cancer=1626 transcripts (≧1.5 fold change from the mean of the controls, Mann Whitney Benjamini Hochberg p<0.01).

| TB | | Active sarcoidosis | | Pneumonia | | Lung cancer | |
|---|---|---|---|---|---|---|---|
| Fold Change | Gene Symbol | Fold Change | Symbol | Fold Change | Gene Symbol | Fold Change | Gene Symbol |
| 21 | ANKRD22 | 8.1 | FCGR1A | 15.8 | OLFM4 | 6.1 | ARG1 |
| 18.5 | FCGR1A | 7.9 | ANKRD22 | 12.7 | LTF | 5.5 | TPST1 |
| 17.4 | SERPING1 | 7.4 | FCGR1C | 12.6 | VNN1 | 5.4 | FCGR1A |
| 15.1 | BATF2 | 7.1 | FCGR1B | 12.4 | HP | 5.2 | C19orf59 |
| 14.9 | FCGR1C | 6.4 | SERPING1 | 12.3 | DEFA4 | 4.6 | SLPI |
| 13.7 | FCGR1B | 6.2 | FCGR1B | 11.3 | OPLAH | 4.5 | FCGR1B |
| 13.3 | ANKRD22 | 6 | BATF2 | 11.2 | CEACAM8 | 4.3 | IL1R1 |
| 13.1 | FCGR1B | 5.5 | GBP5 | 11 | DEFA1B | 4.1 | FCGR1C |
| 10.8 | LOC728744 | 5.3 | GBP1 | 10.1 | ELANE | 4.1 | TDRD9 |
| 10 | IFITM3 | 5.1 | IFIT3 | 9.4 | C19orf59 | 4.1 | SLC26A8 |
| 9.5 | EPSTI1 | 5 | ANKRD22 | 9.2 | ARGI | 4.1 | FCGR1B |
| 8.7 | GBPS | 4.9 | LOC728744 | 8.7 | CDK5RAP2 | 4.1 | CLEC4D |
| 8.7 | IFI44L | 4.8 | GBP1 | 8.6 | DEFA1B | 4 | LOC100132858 |
| 8.4 | GBP6 | 4.8 | EPSTI1 | 8.4 | DEFA3 | 3.9 | SLC22A4 |
| 8.1 | GBP1 | 4.6 | IFI44L | 8.3 | DEFA1B | 3.8 | LOC100133177 |
| 7.8 | LOC400759 | 4.6 | INDO | 8.1 | FCGR1A | 3.7 | SIPA1L2 |
| 7.7 | IFIT3 | 4 | IFITM3 | 7.9 | MMP8 | 3.6 | ANXA3 |
| 7.6 | AIM2 | 4 | GBP6 | 7.4 | FCGR1B | 3.6 | LIMK2 |
| 7.3 | SEPT4 | 4 | RSAD2 | 7.3 | SLPI | 3.5 | TMEM88 |
| 7.1 | C1QB | 3.9 | DHRS9 | 7.2 | SLC26A8 | 3.5 | MMP9 |
| 6.9 | GBP1 | 3.7 | TNFAIP6 | 7.1 | MAPK14 | 3.5 | ASPRV1 |
| 6.9 | RSAD2 | 3.7 | IFIT3 | 7.1 | CAMP | 3.5 | MANSC1 |
| 6.4 | RTP4 | 3.5 | P2RY14 | 6.7 | NLRC4 | 3.5 | TLR5 |
| 6.1 | CARD17 | 3.4 | DHRS9 | 6.4 | FCAR | 3.5 | CD163 |
| 5.9 | IFIT3 | 3.4 | IDO1 | 6.3 | RNASE3 | 3.4 | CAMP |
| 5.6 | CASP5 | 3.3 | STAT1 | 6.3 | FCGR1B | 3.4 | LOC642816 |
| 5.4 | CEACAM1 | 3.3 | WARS | 6.2 | NAIP | 3.4 | DPRXP4 |
| 5.4 | CARD17 | 3.2 | TIMM10 | 6.2 | OLR1 | 3.4 | LOC643313 |
| 5.3 | ISG15 | 3.1 | P2RY14 | 6.1 | FCGR1C | 3.3 | NTN3 |
| 5.2 | IFI27 | 3.1 | LOC389386 | 6.1 | ANXA3 | 3.3 | MRVI1 |
| 5.1 | TIMM10 | 3.1 | FERIL3 | 6 | DEFAI | 3.3 | F5 |
| 5 | WARS | 3 | IFIT3 | 6 | PGLYRP1 | 3.3 | SOCS3 |
| 4.8 | IFI6 | 3 | RTP4 | 6 | TCN1 | 3.3 | TncRNA |
| 4.7 | TNFAIP6 | 3 | SCO2 | 6 | ANKDD1A | 3.3 | MIR21 |
| 4.7 | PSTPIP2 | 3 | GBP4 | 5.8 | COL17A1 | 3.2 | LOC100170939 |
| 4.7 | IFI44 | 2.9 | IFIT1 | 5.8 | SLC26A8 | 3.2 | LOC100129904 |
| 4.6 | SCO2 | 2.9 | LAP3 | 5.8 | IMEM144 | 3.2 | GRB10 |
| 4.6 | FBXO6 | 2.9 | OASL | 5.8 | SAMD14 | 3.2 | ASGR2 |
| 4.5 | FER1L3 | 2.9 | CEACAM1 | 5.8 | MAPK14 | 3.2 | LOC642780 |
| 4.5 | CXCL10 | 2.9 | LIMK2 | 5.7 | RETN | 3.2 | LOC400499 |
| 4.3 | DHR59 | 2.8 | CASP5 | 5.7 | NAIP | 3.1 | FCAR |
| 4.3 | OAS1 | 2.8 | STAT1 | 5.7 | GPR84 | 3.1 | KREMENI |
| 4.3 | STAT1 | 2.8 | CCL23 | 5.6 | CASP5 | 3.1 | SLC2ZA4 |
| 4.2 | HP | 2.8 | WARS | 5.6 | MPO | 3.1 | CR1 |
| 4.2 | DHR59 | 2.7 | ATF3 | 5.6 | MMP9 | 3.1 | LOC730234 |
| 4.2 | CEACAM1 | 2.7 | IFI6 | 5.5 | CR1 | 3.1 | SLC26A8 |
| 4.2 | SLC26A8 | 2.7 | PSTPIP2 | 5.4 | MYL9 | 3.1 | C7orf53 |
| 4.2 | CACNA1E | 2.7 | ASPRVI | 5.2 | CLEC4D | 3.1 | VNN1 |
| 4.1 | OLFM4 | 2.7 | FBXO6 | 5.1 | ITGAX | 3.1 | NLRC4 |
| 4.1 | APOL6 | 2.7 | CXCL10 | 5.1 | ANKRD22 | 3.1 | LOC400499 |

Table 7 (below). The top 50 differentially expressed transcripts unique for each disease as determined by the 4-set Venn diagram (from the present applicants study). Differentially expressed genes were derived from the Training Set by comparing each disease to healthy controls matched for ethnicity and gender (≧1.5 fold change from the mean of the controls, Mann Whitney Benjamini Hochberg p<0.01). A 4-set Venn diagram was used to identify genes that were unique for each disease.

| TB | | Sarcoidosis | | Pneumonia | | Lung Cancer | |
|---|---|---|---|---|---|---|---|
| Fold Change | Gene Symbol | Fold Change | Gene Symbol | Fold Change | Gene Symbol | Fold Change | Gene Symbol |
| 7.1 | C1QB | 2.8 | CCL23 | 12.3 | DEFA4 | 5.5 | TPST1 |
| 5.2 | IFI27 | 2.1 | PIK3R6 | 10.1 | ELANE | 3.3 | MRVI1 |
| 3.5 | SMARCD3 | 2.1 | EMR4 | 7.9 | MMP8 | 3.1 | C7orf53 |
| 3.2 | SOCS1 | 2.0 | CCDC146 | 6.2 | OLR1 | 3.0 | ECHDC3 |
| 3.1 | KCNI15 | 2.0 | KLF4 | 5.8 | COLI7A1 | 2.9 | LOC651612 |
| 2.9 | LPCAT2 | 2.0 | GRINA | 5.7 | RETN | 2.9 | LOC100134660 |
| 2.8 | ZDHHC19 | 1.9 | SLC4A1 | 5.7 | GPR84 | 2.8 | TIAM2 |
| 2.8 | FYB | 1.9 | PLA2G7 | 4.6 | LOC100134379 | 2.8 | KIAA1026 |
| 2.8 | SP140 | 1.9 | GRAMD1B | 4.5 | TACSTO2 | 2.8 | HECW2 |
| 2.6 | IFITM1 | 1.9 | RAPGEF1 | 4.0 | SLC2A11 | 2.7 | TLE3 |
| 2.6 | ALAS2 | 1.8 | NXNL1 | 3.9 | LOC100130904 | 2.7 | TBC1D24 |
| 2.6 | CEACAM6 | 1.8 | TRIM5B | 3.8 | MCTP2 | 2.7 | LOC441193 |
| 2.6 | OAS2 | 1.8 | GABBR1 | 3.7 | AZU1 | 2.7 | CD163 |
| 2.5 | C1QC | 1.7 | TAGLN | 3.6 | DACH1 | 2.6 | RFX2 |
| 2.5 | LOC100133565 | 1.7 | KLI4 | 3.6 | GADD45A | 2.6 | LOC100134688 |
| 2.5 | ITGA2B | 1.7 | MFAP3L | 3.6 | NSUN7 | 2.4 | LOC642342 |
| 2.4 | LY66 | 1.7 | LOC641798 | 3.5 | CR1 | 2.3 | FXBP9L |
| 2.4 | SP140 | 1.7 | RIPK2 | 3.4 | CDK5RAP2 | 2.3 | PHF2DL1 |
| 2.4 | CASP7 | 1.7 | LOC650840 | 3.3 | LOC284648 | 2.3 | LOC402176 |
| 2.4 | GADD45G | 1.7 | FLI43093 | 3.1 | GPR177 | 2.3 | CD163 |
| 2.3 | FRMD3 | 1.7 | ASAP2 | 3.1 | CLECSA | 2.3 | OSBPL1A |
| 2.3 | CMPK2 | 1.7 | C15orf26 | 3.1 | UPB1 | 2.3 | PRMT5 |
| 2.3 | AQP10 | 1.7 | REC8 | 3.1 | SLC2A5 | 2.3 | LIBTD1 |
| 2.3 | CXCL14 | 1.7 | KIAA0319L | 3.1 | GPR177 | 2.3 | ADORA3 |
| 2.3 | I7PRIPL2 | 1.7 | GRINA | 3.1 | APP | 2.2 | SH2D3C |
| 2.3 | FAS | 1.7 | FLI30092 | 3.0 | LAMC1 | 2.2 | RBP7 |
| 2.3 | XK | 1.7 | BTN2A1 | 3.0 | REPS2 | 2.2 | ERGIC1 |
| 2.3 | CARD16 | 1.7 | HIF1A | 3.0 | PIK3CB | 2.2 | TMEM45B |
| 2.3 | SLAMF8 | 1.7 | LOC440313 | 3.0 | SMPDL3A | 2.2 | CUX1 |
| 2.2 | SELP | 1.6 | HOXA1 | 3.0 | UBE2C | 2.2 | TREM1 |
| 2.2 | NDN | 1.6 | LOC645153 | 3.0 | NDUFAF3 | 2.1 | C1GALT1C1 |
| 2.2 | OAS2 | 1.6 | ST3GAL6 | 3.0 | CDC20 | 2.1 | MAML3 |
| 2.2 | TAPBP | 1.6 | LONRF1 | 2.9 | CT5K | 2.1 | C15orf29 |
| 2.2 | BPI | 1.6 | PPP1R3B | 2.9 | RAB13 | 2.1 | DSC2 |
| 2.2 | DHX58 | 1.6 | MPPE1 | 2.9 | LOC651524 | 2.1 | RRP12 |
| 2.1 | GA56 | 1.6 | LOC652699 | 2.9 | TMEM176A | 2.1 | LRP3 |
| 2.1 | CPT1B | 1.6 | LOC646144 | 2.8 | PDGFC | 2.1 | HDAC7A |
| 2.1 | CD300C | 1.6 | SGM51 | 2.8 | ATP9A | 2.1 | FOS |
| 2.1 | LILRA6 | 1.6 | BMP2K | 2.7 | SV2A | 2.0 | C14orf4 |
| 2.1 | USF1 | 1.6 | SLC31A1 | 2.7 | SPOSC1 | 2.0 | LIPN |
| 2.1 | C2 | 1.6 | ARSB | 2.7 | MARCO | 2.0 | MAPILC382 |
| 2.1 | 382310 | 1.6 | CAMKID | 2.6 | CDC109A | 2.0 | LOC400793 |
| 2.1 | NFXL1 | 1.6 | ICAM4 | 2.6 | NUSAP1 | 2.0 | LOC647834 |
| 2.1 | GCH2 | 1.6 | HIF1A | 2.6 | SLCO4C1 | 2.0 | PHF20L1 |
| 2.1 | CCR1 | 1.6 | LOC641996 | 2.6 | CYP27A1 | 2.0 | CCNJL |
| 2.1 | OAS2 | 1.6 | RNASE10 | 2.5 | LOC644615 | 2.0 | SLC1ZA6 |
| 2.0 | CCR2 | 1.6 | PI15 | 2.5 | PKM2 | 2.0 | FL142957 |
| 2.0 | F2RL1 | 1.6 | SLC30A1 | 2.5 | BMX | 2.0 | CCDC147 |
| 2.0 | SNX20 | 1.6 | LOC389124 | 2.5 | PAD14 | 1.9 | SLC25A40 |
| 2.0 | ARAP2 | 1.6 | ATP1A3 | 2.5 | NAMPT | 1.9 | LOC649270 |

Table 8 (below). Top 50 overexpressed genes in the inflammation modules in the good-treatment response sarcoidosis patients

| Fold change | |
|---|---|
| (good response vs no response/ | |
| inadequate response) | Symbol |
| 8.3 | IL1R2 |
| 6.2 | GRB10 |
| 5.4 | CEACAM4 |
| 5.1 | SIPA1L2 |
| 4.5 | BMX |
| 4.3 | IL1RAP |
| 4.0 | REP52 |
| 4.0 | ANXA3 |
| 4.0 | MMP9 |
| 4.0 | PHC2 |
| 3.8 | HAUS4 |
| 3.6 | DUSP2 |
| 3.6 | CA4 |
| 3.4 | SAMSN1 |
| 3.4 | KLHL2 |
| 3.3 | ACSL1 |
| 3.2 | NSUN7 |
| 3.2 | IL18RAP |
| 3.2 | GNG10 |
| 3.1 | 5MAP2 |
| 3.1 | MGAM |
| 3.1 | LIN7A |
| 3.1 | IRAK3 |
| 3.0 | USP10 |
| 3.0 | CEBPD |
| 3.0 | TGFA |
| 3.0 | FOS |
| 3.0 | MANSC1 |
| 2.9 | SLC26A8 |
| 2.8 | ROPN1L |
| 2.8 | GPR97 |
| 2.8 | NAMPT |
| 2.8 | MRVI1 |
| 2.8 | KCNI15 |
| 2.7 | KLHL8 |
| 2.7 | GNG1D |
| 2.7 | MEGF9 |
| 2.7 | GPR160 |
| 2.7 | B4GAL7S |
| 2.7 | STEAP4 |
| 2.7 | LRG1 |
| 2.7 | FS |
| 2.6 | PHTF1 |
| 2.6 | HMGB2 |
| 2.6 | DGATZ |
| 2.6 | SLC11A1 |
| 2.6 | QPCT |
| 2.6 | PANX2 |
| 2.6 | GPR141 |
| 2.6 | LMNB1 |

TABLE 9
Interferon inducible genes from Berry, et al. (5).
Symbol

CD274
CXCL10
GBP1
GBP2
GBP5
IFI16
IFI35
IFI44
IFI44L
IFI6
IFIH1
IFIT2
IFIT3
IFIT5
IFITM1
IFITM3
IRF7
OAS1
OAS2
OAS3
SOCS1
STAT1
STAT2
TAP1
TAP2

FIG. 10B is a Venn diagram comparing the genes that distinguish between Tb, sarcoidosis, pneumonia and lung cancer, versus, Tb, active sarcoidosis, non-active sarcoidosis, pneumonia and lung cancer. The overlapping 1359 genes are included in the attached electronic table.

TABLE 10
List of Genes Downregulated in Tb versus
Active Sarcoid

| | Fold change | |
|---|---|---|
| Symbol | TB vs Active Sarcoid | Regulation |
| MEF2D | −1.6 | DOWN |
| BHLHB2 | −1.7 | DOWN |
| CLC | −2.3 | DOWN |
| FCER1A | −2.5 | DOWN |
| SRGAP3 | −2.6 | DOWN |
| FLJ43093 | −2.8 | DOWN |
| CCR3 | −2.9 | DOWN |
| EMR4 | −3 | DOWN |
| ZNF792 | −3.1 | DOWN |
| C10orf33 | −3.5 | DOWN |
| CACNG6 | −3.8 | DOWN |
| P2RY10 | −4.2 | DOWN |
| GATA2 | −4.6 | DOWN |
| EMR4P | −6.6 | DOWN |
| ESPN | −7 | DOWN |
| EMR4 | −9.3 | DOWN |
| MXD4 | −1.5 | DOWN |
| ZSCAN18 | −1.6 | DOWN |

TABLE 11
List of 87 genes of FIG. 10B.

| ProbeID | Probe_Sequence | Symbol |
|---|---|---|
| 3460168 | GCTGCTTTTAGGTTAACCACAAAGGAACAACTCAGGATCAGTCGTGATTG | PHF20L1 |
| 6180497 | TACTGAAAGACTTTTGCCTAAAGTGGCATTATTGACTGCTGGTGTGATGA | LOC400304 |
| 6400148 | GAATACTTCTCTTGCTGAGAGCCGATGCCCGTCCCCGGGCCAGCAGGGAT | SELM |
| 1850041 | TCAGACTCCCTGCCACCTTTTCCCCTGGGTTCTGCCGTCTTGCCTCACTT | DPM2 |
| 2690561 | CATGGGCTTTGGTCTTTTTGACTAAACCTCTTTTATAACATGTTCAATAA | RPLP1 |
| 1400747 | GCGGAAGAGGAGCCGCTGGAACCAAGACACAATGGAACAGAAGACAGTGA | SF1 |
| 7650451 | AGTGTCCTCGACATCCCAGGGGAAAGCAAGAGCAGTGAGCCTGAGCAGTG | ZNF683 |
| 3850632 | GAGCCGCCAGGAACCCTCCTCCTGTCAATGGGGGTGTAGTATTTTTGCCA | CTTN |
| 4880600 | CCCCTTGAGAATGGTGATCCACCCAGTTACAGGGGCATTTAGGGAGCAGA | PTCRA |
| 1780008 | GCAAGAAAGTCTAACCTATTCCGGTGTTCTCTCTCCCATGAGACAAGCCG | SNORA28 |
| 7400475 | TGTTAGCCCTGAAGATCTGGCTACCCCAATAGGAAGGCTGAAGGTTTCCC | RPGRIP1 |
| 7510367 | TGCCCCCTGACTGATAGCATTTCAGAATGTGTCTTTTGAAGGGCTATACC | GPR160 |
| 1850035 | CAGAGGCAGGAAAAGCAAGGAGCCAGAATTAAGAGGTTGGGTCAGTCTGC | PPIA |
| 4040546 | AGGACGTGATCCTGCTTGGGGACTTCAATGCTGACTGCGCTTCACTGACC | DNASE1L1 |
| 6100424 | GCTGATCTGGCAGGATGCTCTCTTCAAGCATATCCAAAACCAGATGTGCC | HEMGN |
| 4390487 | GAGCAGGGGAGAAATAGCAGAGGGGCTTGGAGGGTCACATAGGTAGATGG | RAB13 |
| 2320047 | ACATGGCCCGCAAGGACAATGAATCCACTCACATTGCAGAACAATTCCGA | NFIA |
| 2600187 | GTGAGCCCAAAGTTCTGAAAGGTGTTGCGGCTCCTTCGCCTTCGTCAAAT | LOC728843 |
| 5090630 | CCCTGCCCTCATGTTGCTTTGGGTCTAGTGGAGGAGAGAGACAGATAAGC | |
| 7610750 | CTCCTGCCACCCAGTGGCCTCTTTAGGCCAAGCTCATGCCTCACAAGGGC | LOC100134660 |
| 3780767 | GAGCAGCTCCCTCGCTGCGATCTATTGAAAGTCAGATCTCCACACAAGGG | LOC100132564 |
| 580484 | TGCAGCCGTCCATAGCAGTACCCCTAAAATCCCACCAGAATACGGGTCCC | HIP1 |
| 3460669 | AGATGTGGCCATTAAGGAGCCCCTAGTGGATGTCGTGGACCCCAAACAGC | PRMT1 |
| 4850327 | GATCCAGCCATTACTAACCTATTCCTTTTTTGGGGAAATCTGAGCCTAGC | PDGFC |
| 2350156 | GCATCAGCGAGTGCGACAGTGTTGGCGTGGATGTTCTTTCGATGGTACTG | NCRNA00085 |
| 3140386 | CTTGCTTCAGTTGAGACCTACGTTTTGGCCAGTCCCAGCAGGAAGATATC | NFATC3 |
| 3420687 | AGGACACAGAGGAAAGGCTGAAACAACGGGAAGAGGTTTTGAGGAAAATC | GIMAP7 |
| 6370110 | ACTGCTCTTTAAGAGGGGACAAGAAATTGGGGGGACCCGAGGCCTTCCAG | LOC100130905 |
| 4780619 | GGGATTGGTACTTTTGGAAATCAGGTTGGATTTGTGAAGCTGGCAGAAGG | AKAP7 |
| 6840047 | TCAACGCCTGGAGGACGCCTTATGGAGCCAGCATATCCCAGTCTAAAGAA | TLE3 |
| 1940368 | ACACCCTACTGTCCTTGTGCCTCACGCCCCCTCCTCATCCTGCACCCCTT | NRSN2 |
| 4280743 | CCTGCGTCACAGGGAAGCAACCTACAGAGAAGCAGCAGCTCCCCAAGAGA | RPL37 |
| 110372 | GCCTCCTTGTTCCCTGTGGCTGCTGATAACCCAACATTCCATCTCTACCC | CSTA |
| 5080544 | AACTAGCGAACCCCAGGGGAAGGTGCCGTGTGGAGAGCACTTTCGGATTC | C20orf107 |
| 670189 | TTGTCATGCTCCCCACAGAGAGCCCAGGACATTTGCCTGATGTATGGTGC | TMEM169 |
| 7560164 | CCGGGTACAGATCTCAGCAGTGCATAGCGAGGAAGACATTGACCGCTGCG | GCAT |
| 5720682 | AGCAGCACTTGCCCATTCCTTACACCCCTTCCCCATCCTGCTCCGCTTCA | TMEM176A |
| 50136 | TTTGCCTATGATGCCTTCAAGATCTACCGGACTGAGATGGCACCCGGGGC | CMTM5 |
| 2030180 | CAAGTTCTTAACCATCCCGGGTTCCAGTGGTTACAGAGTTCTGCCCTGGG | |
| 3610372 | GGAAATGGGAGTGCTCAGTCTGTGCAAGTCAGAATCCTTGAAACTGGGCC | C3orf26 |
| 2690240 | ACTTGTGGACATCATGGATTGTCTAACACCATCACAGTCCCTGGCTCAGG | FANCD2 |
| 770692 | CTGCCTGGCTCCTCCTTGAGGCTGGAACTCTCTCCAGGGTGGTTAACTCT | C9orf114 |
| 7050612 | CAGAGGAACTTTGCTCAAGGCGCAGATCCGTCACCAGTCCCTTGACAGTC | TIAM2 |
| 1110450 | TGGGACACAGCTGGCCAAGAGCGGTTCAAGACAATAACTACTGCCTACTA | LOC644615 |
| 4730746 | CGCTGGGAGACCTTTGGGACGTGGGGTGGAATTTGGGGTATCTGTGCCTT | PADI2 |
| 3800392 | AGTGCTGCCCTCTGGGGACATGCGGAGTGGGGGTCTTATCCCTGTGCTGA | GRINA |
| 4050768 | CAGAGCCCCTGGTGCAATGCGGTCACAGGTTTTATGGGACTTTGGTGAGC | CHST13 |
| 3120326 | ATTCTTGGTGGCTTCTTCATAGCAGGTAAGCCTCTCCTTCTAAAAACTTC | ANGPT1 |
| 1260215 | GTCAGATGCTGTTGGGTCACATAGAAGAACAAGATAAGGTCCTCCACTGC | KIF27 |
| 1850364 | GCCCTTCCTCTCCCATAAGATGGACAAAAGTGTTTCTGTATCACTGTGTC | ZNF550 |
| 5270379 | GCTAATCTTCAGCCCGTACCAAAAAGTAGAGTGGAGCCTCTTTGCACTAC | PIK3C2A |
| 3710450 | GGAACAGACTGAGAAGGGCAAACATTCCTGGGAGCTGGGCAAGGAGATCC | NR1H3 |
| 2970296 | CAATTCTCCCAATGAGCCTTTTGTCTGTGGGAAAAGCAGGAGACGCTTCG | ALG8 |
| 7560541 | CTCCACTTTGCTGGTTCAGCCTTCGTGTGGCTCCTGGTAACGTGGCTCCA | SLC2A5 |
| 2490411 | GACTGTCAGGAAGGGTCGGAGTCTGTAAAACCAGCATACAGTTTGGCTTT | ITGB5 |
| 780021 | CACCTTCCTGGTCTGTTGGATGCCTTATATCGTGATCTGCTTCTTGGTGG | OPN3 |
| 4880376 | GGCTCTCCTAGTGCCCAGAGACAGGCCCAGAGGTTTACAAGTTTTCTAAG | UBE2O |
| 5670301 | AAAGAAGGGCCCGAGCTTAGTTTCCCCAGGACTGGCCTAGGAAGGAGCAC | RIN3 |
| 7320678 | TGCATGAGATCACACAACTAGGCGGTGACTGAGTCCAACACACCAAAGCC | |
| 2900615 | CATCAACAGCTAAACTGCACAGGGAGGAGGATCGAACGGATCCCTCCCGC | LOC100129203 |
| 3400215 | CTTCAAGGGTTCTGGAGGAGGGAAGGGTCTGCAGGTTCCATGGGTGACAG | B3GNT1 |
| 1090286 | CCAGGAGGATCCCTTGATCCCTTGTGGCCAGGAGTTGGGAGACCAGCCTG | NEK8 |
| 4860181 | ATGTGGAGGTGGCTGGTTCCCATGAACGTGGTTGTCAGAGGCGGGGGACA | SLC38A5 |
| 5670437 | CCCATCTCCAACTCGGAAGTAAGCCCAAGAGAACAACATAAAGCAAACAA | GPR183 |
| 5260379 | TCTCCAGGGGCAAACCTCTGATGTCTTCTTTGCAGCCAGTAGCTTGACTG | LOC728748 |
| 2060280 | GTACGACGTTTGATCCATGCCCATCCAAAAGGATGATGAAGTTCAGGTTG | LOC646966 |
| 2030360 | GTGAACACAGGCATGGCGGCAGAAGTGCCAAAAGTGAGCCCTCTCCAGCA | FAM159A |
| 450382 | GCGGCTATCACCGAAGCAGGAGTGGCCAAAATGAAGTTTAATCCCTTTGT | LOC441073 |
| 1770397 | GAGCTGATTTGATCGAGGAGCGCGGTTACCGGACGGGCTGGGTCTATGGT | CCNC |
| 4010735 | CCCTCCAAGGCAGCAAAGCAGAATCGGGAGCAGTGGAGCAGAAATGTGCA | MRPL9 |
| 2140463 | CTGTTCAGCTCAGGCACAGGGGCACAGCAGAGGTTTGGGAAGCGGTCTCC | SLC37A1 |
| 5340458 | ACGTGCTCCCTCTGCCAGGAGGAGAATGAAGACGTGGTGCGAGATGCGCT | NSUN5 |
| 7320193 | AAGAGGCCAAAGAGGCCCCAGCCGACAAGTGATCGCCCACAAGCCTTACT | GHRL |
| 4180768 | TAGGATTCACACCCCACCTGCGCTTCACTTGGGTCCAGGCCTACTCCTGT | ALAS2 |
| 3890228 | GGCTAAATAGTCAAGGGGTAATATGGGCCTGTTGTTTAGTGTCTCCTTCC | MPZL2 |
| 7330441 | GAGTGGGCAGACATCGAAGCCAAACAGCAGTATCCCGGAAGCACTCATGC | RNF13 |
| 4610538 | GACTTTCCAGTTGGCCCTGATTTTCAACCATGTGATTGTTTCACTCCTGG | SUMO1P1 |
| 2970612 | GGGGAGGGTGGAAGAAATGGTGGACTGTATCTCTCACGTTCTGAAGCAGC | UHRF2 |
| 1070079 | GTGTCACTAAAGTTGGTATACAACCCCCCACTGCTAAATTTGACTGGCTT | RNY4 |
| 3170241 | TGGAAAAAGAGTTACCACGTGTTGCAGTGGTTCCTGACGCTGCTGCCCGC | LOC651524 |
| 6370523 | TCAATGTTCAGTGCTCAGGTATGTAGTAAGTACTGTAGTCCTGTGGGGGC | KBTBD8 |
| 1580626 | ACTCGTCTGACCCATCAGAGACGCCACAGCAGAGAAACACCTCTCAAATG | ZNF224 |
| 2030403 | ATGAACGTTCTCATTAACACGCAGGAGTACCGGGAGCCCTGAACCGCCCG | OLIG1 |
| 650328 | ATGCCATGCATACCTCCTGCCCCGCGGGACCACAATAAAAACCTTGGCAG | TNFRSF4 |
| 10451 | CCACAGCTTGGGGTGTTCAGCACTTGAGGACGGGTGGAGCTTGTTCAACC | BEND7 |
| 7400593 | GCACACGTTCTCGGGACCTCCTGAAGCTGCGTCACAGGCACTAATCAAAG | LOC728323 |
| 2260538 | GGCGGCAGAATGCCATCAAGTGTGGGTGGCTGAGGAAGCAAGGAGGCTTT | ARHGAP24 |

TABLE 12
List of 37 genes of FIG. 10B.

| ProbeID | Probe_Sequence | Symbol |
|---|---|---|
| 4250326 | GGGAGGTCTGAGAGCCCTTAGCATGGGTGGTGTGCTGGGAGGTGGTGGGT | LOC442132 |
| 2810139 | GGTTATGCTGGGGGCGCGGTGGGCTCGCCTCAATACATTCACCACTCATA | HOXA1 |
| 60674 | TGGACCTGGAGGGTCTTCTGCTTGCTGGCTGTAGCTCCAGGTGCTCACTC | LOC652102 |
| 2690634 | AGCATACGGGACCAGGTCTACTATCCATGGCCAACTCTGGCCCAAACACC | PPIE |
| 50164 | GATGGCACTGGACTCGCCGTTATCTTGAGGAGCCAGGAGCTGAAATGGCT | C22orf27 |
| 6770044 | TTGGGCCTGAGGAGCTGCCTGTTGTGGGCCAGCTGCTTCGACTGCTGCTT | TEX10 |
| 1240270 | GGATCTTCAGTTATTCGAGGGGAATGAGGCAGGTCAAGCCGATGCTAGCC | LMTK2 |
| 7570184 | GACCGTCGTGCCCCTCATCAAGGAAGAGCCAAGGACCCCAAGGAGAAGAA | LOC283663 |
| 6560079 | GCACTCAGGTCGTCATCAACTCCTTTTACATTGTGACACGGCCTTTGGCC | SUCNR1 |
| 2030400 | GGCCTGGGGAGATGTTGTTTTCATGCTGCTTCCACCATCACACTGGGGTT | COLQ |
| 3450338 | CTCTCTTCCCTGATCCTTGGAGGAGCCCGAACTGATTCTGGAGCTCTGTG | HLA-DOB |
| 4390079 | TGGGAAAGTGTGAGTTAATATTGGACACATTTTATCCTGATCCACAGTGG | SAMSN1 |
| 3370255 | CCGTTTGCTTCTTTAACTCCAGCCGCGGAATGACATTAGTGGAACCGGGC | INPP5E |
| 3990435 | CCAGGAGGCCGAACACTTCTTTCTGCTTTCTTGACATCCGCTCACCAGGC | |
| 6840494 | CAGCTCGGAGGAAGGTCTCCTATACACACAAAGCCTGGCATGCACCTTCG | CYP4F3 |
| 3850010 | CATTATTGGTTGGCTGCCAATGACCCCATATGTTCTGTGAGAATAGTAGC | CRYZ |
| 5810044 | TTCTCTGGATGCCACGAGTACCAAGTTTTTAGAAGTAGAGCCATCCGTCT | CDC14A |
| 3440327 | GCTGGGCTTGGCTGCCAAGAAGAAGGAGATAAACATCACCATCATCAAAG | LOC653061 |
| 2900360 | TCTATTAACACGGCACTTAGACACGTGCTGTTCCACCTTCCCTCGTGCTG | KIR2DL4 |
| 4560435 | CCTGGCAACCAGTGGGAAAAGAAACATGCGAGGCTGTAGGAAGAGGGAAG | PCYOX1L |
| 4780072 | GCTTTAGATGTCAGTCTCGTTACCAGCAGCCTTTTGACCCAACTACGGCG | TCEAL3 |
| 1030079 | GTCCTGACTGCCTGGAGCATATTTGTGAATTCTCACTTGGAAGACTGGGG | FRRS1 |
| 7150189 | GCCTTTATGCCAGCCCGACACCTGCTGTAATTGGGGTGCATGAGCTATGG | PHF17 |
| 3520168 | TTCCAGGGCACGAGTTCGAGGCCAGCCTGGTCCACATGGGTCGGaaaaaa | |
| 3310504 | CAGAAGTCCTAGACAGTGACATTTCTTAATGGTGGGAGTCCAGCTCATGC | PDK4 |
| 2510561 | CCTCCTCCCCTCTCCTGTACCAGAAAGAAGCCACAACTCATCACCGGAGA | LOC440313 |
| 6110541 | CCAGGACTAGCTTTTTGTGCCATGAGTTAGCCATGGTCCTGGACCCAGCA | ZNF260 |
| 5290068 | GAGCCCAGGGGTTAGAGACAAGCCTTGGCAACATAGCAAGATCCTGGCTC | SLFN13 |
| 580465 | TGATGGACCTCCCCGCTCCCTCAAGCTCTGGATGGCTGCAGTGTTGTACT | VASH1 |
| 4280273 | GGGTGGCAAGGACTGGAGTCAGTTGGAGAGTGCATAGCCAGTCTGTGAAG | GM2A |
| 5340646 | CCTGCATCTGTATTTTATAGTCAGCCTTTTGACCACCTGGTGCCAGCTAT | ASAP2 |
| 1500753 | AGTGACTGTGGTGTCCTTGAGATGCTCACATTACTGCCCGGCCTGCCTCC | VARS2 |
| 3930008 | TAAGCCTTTGGATTTAAAGCCTGTTGAGGCTGGAGTTAGGAGGCAGATTG | RPL14 |
| 7200025 | ACTTCAATGTAGTTTTCCATCCTTCAAATAAACATGTCTGCCCCCATGGT | KIR2DL1 |
| 5260717 | CCGGCTTCTGGGTCTTTGAACAGCCGCGATGTCGATCTTCACCCCCACCA | SBDSP |
| 5570187 | CAGCCTTCATCCATTAACTCTACTAGGGAGCCCACAGCCACCATTTCCAC | S1PR3 |
| 650348 | CTGCTTGCTAGGCTCAATTACCACTTCTGTTTGCTTTGTGGATCCTGGGA | METTL1 |

Thus, in certain embodiments, the present invention includes the identification and/or differentiation of pulmonary diseases using the genes in the Tables of the present invention. Specifically, the skilled artisan will be able to differentiate the pulmonary diseases using 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 144, 150, 200, 250, 300, 400, 500, 600, 700, 800, 900, 1,000, 1,100, 1,200, 1,300, 1,400, or even 1,446 genes listed in the tables contained herein and filed herewith (genes, probes, and SEQ ID NOs incorporated herein by reference). The genes may be selected based on ease of use or accessibility, based on the genes that are most predictive (e.g., using the tables of the present invention), and/or based, in order of importance from top to bottom, of the lists provided for use in the analysis.

Study population and inclusion criteria. The majority of the TB patients were recruited from Royal Free Hospital, NHS Health Care Trust, London. The sarcoidosis patients were recruited from Royal Free Hospital, John Radcliffe Hospital in Oxford, St Mary's Hospital, Imperial College NHS Health Care Trust, and Barnet Hospital in London and the Avicenne Hospital in Paris. The pneumonia patients were recruited from Royal Free Hospital, NHS Health Care Trust, London. The lung cancer patients and 5 of the TB patients in the Test Set were recruited by the Lyon Collaborative Network, France. All patients were recruited consecutively over time such that the Training Set was recruited first followed by the Test Set, Validation Set and lastly the patients' samples that were used in the cell purification. Additional blood gene expression data were obtained from pulmonary and latent TB patients recruited and analysed in our earlier study, and additionally reanalysed in the current study, as presented in FIG. 6C (11).

The inclusion criteria were specific for each disease. Pulmonary TB patients: culture confirmed Mycobacterium tuberculosis in either sputum or bronchoalveolar lavage; pulmonary sarcoidosis: diagnosis made by a sarcoidosis specialist, granuloma's on biopsy, compatible clinical and radiological findings (within 6 months of recruitment) according to the WASOG guidelines (9); community acquired pneumonia patients: fulfilled the British Thoracic Society guidelines for diagnosis (10); lung cancer patients: diagnosis by a lung cancer specialist, histological and radiological features consistent with primary lung cancer; healthy controls: their gender, ethnicity and age were similar to the patients, negative QuantiFERON-TB Gold In-Tube (QFT) (Cellestis) test. The exclusion criteria for all patients and healthy controls included significant other medical history (including any immunosuppression such as HIV infection), aged below 18 years or pregnant. Patients were recruited between September 2009 and March 2012. Patients were recruited before commencing treatment unless otherwise stated. This study was approved by the Central London 3 Research Ethics Committee (09/H0716/4), and Ethical permission from CPP Sud-Est IV, France, CCPPRB, Pitié-salpétrierè Hospital, Paris. All participants gave written informed consent.

IFNγ release assay testing. The QFT M. tubercusosis antigen specific IFN-gamma release assay (IGRA) Assay (Cellestis) was performed according to the manufacturer's instructions.

Gene expression profiling. 3 ml of whole blood were collected into Tempus tubes (Applied Biosystems/Ambion) by standard phlebotomy, vigorously mixed immediately after collection, and stored between −20 and −80° C. before RNA extraction. RNA was isolated using 1.5 ml whole blood and the MagMAX-96 Blood RNA Isolation Kit (Applied Biosystems/Ambion) according to the manufacturer's instructions. 250 μg of isolated total RNA was globin reduced using the GLOBINclear 96-well format kit (Applied Biosystems/Ambion) according to the manufacturer's instructions. Total and globin-reduced RNA integrity was assessed using an Agilent 2100 Bioanalyzer (Agilent Technologies). RNA yield was assessed using a NanoDrop8000 spectrophotometer (NanoDrop Products, Thermo Fisher Scientific). Biotinylated, amplified antisense complementary RNA (cRNA) targets were then prepared from 200-250 ng of the globin-reduced RNA using the Illumina CustomPrep RNA amplification kit (Applied Biosystems/Ambion). 750 ng of labelled cRNA was hybridized overnight to Illumina Human HT-12 V4 BeadChip arrays (Illumina), which contained more than 47,000 probes. The arrays were washed, blocked, stained and scanned on an Illumina iScan, as per manufacturer's instructions. GenomeStudio (Illumina) was then used to perform quality control and generate signal intensity values.

Cell purification and RNA processing for microarray. Whole blood was collected in sodium heparin. Peripheral blood mononuclear cells (PBMCs) were separated from the granulocytes/erythrocytes using a Lymphoprep™ (Axis-Shield) density gradient. Monocytes (CD14+), CD4+ T cells (CD4+) and CD8+T cells (CD8+) were isolated sequentially from the PBMCs using magnetic antibody-coupled (MACS) whole blood beads (Miltenyi Biotec, Germany) according to manufacturer's instructions. Neutrophils were isolated from the granulocyte/erythrocyte layer after red blood cell lysis using the CD15+MACS beads (Miltenyi Biotec, Germany). RNA was extracted from whole blood (5′ Prime PerfectPure Kit) or separated cell populations (Qiagen RNeasy Mini Kit). Total RNA integrity and yield was assessed as described above. Biotinylated, amplified antisense complementary RNA (cRNA) targets were then prepared from 50 ng of total RNA using the NuGEN WT-Ovation™ RNA Amplification and Encore BiotinIL Module (NuGEN Technologies, Inc). Amplifed RNA was purified using the Qiagen MinElute PCR purification kit (Qiagen, Germany). cRNA was then handled as described above.

Raw data processing. After microarray raw data were processed using GeneSpring GX version 11.5 (Agilent Technologies) and the following was applied to all analyses. After background subtraction each probe was attributed a flag to denote its signal intensity detection p-value. Flags were used to filter out probe sets that did not result in a ‘present’ call in at least 10% of the samples, where the ‘present’ lower cut off=0.99. Signal values were then set to a threshold level of 10, log 2 transformed, and per-chip normalised using 75th percentile shift algorithm. Next per-gene normalisation was applied by dividing each messenger RNA transcript by the median intensity of all the samples. All statistical analysis was performed after this stage. Raw microarray data has been deposited with GEO (Accession number GSE). All data collected and analysed in the experiments adhere to the Minimal Information About a Microarray Experiment (MIAME) guidelines.

Data analysis. GeneSpring 11.5 was used to select transcripts that displayed expression variability from the median of all transcripts (unsupervised analysis). A filter was set to include only transcripts that had at least twofold changes from the median and present in ≧10% of the samples. Unsupervised analysis was used to derive the 3422-transcripts. Applying a non-parametric statistical filter (Kruskal Wallis test with a FDR (Benjamini Hochberg)=0.01), after the unsupervised analysis, generated the 1446-transcript and 1396-transcript signatures. The two signatures differed only in which groups the statistical filter was applied across; 1446, five groups (TB, sarcoidosis, pneumonia, lung cancer and controls) and 1396, six groups (TB, active sarcoidosis, non-active sarcoidosis, pneumonia, lung cancer and controls).

Differentially expressed genes for each disease were derived by comparing each disease to a set of controls matched for ethnicity and gender within a 10% difference. GeneSpring 11.5 was used to select transcripts that were ≧1.5 fold different in expression from the mean of the controls and statistically significant (Mann Whitney unpaired FDR (Benjamini Hochberg)=0.01). Comparison Ingenuity Pathway Analysis (IPA) (Ingenuity Systems, Inc., Redwood, Calif.) was used to determine the most significant canonical pathways associated with the differentially expressed genes of each disease relative to the other diseases (Fisher's exact FDR (Benjamini Hochberg)=0.05). The bottom x-axis and bars of each comparison IPA graph indicated the log(p-value) and the top x-axis and line indicated the percentage of genes present in the pathway.

Molecular distance to health (MDTH) was determined as previously described (12), and then applied to different transcriptional signatures. Transcriptional modular analysis was applied as previously described (12). The raw expression levels of all transcripts significantly detected from background hybridisation were compared between each sample and all the controls present in that dataset. The percentage of significantly expressed genes in each module were represented by the colour intensity (Student t-test, p<0.05), red indicates overexpression and blue indicates underexpression. The mean percentage of significant genes and the mean fold change of these genes compared to the controls in specified modules were also shown in graphical form. MDTH and modular analysis were calculated in Microsoft Excel 2010. GraphPad Prism version 5 for Windows was used to generate the graphs.

Differentially expressed genes between the Training Set TB patients and active sarcoidosis patients were derived using the non-parametric Significance Analysis of Microarrays (q<0.05) and ≧1.5 fold expression change. Class prediction was performed within GeneSpring 11.5 using the machine learned algorithm support vector machines (SVM). The model was built using sample classifiers ‘TB’ or ‘not TB’. The SVM model should be built in one study cohort and run in an independent cohort to prevent over-fitting the predictive signature. This was possible for all the cohorts from our study. Where the study cohorts used a different microarray platform the SVM model had to be re-built in that cohort. To reduce the effects of over-fitting the same SVM parameters were always used. The kernel type used was linear, maximum iterations 100,000, cost 100, ratio 1 and validation type N-fold where N=3 with 10 repeats.

Univariate and multivariate regression analysis were calculated using STATA 9 (StataCorp 2005. Stata Statistical Software: Release 9. College Station, Tex.; StataCorp LP). In the multivariate regression analysis where there were missing data points (serum ACE and HRCT disease activity) to prevent list-wise deletion dummy variable adjustment was used.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context. In certain embodiments, the present invention may also include methods and compositions in which the transition phrase “consisting essentially of” or “consisting of” may also be used.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. WHO. Global tuberculosis control. World health organisation. 2010.
2. Newman L S, Rose C S, Bresnitz E A, Rossman M D, Barnard J, Frederick M, Terrin M L, Weinberger S E, Moller D R, McLennan G, Hunninghake G, DePalo L, Baughman R P, Iannuzzi M C, Judson M A, Knatterud G L, Thompson B W, Teirstein A S, Yeager H, Jr., Johns C J, Rabin D L, Rybicki B A, Cherniack R. A case control etiologic study of sarcoidosis: Environmental and occupational risk factors. Am J Respir Crit Care Med 2004; 170:1324-1330.
3. Iannuzzi M C, Rybicki B A, Teirstein A S. Sarcoidosis. N Engl J Med 2007; 357:2153-2165.
4. Anderson S R, Maguire H, Carless J. Tuberculosis in london: A decade and a half of no decline [corrected]. Thorax 2007; 62:162-167.
5. Berry M P, Graham C M, McNab F W, Xu Z, Bloch S A, Oni T, Wilkinson K A, Banchereau R, Skinner J, Wilkinson R J, Quinn C, Blankenship D, Dhawan R, Cush J J, Mejias A, Ramilo O, Kon O M, Pascual V, Banchereau J, Chaussabel D, O'Garra A. An interferon-inducible neutrophil-driven blood transcriptional signature in human tuberculosis. Nature 2010; 466:973-977.
6. Pascual V, Chaussabel D, Banchereau J. A genomic approach to human autoimmune diseases. Annu Rev Immunol 2010; 28:535-571.
7. Koth L L, Solberg O D, Peng J C, Bhakta N R, Nguyen C P, Woodruff P G. Sarcoidosis blood transcriptome reflects lung inflammation and overlaps with tuberculosis. Am J Respir Crit Care Med 2011; 184:1153-1163.
8. Maertzdorf J, Weiner J, 3rd, Mollenkopf H J, Bauer T, Prasse A, Muller-Quernheim J, Kaufmann S H. Common patterns and disease-related signatures in tuberculosis and sarcoidosis. Proc Natl Acad Sci USA 2012; 109:7853-7858.
9. WASOG. Consensus conference: Activity of sarcoidosis. Third wasog meeting, los angeles, USA, Sep. 8-11, 1993. Eur Respir J 1994; 7:624-627.
10. Pankla R, Buddhisa S, Berry M, Blankenship D M, Bancroft G J, Banchereau J, Lertmemongkolchai G, Chaussabel D. Genomic transcriptional profiling identifies a candidate blood biomarker signature for the diagnosis of septicemic melioidosis. Genome Biol 2009; 10:R127.
11. Guiducci C, Gong M, Xu Z, Gill M, Chaussabel D, Meeker T, Chan J H, Wright T, Punaro M, Bolland S, Soumelis V, Banchereau J, Coffman R L, Pascual V, Barrat F J. Tlr recognition of self nucleic
12. Bloom C I, Graham C M, Berry M P, Wilkinson K A, Oni T, Rozakeas F, Xu Z, Rossello-Urgell J, Chaussabel D, Banchereau J, Pascual V, Lipman M, Wilkinson R J, O'Garra A. Detectable changes in the blood transcriptome are present after two weeks of antituberculosis therapy. PLoS One 2012; 7:e46191.
13. Oliveros, J. C. (2007) VENNY. An interactive tool for comparing lists with Venn Diagrams. bioinfogp.cnb.csic.es/tools/venny/index.html.

What is claimed is:

1. A method of determining if a human subject is afflicted with pulmonary disease comprising:
obtaining a sample from a subject suspected of having a pulmonary disease;
determining the expression level of six or more genes from each of the following genes expressed in one or more of the following expression pathways:
EIF2 signaling; mTOR signaling; regulation of eIF4 and p70s6K signaling; interferon signaling; antigen presentation pathways; T cell signaling pathways; and other signaling pathways;
comparing the expression level of the six or more genes with the expression level of the same genes from individuals not afflicted with a pulmonary disease, and
determining the level of expression of the six or more genes in the sample from the subject relative to the samples from individuals not afflicted with a pulmonary disease for the genes expressed in the one or more expression pathways,
wherein co-expression of genes in the EIF2 signaling and mTOR signaling pathways are indicative of active sarcoidosis; co-expression of genes in the regulation of eIF4 and p70s6K signaling pathways is indicative of pneumonia; co-expression of genes in the interferon signaling and antigen presentation pathways are indicative of tuberculosis; and co-expression of genes in the T cell signaling pathways; and other signaling pathways is indicative of lung cancer.

2. The method of claim 1, wherein the genes associated with tuberculosis are selected from at least 3, 4, 5 or 6 genes selected from ANKRD22; FCGR1A; SERPING1; BATF2; FCGR1C; FCGR1B; LOC728744; IFITM3; EPSTI1; GBP5; IF144L; GBP6; GBP1; LOC400759; IFIT3; AIM2; SEPT4; C1QB; GBP1; RSAD2; RTP4; CARD17; IFIT3; CASP5; CEACAM1; CARD17; ISG15; IF127; TIMM10; WARS; IF16; TNFAIP6; PSTPIP2; IF144; SCO2; FBXO6; FER1L3; CXCL10; DHRS9; OAS1; STAT1; HP; DHRS9; CEACAM1; SLC26A8; CACNA1E; OLFM4; and APOL6, wherein the genes are evaluated at least one of: in aggregate, in the order listed, aggregated into pathways, or selected from 7, 8, 9, 10, 11, 12, 13, 15, 20, 25, 35, 40, 45, or 49 genes.

3. The method of claim 1, wherein the genes associated with tuberculosis and not active sarcoidosis, pneumonia or lung cancer are selected from C1QB; IF127; SMARCD3; SOCS1; KCNJ15; LPCAT2; ZDHHC19; FYB; SP140; IFITM1; ALAS2; CEACAM6; OAS2; C1QC; LOC100133565; ITGA2B; LY6E; SP140; CASP7; GADD45G; FRMD3; CMPK2; AQP10; CXCL14; ITPRIPL2; FAS; XK; CARD16; SLAMF8; SELP; NDN; OAS2; TAPBP; BPI; DHX58; GAS6; CPT1B; CD300C; LILRA6; USF1; C2; 38231.0; NFXL1; GCH1; CCR1; OAS2; CCR2; F2RL1; SNX20; and ARAP2, wherein the genes are evaluated at least one of: in aggregate, in the order listed, aggregated into pathways, or selected from 7, 8, 9, 10, 11, 12, 13, 15, 20, 25, 35, 40, 45, or 49 genes.

4. The method of claim 1, wherein the genes associated with active sarcoidosis are selected from FCGR1A; ANKRD22; FCGR1C; FCGR1B; SERPING1; FCGR1B; BATF2; GBP5; GBP1; IFIT3; ANKRD22; LOC728744; GBP1; EPSTI1; IF144L; INDO; IFITM3; GBP6; RSAD2; DHRS9; TNFAIP6; IFIT3; P2RY14; DHRS9; ID01; STAT1; WARS; TIMM10; P2RY14; LOC389386; FER1L3; IFIT3; RTP4; SCO2; GBP4; IFIT1; LAP3; OASL; CEACAM1; LIMK2; CASP5; STAT1; CCL23; WARS; ATF3; IF16; PSTPIP2; ASPRV1; FBXO6; and CXCL10, wherein the genes are evaluated at least one of: in aggregate, in the order listed, aggregated into pathways, or selected from 7, 8, 9, 10, 11, 12, 13, 15, 20, 25, 35, 40, 45, or 49 genes.

5. The method of claim 1, wherein the genes associated with active sarcoidosis and not tuberculosis, pneumonia or lung cancer are selected from CCL23; PIK3R6; EMR4; CCDC146; KLF4; GRINA; SLC4A1; PLA2G7; GRAMD1B; RAPGEF1; NXNL1; TRIM58; GABBR1; TAGLN; KLF4; MFAP3L; LOC641798; RIPK2; LOC650840; FLJ43093; ASAP2; C15orf26; REC8; KIAA0319L; GRINA; FLJ30092; BTN2A1; HIF1A; LOC440313; HOXA1; LOC645153; ST3GAL6; LONRF1; PPP1R3B; MPPE1; LOC652699; LOC646144; SGMS1; BMP2K; SLC31A1; ARSB; CAMK1D; ICAM4; HIF1A; LOC641996; RNASE10; PI15; SLC30A1; LOC389124; and ATP1A3, wherein the genes are evaluated at least one of: in aggregate, in the order listed, aggregated into pathways, or selected from 7, 8, 9, 10, 11, 12, 13, 15, 20, 25, 35, 40, 45, or 49 genes.

6. The method of claim 1, wherein the genes associated with pneumonia are selected from OLFM4; LTF; VNN1; HP; DEFA4; OPLAH; CEACAM8; DEFA1B; ELANE; C19orf59; ARG1; CDK5RAP2; DEFA1B; DEFA3; DEFA1B; FCGR1A; MMP8; FCGR1B; SLPI; SLC26A8; MAPK14; CAMP; NLRC4; FCAR; RNASE3; FCGR1B; NAIP; OLR1; FCGR1C; ANXA3; DEFA1; PGLYRP1; TCN1; ANKDD1A; COL17A1; SLC26A8; TMEM144; SAMD14; MAPK14; RETN; NAIP; GPR84; CASP5; MPO; MMP9; CR1; MYL9; CLEC4D; ITGAX; and ANKRD22, wherein the genes are evaluated at least one of: in aggregate, in the order listed, aggregated into pathways, or selected from 7, 8, 9, 10, 11, 12, 13, 15, 20, 25, 35, 40, 45, or 49 genes.

7. The method of claim 1, wherein the genes associated with pneumonia and not tuberculosis, active sarcoidosis, or lung cancer are selected from DEFA4; ELANE; MMP8; OLR1; COL17A1; RETN; GPR84; LOC100134379; TACSTD2; SLC2A11; LOC100130904; MCTP2; AZU1; DACH1; GADD45A; NSUN7; CR1; CDK5RAP2; LOC284648; GPR177; CLEC5A; UPB1; SLC2A5; GPR177; APP; LAMC1; REPS2; PIK3CB; SMPDL3A; UBE2C; NDUFAF3; CDC20; CTSK; RAB13; LOC651524; TMEM176A; PDGFC; ATP9A; SV2A; SPOCD1; MARCO; CCDC109A; NUSAP1; SLCO4C1; CYP27A1; LOC644615; PKM2; BMX; PADI4; and NAMPT, wherein the genes are evaluated at least one of: in aggregate, in the order listed, aggregated into pathways, or selected from 7, 8, 9, 10, 11, 12, 13, 15, 20, 25, 35, 40, 45, or 49 genes.

8. The method of claim 1, wherein the genes associated with lung cancer are selected from ARG1; TPST1; FCGR1A; C19orf59; SLPI; FCGR1B; IL1R1; FCGR1C; TDRD9; SLC26A8; FCGR1B; CLEC4D; LOC100132858; SLC22A4; LOC100133177; SIPA1L2; ANXA3; LIMK2; TMEM88; MMP9; ASPRV1; MANSC1; TLR5; CD163; CAMP; LOC642816; DPRXP4; LOC643313; NTN3; MRVI1; F5; SOCS3; TncRNA; MIR21; LOC100170939; LOC100129904; GRB10; ASGR2; LOC642780; LOC400499; FCAR; KREMEN1; SLC22A4; CR1; LOC730234; SLC26A8; C7orf53; VNN1; NLRC4; and LOC400499, wherein the genes are evaluated at least one of: in aggregate, in the order listed, aggregated into pathways, or selected from 7, 8, 9, 10, 11, 12, 13, 15, 20, 25, 35, 40, 45, or 49 genes.

9. The method of claim 1, wherein the genes associated with lung cancer and not tuberculosis, active sarcoidosis, or pneumonia are selected from TPST1; MRVI1; C7orf53; ECHDC3; LOC651612; LOC100134660; TIAM2; KIAA1026; HECW2; TLE3; TBC1D24; LOC441193; CD163; RFX2; LOC100134688; LOC642342; FKBP9L; PHF20L1; LOC402176; CD163; OSBPL1A; PRMT5; UBTD1; ADORA3; SH2D3C; RBP7; ERGIC1; TMEM45B; CUX1; TREM1; C1GALT1C1; MAML3; C15orf29; DSC2; RRP12; LRP3; HDAC7A; FOS; C14orf4; LIPN; MAP1LC3B2; LOC400793; LOC647834; PHF20L1; CCNJL; SLC12A6; FLJ42957; CCDC147; SLC25A40; and LOC649270, wherein the genes are evaluated at least one of: in aggregate, in the order listed, aggregated into pathways, or selected from 7, 8, 9, 10, 11, 12, 13, 15, 20, 25, 35, 40, 45, or 49 genes.

10. The method of claim 1, wherein the genes associated with lung cancer and not tuberculosis, active sarcoidosis, or pneumonia are selected from Table 1 by:
parsing the genes into the expression pathways, and
determining that the subject is afflicted with a pulmonary disease selected from tuberculosis, sarcoidosis, cancer or pneumonia based on the gene expression from a sample obtained from the subject when compared to the level of expression of the genes in each of the expression pathways.

11. The method of claim 1, wherein the specificity is 90 percent or greater and sensitivity is 80 percent or greater for a diagnosis of tuberculosis or sarcoidosis.

12. The method of claim 1, further comprising a method for displaying if the patient has tuberculosis or sarcoidosis aggregating the expression data from the 3, 4, 5, 6 or more genes into a single visual display of a vector of expression for tuberculosis, sarcoidosis, cancer or an infectious pulmonary disease.

13. The method of claim 1, further comprising the step of detecting and evaluating 7, 8, 9, 10, 12, 15, 20, 25, 35, 50, 75, 90, 100, 125, or 144 genes for the analysis.

14. The method of claim 1, further comprising the step of detecting and evaluating the EIF2 signaling; mTOR signaling; regulation of eIF4 and p70s6K signaling; interferon signaling; antigen presentation pathways; T cell signaling pathways; and other signaling pathways from 7, 8, 9, 10, 12, 15, 20, 25, 35, 50, 75, 90, 100, 125, or 144 genes that are upregulated or downregulated and are selected from UBE2J2; ALPL; JMJD6; FCER1G; LILRA5; LY96; FCGR1C; C10orf33; GPR109B; PROK2; PIM3; SH3GLB1; DUSP3; PPAP2C; SLPI; MCTP1; KIF1B; FLJ32255; BAGE5; IFITM1; GPR109A; IF135; LOC653591; KREMEN1; IL18R1; CACNA1E; ABCA2; CEACAM1; MXD4; TncRNA; LMNB1; H2AFJ; HP; ZNF438; FCER1A; SLC22A4; DISC1; MEFV; ABCA1; ITPRIPL2; KCNJ15; LOC728519; ERLIN1; NLRC4; B4GALT5; LOC653610; HIST2H2BE; AIM2; P2RY10; CCR3; EMR4P; NTN3; C1QB; TAOK1; FCGR1B; GATA2; FKBP5; DGAT2; TLR5; CARD17; INCA; MSL3L1; ESPN; LOC645159; C19orf59; CDK5RAP2; PLSCR1; RGL4; IFI30; LOC641710; GAGGCTTTCAGGTAGGAGGACAATGGTAGCACTGTAGGTCCCCAGTGTCG (SEQ ID NO.: 754); LOC100008589; LOC100008589; SMARCD3; NGFRAP1; LOC100132394; OPLAH; CACNG6; LILRB4; HIST2H2AA4; CYP1B1; PGS1; SPATA13; PFKFB3; HIST1H3D; SNORA73B; SLC26A8; SULT1B1; ADM; HIST2H2AA3; HIST2H2AA3; GYG1; CST7; EMR4; LILRA6; MEF2D; IFITM3; MSL3; DHRS13; EMR4; C16orf57; HIST2H2AC; EEF1D; TDRD9; GPR97; ZNF792; LOC100134364; SRGAP3; FCGR1A; HPSE; LOC728417; LOC728417; MIR21; HIST1H2BG; COP1; SMARCD3; LOC441763; ZSCAN18; GNG8; MTRF1L; ANKRD33; PLAC8; PLAC8; SLC26A8; AGTRAP; FLJ43093; LPCAT2; AGTRAP; S100A12; SVIL; LILRA5; LILRA5; ZFP91; CLC; LOC100133565; LTB4R; SEPT04; ANXA3; BHLHB2; IL4R; IFNAR1; MAZ; GCCCCCTAATTGACTGAATGGAACCCCTCTTGACCAAAGTGACCCCAGAA (SEQ ID NO.: 1379); OSM; and optionally excluding at least one of ADM, SEPT4, IFITM1, FCER1G, MED2F, CDK5RAP2 or CARD16.

15. The method of claim 14, wherein the genes that are downregulated are selected from MEF2D; BHLHB2; CLC; FCER1A; SRGAP3; FLJ43093; CCR3; EMR4; ZNF792; C10orf33; CACNG6; P2RY10; GATA2; EMR4P; ESPN; EMR4; MXD4; and ZSCAN18.

16. The method of claim 14, wherein the interferon inducible genes are selected from CD274; CXCL10; GBP1; GBP2; GBP5; IF116; IF135; IF144; IF144L; IF16; IFIH1; IFIT2; IFIT3; IFIT5; IFITM1; IFITM3; IRF7; OAS1; OAS2; OAS3; SOCS1; STAT1; STAT2; TAP1; and TAP2.

17. The method of claim 1, wherein the sample is a blood, peripheral blood mononuclear cells, sputum, or lung biopsy.

18. The method of claim 1, wherein the expression level comprises a mRNA expression level and is quantitated by a method selected from the group consisting of polymerase chain reaction, real time polymerase chain reaction, reverse transcriptase polymerase chain reaction, hybridization, probe hybridization and gene expression array.

19. The method of claim 1, wherein the expression level is determined using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing.

20. The method of claim 1, wherein the expression level is determined by microarray analysis that comprises use of oligonucleotides that hybridize to mRNA transcripts or cDNAs for the six or more genes, and wherein the oligonucleotides are disposed or directly synthesized on the surface of a chip or wafer.

21. The method of claim 20, wherein the oligonucleotides are about 10 to about 50 nucleotides in length.

22. The method of claim 1, further comprising the step of using the determined comparative gene product information to formulate at least one of diagnosis, a prognosis or a treatment plan.

23. The method of claim 1, wherein the patient's disease state is further determined by radiological analysis of the patient's lungs.

24. The method of claim 1, further comprising the step of determining a treated patient gene expression dataset after the patient has been treated and determining if the treated patient gene expression dataset has returned to a normal gene expression dataset thereby determining if the patient has been treated.

25. A method of determining a lung disease from a patient suspected of sarcoidosis, tuberculosis, lung cancer or pneumonia comprising:
obtaining a sample from the patient suspected of sarcoidosis, tuberculosis, lung cancer or pneumonia;
detecting expression of 3, 4, 5, 6 or more disease genes, markers, or probes of Table 1 (SEQ ID NOS.: 1 to 1446), wherein increased expression of mRNA of upregulated sarcoidosis, tuberculosis, lung cancer and pneumonia markers of Table 1 and/or decreased expression of mRNA of downregulated sarcoidosis, tuberculosis, lung cancer or pneumonia markers of Table 1 relative to the expression of the mRNAs from a normal sample; and
determining the lung disease based on the expression level of the six or more disease markers of Table 1 based on a comparison of the expression level of sarcoidosis, tuberculosis, lung cancer, and pneumonia.

26. The method of claim 25, further comprising the step of selecting 3, 4, 5, 6 or more genes that are differentially expressed between sarcoidosis, tuberculosis, lung cancer, and pneumonia.

27. The method of claim 25, further comprising the step of differentiating between sarcoidosis that is active sarcoidosis and inactive sarcoidosis by determining the expression levels of six or more genes, markers, or probes selected from: TMEM144; FBLN5; FBLN5; ERI1; CXCR3; GLUL; LOC728728; KLHDC8B; KCNJ15; RNF125; CCNB1IP1; PSG9; LOC100170939; QPCT; CD177; LOC400499; LOC400499; LOC100134634; TMEM88; LOC729028; EPSTI1; INSC; LOC728484; ERP27; CCDC109A; LOC729580; C2; TTRAP; ALPL; MAEA; COX10; GPR84; TRMT11; ANKRD22; MATK; TBC1D24; LILRA5; TMEM176B; CAMP; PKIA; PFTK1; TPM2; TPM2; PRKCQ; PSTPIP2; LOC129607; APRT; VAMPS; FCGR1C; SHKBP1; CD79B; SIGIRR; FKBP9L; LOC729660; WDR74; LOC646434; LOC647834; RECK; MGST1; PIWIL4; LILRB1; FCGR1B; NOC3L; ZNF83; FCGBP; SNORD13; LOC642267; GBP5; EOMES; C5; CHMP7; ETV7; ILVBL; LOC728262; GNLY; LOC388572; GATA1; MYBL1; LOC441124; IL12RB1; BRIX1; GAS6; GAS6; LOC100133740; GPSM1; C6orf129; IER3; MAPK14; PROK1; GPR109B; SASP; LOC728093; PROK2; CTSW; ABHD2; LOC100130775; SLITRK4; FBXW2; RTTN; TAF15; FUT7; DUSP3; LOC399715; LOC642161; TCTN1; SLAMF8; TGM2; ECE1; CD38; INPP4B; ID3; CR1; CR1; TAPBP; PPAP2C; MBOAT2; MS4A2; FAM176B; LOC390183; SERPING1; LOC441743; H1F0; SOD2; LOC642828; POLB; TSPAN9; ORMDL3; FER1L3; LBH; PNKD; SLPI; SIRPB1; LOC389386; REC8; GNLY; GNLY; FOLR3; LOC730286; SKAP1; SELP; DHX30; KIAA1618; NQO2; ANKRD46; LOC646301; LOC400464; LOC100134703; C20orf106; SLC25A38; YPEL1; IL1R1; EPHA1; CHD6; LIMK2; LOC643733; LOC441550; MGC3020; ANKRD9; NOD2; MCTP1; BANK1; ZNF30; FBXO7; FBXO7; ABLIM1; LAMP3; CEBPE; LOC646909; BCL11B; TRIM58; SAMD3; SAMD3; MYOF; TTPAL; LOC642934; FLJ32255; LOC642073; CAMKK2; OAS2; RASGRP1; CAPG; LOC648343; CETP; CETP; CXCR7; UBASH3A; LOC284648; IL1R2; AGK; GTPBP8; LEF1; LEF1; GPR109A; IF135; IRF7; IRF7; SP4; IL2RB; ABLIM1; TAPBP; MAL; TCEA3; KREMEN1; KREMEN1; VNN1; GBP1; GBP1; UBE2C; DET1; ANKRD36; DEFA4; GCH1; IL7R; TMCO3; FBXO6; LACTB; LOC730953; LOC285296; IL18R1; PRR5; LOC400061; TSEN2; MGC15763; SH3YL1; ZNF337; AFF3; TYMS; ZCCHC14; SLC6A12; LY6E; KLF12; LOC100132317; TYW3; BTLA; SLC24A4; NCALD; ORAI2; ITGB3BP; GYPE; DOCKS; RASGRP4; LOC339290; PRF1; TGFBR3; LGALS9; LGALS9; BATF2; MGC57346; TXK; DHX58; EPB41L3; LOC100132499; LOC100129674; GDPD5; ACP2; C3AR1; APOB48R; UTRN; SLC2A14; CLEC4D; PKM2; CDCA5; CACNA1E; OSBPL3; SLC22A15; VPREB3; LOC642780; MEGF6; LOC93622; PFAS; LOC729389; CREBZF; IMPDH1; DHRS3; AXIN2; DDX60L; TMTC1; ABCA2; CEACAM1; CEACAM1; FLJ42957; SIAH2; DDAH2; C13orf18; TAGLN; LCN2; RELB; NR1I2; BEND7; PIK3C2B; IF16; DUT; SETD6; LOC100131572; TNRC6A; LOC399744; MAPK13; TAP2; CCDC15; TncRNA; SIPA1L2; HIST1H4E; PTPRE; ELANE; TGM2; ARSD; LOC651451; CYFIP1; CYFIP1; LOC642255; ASCC2; ZNF827; STAB1; LMNB1; MAP4K1; PSMB9; ATF3; CPEB4; ATP5S; CD5; SYTL2; H2AFJ; HP; SORT1; KLHL18; HIST1H2BK; KRTAP19-6; RNASE2; LOC100134393; C11orf82; BLK; CD160; LOC100128460; CD19; ZNF438; MBNL3; MBNL3; LOC729010; NAGA; FCER1A; C6orf25; SLC22A4; LOC729686; CTSL1; BCL11A; ACTA2; KIAA1632; UBE2C; CASP4; SLC22A4; SFT2D2; TLR2; C10orf105; EIF2AK2; TATDN1; RAB24; FAH; DISC1; LOC641848; ARG1; LCK; WDFY3; RNF165; MLKL; LOC100132673; ANKDD1A; MSRB3; LOC100134379; MEFV; C12orf57; CCDC102A; LOC731777; LOC729040; TBC1D8; KLRF1; KLRF1; ABCA1; LOC650761; LOC653867; LOC648710; SLC2A11; LOC652578; GPR114; MANSC1; MANSC1; DGKA; LIN7A; ITPRIPL2; ANO9; KCNJ15; KCNJ15; LOC389386; LOC100132960; LOC643332; SFI1; ABCE1; ABCE1; SERPINA1; OR2W3; ABI3; LOC400759; LOC728519; LOC654053; LOC649553; HSD17B8; C16orf30; GADD45G; TPST1; GNG7; SV2A; LOC649946; LOC100129697; RARRES3; C8orf83; TNFSF13B; SNRPD3; LOC645232; PI3; WDFY1; LOC100133678; BAMBI; POPS; TARBP1; IRAK3; ZNF7; NLRC4; SKAP1; GAS7; C12orf29; KLRD1; ABHD15; CCDC146; CASP5; AARS2; LOC642103; LOC730385; GAR1; MAF; ARAP2; C16orf7; HLA-C; FLJ22662; DACH1; CRY1; CRY1; LRRC25; KIAA0564; UPF3A; MARCO; SRPRB; MAD1L1; LOC653610; P4HTM; CCL4L1; LAPTM4B; MAPK14; CD96; TLR7; KCNMB1; P2RX7; LOC650140; LOC791120; LTF; C3orf75; GPX7; SPRYD5; EEF1B2; CTDSPL; HIST2H2BE; SLC38A1; AIM2; LOC100130904; LOC650546; P2RY10; ILSRA; MMP8; LOC100128485; RPS23; HDAC7; GUCY1A3; TGFA; NAIP; NAIP; NELL2; SIDT1; SLAMF1; MAPK14; CCR3; MKNK1; D4S234E; NBN; LOC654346; FGFBP2; BTLA; LRRN3; MT2A; LOC728790; LOC646672; NTN3; CD8A; CD8A; ZBP1; LDOC1L; CHM; LOC440731; LOC100131787; TNFRSF10C; LOC651612; STX11; LOC100128060; C1QB; PVRL2; ZMYND15; TRAPPC2P1; SECTM1; TRAT1; CAMKK2; CXCR5; CD163; FAS; RPL12P6; LOC100134734; CD36; FCGR1B; NR3C2; CSGALNACT2; GATA2; EBI2; EBI2; FKBP5; CRISPLD2; LOC152195; LOC100132199; DGAT2; SCML1; LSS; CIITA; SAP30; TLR5; NAMPT; GZMK; CARD17; INCA; MSL3L1; CD8A; MIIP; SRPK1; SLC6A6; C10orf119; C17orf60; LOC642816; AKR1C3; LHFPL2; CR1; KIAA1026; CCDC91; FAM102A; FAM102A; UPRT; PLEKHA1; CACNA2D3; DDX10; RPL23A; C2orf44; LSP1; C7orf53; DNAJC5; SLAIN1; CDKN1C; HIATL1; CRELD1; ZNHIT6; TIFA; ARL4C; PIGU; MEF2A; PIK3CB; CDK5RAP2; FLNB; GRAP; BATF; CYP4F3; KIR2DL3; C19orf59; NRG1; PPP2R2B; CDK5RAP2; PLSCR1; UBL7; HES4; ZNF256; DKFZp761E198; SAMD14; BAG3; PARP14; MS4A7; ECHDC3; OCIAD2; LOC90925; RGL4; PARP9; PARP9; CD151; SAAL1; LOC388076; SIGLEC5; LRIG1; PTGDR; PTGDR; NBPF8; NHS; ACSL1; HK3; SNX20; F2RL1; F2RL1; PARP12; LOC441506; MFGE8; SERPINA10; FAM69A; IL4R; KIAA1671; OAS3; PRR5; TMEM194; MS4A1; MTHFD2; LOC400793; CEACAM1; APP; RRBP1; SLCO4C1; XAF1; XAF1; SLC2A6; ZNF831; ZNF831; POLR1C; GLT1D1; VDR; IFIT5; SNHG8; TOP1MT; UPP1; SYTL2; LOC440359; KLRB1; MTMR3; S1PR1; FYB; CDC20; MEX3C; FAM168B; SLC4A7; CD79B; FAM84B; LOC100134688; LOC651738; PLAGL1; TIMM10; LOC641710; TRAF5; TAP1; FCRL2; SRC; RALGAPA1; OCIAD2; PON2; LOC730029; LOC100134768; LOC100134241; LOC26010; PLA2G12A; BACH1; DSC1; NOB1; LOC645693; LOC643313; BTBD11; REPS2; ZNF23; C18orf55; APOL2; APOL2; PASK; FER1L3; U2AF1; LOC285359; SIGLEC14; ARL1; C19orf62; NCR3; HOXB2; RNF135; IFIT1; KLF12; LILRB2; LOC728835; GSN; LOC100008589; LOC100008589; FLJ14213; SH2D3C; LOC100133177; HIST2H2AB; KIAA1618; C21orf2; CREB5; FAS; RSAD2; ANPEP; C14orf179; TXNL4B; MYL9; MYL9; LOC100130828; LOC391019; ITGA2B; KLRC3; RASGRP2; NDST1; LOC388344; IF16; OAS1; OAS1; TRIM10; LIMK2; LIMK2; ATP5S; SMARCD3; PHC2; SOX8; LCK; SAMD9L; EHBP1; E2F2; CEACAM6; LOC100132394; LOC728014; LOC728014; SIRPG; OPLAH; FTHL2; CXorf21; CACNG6; C11orf75; LY9; LILRB4; STAT2; RAB20; SOCS1; PLOD2; UGDH; MAK16; ITGB3; DHRS9; PLEKHF1; ASAP1IT1; PSME2; LOC100128269; ALX1; BAK1; XPO4; CD247; FAM43A; ICOS; ISG15; HIST2H2AA4; CD79A; SLC25A4; TMEM158; GPR18; LAP3; TNFSF13B; TC2N; HSF2; CD7; C20orf3; HLA-DRB3; SESN1; LOC347376; P2RY14; P2RY14; P2RY14; CYP1B1; IFIT3; IFIT3; RPL13L; LOC729423; DBN1; TTC27; DPH5; GPR141; RBBP8; LOC654350; SLC30A1; PRSS23; JAM3; GNPDA2; IL7R; ACAD11; LOC642788; ALPK1; LOC439949; BCAT1; ATPGD1; TREML1; PECR; SPATA13; MAN1C1; ID01; TSEN54; SCRN1; LOC441193; LOC202134; KIAA0319L; MOSC1; PFKFB3; GNB4; ANKRD22; PROS1; CD40LG; RIOK2; AFF1; HIST1H3D; SLC26A8; SLC26A8; RNASE3; UBE2L6; UBE2L6; SSH1; KRBA1; SLC25A23; DTX3L; DOK3; SULT1B1; RASGRP4; ALOX15B; ADM; LOC391825; LOC730234; HIST2H2AA3; HIST2H2AA3; LIMK2; MMRN1; FKBP1A; GYG1; ASF1A; CD248; CD3G; DEFA1; EPHX2; CST7; ABLIM3; ANKRD55; SLC45A3; RAB33B; LILRA6; LILRA6; SPTLC2; CDA; PGD; LOC100130769; ECHDC2; KIF20B; B3GNT8; PYHIN1; LBH; LBH; BPI; GAR1; ST3GAL4; TMEM19; DHRS12; DHRS12; FAM26F; FCRLA; OSBPL7; CTSB; ALDH1A1; SRRD; TOLLIP; ICAM1; LAX1; CASP7; ZDHHC19; LOC732371; DENND1A; EMR2; LOC643308; ADA; LOC646527; LOC643313; GZMB; OLIG2; HLA-DPB1; MX1; THOC3; TRPM6; GK; JAK2; ARHGEF11; ARHGEF11; HOMER2; TACSTD2; CA4; GAA; IFITM3; CLYBL; CLYBL; MME; ZNF408; STAT1; STAT1; PNPLA7; INDO; PDZD8; PDGFD; CTSL1; HOMER3; CEP78; SBK1; ALG9; IL1R2; RAB40B; MMP23B; PGLYRP1; UHRF1; IF144L; PARP10; PARP10; GOLGA8A; CCR7; HEMGN; TCF7; CLUAP1; LOC390735; LOC641849; TYMP; DEFA1B; DEFA1B; DEFA1B; REPS2; REPS2; OSBPL1A; C11orf1; MCTP2; EMR4; LOC653316; FCRL6; MRPS26; RHOBTB3; DIRC2; CD27; PLEKHG4; CDH6; C4orf23; HIST2H2AC; SLC7A6; SLC7A6; SLAMF6; RETN; FAIM3; TMEM99; LOC728411; TMEM194A; NAPEPLD; ACOX1; CTLA4; SCO2; STK3; FLT3LG; VASP; FBXO31; TDRD9; TDRD9; LOC646144; NUSAP1; GPR97; GPR97; GPR97; EMR1; SLAMF6; CCDC106; ODF3B; LOC100129904; PADI4; LOC100132858; PIK3AP1; ZNF792; DIP2A; OSCAR; CLIC3; FANCE; TECPR2; P2RY10; ADORA3; IL18RAP; DEFA3; BRSK1; LOC647691; S1PR5; CPA3; BMX; DDX58; RHOBTB1; TNFRSF25; LOC730387; OLR1; HERC5; STAT1; NELF; STAP1; ZNF516; ARHGAP26; TIMP2; FCGR1A; RHOH; IF144; MTX3; CD74; LCK; TLR4; DSC2; CXorf45; ENPP4; CD300C; OASL; HPSE; MTHFD2; GSTM2; OLFM4; ABHD12B; LOC728417; LOC728417; FCAR; GTPBP3; KLF4; HOPX; THBD; HIST1H2BG; LOC730995; NOP56; ZBTB9; NLRC3; LOC100134083; COP1; CARD16; SP140; CD96; POLD2; IL32; LOC728744; FZD2; ZAP70; PYHIN1; SCARF1; IF127; PFKFB2; PAM; WARS; TCN1; LOC649839; MMP9; TMEM194A; TAP2; C17orf87; LOC728650; PNMA3; CPT1B; LTBP3; CCDC34; PRAGMIN; C9orf91; SMPDL3A; GPR56; C14orf147; SMARCD3; FAM119A; LOC642334; ENOSF1; FAR2; LOC441763; TESC; CECR6; KIAA1598; GPR109B; LRRN3; RNF213; ASGR2; ASGR2; ZSCAN18; MCOLN2; IFIT2; PLCH2; MAP7; GBP4; MGMT; GAL3ST4; C2orf89; TXNDC3; IFIH1; PRRG4; LOC641693; LOC728093; TNFAIP8L1; AP3M2; BACH2; BACH2; C9orf123; CACNA1I; LOC100132287; CAMK1D; ANKRD33; CCR6; ALDH1A1; LOC100132797; CD163; ESAM; FCAR; TCN2; CD6; CD3E; CCDC76; MS4A1; IFIT1; MED13L; SLC26A8; NOV; FLJ20035; UGT1A3; LOC653600; LOC642684; KIAA0319L; KLRD1; TRIM22; C4orf18; TSPAN3; TSPAN3; DNAJC3; AGTRAP; LOC646786; NCALD; TTC25; TSPAN5; ZNF559; NFKB2; LOC652616; HLA-DOA; WARS; GBP2; AUTS2; IGF2BP3; OASL; DYSF; FLJ43093; MS4A14; TGFB1I1; RAD51C; CALD1; LOC730281; MUC1; C14orf124; RPL14; APOL6; KCTD12; ITGAX; IFIT3; LPCAT2; ZNF529; AGTRAP; LOC402112; LOC100134822; SH2D1B; MPO; LOC100131967; LOC440459; FAM44B; ACOT9; LOC729915; PDZK1IP1; S100A12; RAB3IL1; TMEM204; CXCL10; TSR1; MXD3; LILRA5; CKAP4; C6orf190; ECGF1; LDLRAP1; GRB10; FCRL3; LOC731275; ZFP91; BCL6; SAMD3; LOC647436; CLC; GK; LOC100133565; OAS2; LOC644937; SIRPD; GPBAR1; GNL3; CD79B; ELF2; GAA; CD47; NMT2; MATR3; TMEM107; GCM1; RORA; MGAM; LOC100132491; KRT72; SEPT04; ACADVL; ANXA3; MEGF9; MEGF9; PTPRJ; HLA-DRB4; FFAR2; PML; HLA-DQA1; CEACAM8; SH3KBP1; TRPM2; CUX1; SUV39H1; USF1; VAPA; ALOX15; CD79A; DPRXP4; LOC652750; ECM1; ST6GAL1; KLHL3; RTP4; FAM179A; HDC; SACS; C9orf72; C9orf72; LOC652726; PVRIG; PPP1R16B; NSUN7; NSUN7; ZNF783; LOC441013; LOC100129343; OSM; UNC93B1; DNAJC30; FLJ14166; C9orf72; SAMD4A; F5; PARP15; PAFAH2; COL17A1; TYMP; LOC389672; ABCB1; LOC644852; TARP; SLAMF7; FRMD3; LOC648984; PLAUR; LOC100132119; KLRG1; INTS2; MYC; HIST1H4H; C9orf45; GBP6; KIFAP3; HSPC159; SOCS3; GOLGA8B; LOC100133583; ARL4A; ASNS; ITGAX; LOC153561; GSTM1; OAS2; OAS2; TRIM25; ABHD14A; LOC642342; GPR56; C4orf18; AK1; PIK3R6; HSPE1; ASPHD2; DHRS9; GRN; BOAT; LOC100134300; SDSL; TNFAIP6; LOC402176; LOC441019; FAM134B; ZNF573, GGGGTAACACAGAGTGCCCTTATGAAGGAGTTGGAGATCCTgcaaggaag (SEQ ID NO.:69); AAACCCGTCACCCAGATCGTCAGCGCCGAGGCCTGGGGTAGAGCAGGTGA (SEQ ID NO.:87); TGTTCTTCCCCATGTCCTGGATGCCACTGGAAGTGCACACTGCTTGTATG (SEQ ID NO.:93); CCCTGGAAAGCTCCCCGACAACCTCCACTGCCATTACCCACTAGGCAAGT (SEQ ID NO.:95); CCTCCAGTGGTTTAGGCAGGACCCTGGGAAAGGTCTCACATCTCTGTTGC (SEQ ID NO.:174); GCACCATGCATGGAGTCAGCCATTTCTCTAGGAACCTTGATTCCTGTCTG (SEQ ID NO.:193); CCCCACGCCTGTTTGTATTGGGAGCTCTGGACCAATAGTGTCTCTCCTAG (SEQ ID NO.:196); CCAGCCACTCTACTCAAGGGGCATATATTTTGGCATGAGGTGGGATAGAG (SEQ ID NO.:240); gcatgtgtatgatgtgtgtgcgtcggaccgcttctaggctactaagtgtc (SEQ ID NO.:257); AGGGGCAGTATACTCTTATCAGTGCGAGGTAGCTGGGGCCTGTGATAGTT (SEQ ID NO.:299); CAAGCCTGGCAGTAAATCCGAATATCCAGAACCCTGACCCTGCCGTGTAC (SEQ ID NO.:319); CAGCATGTAGGGCAGTGCTTGCACGTAGCATCTGGTGCCTAACCAGTGTT (SEQ ID NO.:336); CTGAGGTTATGTACAACCAACTCTCAGAATTCAGACTTCCTGCAGCTGCC (SEQ ID NO.:370); GTAGGCCCCCAAAGTGCCGTCTTTCCCTAGCATTTTACTCAATGTTTGCC (SEQ ID NO.:392); GAATCAAGGAGGTCAAGTAAGGTCACAGGGGCACTTGGGTTGAGCCAGGG (SEQ ID NO.:437); CCCCAGATGGTTCCAAATATTCCTTACCTCGTTTGGTTCCCAAGTCACAG (SEQ ID NO.:450); GAATAGAAACCAGACAGCAATTCTTTAGTTCCAGCCACCATTCGCCCCAC (SEQ ID NO.:454); TCAACAAAGAGGTGCTGACCTGAGAGTAGGGCACATAACCTCAGCCACTG (SEQ ID NO.:471); ATGTAGATGGGGAGTGACCACCGCCAACAGAAGTGTGGCCATCTTGCCCG (SEQ ID NO.:535); CTTTGGGCACCATTTGGATATAGTTAGTGGTGGTTTAGCTATGGCGTTCC (SEQ ID NO.:609); GGCAAATTCCGGGTATGCACTCAACTTCGGCAAAGGCACCTCGCTGTTGG (SEQ ID NO.:637); GAGGCTTTCAGGTAGGAGGACAATGGTAGCACTGTAGGTCCCCAGTGTCG (SEQ ID NO.:754); AGTAAACCCATATATCCAGAACCCTGACCCTGCCGTGTACCAGCTGAGAG (SEQ ID NO.:800); CCTGTGGCAAGCCAGCAAGATGGCCCTGGTGACAGCAAAAGAAACTGCAC (SEQ ID NO.:837); CCAGGTGCCGCCCACTCTTGACGTGATACTTACCGTCAATGCTCCTTACC (SEQ ID NO.:876); GCCTAAACCAGGTATGCCAATCTGTCTTGTGTCCACATACTAACAGAGGG (SEQ ID NO.:924); AGCCAAGACAGCAGCTCTACATCCTTACCTAGGTAATTCAGGCATGCGCC (SEQ ID NO.:947); CACATGGCAAATGCCTCCTTTCACAATAGAGCATGGTGCTGTTTCCTCAC (SEQ ID NO.:954); TATTGCAGCCATCCATCTTGGGGGCTCATCCATCACACCCGGGTTGCTAG (SEQ ID NO.:1010); CTGGGCTGTGGTATTTGGGTGATCTTTACATTCTTCAGACTCATGTGTGT (SEQ ID NO.:1035); GCTACAAACAAGCTCATCTTTGGAACTGGCACTCTGCTTGCTGTCCAGCC (SEQ ID NO.:1081); CCTACTCCTACAGTGCCTTGCATTCCGTAGCTGCTCAGTACATTAACCCA (SEQ ID NO.:1116); CAGGGTATGAAAGTGCCCATTTCTAGCCAACATTAGATACCCTCAGTCTC (SEQ ID NO.:1157); TGGCCACATTTGTCTCAAACTCAAGTCTACACATTTCTCTCTCTTTTCCC (SEQ ID NO.:1227); GTACCGTCAGCAACCTGGACAGAGCCTGACACTGATCGCAACTGCAAATC (SEQ ID NO.:1276); and Gccccctaattgactgaatggaacccctcttgaccaaagtgaccccagaa (SEQ ID NO.:1379).

28. The method of claim 25, further comprising the step of differentiating between sarcoidosis and tuberculosis, lung cancer or pneumonia by determining the expression levels of the following genes, markers, or probes: PHF20L1; LOC400304; SELM; DPM2; RPLP1; SF1; ZNF683; CTTN; PTCRA; SNORA28; RPGRIP1; GPR160; PPIA; DNASE1L1; HEMGN; RAB13; NFIA; LOC728843; LOC100134660; LOC100132564; HIP1; PRMT1; PDGFC; NCRNA00085; NFATC3; GIMAP7; LOC100130905; AKAP7; TLE3; NRSN2; RPL37; CSTA; C20orf107; TMEM169; GCAT; TMEM176A; CMTM5; C3orf26; FANCD2; C9orf114; TIAM2; LOC644615; PADI2; GRINA; CHST13; ANGPT1; KIF27; ZNF550; PIK3C2A; NR1H3; ALG8; SLC2A5; ITGB5; OPN3; UBE2O; RIN3; LOC100129203; B3GNT1; NEK8; SLC38A5; GPR183; LOC728748; LOC646966; FAM159A; LOC441073; CCNC; MRPL9; SLC37A1; NSUN5; GHRL; ALAS2; MPZL2; RNF13; SUMO1P1; UHRF2; RNY4; LOC651524; ZNF224; OLIG1; TNFRSF4; BEND7; LOC728323; ARHGAP24; CCCTGCCCTCATGTTGCTTTGGGTCTAGTGGAGGAGAGAGACAGATAAGC (SEQ ID NO.:1447); CAAGTTCTTAACCATCCCGGGTTCCAGTGGTTACAGAGTTCTGCCCTGGG; (SEQ ID NO.:1448) and TGCATGAGATCACACAACTAGGCGGTGACTGAGTCCAACACACCAAAGCC (SEQ ID NO.:1449).

29. The method of claim 25, further comprising the step of differentiating between sarcoidosis that is active and sarcoidosis that is inactive by determining the expression levels of the following genes, markers, or probes: LOC442132; HOXA1; LOC652102; PPIE; C22orf27; TEX10; LMTK2; LOC283663; SUCNR1; COLQ; HLA-DOB; SAMSN1; INPP5E; CYP4F3; CRYZ; CDC14A; LOC653061; KIR2DL4; PCYOX1L; TCEAL3; FRRS1; PHF17; PDK4; LOC440313; ZNF260; SLFN13; VASH1; GM2A; ASAP2; VARS2; RPL14; KIR2DL1; SBDSP; S1PR3; and METTL1; CCAGGAGGCCGAACACTTCTTTCTGCTTTCTTGACATCCGCTCACCAGGC (SEQ ID NO.:1452), and TTCCAGGGCACGAGTTCGAGGCCAGCCTGGTCCACATGGGTCGGaaaaaa (SEQ ID NO.:1451).

30. The method of claim 25, further comprising the step of using 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 144, 150, 200, 250, 300, 400, 500, 600, 700, 800, 900, 1,000, 1,100, 1,200, 1,300, 1,400, or 1,446 genes selected from SEQ ID NOS.: 1 to 1446 to determine if the patient has at least one of tuberculosis, sarcoidosis, cancer or pneumonia.

31. A method for determining the effectiveness of a treating a sarcoidosis patient comprising:
obtaining a sample from a subject suspected of having a pulmonary disease;
determining the expression level of 3, 4, 5, 6 or more genes selected from IL1R2; GRB10; CEACAM4; SIPA1L2; BMX; IL1RAP; REPS2; ANXA3; MMP9; PHC2; HAUS4; DUSP1; CA4; SAMSN1; KLHL2; ACSL1; NSUN7; IL18RAP; GNG10; SMAP2; MGAM; LIN7A; IRAK3; USP10; CEBPD; TGFA; FOS; MANSC1; SLC26A8; ROPN1L; GPR97; NAMPT; MRVI1; KCNJ15; KLHL8; GNG10; MEGF9; GPR160; B4GALT5; STEAP4; LRG1; F5; PHTF1; HMGB2; DGAT2; SLC11A1; QPCT; PANX2; GPR141; or LMNB1; wherein overexpression of the genes is indicative of a reduction in sarcoidosis.

32. A method of identifying a subject with a pulmonary disease comprising:
obtaining a sample from a subject suspected of having a pulmonary disease;
determining the expression level of six or more genes from each of the following genes selected from: UBE2J2; ALPL; JMJD6; FCER1G; LILRA5; LY96; FCGR1C; C10orf33; GPR109B; PROK2; PIM3; SH3GLB1; DUSP3; PPAP2C; SLPI; MCTP1; KIF1B; FLJ32255; BAGE5; IFITM1; GPR109A; IF135; LOC653591; KREMEN1; IL18R1; CACNA1E; ABCA2; CEACAM1; MXD4; TncRNA; LMNB1; H2AFJ; HP; ZNF438; FCER1A; SLC22A4; DISC1; MEFV; ABCA1; ITPRIPL2; KCNJ15; LOC728519; ERLIN1; NLRC4; B4GALT5; LOC653610; HIST2H2BE; AIM2; P2RY10; CCR3; EMR4P; NTN3; C1QB; TAOK1; FCGR1B; GATA2; FKBP5; DGAT2; TLR5; CARD17; INCA; MSL3L1; ESPN; LOC645159; C19orf59; CDK5RAP2; PLSCR1; RGL4; IFI30; LOC641710; GAGGCTTTCAGGTAGGAGGACAATGGTAGCACTGTAGGTCCCCAGTGTCG (SEQ ID NO.: 754); LOC100008589; LOC100008589; SMARCD3; NGFRAP1; LOC100132394; OPLAH; CACNG6; LILRB4; HIST2H2AA4; CYP1B1; PGS1; SPATA13; PFKFB3; HIST1H3D; SNORA73B; SLC26A8; SULT1B1; ADM; HIST2H2AA3; HIST2H2AA3; GYG1; CST7; EMR4; LILRA6; MEF2D; IFITM3; MSL3; DHRS13; EMR4; C16orf57; HIST2H2AC; EEF1D; TDRD9; GPR97; ZNF792; LOC100134364; SRGAP3; FCGR1A; HPSE; LOC728417; LOC728417; MIR21; HIST1H2BG; COP1; SMARCD3; LOC441763; ZSCAN18; GNG8; MTRF1L; ANKRD33; PLAC8; PLAC8; SLC26A8; AGTRAP; FLJ43093; LPCAT2; AGTRAP; S100A12; SVIL; LILRA5; LILRA5; ZFP91; CLC; LOC100133565; LTB4R; SEPT04; ANXA3; BHLHB2; IL4R; IFNAR1; MAZ; gccccctaattgactgaatggaacccctcttgaccaaagtgaccccagaa (SEQ ID NO.: 1379);
comparing the expression level of the 3, 4, 5, 6 or more genes with the expression level of the same genes from individuals not afflicted with a pulmonary disease, and
determining the level of expression of the six or more genes in the sample from the subject relative to the samples from individuals not afflicted with a pulmonary disease for the genes expressed in the one or more expression pathways, selected from: EIF2 signaling and mTOR signaling pathways are indicative of active sarcoidosis; co-expression of genes in the regulation of eIF4 and p70s6K signaling pathways is indicative of pneumonia; co-expression of genes in the interferon signaling and antigen presentation pathways are indicative of tuberculosis; and co-expression of genes in the T cell signaling pathways; and other signaling pathways is indicative of lung cancer.

33. The method of claim 32, wherein the genes that are downregulated are selected from MEF2D; BHLHB2; CLC; FCER1A; SRGAP3; FLJ43093; CCR3; EMR4; ZNF792; C10orf33; CACNG6; P2RY10; GATA2; EMR4P; ESPN; EMR4; MXD4; and ZSCAN18.

34. The method of claim 32, further comprising a method for displaying if the patient has tuberculosis, sarcoidosis, cancer or pneumonia by aggregating the expression data from the six or more genes into a single visual display of a vector of expression for tuberculosis, sarcoidosis, cancer or pneumonia.

35. The method of claim 32, further comprising the step of detecting and evaluating 7, 8, 9, 10, 12, 15, 20, 25, 35, 50, 75, 90, 100, 125, or 144 genes for the analysis.

36. The method of claim 32, wherein the sample is a blood, peripheral blood mononuclear cells, sputum, or lung biopsy.

37. The method of claim 32, wherein the expression level comprises an mRNA expression level and is quantitated by a method selected from the group consisting of polymerase chain reaction, real time polymerase chain reaction, reverse transcriptase polymerase chain reaction, hybridization, probe hybridization and gene expression array.

38. The method of claim 32, wherein the expression level is determined using at least one technique selected from polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing.

39. The method of claim 32, wherein the expression level is determined by microarray analysis that comprises use of oligonucleotides that hybridize to mRNA transcripts or cDNAs for the six or more genes, and wherein the oligonucleotides are disposed or directly synthesized on the surface of a chip or wafer.

40. The method of claim 39, wherein the oligonucleotides are about 10 to about 50 nucleotides in length.

41. The method of claim 32, further comprising the step of using the determined comparative gene product information to formulate at least one of diagnosis, a prognosis or a treatment plan.

42. The method of claim 32, wherein the patient's disease state is further determined by radiological analysis of the patient's lungs.

43. The method of claim 32, further comprising the step of determining a treated patient gene expression dataset after the patient has been treated and determining if the treated patient gene expression dataset has returned to a normal gene or a changed gene expression dataset thereby determining if the patient has been treated.

44. The method of claim 32, wherein a non-overlapping set of genes is used to distinguish between Tb, sarcoidosis, pneumonia and lung cancer, versus, Tb, active sarcoidosis, non-active sarcoidosis, pneumonia and lung cancer are selected from Table 11, 12 or both.

45. A computer readable medium comprising computer-executable instructions for performing the method of claim 1.
